(19) United States
(12) Patent Application Publication
(10) Pub. No.: US 2024/0368633 A1
(43) Pub. Date: Nov. 7, 2024

(54) METHOD FOR IMPROVING GENOME EDITING

(71) Applicant: The Regents of the University of California, Oakland, CA (US)

(72) Inventors: Valentino GANTZ, La Jolla, CA (US); Alena Lauren BISHOP, Berkeley, CA (US); Alexis KOMOR, San Diego, CA (US); Zsolt BODAI, San Diego, CA (US)

(73) Assignee: The Regents of the University of California, Oakland, CA (US)

(21) Appl. No.: 18/689,676

(22) PCT Filed: Sep. 9, 2022
(86) PCT No.: PCT/US2022/043004

§ 371 Date: Mar. 6, 2024

Related U.S. Application Data
(60) Provisional application No. 63/243,260, filed on Sep. 13, 2021.

(51) Int. Cl. C12N 15/90; C12N 9/22; C12N 15/11

(52) U.S. Cl. CPC C12N 15/907; C12N 9/22; C12N 15/11; C12N 15/111; C12N 15/902; C12N 2310/20

(57) ABSTRACT

The present disclosure provides a “double tap” method to improve genome editing efficiencies that takes advantage of the reproducible nature of indel sequences. The “double tap” method uses multiple gRNAs: a primary gRNA that targets the wild-type genomic sequence, and one or more secondary or tertiary or subsequent gRNAs that target the most common indel sequence(s), which provides a “second/third chance” at editing. The “double tap” method also improves gene drive efficiency by recycling resistance alleles. The “double tap” method can be readily implemented in any CRISPR-based gene drive and in a subject that has HDR as a DNA repair mechanism and/or a system suffering from low HDR frequencies to improve performance by boosting efficient gene editing.

### CROSS-REFERENCE

This application claims the benefit of U.S. Provisional Application No. 63/243,260, filed Sep. 13, 2021, the entire content of which is incorporated herein by reference.

### STATEMENT REGARDING FEDERALLY SPONSORED RESEARCH OR DEVELOPMENT

This invention was made with government support under OD023098 and AI162911 awarded by the National Institutes of Health and under MCB-2048207 awarded by the National Science Foundation. The government has certain rights in the invention.

### FIELD AND BACKGROUND

The present disclosure relates to genome editing. More specifically, the present disclosure provides an improved method to boost precision genome editing efficiencies, particularly in systems suffering from low HDR frequencies, such as mammalian cells or mouse germline transformations. The improved genome editing method also improves any CRISPR-based gene drive efficiency by recycling resistance alleles, such improved gene drive also efficiently spreads in caged populations.

The detailed background information related to the state of art genome editing is described in the introductions under EXAMPLES 1 and 2, respectively, of the present disclosure.

### SUMMARY

The present disclosure provides an improved method of CRISPR-based gene editing. The method disclosed herein, termed “double-tap”, uses additional gRNAs (called secondary or tertiary gRNAs, or multiple secondary or tertiary gRNAs) to target high frequency indel products created by end joining pathways during an attempted HDR event (FIG. 1a). Normally, these indel products cannot be processed by Cas9 as they do not match the original gRNA sequence. However, when complemented with secondary or tertiary gRNAs, these sequences can be re-targeted, providing a second or third or multiple opportunity for the DSB to be processed by HDR using the same donor template. These secondary or tertiary or multiple secondary or tertiary gRNAs could decrease unwanted indel products and increase the desired precision genome editing outcome.

In certain embodiments, the double tap method was tested in multiple human cell lines at 15 different genomic loci. Secondary gRNAs were designed and tested to targeted indel sequences with a wide range of frequencies and larger improvements in HDR-mediated genome editing efficiencies were observed when targeting higher frequency indel sequences, with no increases in indel rates (in many instances, decreases in indel rates were in fact observed). The present disclosure demonstrates the ability of the double tap method to improve HDR-mediated genome editing efficiencies for the installation of point mutations, small insertions, and deletions with ssODNs, as well as for gene knock-in using dsDNA donor templates. The double tap method disclosed herein can be easily integrated into any routine HDR experiment to boost precision editing efficiencies by characterizing the sequences of the most common indel products and incorporating secondary or tertiary or subsequent gRNAs to target these sequences. Therefore, the double tap method could be implemented in a subject, such as any animals (fly, mice, rats, etc.), plants, or fungi, that has HDR as a DNA repair mechanism and/or a system where HDR conversion is less efficient, such as primary human cells or other mammalian cells, and/or mouse embryos or germline transformations, to boost efficient gene editing for human diseases and/or agriculture.

The present disclosure further provides the double tap homing gene-drive strategy to combat the prevalent resistance alleles that prevent drive spread. In certain embodiments, the double tap gene drive method uses additional, secondary or tertiary or multiple secondary or tertiary gRNAs targeting the resistance alleles to recycle them as new templates for an additional round of gene conversation, ultimately, improving gene drive efficiency. Therefore, the double tap method disclosed herein could be universally applied to increase the efficiency of CRISPR-based gene-drive systems suffering from resistance allele generation. In other embodiments, the double tap gene drive method also improves the ability of the drive to spread in a population.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Many aspects of the present disclosure can be better understood with reference to the drawings. The components in the drawings are not necessarily to scale, emphasis instead being placed upon clearly illustrating the principles of the present disclosure. Moreover, in the drawings, like reference numerals designate corresponding parts throughout the several views. A better understanding of the features and advantages of the invention will be obtained by reference to the following detailed description that sets forth illustrative embodiments, in which the principles of the invention are utilized, and the accompanying drawings (also “Figure” and “FIG.” herein), of which:

FIGS. 1a-1d. Schematic and initial results of the double tap method. FIG. 1a. Schematic overview of the double tap method. Cas9 introduces a DSB at a locus of interest using the primary guide RNA. HDR processes a subset of the DSBs into the desired outcome using a donor template. Concurrently, indels are introduced at the DSB site via end-joining pathways. These undesired indel sequences are subsequently targeted with secondary gRNAs to improve overall yields of the desired outcome through a second DSB introduction and sequential HDR repair. FIG. 1b. Indel sequences and their corresponding introduction efficiencies at the MMACHC site after transfecting HEK293T cells with Cas9 and a non-targeting gRNA (top), the primary gRNA plus a non-targeting gRNA (middle), or the primary gRNA plus a secondary gRNA targeted to the indel sequence indicated with the black arrow (bottom). Figure discloses SEQ ID NOS 26, 26-31, 26, 32-34, 26, 33, 32, 35 and 31, respectively, in order of appearance. FIG. 1c. HDR-mediated genome editing efficiencies at the FANCF (in which a low-frequency indel was targeted), APOB1, and MMACHC sites when HEK293T cells are transfected with an ssODN and plasmids encoding Cas9, the primary gRNA, and either a non-targeting gRNA (NT, left) or secondary gRNA(s) (DT for “double tap”, right; two secondary gRNAs were used with the FANCF primary gRNA, and one secondary gRNA was used at the other two sites). Plotted are the percent of total DNA sequencing reads with the desired modification introduced (perfect HDR products without indels). FIG. 1d. HDR-mediated genome editing efficiencies at the RNF2locus when HEK293T cells are transfected with an ssODN and plasmids encoding Cas9, the primary gRNA, and either a non-targeting gRNA (NT, far left) or one (1×DT), two (2×DT), or three (3×DT) secondary gRNAs. Values on whisker plots represent the lowest observation, lower quartile, median, upper quartile, and the highest observation of three independent replicates. Data were analyzed with univariate statistics (one-way ANOVA [one-sided]), and p-values are labelled on the graphs.

FIGS. 2a-2d. Improvements in HDR-mediated genome editing with ssODNs using the double tap method. FIG. 1a. Shown are the percent of DNA sequencing reads with the desired modification introduced (perfect HDR products without indels) for cells treated with primary gRNA and a non-targeting gRNA (NT, left), or primary gRNA and secondary gRNA(s) (DT, right; three secondary gRNAs were used at the HIRA and RNF2 sites, two secondary gRNAs were used at the HEK2, HEK3 and FANCF sites, and one secondary gRNA was used at the APOB1, APOB2, PSMB, PCSK, SEC61B and MMACHC sites). Values on the whisker plots represent the lowest observation, lower quartile, median, upper quartile and the highest observation of three independent replicates. Data were analyzed with univariate statistics (one-way ANOVA [one-sided]) and p-values are labelled on the graphs. FIG. 1b. Average fold-change values plotted against the average of the total initial rates of the indels targeted by secondary gRNAs for all of the genomic loci tested in FIGS. 1a-1d and 2a-2d. Error bars represent the propagation of uncertainty of the SD for n=3 biological replicates. FIG. 2c. Shown are the relative changes in HDR (light grey) and NHEJ (dark grey) frequencies relative to the primary and non-targeting gRNA samples. Values and error bars represent the mean and propagation of uncertainty of the SD for n=3 biological replicates. FIG. 2d. Shown are total indel rates of all samples, with the specific indels targeted by secondary gRNAs shown in light grey. Dark grey represents indels not targeted by secondary gRNAs. Values and error bars represent the mean of the number of sequencing reads with indel sequences divided by the total number of sequencing reads±SD for n=3 biological replicates. In FIG. 1c and FIG. 1d, when the ssODN encoded a blocking mutation, the site is labelled with an “_B”.

FIGS. 3a-3d. Further characterization of the double tap method. FIG. 3a. Additive effects of double tap and previously developed HDR-improving methods were investigated at the MMACHC site. Shown are the percent of DNA sequencing reads with the desired modification introduced (perfect HDR products without indels) for cells treated with primary gRNA and a non-targeting gRNA (NT, left), or primary gRNA and secondary gRNA (DT, right; only one secondary gRNA was used at the MMACHC site). NT and DT samples were additionally treated with the small molecule HDR enhancer (Alt-R) or with a Cas9-CtIP fusion construct (Cas9-HE). DMSO-treated and no additive samples served as a base line for comparison (the Alt-R molecule is dissolved in a DMSO solution). FIG. 3b. Shown are total indel rates of samples from a, with the specific indels targeted by the secondary gRNA shown in light grey. Dark grey represents indels not targeted by a secondary gRNA. Values and error bars represent the mean of the number of sequencing reads with indel sequences divided by the total number of sequencing reads ±SD for n=3 biological replicates. FIG. 3c. Double tap improvements using Cas9:gRNA RNP complex at 3 sites. Shown are the percent of DNA sequencing reads with the desired modification introduced (perfect HDR products without indels) for cells treated with primary gRNA and a non-targeting gRNA (NT, left), or primary gRNA and secondary gRNA(s) (DT, right; three secondary gRNAs were used at the RNF2 site, two secondary gRNAs were used at the HEK3 site, and one secondary gRNA was used at the MMACHC site). FIG. 3d. Analysis of zygosity of genome edited isogenic cells (n=41 for each group) at the MMACHC locus. Shown are the frequency of the indicated genome editing outcomes from each set of edited cells. Samples in (FIGS. 3a-3c) were analyzed by NGS after 72 hours, and samples in (FIG. 3d) were clonally expanded and genotyped by NGS after 3 weeks. (FIGS. 3a & 3c) Values on the whisker plots represent the lowest observation, lower quartile, median, upper quartile and the highest observation of three independent replicates. Data were analyzed with univariate statistics (one-way ANOVA [one-sided]) and p-values are labelled on the graphs.

FIGS. 4a-4c. Improvements in gene knock-in with dsDNA donor templates using the double tap method. FIGS. 4a and 4b. Selected scatter plots of GFP fluorescence (y-axis) and cell forward scatter (x-axis), showing gating for GFP fluorescence for HEK293T cells transfected with plasmids encoding dsDNA donor template, Cas9, and non-targeting gRNA only (top), primary and non-targeting gRNAs (middle), or primary and secondary gRNAs (bottom) for the ACTB gene (FIG. 4a) and the LMNA gene (FIG. 4b). FIG. 4c. Quantification of the percent of cells with GFP fluorescence in the GFP knock-in experiment for the ACTB (top) and LMNA (bottom) genes. NT stands for non-targeting, OG+NT stands for primary with non-targeting, and OG+DT stands for primary and gRNAs. One secondary gRNA was used at both sites. Values on the whisker plots represent the lowest observation, lower quartile, median, upper quartile and the highest observation of three independent replicates. Data were analyzed with univariate statistics (one-way ANOVA [one-sided]) and p-values are labelled on the graphs.

FIGS. 5a-5b. Improvements in HDR-mediated genome editing with ssODNs using the double tap method in human erythroleukemic (K562) and human cervical cancer (HeLa) cell lines. FIG. 5a. HeLa or K562 cells were transfected with ssODN, Cas9-p2A-GFP plasmid, and gRNA plasmids. After 72 hours, cells were enriched with FACS and analyzed by NGS and HDR-mediated genome editing efficiencies were quantified. Shown are the percent of DNA sequencing reads with the desired modification introduced (perfect HDR products without indels) for cells treated with primary gRNA and a non-targeting gRNA (NT, left), or primary gRNA and secondary gRNA(s) (DT, right; one secondary gRNA was used at both sites). Data from the MMACHC site are on the left and those from the APOB1 site are on the right. Data acquired from K562 cells are on the top and those from HeLa cells are on the bottom. Values on the whisker plots represent the lowest observation, lower quartile, median, upper quartile and the highest observation of three independent replicates. Data were analyzed with univariate statistics (one-way ANOVA [one-sided]) and p-values are labelled on the graphs. FIG. 5b. Shown are total indel rates of all samples, with the specific indels targeted by secondary gRNAs shown in light grey. Dark grey represents indels not targeted by secondary gRNAs. Values and error bars represent the mean of the number of sequencing reads with indel sequences divided by the total number of sequencing reads ±SD for n=3 biological replicates. Data points are marked as circles when the ssODN encoded an extra blocking mutation, and as triangles when no additional mutation was installed.

FIGS. 6a-6c. Installation of disease relevant mutations in the HBB and HEXA genes using the double tap method. FIG. 6a. Shown are the percent of DNA sequencing reads with the desired modification introduced (perfect HDR products without indels) for cells treated with primary gRNA and a non-targeting gRNA (NT), or primary gRNA and secondary gRNA(s) (DT; three secondary gRNAs were used at the HBB5 site, and one secondary gRNA was used at the HBB1, HEXA2 and HEXA5 sites). FIG. 6b. Shown are total indel rates of all samples from FIG. 6a, with the specific indels targeted by secondary gRNAs shown in light grey. Dark grey represents indels not targeted by secondary gRNAs. FIG. 6c. HEK293T cells were transfected with plasmids encoding the prime editor and pegRNA only (PE2 sample), or pegRNA and nicking gRNA (PE3 sample) to introduce the same mutations as in FIG. 6a. After 72 hours, cells were analyzed by NGS to determine the efficiencies of introduction of the intended edit. Shown are the percent of DNA sequencing reads with the desired modification introduced (perfectly edited products without indels) for double tap samples from FIG. 6a (labeled as DT), PE2 treated cells (labeled as PE2), or PE3 treated cells (labeled as PE3). Values on the whisker plots in FIG. 6a and FIG. 6c represent the lowest observation, lower quartile, median, upper quartile and the highest observation of three independent replicates. Data were analyzed with univariate statistics (one-way ANOVA [one-sided]) and p-values are labelled on the graphs. Values and error bars in FIG. 6b represent the mean of the number of sequencing reads with indel sequences divided by the total number of sequencing reads ±SD for n=3 biological replicates. Data points are marked as circles when the ssODN encoded an extra blocking mutation, and as triangles when no additional mutation was installed. Data points are marked as squares for prime editing samples.

FIGS. 7a-7b. Assessment of off-target editing due to the double tap method. FIG. 7a. HEK293T cells were transfected with Cas9 and gRNA plasmids (non-targeting, primary, or secondary gRNAs). After 72 hours, cells were analyzed by NGS at the primary (on-target) and all predicted off-target loci. Shown are total indel rates of all samples. The primary (on-target) loci are labelled as OG, while predicted off-target sites for primary gRNAs are labelled as OG_OT and predicted off-target sites for secondary gRNAs are labelled as DT_OT on the y axis. The label on the x-axis indicates which gRNA the cells were transfected with; the secondary (DT), non-targeting (NT) or primary (OG). Only one gRNA was used at a time. FIG. 7b. HEK293T cells were transfected with plasmids encoding Cas9-p2A-GFP, primary gRNA, and either non-targeting gRNA or secondary gRNA(s). As a control, HEK293T were transfected with plasmids encoding Cas9-P2A-GFP and non-targeting gRNA only. After 72 hours cells were stained with propidium iodide to quantify cell viability FACS. The percentage of transfected cells (as determined by GFP fluorescence) that were viable are plotted with respect to the primary gRNA used (RNF2, HBB5, APOB1 and MMACHC). Samples with primary and non-targeting gRNAs are shown in blue, while those with primary and secondary gRNAs are in pink. Three secondary gRNAs were used with the RNF2 and HBB5 primary gRNA, and one secondary gRNA was used at APOB1 and MMACHC sites. Values and error bars represent the mean and standard deviation of viable cells within the transfected population for n=3 biological replicates.

FIGS. 8a-8w. InDelphi predictions and experimentally determined indel sequences for all genomic loci studied in this work. The InDelphi figures show predicted indel sequences in HEK293T cells, except for the APOB1 and MMACHC sites, where predicted indels for both HEK293 and K562 cells are shown. The CRISPResso analysis of HTS data from treated HEK293T cells using the indel output is shown on the right. All sites studied in the paper are listed and labelled at the top. Indel sequences with rates above 5% for both the experimental samples and the InDelphi predictions are marked with arrows. Figure discloses SEQ ID NOS 36-48, 48-95, 84-94, 96-97, 98, 97, 99-128, 128-156, 156-206, 206-239, 239-378, 378-411, 409, 412-431, 431-475, 475-550, 550-583, 583-606, 606-636, 636-652, 648, 653-685, 683, 686-735, 724-725, 727, 726, 728-732, 734-738, 737, 739-761, 761-784, 784-832, 832-884 and 884-898, respectively, in order of appearance.

FIG. 9. Improvements in HDR-mediated genome editing with ssODNs using the double tap method at the HEK2 site. HEK293T cells were transfected with ssODN, Cas9 plasmid, and gRNA plasmids. After 72 hours, cells were analyzed by NGS and HDR-mediated genome editing efficiencies were quantified. Shown are the percent of DNA sequencing reads with the desired modification introduced (perfect HDR products without indels) for cells treated with primary gRNA and a non-targeting gRNA (NT, left), or primary gRNA and secondary gRNA(s) (DT, right). Values on the whisker plots represent the lowest observation, lower quartile, median, upper quartile and the highest observation of three independent replicates. Each replicate is marked individually. Data were analyzed with univariate statistics (ANOVA) and p-values are marked as following: p≥0.05 not significant (n.s.), p=0.01-0.05 significant (*), p=0.001-0.01 very significant (**), if p≤0.001 extremely significant (***). Data points are marked as circles when the ssODN encoded an extra blocking mutation, and as triangles when no additional mutation was installed.

FIG. 10. Improvements in HDR to NHEJ ratios using the double tap method. HEK293T cells were transfected with ssODN, Cas9 plasmid, and gRNA plasmids. After 72 hours, cells were analyzed by NGS and HDR-mediated genome editing efficiencies and indel frequencies were quantified. Shown are the percent of DNA sequencing reads with the desired modification introduced (perfect HDR products without indels) divided by the percent of DNA sequencing reads with indels for cells treated with primary gRNA and a non-targeting gRNA (NT), or primary gRNA and secondary gRNA(s) (DT). Error bars represent the propagation of uncertainty of the changes of the ratios of three independent replicates.

FIG. 11. Combined improvements in HDR-mediated genome editing using the double tap method and ssODN blocking mutations at the FANCF (in which a low-frequency indel was targeted), APOB1 and MMACHC sites. HEK293T cells were transfected with ssODN, Cas9 plasmid, and gRNA plasmids. After 72 hours, cells were analyzed by NGS and HDR-mediated genome editing efficiencies were quantified. Shown are the percent of DNA sequencing reads with the desired modification introduced (perfect HDR products without indels) for cells treated with primary gRNA and a non-targeting gRNA (NT), or primary gRNA and secondary gRNA(s) (DT). Donor templates with blocking mutations (Block) and without were tested. Values on the whisker plots represent the lowest observation, lower quartile, median, upper quartile and the highest observation of three independent replicates. Each replicate is marked individually. Data points are marked as circles when the ssODN encoded an extra blocking mutation, and as triangles when no additional mutation was installed.

FIGS. 12a-12b. Morphology changes of HEK293T cells after dimethyl sulfoxide (DMSO) and Alt-R™ HDR Enhancer V2 treatment 24 hours after transfection. All the Alt-R™ HDR Enhancer V2 treated samples displayed the morphological changes displayed above. Removal of the Alt-R™ HDR Enhancer V2-containing media followed by replating of the cells resulted in a return to normal morphology after 24 hours.

FIG. 13. Indel frequencies for Cas9 ribonucleoprotein (RNP)-treated cells. HEK293T cells were transfected with Cas9 with primary gRNA and a non-targeting gRNA (NT, left), or primary gRNA and secondary gRNA(s) (DT, right) and ssODNs. After 72 hours, cells were analyzed by NGS and indels were quantified with CRISPResso2. Dark grey bars show the absolute (or total) indel NGS read frequency and light grey bars show the indel frequencies of potential target with double tap gRNAs. Values and error bars represent the mean of the number of sequencing reads with indel sequences divided by the total number of sequencing reads ±SD for n=3 biological replicates.

FIG. 14. Secondary and alternative secondary gRNAs for the APOB1 site to target the most frequent indel (a 1-bp insertion product). Note for ease of design, we would use the Cas9-NG variant, which recognizes an NG PAM (relaxed from NGG). FIG. 14 discloses SEQ ID NOS 899-900, respectively, in order of appearance.

FIGS. 15A-15D. Indel frequencies generated with candidate primary gRNAs at the HBB and HEXA loci. FIG. 15A. HEK293T cells were transfected with Cas9 and gRNA plasmids. After 72 hours, cells were analyzed by NGS and HDR-mediated genome editing efficiencies were quantified. Shown are total indel rates of all samples, with the top three frequency indels shown in light grey. Dark grey represents the remaining indels. FIG. 15B. HEK293T cells were transfected with ssODN and plasmids encoding Cas9 and candidate primary gRNAs selected from FIG. 15A. After 72 hours, cells were analyzed by NGS and HDR-mediated genome editing efficiencies were quantified. Shown are the percent of DNA sequencing reads with the desired modification introduced (perfect HDR products without indels). FIGS. 15C and 15D. Genomic DNA sequences of the HBB (FIG. 15C) and HEXA (FIG. 15D) loci, with the modification of interest indicated, and the candidate primary gRNA protospacers indicated as arrowed lines, with their respective cut sites indicated with dotted lines. Results show a single experiment. Selected candidate primary RNAs were further tested to confirm high frequency of selected indel products. The underlined base pair indicates a SNV in the HEK293T cell line. Figure discloses SEQ ID NOS 901-904, respectively, in order of appearance.

FIG. 16. Secondary gRNA designs for the HBB1 (SEQ ID NOS 905-906, respectively, in order of appearance), HBB5 (SEQ ID NOS 907-910, respectively, in order of appearance), HEXA2 (SEQ ID NOS 911-912, respectively, in order of appearance) and HEXA5 (SEQ ID NOS 913-914, respectively, in order of appearance) primary gRNAs. The underlined base pair indicates a SNV in the HEK293T cell line.

FIG. 17. Alternative secondary gRNA designs at the RNF2site to avoid unwanted off-target editing. Figure discloses SEQ ID NOS 915-916, respectively, in order of appearance.

FIG. 18. Sequences of primary and secondary protospacers and PAMs, and their respective off-target sites that evaluated for the APOB1 APOB1 (SEQ ID NOS 917-924, respectively, in order of appearance), MMACHC (SEQ ID NOS 927-934, respectively, in order of appearance) and HBB5 (SEQ ID NOS 925-926, respectively, in order of appearance)_sites. Bases that are explicitly written in the off-target sites represent a mismatch, and bases in bold indicated bulges.

FIG. 19. Assessment of the effect of secondary gRNAs on cell viability. HEK293T cells were transfected with plasmids encoding Cas9-P2A-GFP, primary gRNA, and either non-targeting gRNA or secondary gRNA(s). As a control, HEK293T were transfected with plasmids encoding Cas9-P2A-GFP and non-targeting gRNA only. After 72 hours cells were stained with propidium iodide to quantify cell viability FACS. The percentage of all cells (both transfected and non-transfected) that were viable are plotted with respect to the primary gRNA used (RNF2, HBB5, APOB1 and MMACHC). Samples with primary and non-targeting gRNAs are labeled as “_OG”, while those with primary and secondary gRNAs are labeled as “_DT”. The sample with non-targeting gRNA only is the first bar. OG stands for primary gRNA and DT stands for secondary gRNA. Error bar represents the standard deviation of the transfected viable population (n=3).

FIG. 20. The design of secondary gRNAs when indels with small deletions (likely facilitated by MMEJ) are targeted can result in a secondary gRNA that targets the original DNA sequence, but with an undesired alternate cut site. One such example (the HEXA5 primary gRNA produces a 7-bp deletion indel with a high frequency) is explicitly shown. If a secondary gRNA is designed for the indel shown using the same PAM as the primary gRNA, it can target the original DNA sequence using a different PAM. To avoid this, an alternative PAM can be used. Figure discloses SEQ ID NOS 995-996, respectively, in order of appearance.

FIGS. 21A-21C. Example of flow cytometry and FACS gating. Doublets were gated out using forward and side scattering width against area, and GFP gates were set using untransfected cells.

FIGS. 22a-22e. Double-tap trans-complementing gene drive (DT-tGD) experimental setup and inheritance analysis. FIG. 22a. Schematic of the DT-tGD arrangement in which the Cas9 and gRNA elements are kept as two separate transgenic lines; gRNA-1 and gRNA-2 target the loci at which the Cas9 and gRNA elements are inserted, respectively. When crossed, Cas9 combines with gRNA-1 and gRNA-2 to generate double-strand breaks at each of the wildtype alleles. Repair by end-joining (EJ) pathways rather than homology-directed repair (HDR) would ordinarily halt gene-drive spread. Upon generation of a predicted resistance allele, Cas9 together with double-tap gRNAs gRNA-1 b and gRNA-2b can regenerate double-strand breaks at these loci, providing a second chance for the drive elements to be copied by HDR. FIG. 22b. gRNAs used in this system. The y1-gRNA and w2-gRNA target the wildtype yellow and white loci, respectively. The y1b-gRNA and w2b-gRNA target a single base pair deletion of the most common indel generated at the yellow and white loci, respectively. Figure discloses SEQ ID NOS 935-938, respectively, in order of appearance. FIG. 22c. Cross scheme used in this experiment. Males carrying the DsRed-marked Cas9 transgene inserted at the yellow locus are crossed to virgin females carrying the GFP-marked gRNA element inserted at the white locus. Trans-heterozygous virgin F₁ females are single-pair crossed to wildtype males, and the resulting progeny are scored for green and red fluorescence as markers of transgene inheritance. The dark grey half arrows represent the male Y chromosome. FIG. 22d. Transgenic fly lines used in this experiment. vasa-driven Cas9 is marked with DsRed and inserted in the yellow locus. Various gRNA combinations, in which each gRNA is driven by a U6 promoter, are marked with EGFP and inserted in the white locus. FIG. 22e. Single female germline inheritance rates as measured by fluorescence phenotypes detected in the F₂ progeny. Black bars represent the average inheritance rates, and blue shaded boxes indicate the deviation from the normally expected 50% Mendelian inheritance. Pie charts represent the percentage of crosses that resulted in 100% inheritance of that transgene.

FIGS. 23a-23c. Maternal effect in the double-tap gene drive. FIG. 23a. Paternal inheritance cross scheme. F₀ males carrying both the DsRed-marked Cas9 element in yellow and the GFP-marked gRNA element in white are crossed to wildtype virgin females. Heterozygous F₁ virgin females are single-pair crossed to wildtype males, and F₂ flies are scored for red and green fluorescence as markers of transgene inheritance. FIG. 23b. Maternal inheritance cross scheme. Homozygous F₀ females carrying both Cas9 and gRNA elements are crossed to wildtype males. F₁ cross and F₂ scoring are the same as in panel FIG. 23a. FIG. 23c. Single female germline inheritance rates as measured by fluorescence detection in F₂ progeny. Black bars represent the average inheritance rates, and blue shaded boxes indicate the deviation from the normally expected 50% Mendelian inheritance. Pie charts represent the percentage of crosses that resulted in 100% inheritance of that transgene.

FIGS. 24a-24d. Specificity analysis of the gRNAs used in the double-tap system. FIG. 24a. Transgenic fly lines generated to test specificity of gRNAs. Different combinations of gRNAs driven by U6 promoters are marked with 3×P3-EGFP and inserted at the white locus—the same as all other gRNA lines used in this work. FIGS. 24b & b′. Cross scheme used for experiments in panel FIG. 24d. Males carrying DsRed-marked Cas9 inserted at the yellow locus are crossed to virgin females carrying one of the two GFP-marked gRNA elements inserted at the white locus. Trans-heterozygous F₁ virgin females are single-pair crossed to wildtype males, and the resulting F₂ progeny are scored for red and green fluorescence as markers of transgene inheritance. Symbols are the same as FIG. 23c. FIGS. 24c & c′. Cross scheme used for experiments in panel FIG. 24e. F₀ males carrying both the DsRed-marked Cas9 transgene and one of the two GFP-marked gRNA elements are crossed to virgin females homozygous for y1b (yellow box) and w2b (light brown box) alleles, which are single base pair deletions at each locus targetable by the y1b- and w2b-gRNAs, respectively. Heterozygous F₁ virgin females are crossed to wildtype males and the resulting F₂ progeny are scored for red and green fluorescence as markers of transgene inheritance. FIG. 24d. Single female germline inheritance rates as measured by scoring fluorescence in F₂ progeny. Results from the FIG. 24b & b′ crosses. Graph labeled as in FIG. 22e. FIG. 24e. Same as FIG. 24d for the results from the FIG. 24c & c′ crosses.

FIGS. 25a-25c′/d. tGD(y1,w2) and DT-tGD(y1,w2, y1b, w2b) performance in caged populations. FIG. 25a. Schematic of the yellow and white genomic loci, indicating the locations targeted by the y1- and w2-gRNAs (triangles) and the y^{EX1} and w^{EX1} mutations (asterisks). An approximate location of yellow and white on the X chromosome is shown on the top right of the panel. FIG. 25b. Schematic of population experiment. Cages are seeded with 100 flies, including 10 males that carry both the Cas9 and gRNA drive elements. After 5 days, the adult flies are discarded, and the larvae are allowed to develop. On day 18, flies are removed from the cage and split into two groups—approximately 200 flies are randomly selected to seed the next generation; the remaining flies are scored for red and green fluorescence as markers of transgene inheritance. FIG. 25c. DsRed-marked Cas9 and FIG. 25c′/d. GFP-marked gRNA transgene prevalence in 3 independent populations per condition, tracked over 15 generations by scoring the two fluorescent markers. Dotted lines represent 3 independent cages. Fat solid lines represent the moving average of the 3 cages' average.

FIGS. 26a-26b. Resistant allele sequences Resistant allele sequences recovered at the white locus (FIG. 26a) and yellow locus (FIG. 26b) with sections for each construct used. gRNAs present in each construct are in parentheses. Sequence complementary to the w2-gRNA (FIG. 26a) or the y1-gRNA (FIG. 26b) is in blue, PAM is in red, and sequence is split at the cut site. Wild-type sequence for comparison at the top of each panel. Dots represent missing bases; insertions are shown in green. The number of bases missing and/or inserted is noted to the right of each sequence. Flies that were w+ or y+ are marked as such. The number of flies and number of individual crosses from which each allele was recovered in each experiment are on the far right. The w2b allele is highlighted in pink and the y1b allele is highlighted in yellow. Figure discloses SEQ ID NOS 939-951, 941, 952, 942, 953-959, 950, 952, 942, 960-977, 962, 978, 963, 979-983, 966, 984-989, 962, 981, 990-991, 967, 969-970, 992 and 997, respectively, in order of appearance.

FIGS. 27a-27d—Testing double-tap in a condition in which the total number of gRNAs is held constant. FIG. 27a. Transgenic fly lines used in this experiment. Various gRNAs driven by U6 promoters and marked with 3×P3-EGFP are inserted at the white locus. FIG. 27b. Sequence of the w2-gRNA aligned with white locus of the wild-type and w^{Δ13} strain. FIG. 27c & c′. Cross schemes used in this experiment. F₀ males carrying DsRed-marked Cas9 inserted at the yellow locus and the w^{Δ13} allele are crossed to virgin females carrying either the single-cutting (FIG. 27c) or double-tap (FIG. 27c′) pair of GFP-marked gRNAs inserted at the white locus. Transheterozygous F₁ virgin females are single-pair crossed to wild-type males and the resulting progeny are scored for red and green fluorescence as markers of transgene inheritance. FIG. 27d. Single female germline inheritance rates as measured by fluorescence markers in the F₂ flies. Graph labeled the same as FIG. 22e. Figure discloses SEQ ID NOS 937, 993-994, respectively in order of appearance.

FIGS. 28a-28b. Analysis of indel generation during the spread of tGD and DT-tGD in caged populations. Analysis of indel allele generation at (FIG. 28a) the yellow and (FIG. 28b) white loci. In all conditions a genomic pool containing of 50 alleles (from 50 random males) was sampled, except for samples marked with an asterisk (*): 1) Population 2/F8, where 30 alleles were sampled for yellow and 42 for white; and 2) Population 3/F8 where 39 alleles were sampled for yellow.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a catalyst,” “a metal,” or “a substrate,” includes, but are not limited to, mixtures or combinations of two or more such catalysts, metals, or substrates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Unless otherwise specified, temperatures referred to herein are based on atmospheric pressure (i.e., one atmosphere).

In certain embodiments, the present disclosure provides a general strategy (the “double tap” method) to improve HDR-mediated precision genome editing efficiencies that takes advantage of the reproducible nature of indel sequences. The method simply involves the use of multiple gRNAs: a primary gRNA that targets the wild-type genomic sequence, and one or more secondary or tertiary or multiple secondary or tertiary gRNAs that target the most common indel sequence(s), which in effect provides a “second chance” at HDR-mediated editing. The studies described herein, particularly in EXAMPLE 1 below presents the double tap method as a simple yet effective option for enhancing precision editing in mammalian cells.

Homing CRISPR gene drives could aid in curbing the spread of vector-borne diseases and controlling crop pest and invasive species populations due to an inheritance rate that surpasses Mendelian laws. However, this technology suffers from resistance alleles formed when the drive-induced DNA break is repaired by error-prone pathways, which creates mutations that disrupt the gRNA recognition sequence and prevent further gene-drive propagation. To counteract this, the present disclosure, particularly in EXAMPLE 2, provides that the double tap method disclosed herein improves drive efficiency by encoding additional gRNAs into the gene drive that target the most commonly generated resistance alleles, allowing a second or third or subsequent opportunity at gene-drive conversion and recycling resistance alleles. The double tap drive also efficiently spreads in caged populations, outperforming the control drive. Overall, the double tap method disclosed herein can be readily implemented in any CRISPR-based gene drive to improve performance, and similar approaches could benefit other systems suffering from low HDR frequencies, such as mammalian cells or mouse germline transformations.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Targeting Double-Strand Break Indel Byproducts with Secondary Guide RNAs Improves Cas9 HDR-Mediated Genome Editing Efficiencies

### INTRODUCTION

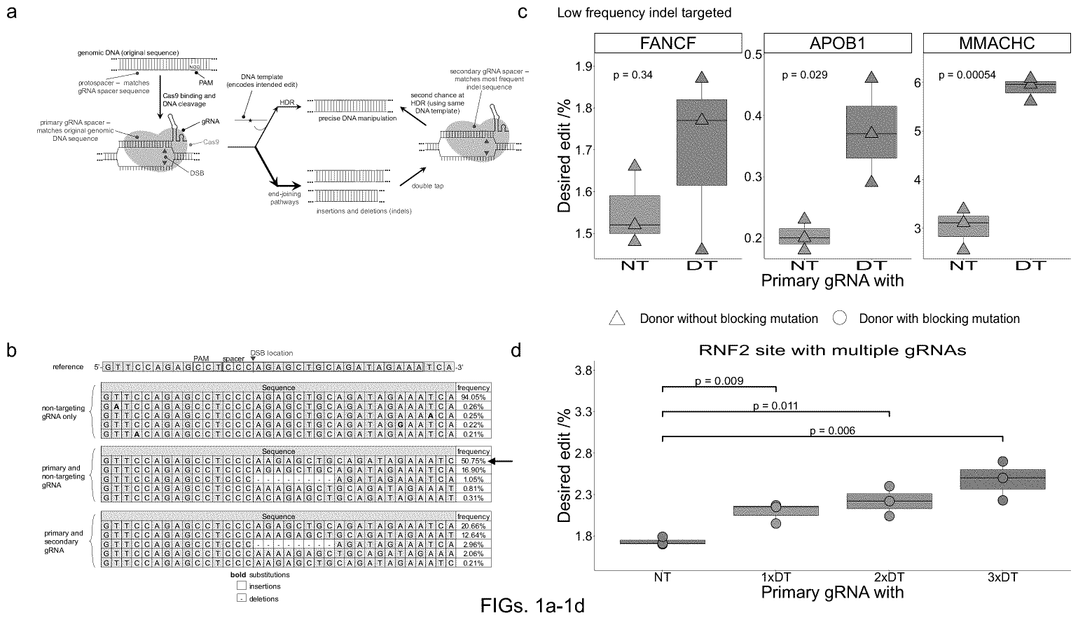

Clustered regularly interspaced short palindromic repeat (CRISPR) systems have revolutionized the genome editing field over the past decade. The most widely used type II CRISPR system consists of two main elements: an engineered chimeric single guide RNA (gRNA) and the DNA endonuclease protein Cas9 (CRISPR-associated protein 9)¹. The gRNA is easily programmed as it facilitates Cas9 to bind to a target site of interest via sequence complementarity with the target DNA sequence (called the protospacer), which must be directly next to a protospacer adjacent motif (PAM). In the Streptococcus pyogenes (Sp) system (used in this work), the protospacer is 20 bases long, and the PAM sequence is NGG (FIG. 1a). After successful DNA binding, the SpCas9 protein cleaves the DNA backbone to introduce a double-strand break (DSB) at the desired genomic locus.

The DSB can be repaired via two main pathways: either re-ligation of the broken ends by end-joining pathways, or templated repair via homology-directed repair (HDR). Re-ligation is mainly mediated by non-homologous end joining (NHEJ) or microhomology-mediated end joining (MMEJ), which result in insertion and deletion (indel) sequences at the site of the DSB under genome editing conditions. In contrast, HDR uses a sister chromatid as a template to repair the DSB in a precise manner². The endogenous HDR pathway can be manipulated to precisely insert DNA sequences by providing the cell with an artificial donor template harboring modifications of interest. Under typical genome editing conditions, both pathways are active and compete to process the DSB intermediate, resulting in mixtures of precision HDR-mediated products as well as end-joining-mediated indel products.

Since the initial demonstration of HDR-mediated genome editing using Cas9 in human cells³⁻⁶, there have been numerous studies that have improved the ratio of HDR-mediated to end-joining-mediated genome editing products.^{7, 8} Specifically, a variety of strategies involving donor template modifications have improved HDR-mediated editing efficiencies, including: (1) phosphorothioate end modification of the template, potentially due to the longer residence time within the cells of the template when modified⁹; (2) optimization of homology arm length of the donor template when using a single-stranded oligodeoxynucleotide (ssODN) template, both with symmetric¹⁰ and asymmetric homology arms¹¹; (3) fusion of the ssODN donor template to the Cas9 protein, potentially due to enhanced nuclear import of the donor template when covalently attached to Cas9^{12, 13}; and (4) installation of silent mutations in the PAM or PAM-proximal regions of the protospacer, which prevents the Cas9:gRNA complex from binding and re-cutting the genomic DNA following a successful HDR event¹⁴. Additionally, as HDR is primarily limited to the synthesis (S) and gap 2 (G2) phases of the cell cycle, methods to manipulate cell cycle phases have been shown to impact HDR outcomes^{15,16}. In addition, small molecules have been used to inhibit end-joining pathways (by targeting key end-joining repair proteins such as DNA Ligase IV¹⁷, DNA-PKcs¹⁸, and 53BP1¹⁹) to increase relative HDR to end-joining ratios as well. Finally, fusion of Cas9 to different DNA repair proteins, such as CtIP²⁰ and Rad51²¹, have also been shown to enhance HDR-mediated editing efficiencies.

Motivated by this need to enhance the efficiency of precision genome editing outcomes, other CRISPR-based genome editing technologies have emerged recently, such as base editing^{22,23} and prime editing²⁴. Although these technologies enable genome editing with greatly enhanced precision, they have certain restrictions and limitations that are not an issue with traditional HDR-based methods. For example, base editors can only install transition mutations and have strict protospacer design requirements that prevent certain bases from being viable base editor targets. Furthermore, if multiple target bases are present within the “base editing window” for a given protospacer, they may all become edited at once, reducing the precision of base editing (referred to as bystander editing). Although prime editing can overcome these issues, editing efficiency is often low without use of additional “nicking gRNAs,” which has the undesired side effect of increasing indel formation at the target site. Additionally, the sheer possible number of prime editing gRNA (pegRNA)-nicking gRNA combinations for a given modification of interest makes finding the optimal construct cumbersome. Finally, neither base editing nor prime editing can facilitate the insertion of large DNA sequences such as gene knock-ins²⁵⁻²⁷, and certain specialized applications, such as gene drive technologies²⁸, explicitly require HDR and therefore cannot be performed with base editing or prime editing.

It has recently been acknowledged that indel sequences arising from a given DSB are generally reproducible and depend on the sequence surrounding the DSB. Sites with low microhomology (<4-nt of homology) are thought to be mainly processed by NHEJ, which often generates one base pair insertions^{29,30}. In contrast, sites with high microhomology (5- to 25-nt of microhomology) are efficiently processed by MMEJ, which results in well-defined deletions of the bases between the microhomology sites. Inspired by these observations, researchers have developed algorithms to predict indel products. One such software, “Microhomology-Predictor,” can predict MMEJ deletion outcomes, and was developed to help researchers identify optimal cut sites that avoid MMEJ-mediated deletions that do not result in frame-shift mutations³¹. Another, inDelphi, was generated using machine learning based off a dataset of 2,000 gRNA-DNA target site pairs and corresponding indel sequences and can predict indel sequence outcomes (including both NHEJ-mediated insertions and deletions, as well as MMEJ-mediated deletions) in different cell lines³². In addition, inDelphi can predict the distribution frequency of indel products. While for many sites, indel products are heterogenous, it is estimated that 5-11% of gRNAs produce a single repair outcome that represents more than 50% of repair products, and 27-47% of gRNAs produce a single repair outcome that represents more than 30% of repair products. Therefore, it is needed to develop a method that takes advantage of the reproducible and predictable nature of these high frequency indel sequences to improve HDR-mediated genome editing.

### Materials and Methods

### Cloning and Constructs

JDS246 (NGG-WT-Cas9, Addgene plasmid #43861), pCMV_ABEmax_P2A_GFP (Addgene plasmid #112101), pCMV-PE2 (Addgene plasmid #132775), pFYF1320 (gRNA expression plasmid, Addgene plasmid #47511), pX330 (Addgene plasmid #42230), pCas9-HE (Addgene plasmid #109400), and the donor plasmid for the ACTB knock-in experiments (AICSDP-15:ACTB-mEGFP, Addgene plasmid #87425) were obtained from Addgene. pCMV_ABEmax_P2A_GFP was used as a template to create Cas9-P2A-GFP and Cas9-NG-P2A-GFP constructs using USER cloning, following New England Biolabs (NEB) protocols⁵².

Two Bsmbl (a type IIS restriction enzyme) recognition sites were installed into the spacer region of the pFYF1320 plasmid using USER cloning, following NEB protocols, to produce the gRNA destination vector pU6-sgRNA-Bsmbl. Custom guide RNA plasmids for each target site were then generated from pU6-sgRNA-Bsmbl using Golden Gate assembly protocols as described by NEB. Briefly, pU6-sgRNA-Bsmbl was digested with BsMBI-v2 (NEB #0739) overnight following the manufacturer's instructions. The digested backbone was gel purified using a QIAquick Gel Extraction kit (#QIAGEN 28704), and inserts encoding custom spacer sequences were annealed and ligated into the backbone with T4 DNA ligase (NEB #M0202) following the manufacturer's instructions. As GFP tagging of LMNA was previously done in our lab, those plasmids were cloned into a different backbone. The LMNA primary gRNA was cloned into the pX330 backbone (which has Bbsl recognition sites), creating pU6_LMNA_SpCas9. Briefly, the pX330 backbone was digested with Bbsl (NEB #R3539S) following the manufacturer's instructions, gel extracted, and the annealed inserts encoding custom spacer sequences were ligated into the digested, purified backbone with T4 DNA ligase. pLMNA_HA_donor_GFP plasmid was cloned in multiple steps: first the LMNA homology arms were amplified from genomic DNA using primers, then the PCR product was TOPO cloned into the pCR2.1 TOPO backbone (ThermoFisher #K450002) to make a pLMNA_reservoir plasmid following the manufacturer's instructions. The entirety of the pLMNA_reservoir plasmid was then amplified by PCR using primers, which created a linearized DNA product. The linearized product was assembled with TurboGFP (synthesized gene block) using Gibson assembly following the NEB protocol #E2611.

Prime editing gRNAs were generated in two steps. First the spacer sequence was incorporated into the pU6-sgRNA-Bsmbl plasmid as previously described to generate a stepping-stone plasmid, followed by incorporation of the reverse transcriptase template (RTT) and primer binding sequence (PBS) sequences using site directed mutagenesis. Site directed mutagenesis primers designed to install the RTT and PBS sequences were obtained from integrated DNA technologies, and 5′ phosphorylated using T4 Polynucleotide Kinase (NEB #M0201) following the manufacturer's instructions. PCR was then performed with Phusion High-Fidelity DNA Polymerase (NEB #M0530) with the phosphorylated primers and the stepping-stone plasmid as a template. PCR products were purified using the QIAquick PCR purification kit (QIAGEN #28104) following the manufacturer's instructions. PCR products were ligated using Quick Ligase (NEB #M2200), and ligation products were transformed into NEB 10-beta (NEB #C3019H) cells following the manufacturer's instructions. Endotoxin-free plasmids were prepared using either the Zymo mini (Zymo #D4037) or midiprep (Zymo #11-550B) kit following the manufacturer's instructions. Plasmids generated using USER cloning were fully sequenced with Sanger sequencing, while gRNA plasmids generated using Golden Gate cloning were sequenced around the insert to confirm correct ligation. Protospacer sequences for all gRNA plasmids are available. The selected primary gRNAs were either previously used in prior publications^{22, 24, 25, 27, 32} or designed to have cut sites within 15 bp of the intended mutation and to be “high precision” protospacers by inDelphi (i.e. those predicted to produce outcomes in which the top three indel sequences would represent >40% of products).

### Cell Culture and Transfections

All cells were cultured at 37° C. with 5% CO₂ in a humidified environment. HEK293T (ATCC CRL-3216), HeLa (ATCC CCL-2), and K562 (ATCC CCL-243) cells were obtained from ATCC. HEK293T and HeLa cells were maintained in Dulbecco's Modified Eagle's Medium (DMEM, Gibco #10566-016) supplemented with 10% (V/V) fetal bovine serum (FBS, Gibco #10437-028), while K562 cells were maintained in Roswell Park Memorial Institute 1640 (RPMI 1640, Gibco #11875-093) media supplemented with 10% (V/V) FBS. HEK293T and HeLa cells were plated at a density of 100,000 cells per well in 48-well plates in a total volume of 250 μL per well and transfected four hours after plating using 1.5 μl Lipofectamine 2000 (Invitrogen #11668-019) and a custom DNA mixture (described below) in 25 μL total volume, made up with Opti-MEM (Gibco #31985-070). For PE2 experiments, 750 ng of PE2 plasmid and 250 ng of pegRNA plasmid were used per transfection. For PE3 experiments, 750 ng of PE2 plasmid, 250 ng of pegRNA plasmid, and 83 ng of nicking gRNA plasmid were used per transfection. For ssODN double tap experiments, 750 ng of Cas9-P2A-GFP plasmid (except for experiments involving the SEC61B, HEXA, and HBB loci, in which case Cas9-NG-P2A-GFP was used) or 750 ng of Cas9-HE plasmid, 300 ng of gRNA plasmid, and 10 nM final concentration of ssODN were used per transfection. The gRNA plasmid mixture was comprised of 200 ng of primary gRNA and 100 ng of non-targeting gRNA or secondary gRNA(s), except for non-targeting negative control samples, in which case 300 ng of non-targeting gRNA was used. For the LMNA knock-in experiment, Cas9 and primary gRNA were expressed from the same plasmid (pU6_LMNA_SpCas9). In this case, 1,000 ng of pU6_LMNA_SpCas9, 100 ng of non-targeting or secondary gRNA plasmid, and 300 ng dsDNA donor plasmid (pLMNA_HA_donor_GFP) was used. For the ACTB knock-in experiment, 750 ng of JDS246 plasmid (Cas9 expression without GFP), 300 ng of gRNA plasmid, and 300 ng of dsDNA donor plasmid was used. The gRNA plasmid mixture was comprised of 200 ng primary gRNA and 100 ng non-targeting or secondary gRNA. For off-target analysis experiments, 750 ng of Cas9-P2A-GFP plasmid and 200 ng of gRNA plasmid (either non-targeting gRNA, primary gRNA, or secondary gRNA only) was used. K562 cells were plated at a density of 1×10⁶ cells per well in 6-well plates in a total volume of 2.5 mL per well and transfected four hours after plating using 15 μl Lipofectamine 2000 (Invitrogen #11668-019) and a custom DNA mixture (described below) in 250 μL total volume, made up with Opti-MEM (Gibco #31985-070). For these experiments, 3750 ng Cas9-P2A-GFP plasmid, 1500 ng gRNA plasmid, and 10 nM final concentration of ssODN were used per transfection. The gRNA plasmid mixture was comprised of 1,000 ng primary gRNA and 500 ng non-targeting or secondary gRNA. When the small molecule Alt-R™ HDR Enhancer V2 (Integrated DNA Technologies IDT #10007910) was tested, 0.435 μl of the Alt-R enhancer was diluted in Opti-MEM (Gibco #31985-070) to 25 μl and added immediately after the transfection. The same volume of DMSO was diluted in Opti-MEM (Gibco #31985-070) and added to a separate well as a control. The media was replaced 24 hours after transfection to reduce cytotoxicity.

For the RNP transfections, Cas9 (TrueCut v2, #A36497) and custom TrueGuide synthetic sgRNAs (with the same spacer sequences that were used with the plasmid-based delivery samples) were purchased from Thermo Fisher. Transfection was performed into HEK293T cells plated in 48 well as described above. First 750 ng TrueCut Cas9 was complexed with 4.5 pmoles TrueGuide gRNA. The gRNA mixture was comprised of 3 pmoles of primary gRNA and 1.5 pmoles of non-targeting gRNA or secondary gRNA(s). After RNP complex generation, ssODNs were added as described above (10 nM final concentration) and transfected with 1.5 μl Lipofectamine 2000 (Invitrogen #11668-019) with Opti-MEM (Gibco #31985-070) as described above. Samples from the ssODN experiments were harvested three days after transfection and processed for NGS analysis while GFP knock-in experiments were continuously passaged for fourteen days followed by flow cytometry analysis.

### Flow Cytometry and Fluorescence Activated Cell Sorting (FACS)

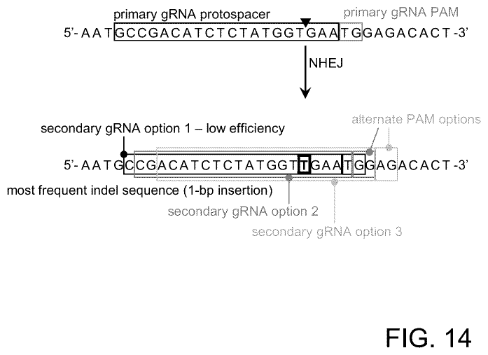

HEK293T cells were analyzed via flow cytometry to assess GFP knock-in efficiency fourteen days after transfection. Cells were washed with 250 μL phosphate buffered saline (PBS, Gibco #10010-023) in the plate and then detached from the plate with Accumax (Innovative-Cell Technology #AM-105) according to the manufacturer's instructions. After harvesting, cells were resuspended in 500 μL PBS. Samples were filtered into FACS tubes (Falcon, #352235) and kept on ice until analysis. A S3e cell sorter (Bio-Rad) equipped with 488 nm, 561 nm and 640 nm lasers was used for all analysis. The instrument was calibrated and quality control checked before each flow cytometry or FACS experiment. GFP positive samples were quantified using the 525/30 nm channel. Single color (pool of the transfected samples for each group) and no color (untransfected cells) control cell populations were used to set up gating. Single color (GFP positive cells for knock-in) had higher intensity than the untransfected cells for the corresponding channels (GFP channel for knock-in). The GFP population was selected based on untransfected cells. Gates were set up or checked with the untransfected and single color controls for each flow cytometry or FACS experiment. Example of the gates are shown in FIG. 14. Doublets were gated out using forward and side scattering width against area, and 20,000 events were analyzed. HEK293T cell viability for off-target experiments was also analyzed via flow cytometry 72 hours after transfection. Cells were washed with 250 μL PBS on the plate and then detached from the plate with Accumax (Innovative-Cell Technology #AM-105) according to the manufacturer's instructions. After harvesting, cells were resuspended in propidium iodide staining buffer (PI, Invitrogen #1304MP) following the manufacturer's instructions. Samples were filtered into FACS tubes and kept on ice until analysis. Cells stained with PI were quantified using the 615/25 nm channel and GFP samples were monitored on the 525/30 nm channel. Single color (non-transfected cells stained with PI, and separately transfected cells without PI staining) and no color (no transfection) control cell populations were used to set up gating. Doublets were gated out using forward and side scattering width against area, and 20,000 events were analyzed.

Isogenic cells for the zygosity experiment were generated using FACS. Cells were prepared for sorting as described above. Samples were gated against untransfected samples as described above. Single GFP positive cells (cells expressing Cas9) were sorted into 96 well plates 48 hours post transfection using a BD Ariall cell sorter. Prior to sorting, wells were filled with 200 μL of 30% (V/V) FBS DMEM media and incubated at 37° C. After sorting, plates were kept in the incubator for 3 weeks for clonal expansion, then harvested for NGS analysis.

All HeLa and K562 cell experiments required FACS (using GFP fluorescence) before NGS analysis. HeLa cells were prepared the same as the HEK293T cells described above. For K562 cells, cells were spun down at 300 g for 5 minutes, the supernatant was decanted, and cells were washed with another 500 μL PBS. Following the second wash, the cell pellets were resuspended in 500 μL PBS and kept on ice until sorting. The 525/30 nm channel was used to identify cells with GFP fluorescence, and untransfected cells were used as negative controls to set up gating. Doublets were gated out using forward and side scattering width against area, and 40,000 GFP positive cells were collected using purity mode. K562 cells were collected into RPMI 1640 supplemented with 20% (V/V) FBS, and HeLa cells were collected into DMEM supplemented with 20% (V/V) FBS. Both cell lines were then spun down, washed with 500 μL PBS, and then prepped for NGS.

### Next-Generation Sequencing

After 72 hours of editing, cells were washed with PBS either on the plate (HEK239T cells) or after FACS (HeLa and K562 cells), followed by proteinase K digestion (in a buffer made up of 10 mM Tris, pH 7.5; 0.05% SDS, and 25 μg/mL freshly added proteinase K) at 37° C. for 1 hour, followed by an 80° C. heat treatment for 30 minutes. HEK293T cells were digested in 100 μL total volume of buffer while the sorted HeLa and K562 cells were digested in 50 μL total volume of buffer. After the lysis, genomic loci of interest were PCR amplified using locus-specific primers. These primers were designed to contain an adapter sequence, allowing for sample barcoding with a second round of PCR. PCR reactions were performed using Phusion High-Fidelity DNA Polymerase following the manufacturer's instructions with the following modifications: all PCR reactions were performed using GC buffer, 3% DMSO was utilized, and 25% of the recommended primer amount was used to reduce the amount of primer dimers. 25 cycles of amplification were used for round one PCRs, while 15 cycles of amplification were used for round two PCRs. An annealing temperature of 61° C., and an extension time of 45 seconds was used for both rounds. 0.5 μL of genomic DNA was used a template for round one PCRs, and 0.5 μL of round one PCR product was used as a template for round two PCRs at 10 μL total reaction volume. Second round PCR products were pooled together based on the amplicon size and purified from a 2% agarose gel using the QIAGEN gel extraction kit (QIAGEN #28704) following the manufacturer's instructions. The resulting purified libraries were quantified with the Qubit dsDNA high sensitivity kit (Thermo Fisher #Q32851) and diluted to 1.8 pM following Illumina's sample preparation guidelines. The final library was mixed with 1.8 pM PhiX in a nine to one ratio. Samples were then sequenced on a MiniSeq (Illumina) via paired end sequencing.

### Data Analysis and Statistics

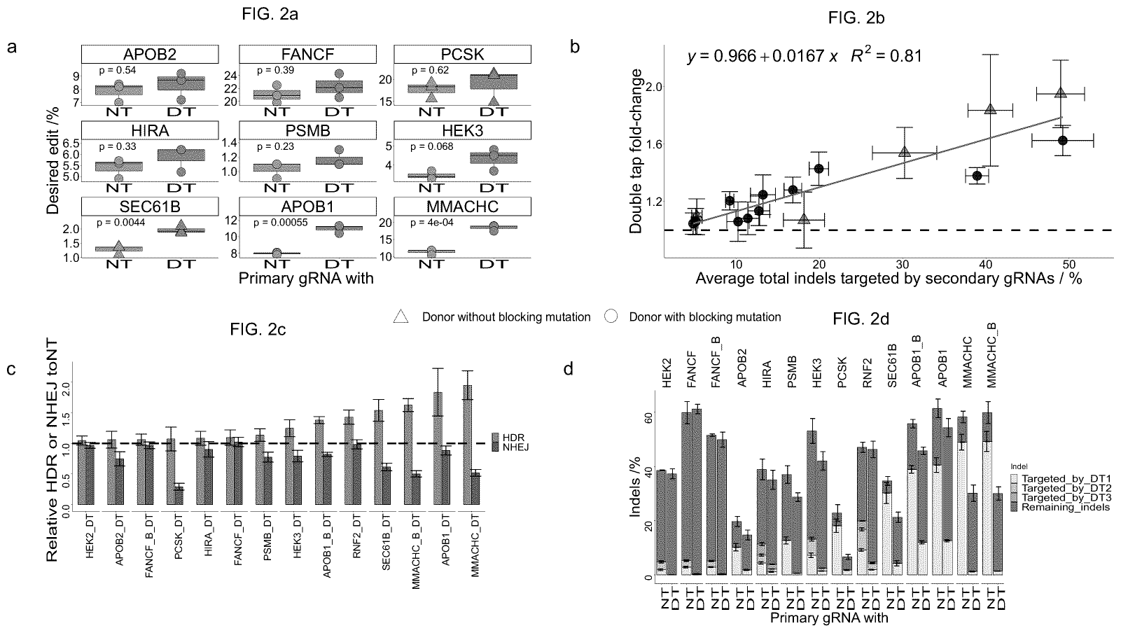

NGS samples were processed in CRISPResso2⁵³ (version 2.0.20b) using the default and HDR outputs. Values from the CRISPResso2 were further processed in R Studio (version 1.4.1717) and plotted with the “ggplot2” ⁵⁴ package. Univariate statistics were performed in R Studio using the “ggpubr” package. FACS data was analyzed with FlowJo (version 10.7.2) to assess knock-in efficiencies. InDelphi³² (version 0.18.1) was used to predict insertions and deletions at the Cas9 cut site. Indel frequency values and errors were calculated as follows: values represent the mean of the number of sequencing reads with the indel sequence of interest (or any indel, when calculating total indel rates) divided by the total number of sequencing reads ±standard deviation (SD) for n=3 biological replicates. For biological replicates, cells were plated into three different wells on the same day. Transfection reagents were prepared in three different tubes and transfected into independent replicates. Day to day transfection variability (from different splits of the same HEK293T cells) is demonstrated in FIGS. 2a-2d & 3a-3d at the MMACHC site.

Fold-change values and errors were calculated as follows: values represent the mean of the number of sequencing reads with perfect HDR outcomes divided by the total number of sequencing reads for double tap samples divided by that of the samples with primary and non-targeting gRNA±propagation of uncertainty of the SD for n=3 biological replicates.

Percent decrease values and errors were calculated as follows: The mean total indel rates were first calculated for the sample with primary and non-targeting gRNA and for the sample with primary and secondary gRNA(s) (as described above). Then the difference of these two values were calculated and then divided by the mean total indel rate of the primary and non-targeting gRNA sample, multiplied by 100±propagation of uncertainty of the SD for n=3 biological replicates.

### Data Availability

The high-throughput sequencing data generated in this study have been deposited in the NCBI Sequencing Read Archive database under Accession Number PRJNA819982.

### Results

### A. Initial Testing of the Double Tap Method in HEK293T Cells for Introducing Small Edits

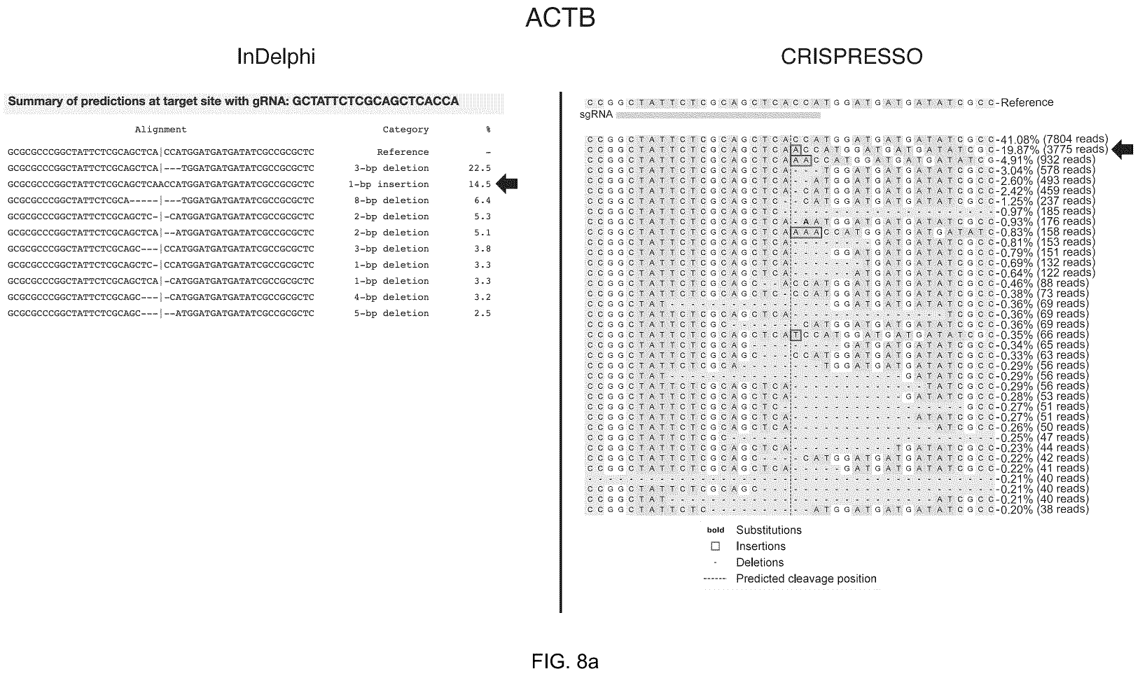

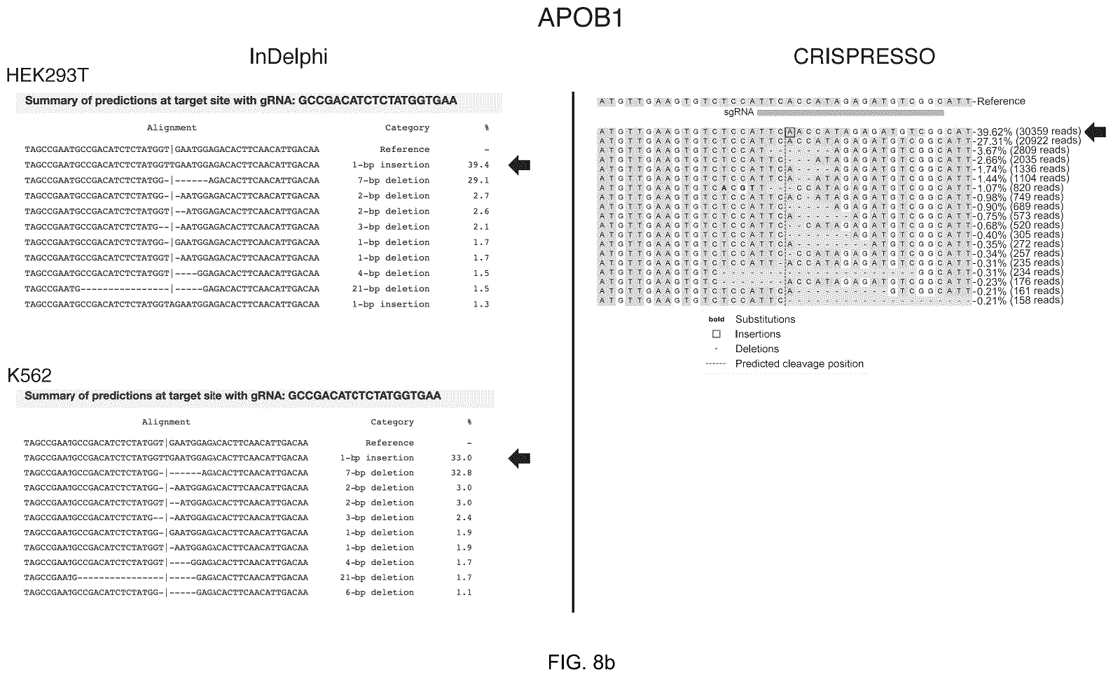

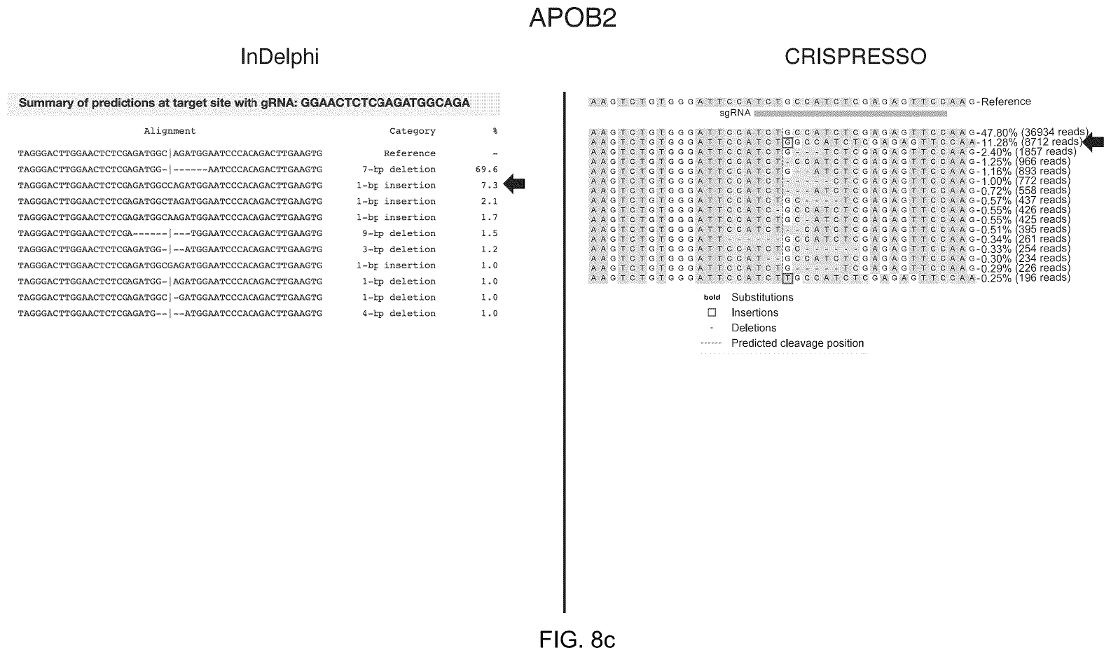

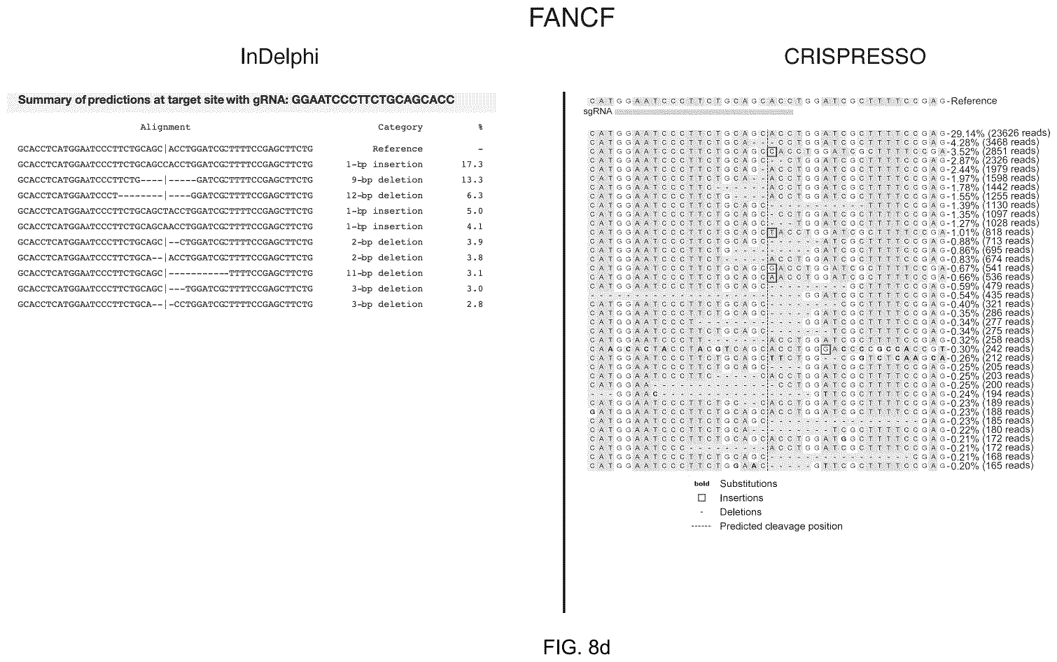

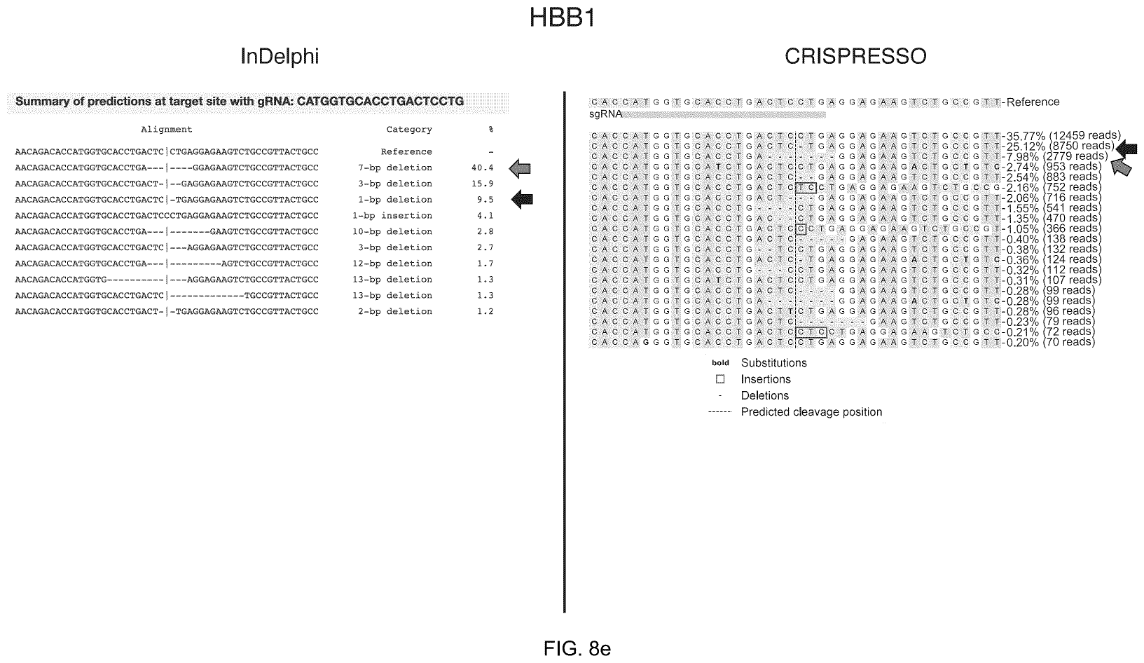

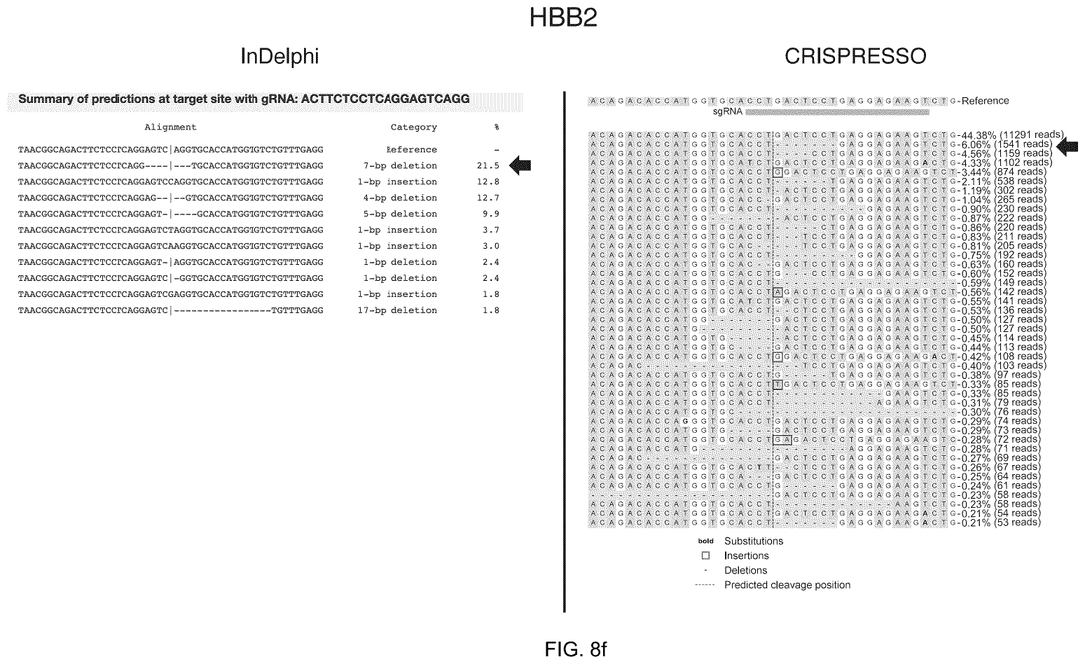

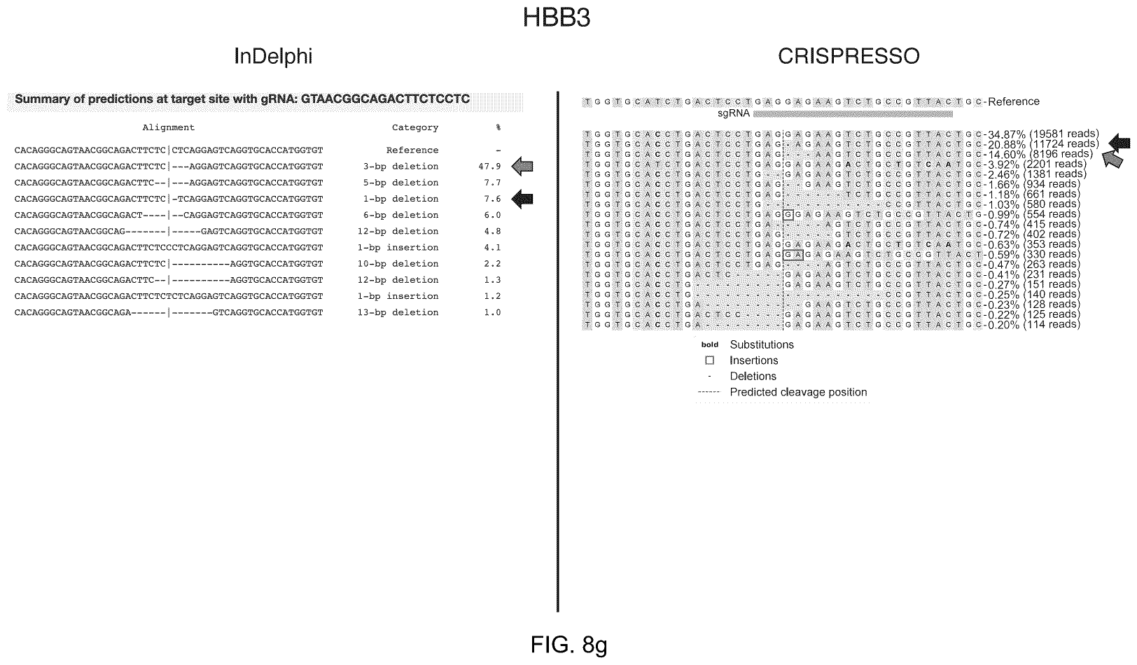

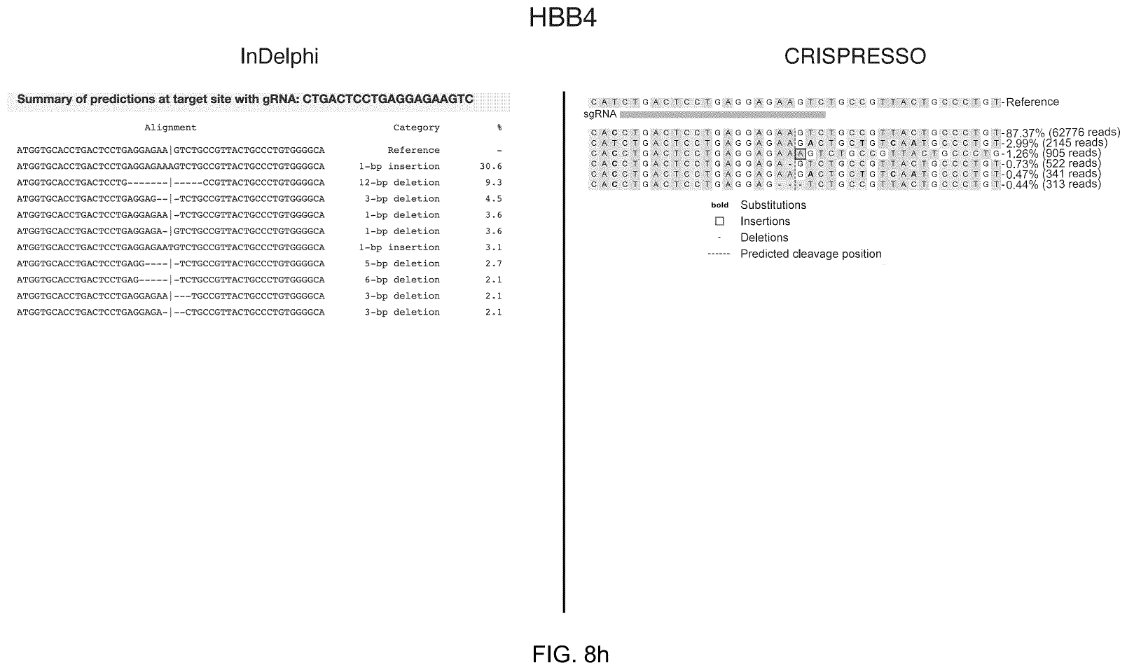

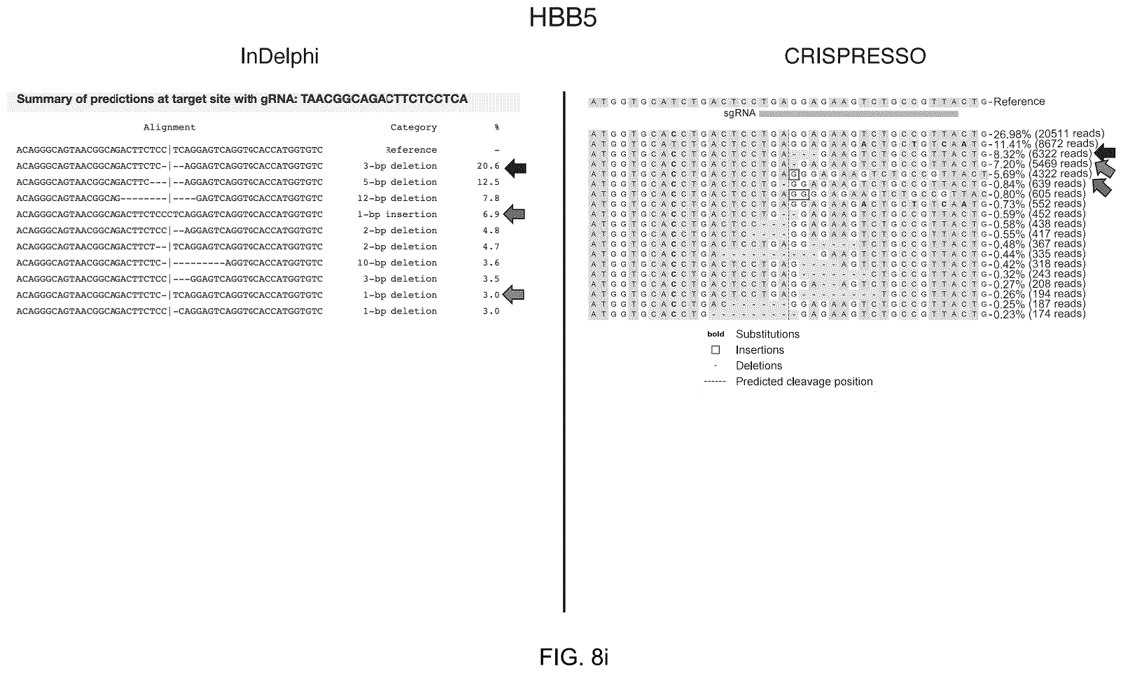

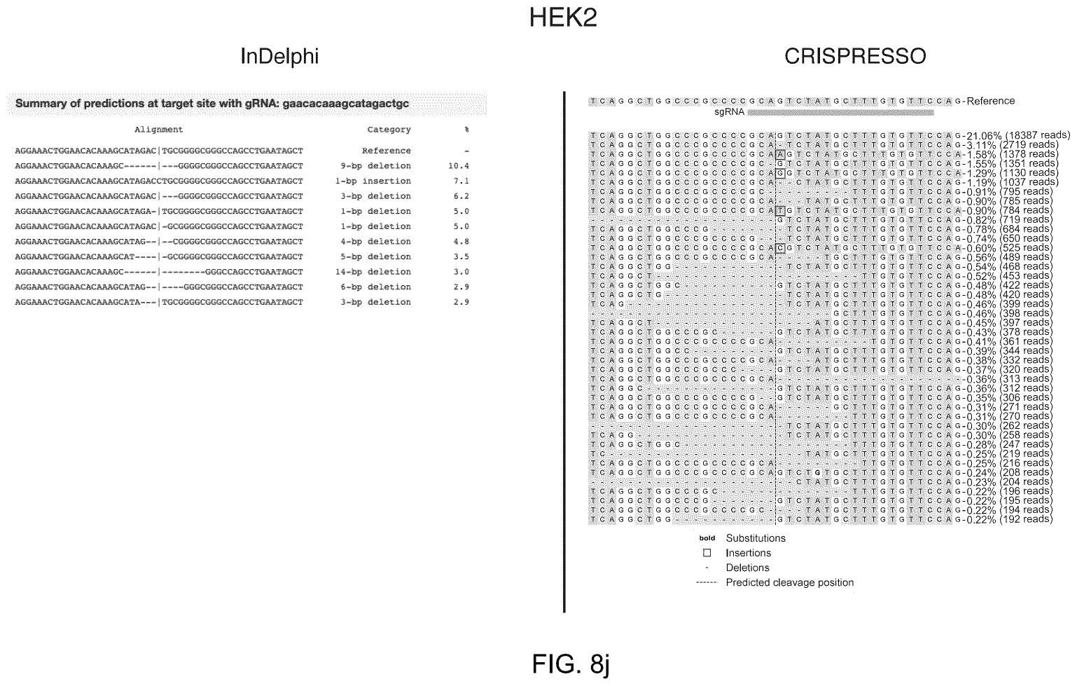

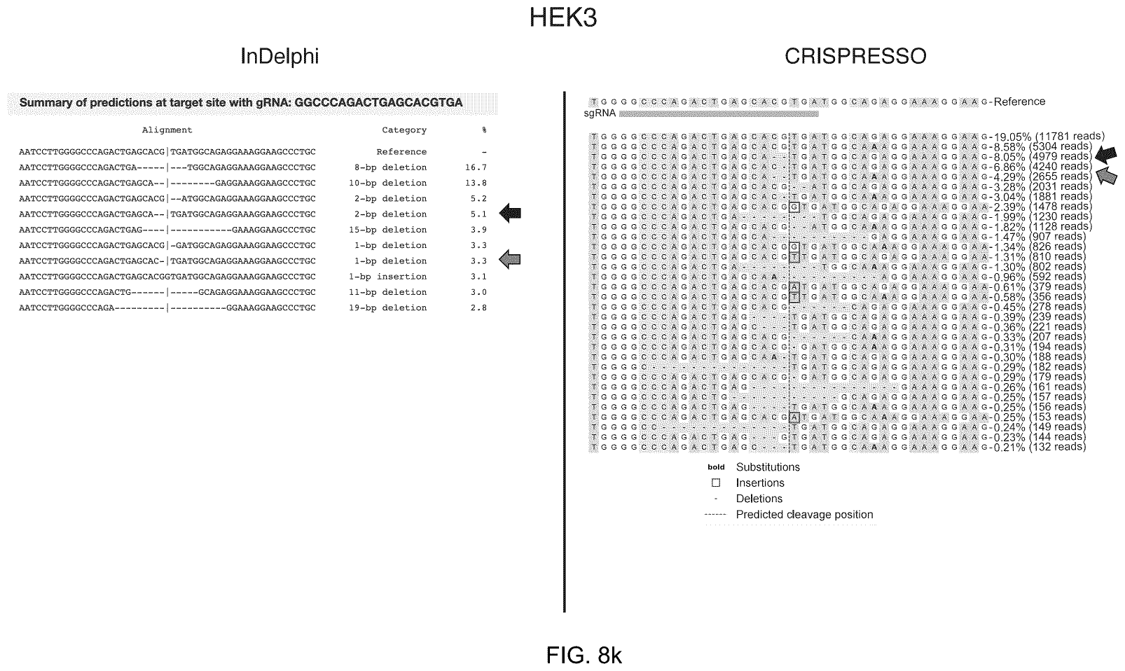

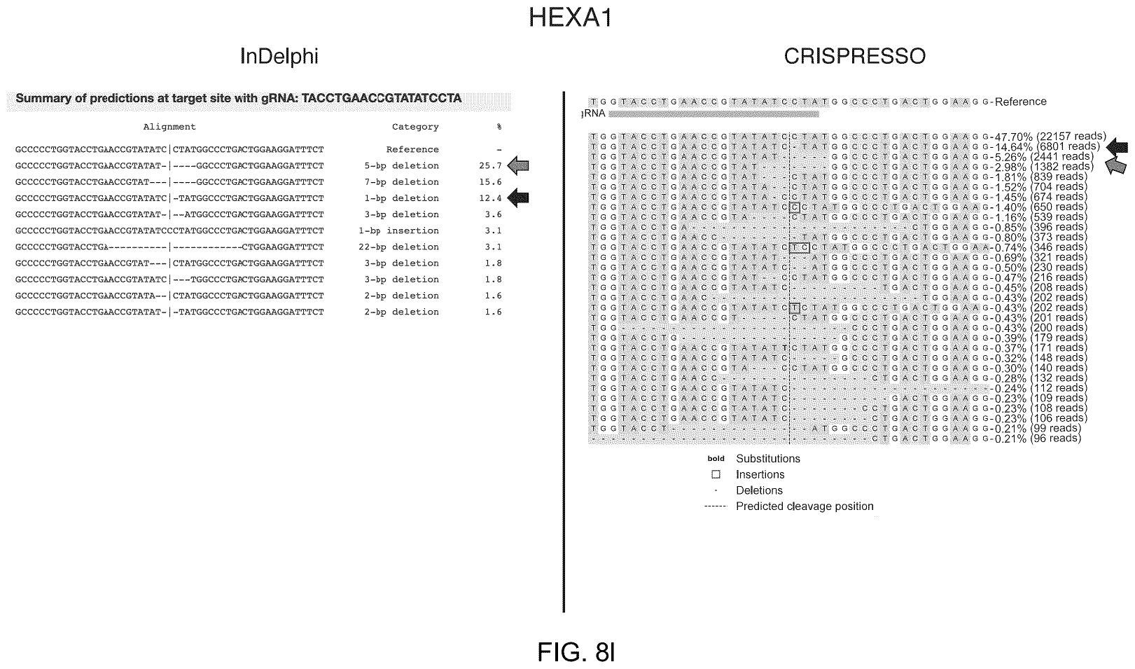

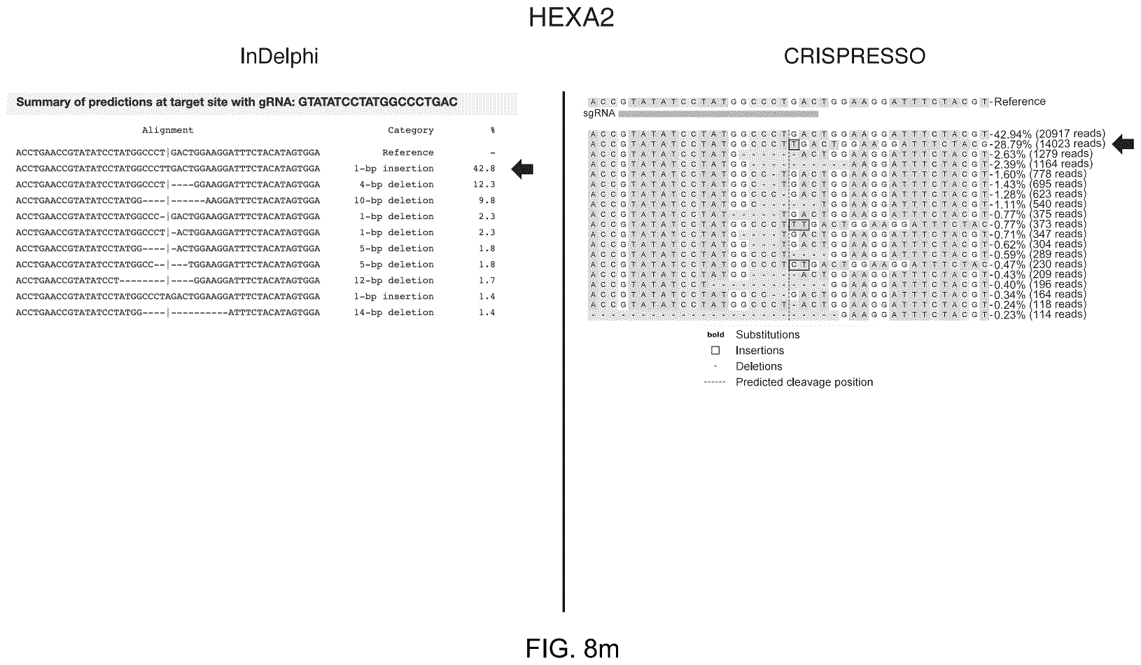

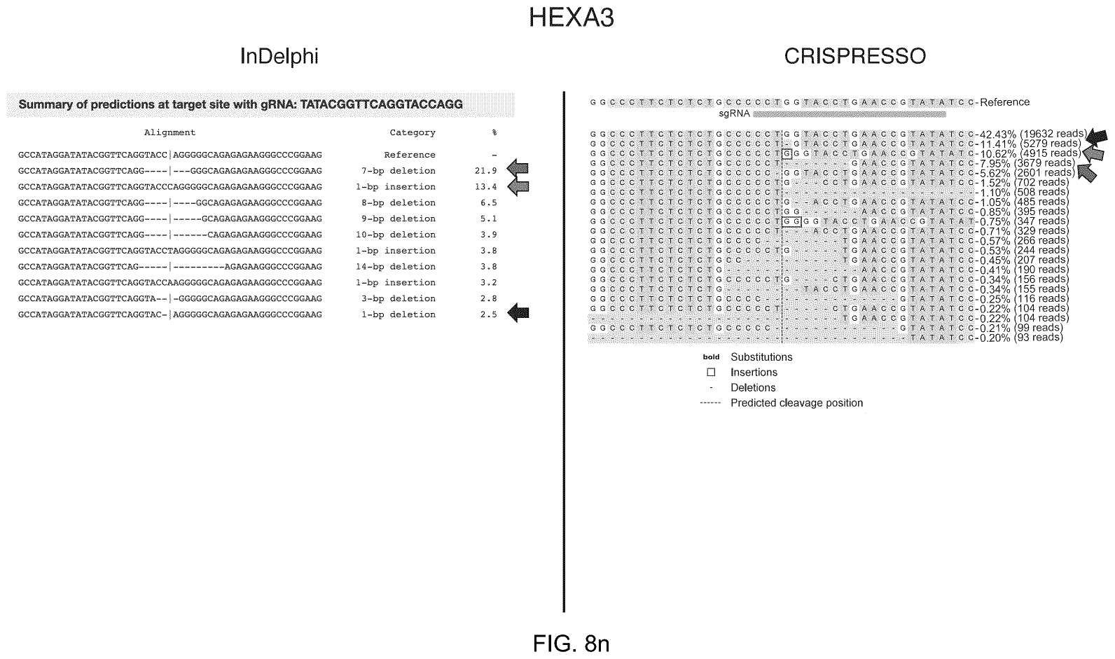

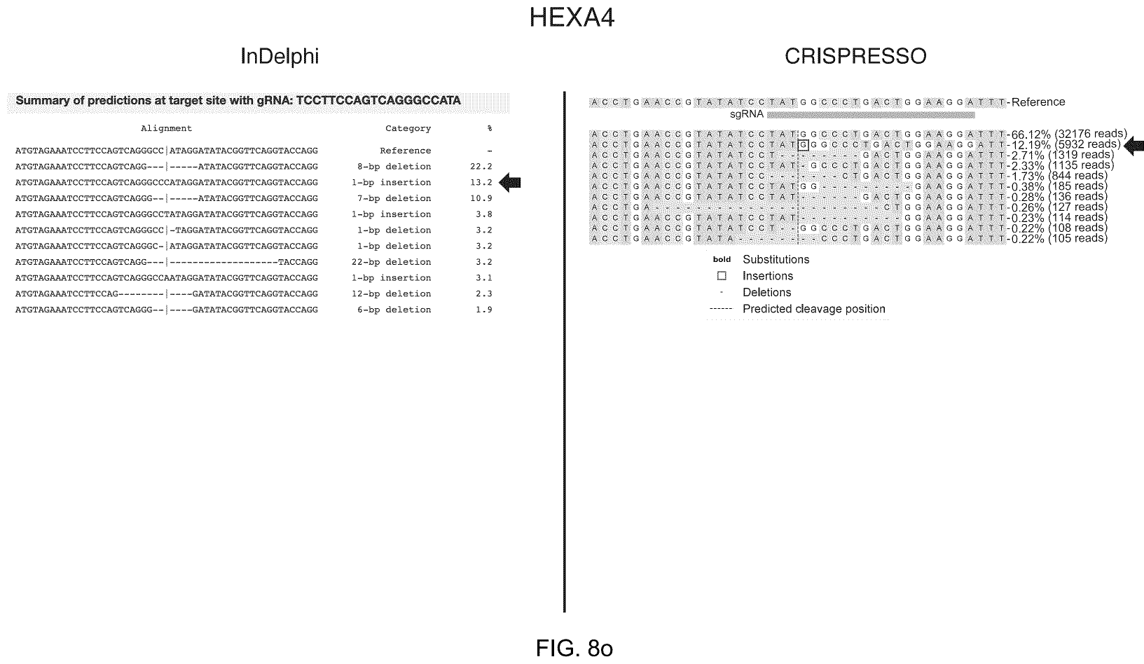

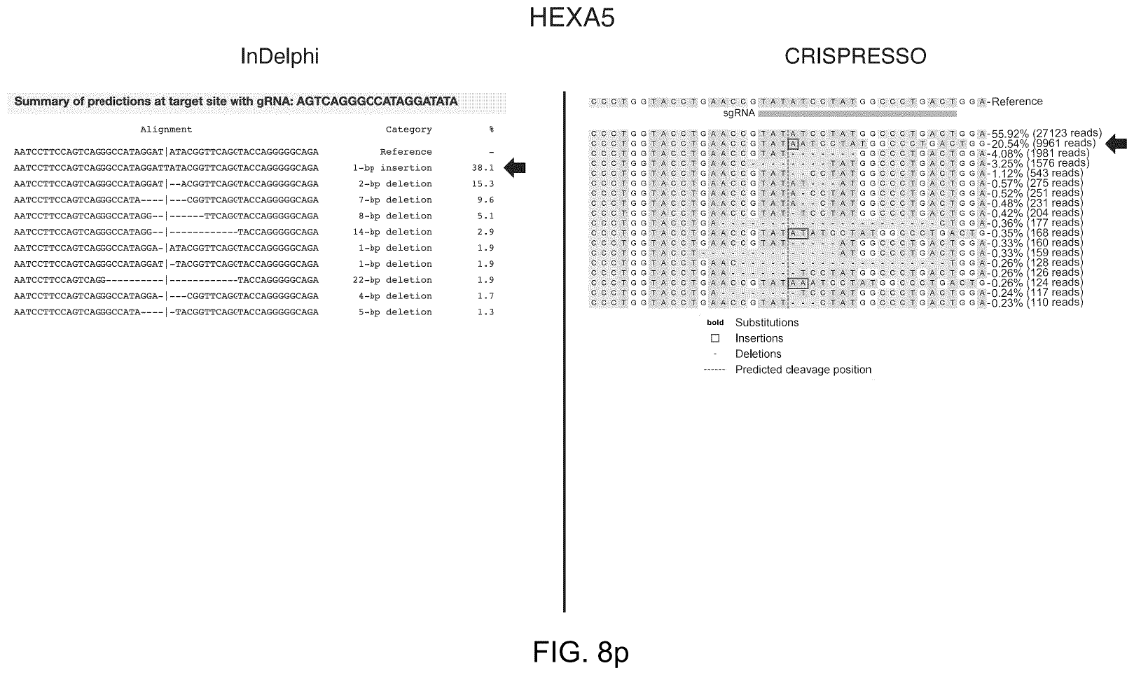

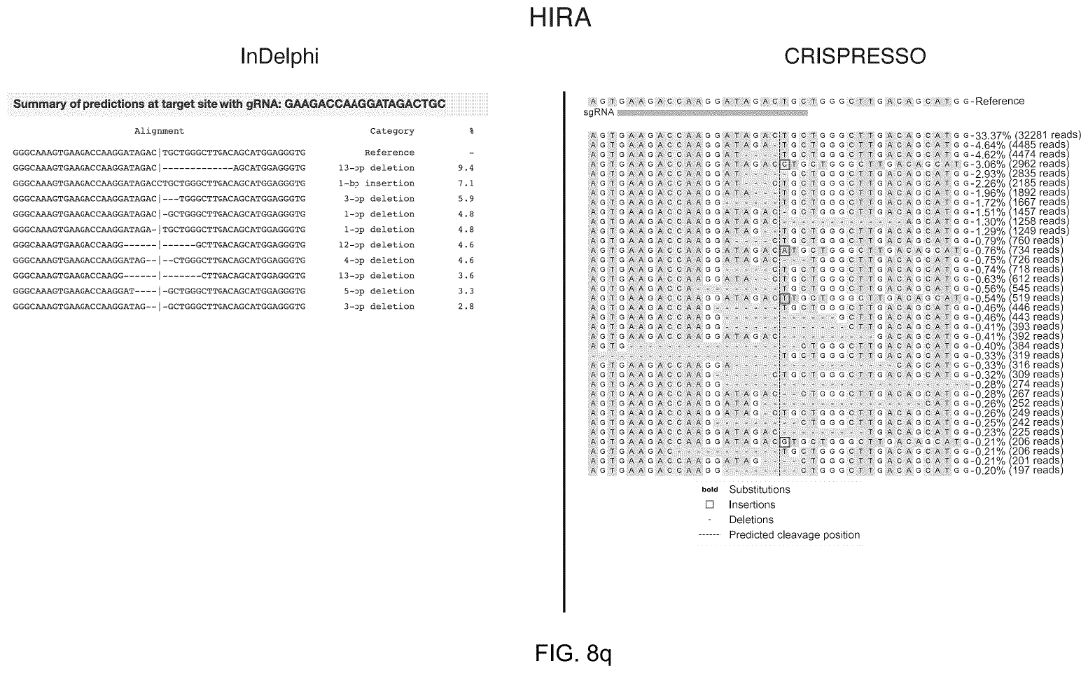

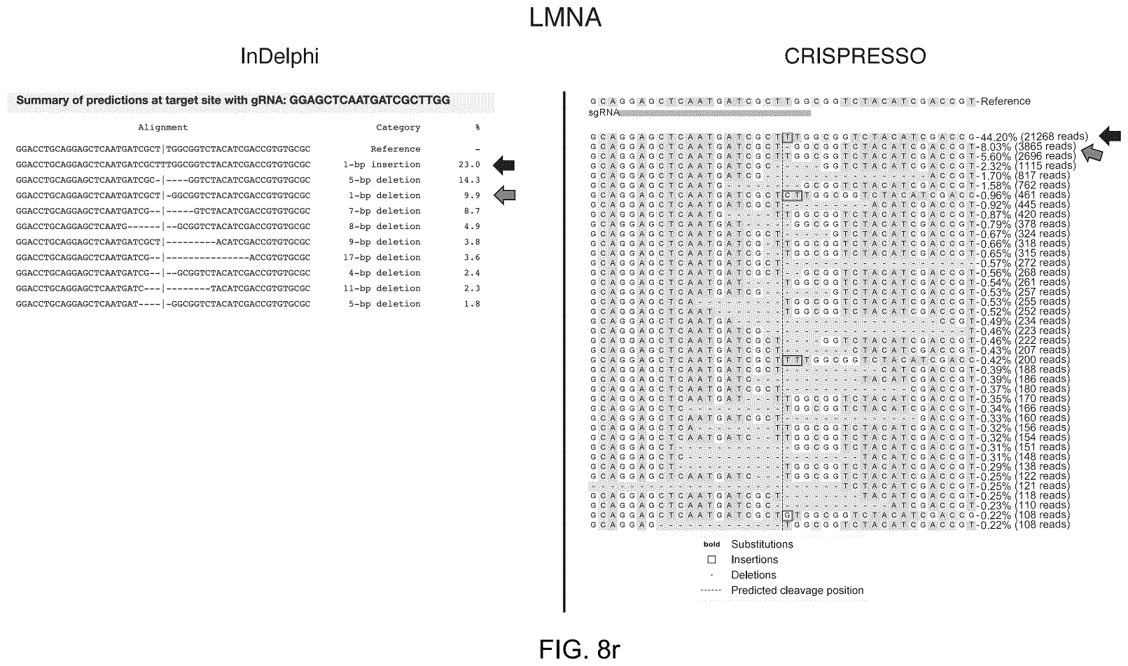

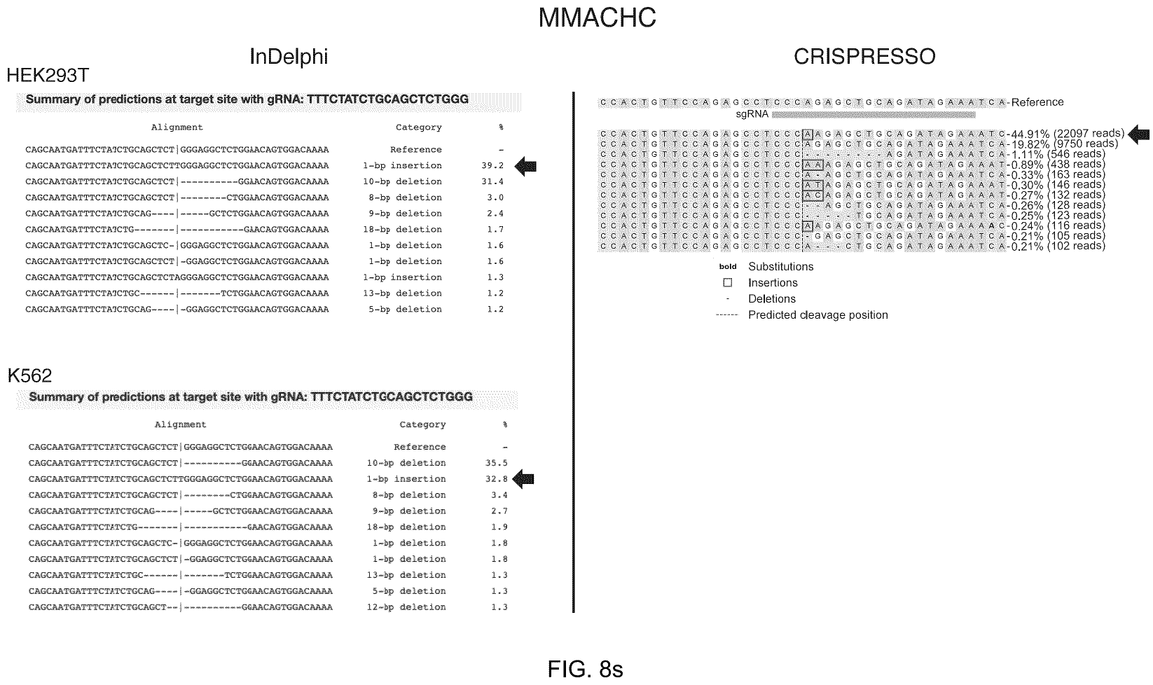

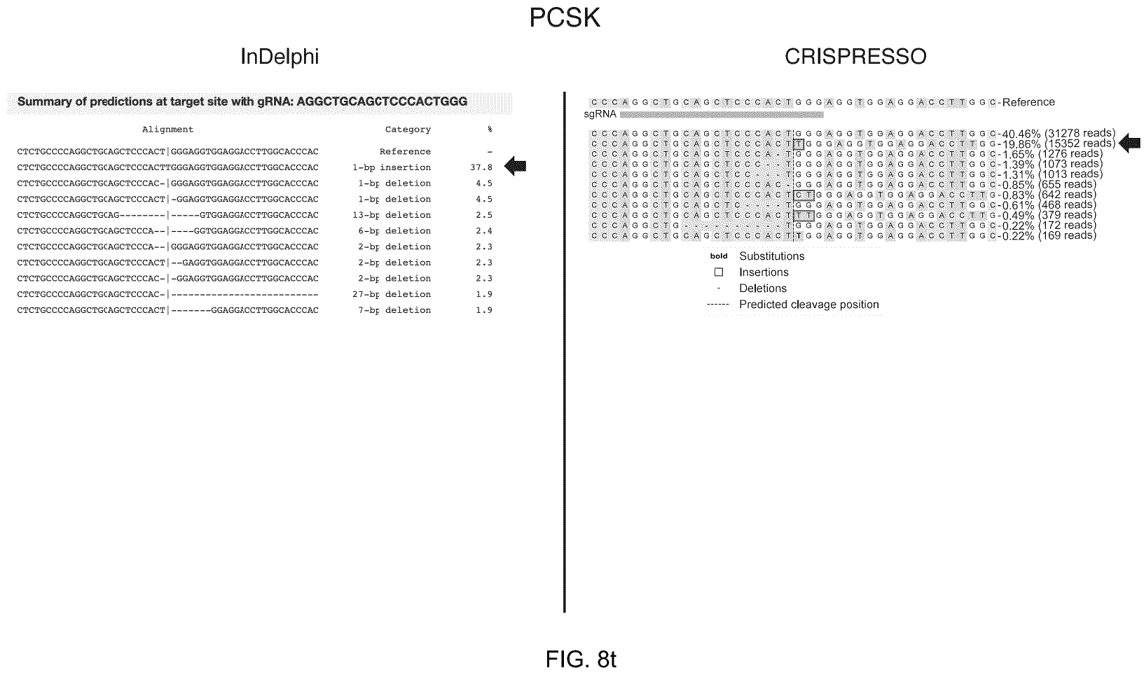

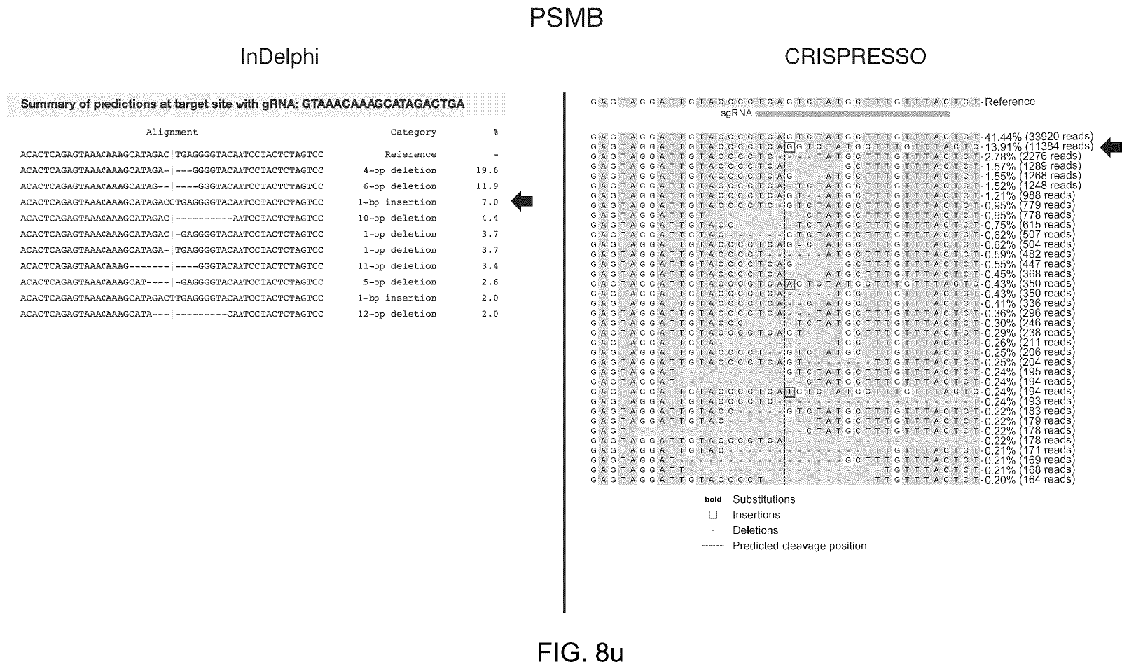

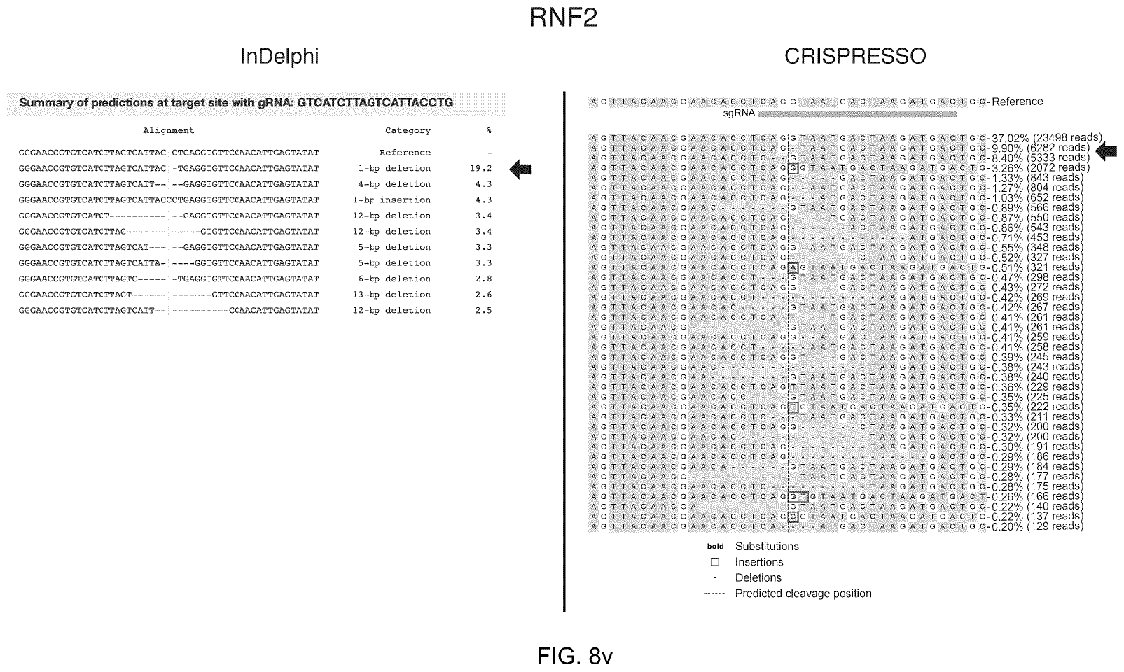

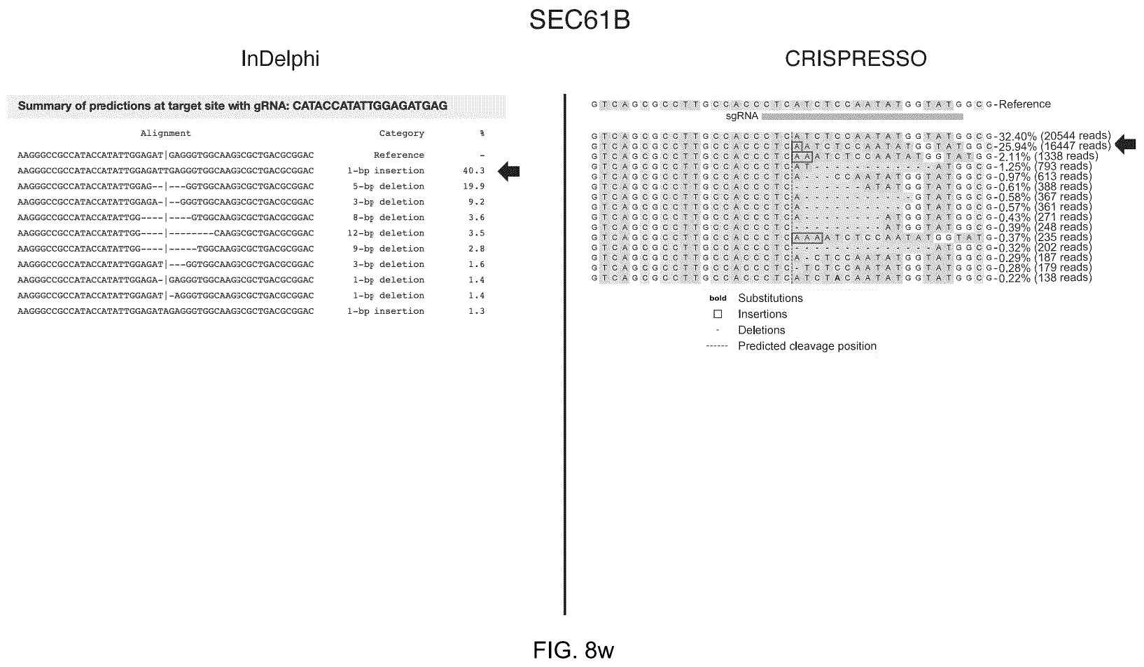

Four well-characterized genomic loci were first selected to test that targeting reproducible indel sequences with secondary gRNAs could boost HDR-mediated genome editing efficiencies. Specifically, previously validated protospacers that target loci within the APOB, MMACHC, RNF2, and FANCF genes (hereafter referred to as the APOB1, MMACHC, RNF2, and FANCF loci or sites, respectively)^{22,32} were chosen. To characterize the most common indel sequences introduced using these primary gRNAs, human embryonic kidney (HEK293T) cells were transfected with plasmids encoding Cas9 and primary gRNA. After 72 hours, cells were lysed, genomic DNA (gDNA) was extracted, and loci of interest were amplified, sequenced using next-generation sequencing (NGS), and analyzed with CRISPResso2 to identify recurrent indels. The experimentally determined and predicted indel sequences (using inDelphi) are shown in FIG. 1b (for the MMACHC locus) and FIGS. 8b, 8d, 8s and 8v, the indel introduction efficiencies in these non-double tap experiments were hereinafter referred to as “initial indel rates”. Based on these indel data one secondary gRNA each for the APOB1 and MMACHC sites, two for the FANCF site, and three for the RNF2 site were designed. The particular indel sequences as they were reproducible (occurred in all replicates and the inDelphi analysis) and represented a large range of initial indel rates (from 3% to 50%) were targetted, allowing to investigate the relationship between initial indel rate(s) of the targeted indel(s) and enhancement of editing efficiency after implementing the double tap method.

ssODN templates to install either a point mutation (for the RNF2 and MMACHC sites) or a small insertion (for the FANCFand APOB1 sites) were then designed so that editing efficiencies could be monitored. Unless explicitly noted otherwise, all the ssODNs were designed symmetrically, with 50 or 70-nt homology arms. HEK293T cells with ssODN and plasmids encoding Cas9, primary gRNA, and either non-targeting gRNA (to keep the total amount of gRNA plasmid constant when comparing to the double tap experiments) or secondary gRNA(s) were transfected. After 72 hours, cells were lysed and analyzed via NGS and CRISPResso2 to determine HDR and indel introduction efficiencies. Increases in absolute HDR-mediated genome editing efficiencies were observed in all cases, with the relative size of the increase roughly correlated to the initial rates of the indel sequences that were targeted with the secondary gRNAs (expand dataset and further analysis of this relationship are shown in FIG. 2b). Specifically, when using secondary gRNAs targeted to indels with high (40.5±2.7% for APOB1 and 50±2.9% for MMACHC) initial rates, the average HDR-mediated editing efficiency improved 1.8±0.4-fold for APOB1, and 2.0±0.2-fold for MMACHC (FIG. 1c). When targeting indel products with moderate (9.2±0.5% for the RNF2 site) initial rates, the average overall HDR-mediated genome editing efficiency improved 1.2±0.1-fold (FIG. 1d). As the RNF2 site had two additional indel products with high frequencies, the impact of using two and three secondary gRNAs were further tested. When using two secondary gRNAs whose corresponding indels collectively had initial rates of 17.0±1.1%, the double tap method boosted the average HDR-mediated editing efficiency by 1.3±0.1-fold (FIG. 1d). Using three secondary gRNAs that collectively corresponded to initial indel rates of 19.9±1.2%, the average HDR-mediated genome editing efficiency improved 1.4±0.1-fold (FIG. 1d). Finally, when targeting indel products with low (5.3±0.6% for the FANCFsite) initial rates, only a 1.1±0.1-fold improvement was observed (FIG. 1c; note two secondary gRNAs were used in this case to target two indels whose initial indel rates summed to 5.3±0.6%). These data show that the double tap method can improve precision genome editing efficiencies. Additionally, these results suggest that the use of secondary gRNAs targeted to indel sequences with higher frequencies leads to larger improvements in HDR-mediated genome editing efficiencies than secondary gRNAs targeted to indel sequences with lower frequencies.

### B. Characterization of the Double Tap Method

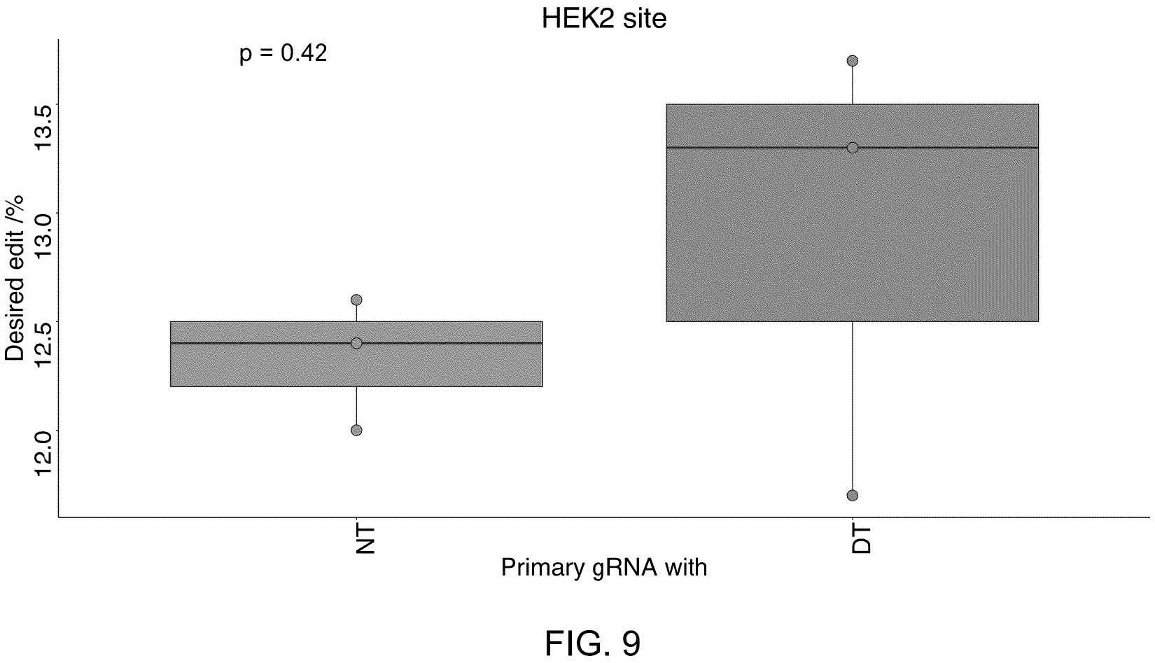

For further characterization and validation, the double tap method was tested at seven additional protospacers (within the LOC1 10120638, LINC01509, HIRA, PSMB2, PCSK9, APOB, and SEC61B genes, hereinafter referred to as the HEK2, HEK3, HIRA, PSMB, PCSK, APOB2, and SEC61Bloci or sites, respectively), using HDR to install point mutations, small deletions, and small insertions. Again, the double tap method increased HDR-mediated genome editing efficiencies at all tested sites, with larger fold-change values when using secondary gRNAs targeted to indel sequences with larger initial rates (FIGS. 2a-2c and FIG. 9). Fold-change values were graphed as a function of the collective initial indel rates targeted by the secondary gRNAs (FIG. 2b) to better visualize the correlation between these two factors. With this larger dataset, it is confirmed that larger increases in HDR efficiencies occur when targeting indels with larger initial rates. In fact, these data can be fit with a linear regression model (fold-change=0.966+0.0167*[initial rate of indel(s) targeted with secondary gRNA(s)], r²=0.81 FIG. 2b), allowing for the approximation of fold-change values in future experiments (see Disease modeling and comparison to prime editing discussed herein).

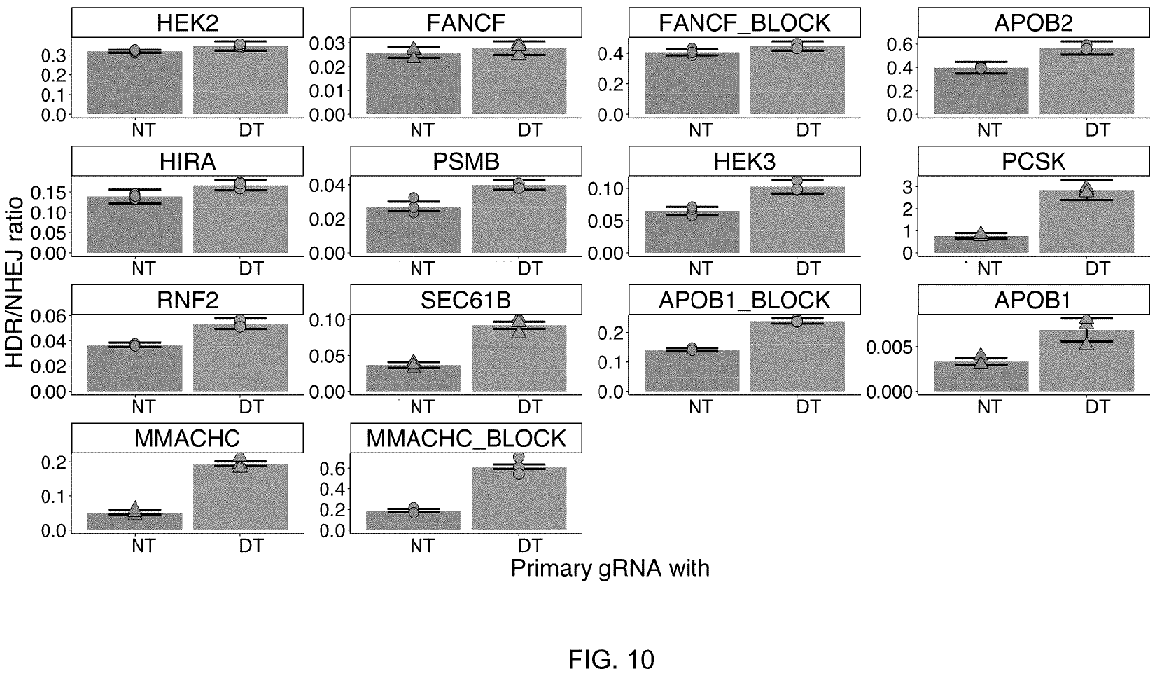

Furthermore, decreases in the total absolute indel rates were also observed when using the double tap method in ten out of eleven cases (FIGS. 2c and 2d). In all cases, introduction rates of the specific indels targeted with secondary gRNAs decreased (FIG. 2d). At the same time, certain indel sequences that were present in the primary gRNA-only experiments at very low (generally <1%) frequencies increased in the double tap samples. In general, sites with lower initial indel rates targeted by secondary gRNAs showed smaller decreases in total indel rates (for example, at the APOB2 site, an indel with an initial rate of 10.2±1.3% was targeted with a secondary gRNA, and overall indel rates decreased by 25.4±13.4%). Conversely, sites with higher initial indel rates targeted by secondary gRNAs showed larger decreases in total indel rates (for example, at the MMACHC site, a 1-bp insertion with an initial rate of 49.2±3.7% was targeted with a secondary gRNA, and overall indel rates decreased by 48±7%). However, these percent decreases in overall indel rates were not as well-correlated with initial indel rates as the fold-changes in HDR efficiencies. For example, at the ABOB1 site, a 1-bp insertion with an initial rate of 40.5±2.7% was targeted with a secondary gRNA, and the total indel rate decreased only by 12±8% (while the HDR efficiency was improved 1.8±0.4-fold). This relatively small decrease in the total indel rate is partially because the targeted indel was still present with a rate of 12.6±0.4% in the double tap sample (in all other cases, the rates of the targeted indel(s) decreased to below 5%). Additionally, a 2-bp insertion present in the primary gRNA-only experiment at a rate of 0.14±0.02% increased to 6.8±1.1% in the double tap sample. The incomplete elimination of the 1-bp insertion secondary gRNA target in combination with the generation of this new indel product caused the overall indel rate to decrease only slightly. On the other hand, at the PCSK site, a 1-bp insertion indel with an initial rate of 18.1±2.5% was targeted with a secondary gRNA, and the total indel rate decreased by 71±17% (while the HDR efficiency improved only 1.1±0.2-fold). However, while the double tap method does seem to decrease rates of small indels, the frequency of large on-target deletions may be changing 3334. No any large deletions within the sequenced amplicon was observed, but deletions that occur outside of the PCR primer binding sequences would not be detected, and may account for the apparent decrease in small indel efficiencies at certain sites that were not accompanied by a significant increase in HDR rates. Nevertheless, the relative HDR to NHEJ ratios for all sites tested was either within error of the non-double tap samples (at two out of eleven sites) or improved up to 3.8±0.6-fold (FIG. 10). Overall, these data show that the double tap method not only improves HDR efficiencies but may also decrease overall indel rates.

C. Additive Effects Combining Double Tap with Other Methods to Improve Precision Editing Outcomes

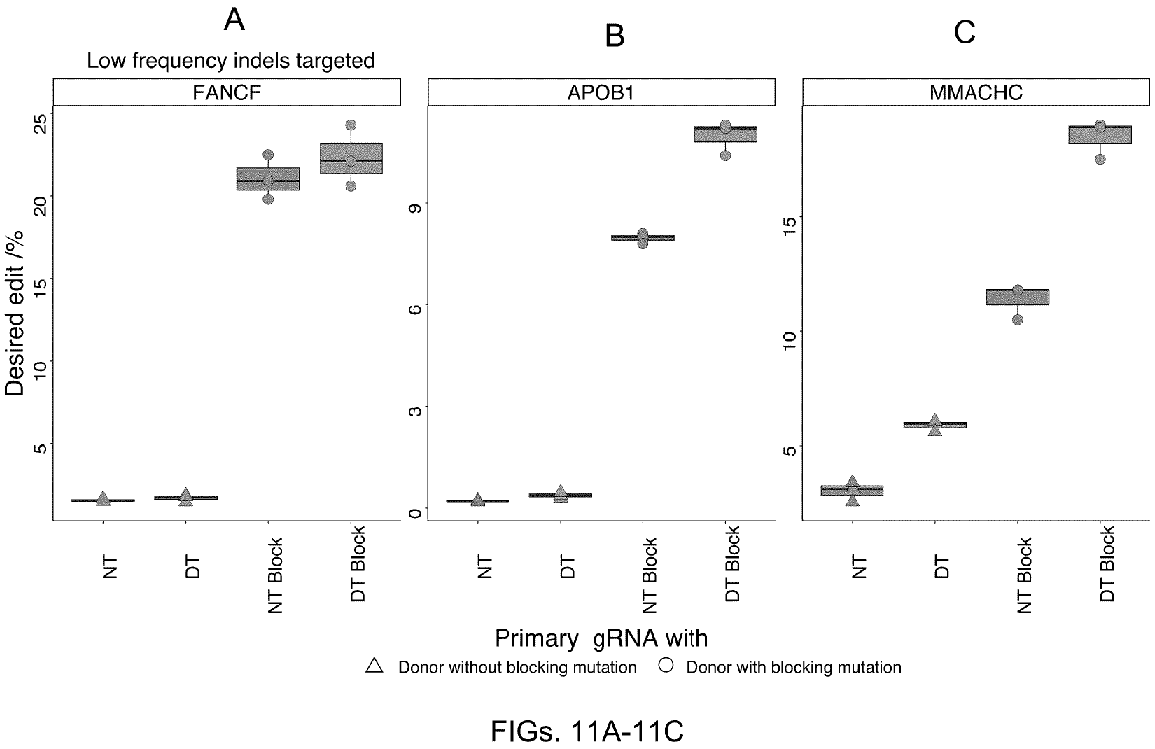

The use of blocking mutations at the PAM or the PAM-proximal region of the protospacer has been shown to improve HDR-mediated genome editing yields¹⁴, and combine this method with the double tap method may improve HDR efficiencies even further. Therefore, the double tap method was tested at the FANCF, APOB1, and MMACHC sites (which were previously tested without blocking mutations, FIG. 1c) using ssODNs identical to those used previously but with additional mutations incorporated to block re-cleavage of the target genomic locus by Cas9 after a successful editing event. Consistent with prior studies, it was found that the use of blocking mutations boosted HDR yields considerably (FIGS. 1c and 2a, and FIG. 11). Furthermore, it was found that the use of secondary gRNAs facilitated similar fold-improvements in HDR efficiencies as observed previously when using ssODNs without blocking mutations. Specifically, double tapping produced a 1.1±0.1-fold improvement at the FANCFsite (compared to 1.1±0.1-fold when using an ssODN lacking a blocking mutation), a 1.4±0.1-fold improvement at the APOB1 site (compared to 1.8±0.4-fold with an ssODN lacking a blocking mutation), and a 1.6±0.1-fold improvement at the MMACHC site (compared to 2.0±0.2-fold with an ssODN lacking a blocking mutation). When comparing HDR efficiencies of samples with primary gRNAs only used with ssODNs without blocking mutations to samples with secondary gRNAs used with ssODNs with blocking mutations, a 14.4±1.2-fold improvement at the FANCFsite, a 53.9±5.8-fold improvement at the APOB1 site, and a 6.1±0.7-fold improvement at the MMACHC site were observed (FIG. 11). While the double tap method can be used independently to improve HDR yields without requiring additional mutations, these data demonstrate that the double tap method can be combined with blocking mutations to further improve HDR efficiencies. Importantly, in both cases HDR rates are improved without perturbing gene expression levels or the cell cycle.

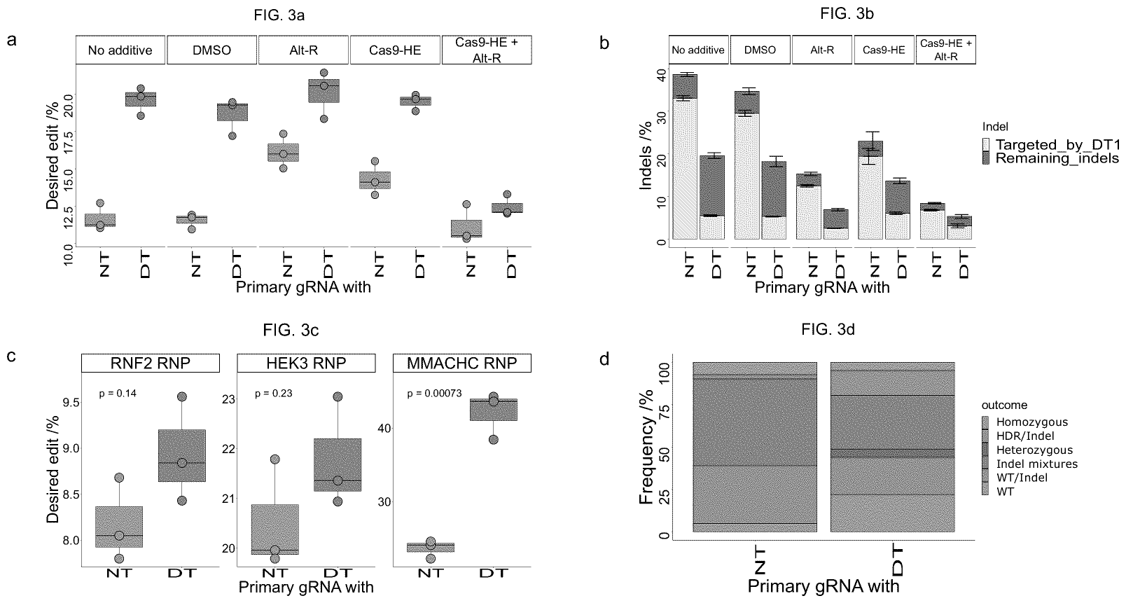

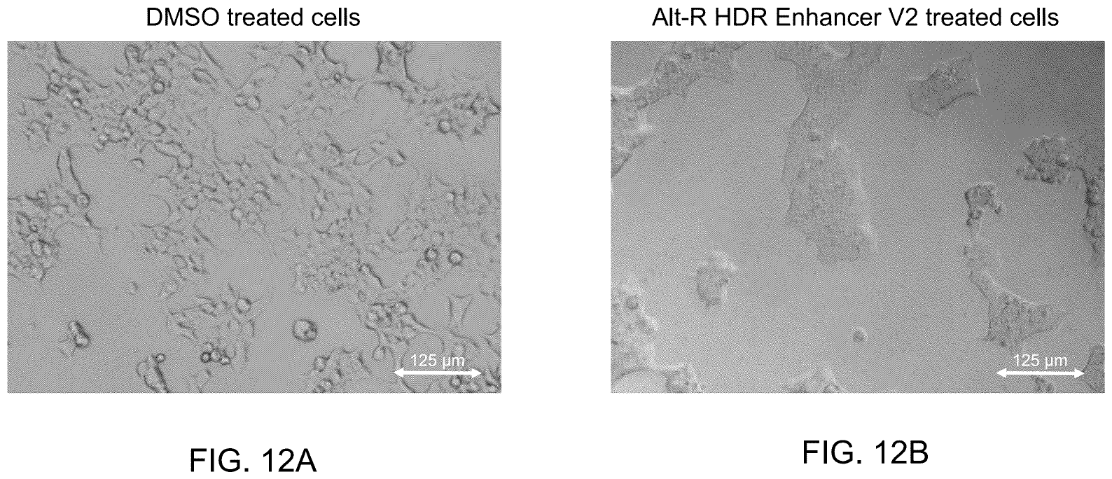

To further investigate potential synergistic effects of the double tap method with existing methods to improve HDR:NHEJ ratios, the double tap method was compared and combined with a small molecule inhibitor of NHEJ and a Cas9-CtIP fusion construct. Specifically, IDT's “Alt-R™ HDR Enhancer V2” (which hereinafter referred to as Alt-R) and the Cas9-HE fusion protein (wherein Cas9 is tethered to the HDR enhancer domain of the CtIP protein) were used and tested at the MMACHC site using primary gRNA with additional non-targeting or secondary gRNA to compare them to and evaluate their additive effects with the double tap method. Both the Alt-R molecule and the Cas9-HE increased HDR rates relative to the wild-type Cas9 (wtCas9) with primary and non-targeting gRNA sample with no additives or dimethyl sulfoxide (DMSO) added (the Alt-R molecule is dissolved in a DMSO solution, FIG. 3a). Specifically, a 1.4±0.1-fold improvement with the Alt-R sample and a 1.2±0.1-fold improvement with the Cas9-HE sample relative to the no additive sample (which was within error of the DMSO sample) were observed. Notably, both samples had absolute HDR rates below that of the wtCas9 double tap sample with no additives (which improved the HDR rate 1.7±0.1-fold compared to the wtCas9 primary and non-targeting gRNA sample, FIG. 3a). Both methods decreased overall indel rates as well (from 38.6±0.5% to 15.3±0.4% with the Alt-R, and to 23.0±2.2% with Cas9-HE), resulting in similar overall indel rates to the wtCas9 double tap sample with no additives (FIG. 3b). However, it is noted that when targeted a particularly high efficiency indel with a secondary gRNA at this site, and other sites with lower efficiency indels may benefit more from the Alt-R molecule of Cas9-HE than they would from the double tap method. Interestingly, combining both HDR enhancer methods (Alt-R and Cas9-HE) with each other did not improve HDR rates (1.0±0.1-fold improvement to the no additive sample, FIG. 3a). Significantly, the combination of either the Alt-R molecule or the Cas9-HE construct with the double tap method further increased precision genome editing compared to their respective primary and non-targeting gRNA sample. Specifically, a 1.7±0.2-fold improvement with the Alt-R double tap sample and a 1.7±0.1-fold improvement with the Cas9-HE double tap sample relative to the no additive sample (which are both within error of the double tap sample with no additives, but the overall indel rates were decreased in these combination treatments, FIG. 3a-3b). were observed. These combinations additionally further reduced the overall indel rates compared to the no additive double tap sample. In particular, the Alt-R double tap combination yielded the lowest overall indel rates (6.9±0.3%, FIG. 3b). However, the usage of the Alt-R molecule induced changes in the morphology of the cells (FIGS. 12a-12b). As these methods manipulate the cell cycle and/or expression levels of DNA repair pathways, the cells' ability to perform native DNA repair functions may be impaired, leading to additional, unwanted genomic modifications elsewhere in the genome. This may be responsible for the significantly reduced editing yields in the Alt-R Cas9-HE combination samples. Importantly, these data show that the double tap method can be combined with additional HDR-enhancing methods to further improve precision genome editing rates and decrease the rates of unwanted indels.

### D. Double Tap Using Cas9 Ribonucleoprotein (RNP) Complexes

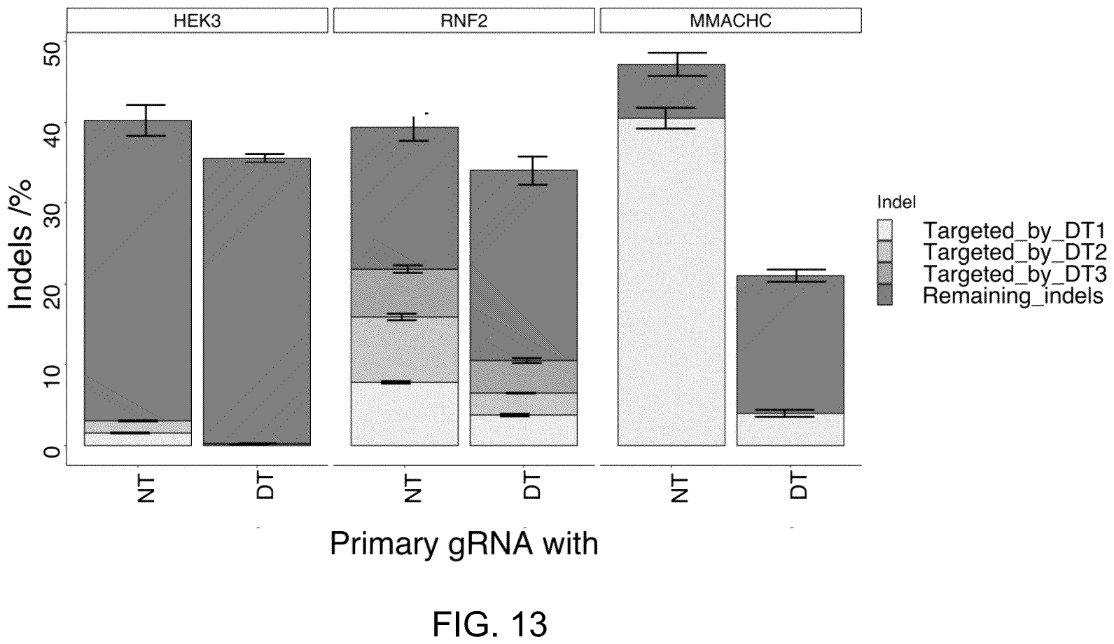

The Cas9:gRNA complex is often delivered into cells as a ribonucleoprotein (RNP) complex due to lower toxicity, decreased off-target editing efficiencies, and enhanced on-target editing efficiencies. To assess if RNP delivery is compatible with the double tap method, HEK293T cells were transfected with purified Cas9 RNP complexes targeting the HEK3, RNF2, or MMACHC sites (using the same primary gRNA and non-targeting or secondary gRNA(s) as used previously) and the same ssODNs as used previously. Similar results were observed when utilizing RNP delivery as those when using plasmid-based delivery; HDR rates increased and rates of indel products decreased (FIG. 3c). The average HDR-mediated genome editing efficiencies improved 1.1±0.1-fold at the HEK3 site, 1.1±0.1-fold at the RNF2 site, and 1.8±0.1-fold at the MMACHC site. a decrease in overall indel rates was also observed for double tap samples, driven by large decreases in introduction efficiencies of the specific indels targeted by the secondary gRNAs. Specifically, the collective indel frequencies of the indels targeted by secondary gRNAs decreased from 3.0±0.1% to 0.2±0.05% at the HEK3 site, from 21.9±0.9% to 10.6±0.4% at the RNF2 site, and from 40.5±1.3 to 4.0±0.4% at the MMACHC site (FIG. 13). These data demonstrate that the double tap method can be implemented with RNP delivery to enhanced HDR efficiencies and decreases unwanted indel frequencies, albeit with slightly less drastic improvements as when using plasmid-based delivery.

### E. Analysis of Zygosity of Double Tap Edited Cells

Isogenic cell lines are useful model systems with which to study the effects of mutations. Generation of such models can often be hampered by “hemizygous-like” clones, in which one allele contains the edit of interest, and the other an indel¹⁴. Therefore, the zygosity of cell lines generated using the double tap method were characterized. HEK293T cells were transfected with ssODN, Cas9-p2A-GFP plasmid, and gRNA plasmids (primary gRNA with non-targeting or secondary gRNA plasmid) to target the MMACHC locus. After 72 hours, individual GFP-positive cells were sorted into separate wells of a well-plate using fluorescence activated cell sorting (FACS) and clonally expanded. Cells were expanded for 21 days, and 41 colonies per experiment (non-targeting or secondary gRNA) were genotyped via NGS. As the HEK293T cell line is pseudotriploid^{35,36} (the MMACHC locus resides on chromosome 1, which is triploid), a variety of zygosities were observed and simplified into the categories of homozygous (all copies have the HDR edit, with no indels), heterozygous (mixture of wild-type and HDR edits, with no indels), HDR/indel products (mixture of HDR edits and indels), indel mixtures (all copies have indels), WT/indel (mixture of wild-type and indels), and WT (all copies unedited). The breakdown can be seen in FIG. 3d. Importantly, while only two homozygous colonies were identified from the primary plus non-targeting gRNA samples, nine were identified from the double tap samples, representing an increase in frequency from 5% to 22% using the double tap method, or a >4-fold improvement. Consistent with previous reports¹⁴, no heterozygous clones were obtained from the primary plus non-targeting gRNA sample. However, two (2) heterozygous clones were obtained in the double tap samples, which may have originated from a WT/indel-containing cell. Large decreases in the number of clones were also observed with indel mixture genotypes and HDR/indel genotypes in the double tap samples compared to the primary plus non-targeting samples (FIG. 3d). Specifically, the frequency of indel mixture colonies decreased from 51% to 32%, and the HDR/indel-mixed genotype clone frequency decreased from 34% to 22%. These data show that the double tap method can be used to improve the frequency of homozygous and heterozygous isogenic clones when using HDR to generate disease-relevant model systems.

F. Double Tap Using dsDNA Donor Templates to Perform Gene Knock-In

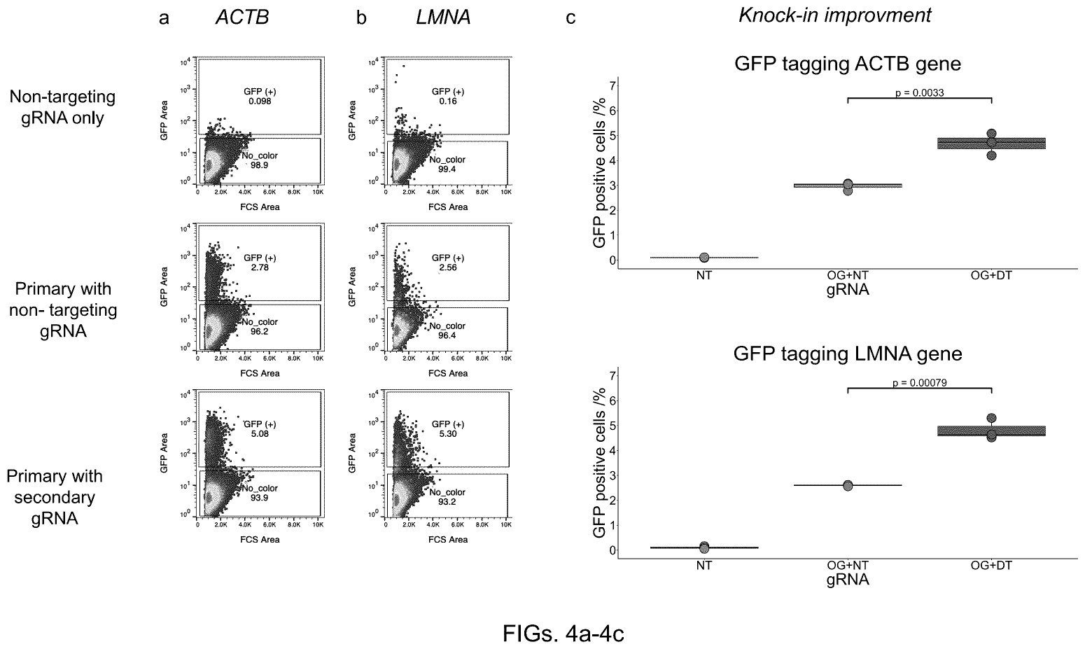

The installation of small modifications is typically carried out using ssODNs as a donor template. However, the introduction of larger (typically, >100 bps) modifications, such as knocking-in a gene to a targeted locus, is usually carried out using dsDNA donor templates. These two precision genome editing methods have been shown to function via different mechanisms (ssODN-mediated knock-in occurs in a Rad51-independent manner, while dsDNA donor-mediated knock-in occurs in a Rad51-dependent manner⁷). To determine if the double tap method was compatible with both, the double tap method was used to knock-in the green fluorescent protein (GFP) gene just after the start codon of two different genes (ACTB and LMNA) using dsDNA donor plasmids. Donor template and primary gRNA designs that had been described previously for ACTB²⁵ were used, as well as for LMNA²⁷. To design secondary gRNAs, HEK293T cells were first transfected with plasmids encoding Cas9 and primary gRNA, then the genomic loci of interest was analyzed with NGS after 72 hours to determine the indel product distribution (FIGS. 8a & 8r). One secondary gRNA was designed for each site, as the initial rates of the most frequent indel product at each site was >4 times larger than that of the next most frequent indel product (FIGS. 8a & 8r). HEK293T cells were then transfected with plasmids encoding the dsDNA donor, Cas9, and gRNA (either non-targeting gRNA only, primary and non-targeting gRNAs, or primary and secondary gRNAs). Knock-in of GFP was monitored by flow cytometry fourteen days post-transfection, after continuous passaging of the cells. At this time, all negative control samples (untransfected cells, and cells transfected with Cas9, dsDNA donor, and non-targeting gRNA only) showed minimal GFP fluorescence (<0.2% of cells with GFP fluorescence). GFP knock-in to the ACTB gene increased 1.6±0.1-fold when using the double tap method, and GFP knock-in to the LMNA gene increased 1.9±0.1-fold when using the double tap method (FIG. 4). These data show that the double tap method can be used successfully independently of the donor template type (ssODNs and dsDNA templates).

### G. Double Tap in K562 and HeLa Cell Lines

The double tap method was also tested in human erythroleukemic (K562) and human cervical cancer (HeLa) cell lines using the APOB1 and MMACHC primary gRNAs and secondary gRNAs that previously validated in HEK293T cells. Cells were transfected with ssODN, Cas9-p2A-GFP plasmid, and gRNA plasmids. After 72 hours, GFP positive cells were enriched using fluorescence activated cell sorting (FACS) and analyzed by NGS (FACS enrichment was used due to the significantly lower transfection efficiencies of these cell lines as compared to HEK293T cells).

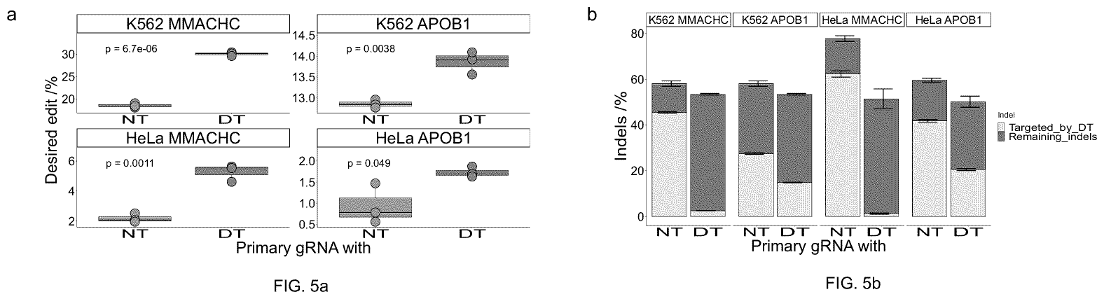

At the MMACHC site, the average HDR-mediated genome editing efficiency improved 1.6±0.04-fold in K562 cells and 2.4±0.3-fold in HeLa cells (compared to 1.6±0.1-fold in HEK239T cells, FIG. 5a). At the APOB1 site, the average HDR-mediated genome editing efficiency improved 1.1±0.02-fold in K562 cells and 1.9±0.8-fold in HeLa cells (compared to 1.4±0.1-fold in HEK239T cells, FIG. 5a). The slight differences in fold-change values for a given target site among the different cell lines may be attributed to the differences in initial rates of the double tap-targeted indels (FIGS. 2d and 5b). While in general indel sequences are reproducible among different cell lines, their relative introduction rates fluctuate³² (FIGS. 8b & 8s), which impacts the effect of the double tap method. Furthermore, near complete disappearance of the double tap-targeted indel at the MMACHC site was observed (in HeLa cells, the rate of this indel dropped from 62.3±1.4% to 1.2±0.3% after double tapping, with similar results in K562 cells FIG. 5b), while the indel at the APOB1 site persisted (in HeLa cells, the rate of this indel dropped from 41.8±0.6% to 20.5±0.4% after double tapping, with similar results in K562 cells, FIG. 5b). This incomplete disappearance of the APOB1 double tap-targeted indel is potentially responsible for the reduced fold-improvement observed at this site and suggests that the double tap method is most effective when targeting indel sequences with high rates, and when the corresponding secondary gRNAs are highly efficient at targeting their respective sequences. CRISPick³⁷ was used to analyze the predicted efficiencies of all secondary gRNAs, but did not find the efficiency score of the APOB1 secondary gRNA to be significantly lower than those of the secondary gRNAs that effectively targeted their respective indels. Therefore, explicitly testing all secondary gRNAs for efficiency and re-designing if necessary are recommended. In the case of the APOB1 secondary gRNA, an alternative protospacer/PAM could be used to target this indel (FIG. 14) and may facilitate greater fold-improvements. Importantly, these data show that the double tap method can be used in a variety of human cell lines.

### H. Disease Modeling and Comparison to Prime Editing

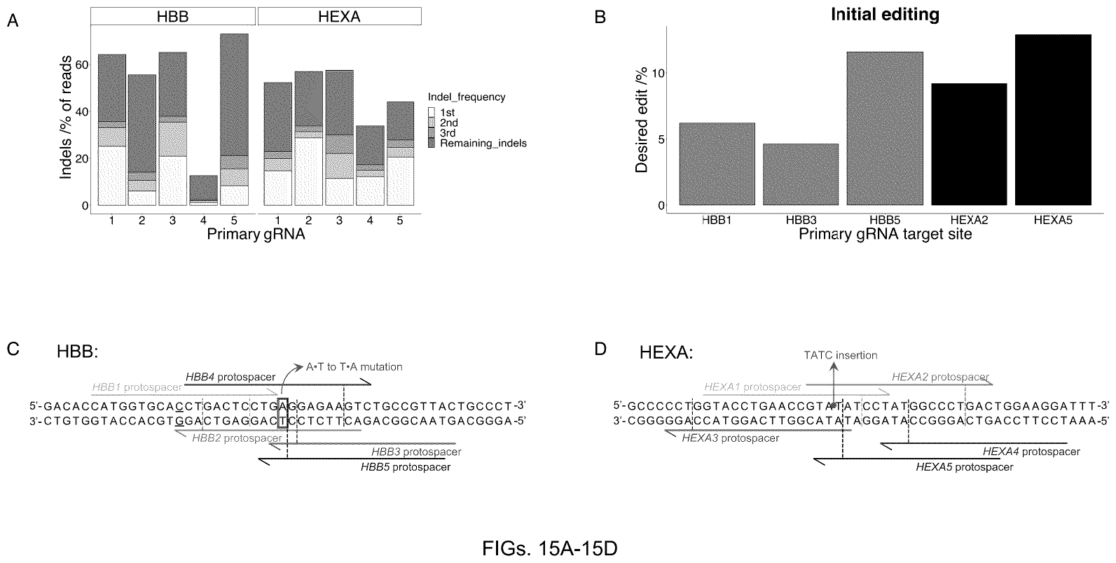

The ability of the double tap method to install two disease-relevant mutations to demonstrate its utility for generating disease models and to compare its performance with that of prime editing was also tested. The sickle cell-relevant mutation E6V in hemoglobin, which is an A to T transversion mutation in the HBB gene, and the Tay-Sachs disease-relevant TATC 4-bp insertion in the HEXA gene were chosen as pegRNA-nicking gRNA combinations have already been optimized to introduce these mutations with prime editing. Five potential primary gRNAs (referred to as HBB1, HBB2, etc. and HEXA1, HEXA2, etc. primary gRNAs) were designed for each site using inDelphi to aid in identifying “high precision” protospacers (i.e., those predicted to produce outcomes in which the top three indel sequences would represent >40% of products) with cut sites within 15 bp of the intended mutation (FIGS. 15A-15D). HEK293T cells were transfected with plasmids encoding Cas9-NG (a variant of Cas9 that has a relaxed PAM requirement of NG instead of NGG) and each of these candidate primary gRNAs, lysed the cells after 72 hours, and analyzed genomic loci of interest with NGS and CRISPResso2. The total indel rates, as well as the individual introduction efficiencies of the top three indel sequences acquired with each of the candidate primary gRNAs are shown in FIG. 15A. The sequences and efficiencies of the individual indels, along with the inDelphi predictions, are shown in FIGS. 8e-8i & 81-8p. It was found that five out of the ten protospacers closely matched the inDelphi predictions; that is, these gRNAs (HBB1, HBB3, HEXA2, HEXA4, and HEXA5) generated the top three inDelphi predicted indels, and their collective introduction efficiencies represented >40% of all repair products. One protospacer (HBB4) was inefficient and therefore precluded an accurate analysis of indel products, two protospacers (HBB2and HEXA1) produced the top three inDelphi predicted indels, but their collective introduction efficiencies represented <25% of all repair products, and two protospacers (HBB5 and HEXA3) produced only one or two of the top three inDelphi predicted indels. Overall, inDelphi is recommended to be used to guide protospacer design for identifying “high precision” protospacers, but additional tests for multiple gRNAs for a given target site are also recommended, given the 50% success rate observed here (and with the protospacers tested earlier). Two primary gRNAs were chosen per site to proceed with preliminary double tap experiments; primary gRNAs that produced high frequencies of a (preferably) single indel product were chosen (the HBB1, HBB3, HEXA2, and HEXA5 primary gRNAs). ssODNs were then designed to be compatible with both primary gRNA options for each site (cut sites were within 15 bases of each other). In the case of the HBB mutation, a silent blocking mutation was added to boost HDR efficiencies. For the HEXA mutation, additional silent mutations were not deemed necessary as the 4-bp insertion disrupted both protospacers. To assess initial HDR efficiencies when using these primary gRNAs, HEK293T cells were transfected with ssODNs and plasmids encoding Cas9 and primary gRNA. After 72 hours, cells were lysed and analyzed via NGS and CRISPResso2 to determine HDR and indel introduction efficiencies. A low (<5%) HDR efficiency was observed with the HBB3 primary gRNA (FIG. 15B), the experiment was then repeated to assess the initial HDR efficiency with the next best candidate primary gRNA (the HBB5 primary gRNA). The initial HDR efficiency with the HBB5 primary gRNA was almost 3-fold higher, so the experiment was proceeded with this primary gRNA. Indeed, using the equation from FIG. 2b, an improvement of 1.6-fold for HBB3 (which would result in an increase in HDR efficiency from 4.6% to 7.4%), and an improvement of 1.3-fold for HBB5 (which would result in an increase in HDR efficiency from 11.6% to 15.3%) were estimated. This experiment highlights the importance of balancing the initial HDR efficiency with the indel distribution of a putative primary gRNA when assessing its potential for the double tap method.

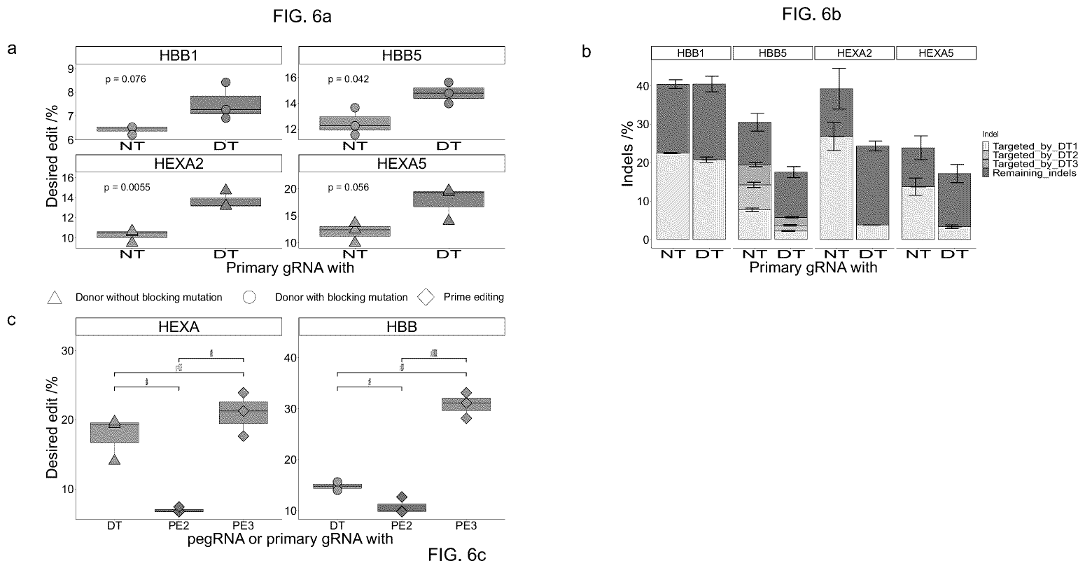

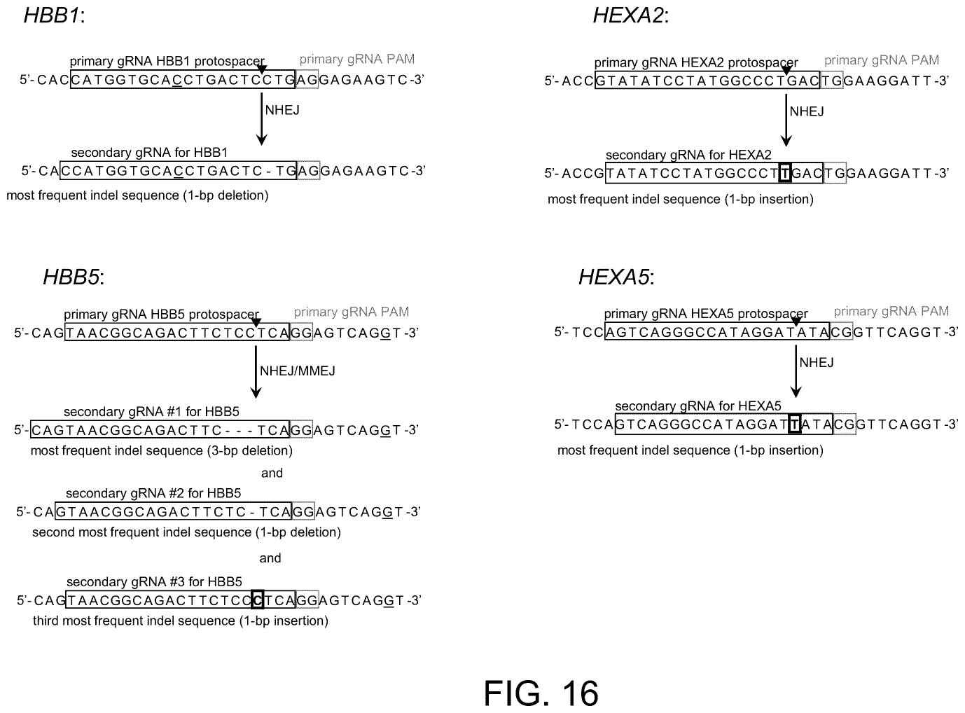

One secondary gRNA was then designed for both HEXA primary gRNAs, one secondary gRNA was designed for the HBB1 primary gRNA, and three secondary gRNAs were designed for the HBB5 primary gRNA (FIG. 16), and HEK293T cells were then transfected with ssODNs and plasmids encoding Cas9, primary gRNA, and either non-targeting gRNA or secondary gRNA(s). After 72 hours, cells were lysed and analyzed via NGS and CRISPResso2 to determine HDR and indel introduction efficiencies. Improvements in all four double tap samples were observed as compared to samples without secondary gRNAs. Using the equation from FIG. 2b, a rough estimate of the expected improvement was first calculated. While a perfect match was not expected as the coefficient of determination (R²) was only 0.81, improvements of 1.3-fold for the HBB1 secondary gRNA, 1.3-fold for the HBB5 secondary gRNAs, 1.4-fold for the HEXA2 secondary gRNA, and 1.2-fold for the HEXA5 secondary gRNA were calculated given the initial indel rates of the respective indels targeted with these secondary gRNAs. Improvements of 1.2±0.1-fold for the HBB1 secondary gRNA, 1.2±0.1-fold for the HBB5 secondary gRNAs, 1.3±0.1-fold for the HEXA2 secondary gRNA, and 1.5±0.3-fold for the HEXA5 secondary gRNA were observed (FIG. 6a), all of which are within error of the calculated values. As with previous experiments, decreases in both the total indel frequencies as well as the efficiencies of the specific indels targeted by the secondary gRNAs were observed in all cases except the HBB1 sample (FIG. 6b). The average absolute total indel frequency as well as that of the double tap targeted indel did not change in this sample, suggesting the secondary gRNA may be inefficient at facilitating Cas9 binding and/or cleavage (although we did observe an increase in the HDR efficiency for this sample). However, in all other samples robust decreases were observed in overall indel frequencies (a decrease from 30.5±2.3% to 17.6±1.4% for the HBB5 secondary gRNAs sample, from 30.5±2.3 to 17.6±1.4% for the HEXA2 secondary gRNA sample, and from 39.2±5.3% to 24.3±1.3% for the HEXA5 secondary gRNA sample). These data further demonstrate the ability of the double tap method to simultaneously enhance HDR-mediated genome editing efficiencies and decrease overall indel rates using gRNAs targeted to high frequency indel sequences. Furthermore, this increase in genome editing precision does not require cell perturbations of any kind and can easily be implemented by simply including additional gRNAs in classic HDR experiments. Importantly, the enhanced precision of this method would greatly aid researchers with generating disease models.

To further compare the performance of the double tap method to that of prime editing, previously reported pegRNAs and nicking gRNAs to install these mutations²⁴ were used. It is important to mention that these two pegRNA-nicking gRNA combinations were extensively optimized; specifically, to identify the HEXA combination, 43 pegRNAs and three nicking gRNAs were tested (for a total of 129 different combinations tested). In contrast, for the double tap method, only five primary gRNAs were screened per site, and all double tap experiments that were performed displayed improvements in HDR efficiency. HEK293T cells were transfected with plasmids encoding PE2 and pegRNA only (PE2 sample), or pegRNA and nicking gRNA (PE3 sample). After 72 hours, cells were lysed and analyzed via NGS and CRISPResso2 to determine the efficiency of introduction of the intended edit. We found that intended edit introduction efficiencies with PE2 were lower than that with the double tap method (10.8±1.3% at the HBB site, and 7±0.3% at the HEXA site), while those with PE3 were similar at the HEXA site (20.9±2.6%), and higher at the HBB site (30.8±2.1%, FIG. 6c). These results demonstrate the utility and simplicity of the double tap method for disease modeling.

### I. Off-Target Editing Assessment

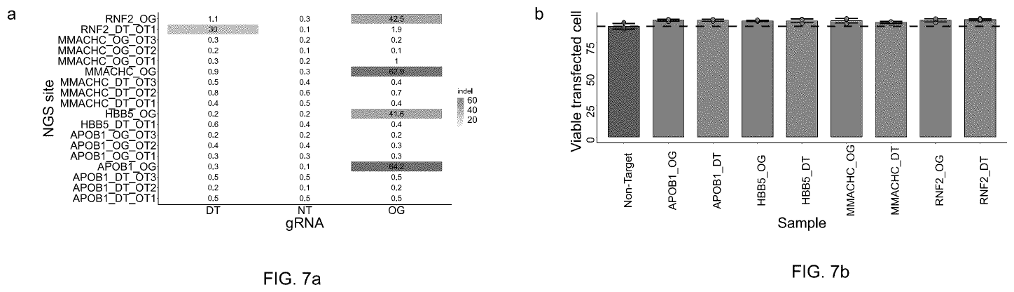

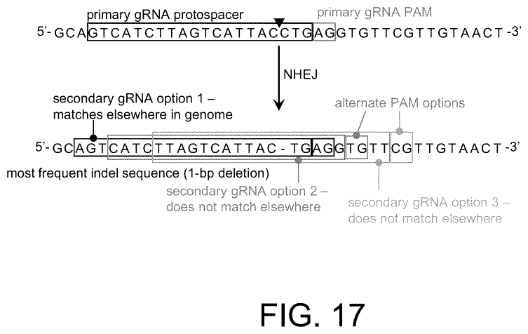

It was recognized that a potential drawback of the double tap method is the possibility of introducing DSBs at additional off-target sites compared to when only a single gRNA is used. Indeed, the introduction of multiple DSBs within a given cell can cause cytotoxicity and chromosomal rearrangements³⁸⁻⁴¹. Therefore, all secondary gRNAs used in this study were first analyzed for potential full matches with other sites in the human genome. It was found only one secondary gRNA (one of the RNF2 secondary gRNAs) that fully matched a location in the human reference genome that is directly next to an NGG PAM sequence (this locus is labeled RNF2_DT_OT1). To quantify editing at this site, HEK293T cells were transfected with plasmids encoding Cas9 and either a non-targeting gRNA, the RNF2 primary gRNA, or the RNF2 secondary gRNA, then cells were lysed after 72 hours, and the primary on-target and the secondary matched loci of all samples were analyzed for indel frequencies using NGS and CRISPResso2. Unsurprisingly, a 30% indel introduction efficiency was observed with the RNF2 secondary gRNA at its fully matched locus (FIG. 7a). Additionally, the RNF2 primary gRNA (which differs from the RNF_DT_OT1 locus sequence by a 1-bp deletion) introduced indels at this locus with an efficiency of 1.9% (FIG. 7a). These data demonstrate that secondary gRNAs should always be analyzed for matching sequences elsewhere in the genome when using this method. When/if this occurs, use another PAM sequence nearby (if possible) to target a given indel sequence (FIG. 17).

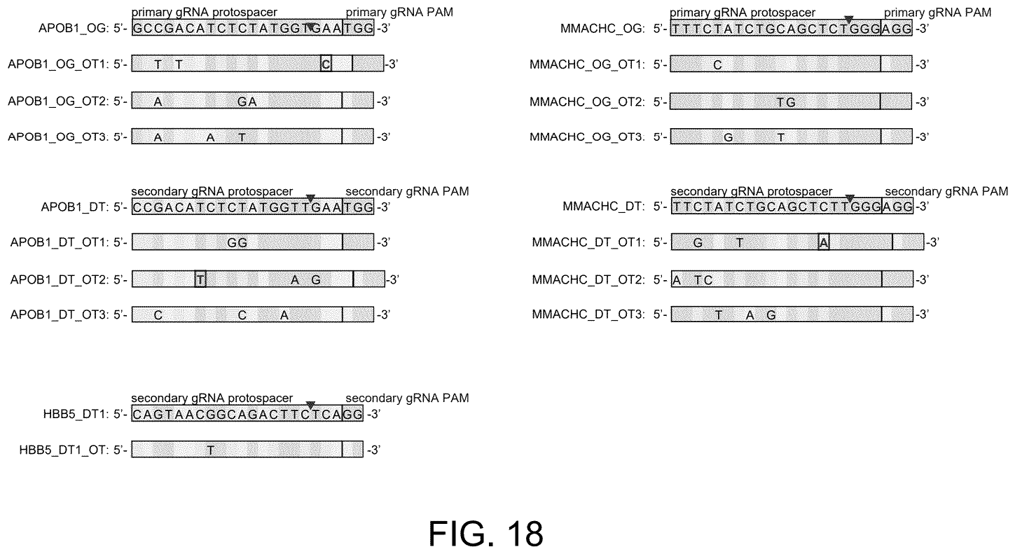

Additionally, all secondary gRNAs were analyzed for putative off-targets containing a single mismatch using Cas-OFFinder⁴², as these types of off-targets are the most common⁴³. It was found that only one secondary gRNA (one of the HBB5 secondary gRNAs) had a potential off-target with a single mismatch (this locus is labeled as HBB5_DT_OT1). Two additional sets of primary and secondary gRNAs (those for the APOB1 and MMACHC sites) were then chosen and their predicted off-target sites in silico were identified and analyzed using a combination of Cas-OFFinder (to identify putative off-target sites with up to five mismatches and three bulges)⁴² and Benchling⁴⁴ (to assess their predicted off-target scores). The MMACHC primary gRNA had the highest predicted off-target site (labelled as MMACHC_OG_OT1) with only a single mismatch, and a predicted off-target score of 100 (out of a highest possible score of 100). All other putative off-target sites had predicted off-target scores of less than 6 (the closest predicted off-target sites had at least two mismatches). Nevertheless, three predicted off-target sites were selected for each gRNA based off these two analyses for both the primary gRNAs (which were called as the original guide off-target sites, or OG_OT), as well as for the secondary gRNAs (which were called as the double tap guide off-target sites, or DT_OT, FIG. 18). HEK293T cells were transfected with plasmids encoding Cas9 and either a non-target gRNA, the primary gRNA, or the secondary gRNA. Cells were lysed after 72 hours, and the on-target and all off-target loci were analyzed for indel frequencies using NGS and CRISPResso2. Surprisingly, no indel rates above non-targeting controls were observed at any off-target loci (FIG. 7a). While an unbiased off-target identification method (such as GUIDE-seq, Digenome-seq, or DISCOVER-seq^{30, 43, 45}) is required to fully evaluate the extent of off-target editing with the double tap method, these data suggest that the extent of off-target editing with the double tap method is similar to that of experiments using single gRNAs, unless the secondary gRNA fully matches a site in the genome.

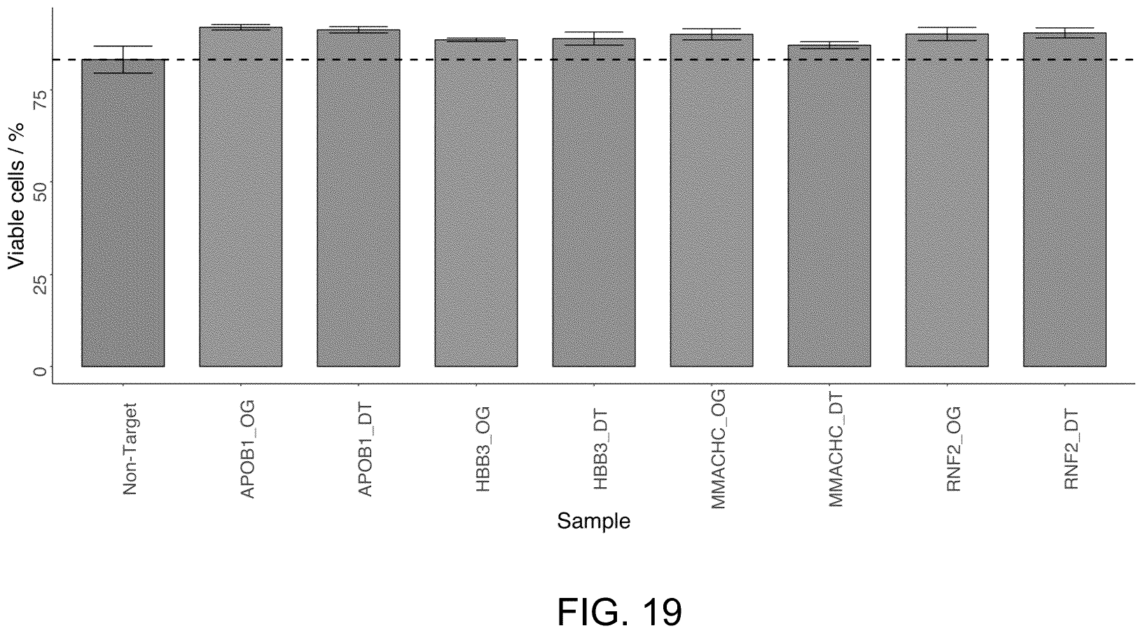

Since as previously stated, multiplexed DSB introduction can cause cytotoxicity, if the use of secondary gRNAs causes significant off-target editing, reduced viability of the cells would be observed. Therefore, to further qualify cell viability, primary and secondary gRNAs for the RNF2 (in which case all three secondary gRNAs, including the one that has a fully matched site in the genome were used), HBB5, APOB1 and MMACHC sites were chosen, as these had been previously evaluated for indel introduction efficiencies at putative off-target sites. HEK293T cells were then transfected with plasmids encoding Cas9-P2A-GFP (to allow for identification of transfected cells using GFP fluorescence) and gRNA (non-targeting gRNA only as a control, primary and secondary gRNA, or primary and non-targeting gRNA) and stained the cells with propidium iodide to monitor cell viability after 72 hours (FIG. 7b). No decrease in viability was observed as compared to the non-targeting gRNA samples; all samples had >80% total viability (FIG. 19), with >90% viability of transfected cells (as determined by cells with GFP fluorescence, FIG. 7b), even with the RNF2 sample, which utilized three secondary gRNAs. These data show that the use of secondary gRNAs does not introduce off-target DSBs at a level that impacts cell viability.

Off-target editing remains a key challenge for all genome editing agents, and the use of high-fidelity Cas enzymes has been shown to alleviate off-target editing by CRISPR nucleases⁴⁶⁻⁵¹. The use of these high-fidelity variants in combination with off-target score prediction software could minimize unwanted off-target editing for the double tap method. However, in silico off-target identification has major limitations, and thus in cases where off-target editing must be completely eliminated, the use of unbiased experimental methods to identify putative off-target edits would be required.

### Discussion & Summary

The studies in EXAMPLE 1 of the present disclosure describe the development and characterization of the double tap method to improve HDR-mediated genome editing efficiencies in human cell lines. The double tap method takes advantage of the modularity of the Cas9 system and the reproducibility of indel sequences by using additional secondary gRNAs that target unwanted, high-frequency indel sequences generated during the end-joining repair of DSBs. In this manner, the double tap method provides researchers with a second chance at a successful HDR event when performing precision genome editing at a locus of interest. Importantly, the double tap method does not perturb the cell by modulating gene expression levels or synchronizing the cell cycle phase which may introduction additional artifacts to the system being studied.

In this EXAMPLE 1, the impact of the double tap method was characterized by first quantifying the improvements in HDR-mediated genome editing efficiencies following the use of secondary gRNAs targeted to indel sequences with a wide range of frequencies (ranging from 4.8±0.2% to 49.2±3.7%). A direct correlation was found between the fold-improvement afforded by this method and the collective frequencies of the indels targeted by secondary gRNAs; this correlation allows a user to estimate a fold-change in HDR efficiency for the double tap method following analysis of indel distribution frequencies for a particular gRNA of interest. It is noted that initial HDR efficiencies can vary drastically depending on the primary gRNA used, and thus this value needs to be balanced with the estimated fold-change to identify the ideal conditions to maximize absolute HDR efficiencies. Overall indel rates were found to be decreased when using the double tap method, mostly driven by large decreases in the frequencies of the indels that were targeted by secondary gRNAs. Overall, this led to enhancements in HDR:NHEJ ratios up to 3.8-fold. However, the targeted amplicon sequencing methods may miss larger deletion products that occur outside the sequencing primer binding sites.

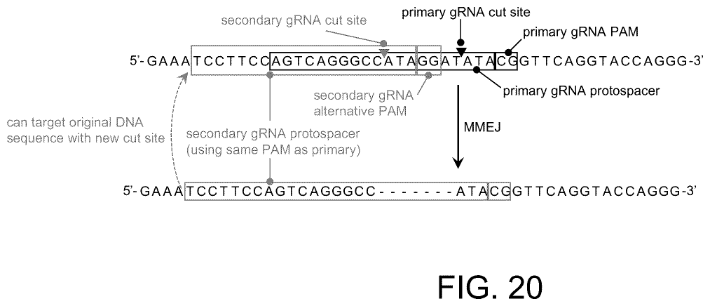

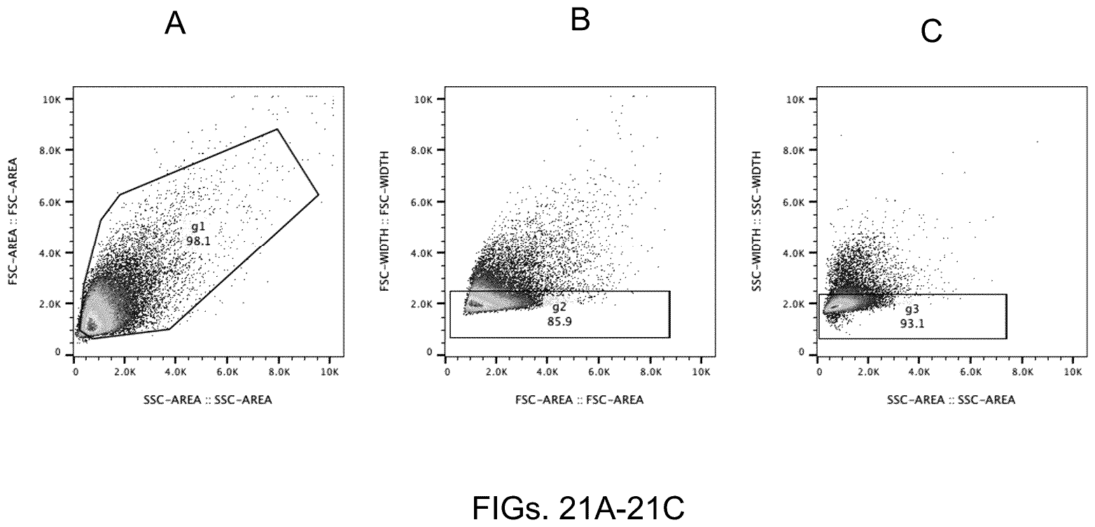

The double tap method was found to be compatible with multiple cell lines, RNP delivery, and with both small modifications (using ssODN donors) and large insertions (using dsDNA donors). The design of secondary gRNAs is straight-forward when 1-bp insertions or deletions are targeted, in which case the original PAM can be used, and the resulting secondary gRNA will rarely match the original sequence. However, in certain instances when small deletions (likely facilitated by MMEJ) were targeted, using the original PAM would result in a secondary gRNA that could target the original DNA sequence, but with an unwanted alternate cut site (FIG. 20). In these cases, unwanted targeting should be avoided by using a secondary gRNA with an alternate PAM (FIG. 20 for an example). Overall, it is important to analyze each putative secondary gRNA for a full match with the original target sequence, or indeed with any other locations in the genome (as with the RNF2 example).

Overall, this EXAMPLE 1 describes that the double tap method was tested with 23 different primary protospacer sequences and compared their experimentally determined indel sequence distribution outcomes with their inDelphi predictions (FIGS. 8a-8w). Sixteen of the tested primary gRNAs are predicted to be “high precision” protospacers by inDelphi (i.e., those predicted to produce outcomes in which the top three indel sequences would represent >40% of products). Out of these 16 gRNAs, ten of them were experimentally determined to be “high precision”, with the same three inDelphi-predicted indel sequences representing >40% of repair products. Due to this high rate (>50%) of successfully predicting “high precision” sites, it is recommended using inDelphi to guide the design of protospacers to use with the double tap method, with additional testing at least 2-3 primary gRNAs per experiment.

EXAMPLE 1 further demonstrates that the double tap method can be combined with existing HDR-enhancing methods to further improve precision genome editing efficiencies. Combining the use of secondary gRNAs with additional blocking mutations on the ssODN (to prevent Cas9 from re-cutting the target site after a successful HDR event) was found to produce additive improvements in HDR efficiencies. As neither of these methods disturb the cell cycle or DNA repair protein levels, this represents a simple and robust non-perturbative method for improving precision editing outcomes. Further, it is also demonstrated that the double tap method can be combined with DNA repair pathway alteration methods to achieve higher HDR:NHEJ ratios compared to using any of these strategies in isolation. The double tap method represents a simple yet effective strategy that can be effortlessly implemented into existing HDR-enhancing pipelines to further improve genome editing outcomes.

Moreover, the utility of the double tap method for generating of isogenic cell lines was also demonstrated. Overall success rates of generating homozygous and heterozygous cell lines were improved, as the secondary gRNAs provides a “second chance” to convert indel-containing alleles into the desired edit. This improvement would allow for a decrease in the number of colonies screened during isogenic cell line generation, as well as an increase in the throughput of cell line generation, which is incredibly valuable for laboratories studying the functional effects of genetic variants. This method could be particularly useful for genome editing in organisms with high chromosomal copy numbers such as plants or applications that cannot take advantage of precision editing-enhancing strategies such as base editing, prime editing, and cell cycle/DNA repair manipulation, including gene drive applications. In fact, the double tap method has been applied to improve gene drive efficiencies by recycling resistance alleles.

It was also demonstrated the utility of the double tap method by installing two disease-relevant mutations (an A to T point mutation in the HBB gene that causes sickle cell disease, and a 4-bp insertion in the HEXA gene that causes Tay-Sachs disease). For both mutations, secondary gRNAs were identified to boost HDR efficiencies. The double tap method can therefore be easily integrated into researchers' current HDR experiments by simply analyzing their DNA sequencing data to identify high-frequency indel products. For experiments such as disease modeling (particularly for the generation of isogenic cell lines), absolute HDR rates are often the most important factor, and dictate whether homozygous variants can be obtained. The double tap method was shown to improve HDR yields up to 2.4-fold in the present disclosure, and because fold-changes can be estimated based on the initial indel frequencies, HDR rates can potentially be modulated if heterozygous models are desired. The decrease in indel rates facilitated by the double tap method of the present disclosure is also an important factor and can help to avoid generating cells in which the mutation of interest is present at one allele and an indel is present at the other. Enhancements in absolute HDR efficiencies are invaluable for modeling of polygenic disorders, in which the introduction of multiple mutations is necessary. In these cases, the increase in likelihood of successfully generating the model is proportional to the product of the individual increases in HDR rates for each mutation.

Off-target editing is always a factor to consider with genome editing experiments and the usage of additional gRNAs increases the number of potential off-target edits, and therefore the possibility of translocations, large-scale deletions, and chromothripsis. This scales with the number of gRNAs, thus experiments that require multiple secondary or tertiary gRNAs have an increased probability of suffering from off-target issues. While in silico off-target prediction tools have been developed and can identify certain putative off-target loci for a given gRNA (including secondary gRNAs), for experiments in which off-target editing is unacceptable, each gRNA needs to be individually assessed using unbiased methods. High-fidelity Cas9 variants have also been used to reduce or eliminate off-target editing in DSB-reliant genome editing experiments, and these mutants could also be used successfully with the double tap method. It is imperative to analyze secondary gRNAs to assess if they are a perfect match with other sites in the genome prior to using them. If this is the case, re-designing the secondary gRNA to use a different PAM nearby is recommended if this is possible (FIG. 17 for an example). Nevertheless, for each experiment, an analysis of the risks (in terms of potential off-target editing) versus the benefits (the extent to which a secondary gRNA could enhance the HDR efficiency) of the double tap method will need to be performed by the researcher.

There are now a variety of “next-generation” genome editing tools for researchers to choose from, such as base editors and prime editors, and each editor comes with its own unique pros and cons. When directly compared the double tap method to prime editing to introduce small modifications, it was found that with minimal optimization, the double tap method can be used to approach PE3 efficiencies and surpass PE2 efficiencies. A drawback of prime editing is the requirement of extensive optimization of the length of the primer binding region and the reverse transcription template portions of the peg RNAs to find a combination with satisfactory efficiency for each protospacer option (and there are often multiple protospacer options for a given modification of interest). Additionally, again with minimal optimization, the efficiencies of GFP knock-in with the double tap method were improved up to 90%. Next-generation genome editing technologies such as base editing and prime editing are unable to facilitate such large insertions. Overall, a major benefit of the double tap method disclosed herein is the simplicity of its implementation; a handful of candidate primary gRNAs can be tested and analyzed for initial HDR efficiencies and indel distributions, and fold-changes can then be estimated to identify the optimal primary-secondary gRNA combination to maximize HDR yields. Overall, this significantly reduces the time and resources required for construct optimization as compared to prime editing.

In summary, the double tap method disclosed in EXAMPLE 1 presents researchers with an easily implemented method to increase HDR-mediated genome editing efficiencies using a combination of a primary gRNA that produces high frequency indel products with a secondary gRNA that targets these indel sequences. A major benefit of the double tap method disclosed herein is its ease of integration with any previously developed HDR system; minimal optimization is required. The double tap method disclosed herein can be used for boosting efficient genome editing in agriculture, plants, animals (e.g., fruit fly, mice, rats, etc.), fungi, mammalian cells, animal germlines and embryos, and/or in vivo animal models for human diseases.

### REFERENCES FOR EXAMPLE 1

1. Jinek, M. et al. A Programmable Dual-RNA-Guided DNA Endonuclease in Adaptive Bacterial Immunity. Science 337, 816-821 (2012).
2. Ranjha, L., Howard, S. M. & Cejka, P. Main steps in DNA double-strand break repair: an introduction to homologous recombination and related processes. Chromosoma 127,187-214 (2018).
3. Mali, P. et al. RNA-Guided Human Genome Engineering via Cas9. Science 339, 823-826 (2013).
4. Cong, L. et al. Multiplex Genome Engineering Using CRISPR/Cas Systems. Science 339, 819-823 (2013).
5. Jinek, M. et al. RNA-programmed genome editing in human cells. eLife 2, e00471 (2013).
6. Cho, S. W., Kim, S., Kim, J. M. & Kim, J.-S. Targeted genome engineering in human cells with the Cas9 RNA-guided endonuclease. Nat. Biotechnol. 31, 230-232 (2013).
7. Yeh, C. D., Richardson, C. D. & Corn, J. E. Advances in genome editing through control of DNA repair pathways. Nat. Cell Biol. 21, 1468-1478 (2019).
8. Liu, M. et al. Methodologies for Improving HDR Efficiency. Front. Genet. 9, 691 (2019).
9. Liang, X., Potter, J., Kumar, S., Ravinder, N. & Chesnut, J. D. Enhanced CRISPR/Cas9-mediated precise genome editing by improved design and delivery of gRNA, Cas9 nuclease, and donor DNA. J. Biotechnol. 241, 136-146 (2017).
10. Yang, L. et al. Optimization of scarless human stem cell genome editing. Nucleic Acids Res. 41, 9049-9061 (2013).
11. Richardson, C. D., Ray, G. J., DeWitt, M. A., Curie, G. L. & Corn, J. E. Enhancing homology-directed genome editing by catalytically active and inactive CRISPR-Cas9 using asymmetric donor DNA. Nat. Biotechnol. 34, 339-344 (2016).
12. Aird, E. J., Lovendahl, K. N., St. Martin, A., Harris, R. S. & Gordon, W. R. Increasing Cas9-mediated homology-directed repair efficiency through covalent tethering of DNA repair template. Commun. Biol. 1, 54 (2018).
13. Savic, N. et al. Covalent linkage of the DNA repair template to the CRISPR-Cas9 nuclease enhances homology-directed repair. eLife 7, e33761 (2018).
14. Paquet, D. et al. Efficient introduction of specific homozygous and heterozygous mutations using CRISPR/Cas9. Nature 533, 125-129 (2016).
15. Lin, S., Staahl, B. T., Alla, R. K. & Doudna, J. A. Enhanced homology-directed human genome engineering by controlled timing of CRISPR/Cas9 delivery. eLife 3, e04766 (2014).
16. Wienert, B. et al. Timed inhibition of CDC7 increases CRISPR-Cas9 mediated templated repair. Nat. Commun. 11, 2109 (2020).
17. Maruyama, T. et al. Increasing the efficiency of precise genome editing with CRISPR-Cas9 by inhibition of nonhomologous end joining. Nat. Biotechnol. 33, 538-542 (2015).
18. Riesenberg, S. et al. Simultaneous precise editing of multiple genes in human cells. Nucleic Acids Res. 47, e116-e116 (2019).
19. Canny, M. D. et al. Inhibition of 53BP1 favors homology-dependent DNA repair and increases CRISPR-Cas9 genome-editing efficiency. Nat. Biotechnol. 36, 95-102 (2018).
20. Charpentier, M. et al. CtIP fusion to Cas9 enhances transgene integration by homology-dependent repair. Nat. Commun. 9, 1133 (2018).
21. Rees, H. A., Yeh, W.-H. & Liu, D. R. Development of hRad51-Cas9 nickase fusions that mediate HDR without double-stranded breaks. Nat. Commun. 10, 2212 (2019).
22. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
23. Gaudelli, N. M. et al. Programmable base editing of A·T to G·C in genomic DNA without DNA cleavage. Nature 551, 464-471 (2017).
24. Anzalone, A. V. et al. Search-and-replace genome editing without double-strand breaks or donor DNA. Nature 576, 149-157 (2019).
25. Roberts, B. et al. Systematic gene tagging using CRISPR/Cas9 in human stem cells to illuminate cell organization. Mol. Biol. Cell 28, 2854-2874 (2017).
26. Koch, B. et al. Generation and validation of homozygous fluorescent knock-in cells using CRISPR-Cas9 genome editing. Nat. Protoc. 13, 1465-1487 (2018).
27. Lackner, D. H. et al. A generic strategy for CRISPR-Cas9-mediated gene tagging. Nat. Commun. 6, 10237 (2015).
28. Gantz, V. M. & Bier, E. The mutagenic chain reaction: A method for converting heterozygous to homozygous mutations. Science 348, 442-444 (2015).
29. Shou, J., Li, J., Liu, Y. & Wu, Q. Precise and Predictable CRISPR Chromosomal Rearrangements Reveal Principles of Cas9-Mediated Nucleotide Insertion. Mol. Cell 71, 498-509.e4 (2018).
30. Wienert, B. et al. Unbiased detection of CRISPR off-targets in vivo using DISCOVER-Seq. Science 364, 286-289 (2019).
31. Bae, S., Kweon, J., Kim, H. S. & Kim, J.-S. Microhomology-based choice of Cas9 nuclease target sites. Nat. Methods 11, 705-706 (2014).
32. Shen, M. W. et al. Predictable and precise template-free CRISPR editing of pathogenic variants. Nature 563, 646-651 (2018).
33. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
34. Leibowitz, M. L. et al. Chromothripsis as an on-target consequence of CRISPR-Cas9 genome editing. Nat. Genet. 53, 895-905 (2021).
35. Bylund, L., Kytölä, S., Lui, W.-O., Larsson, C. & Weber, G. Analysis of the cytogenetic stability of the human embryonal kidney cell line 293 by cytogenetic and STR profiling approaches. Cytogenet. Genome Res. 106, 28-32 (2004).
36. Lin, Y.-C. et al. Genome dynamics of the human embryonic kidney 293 lineage in response to cell biology manipulations. Nat. Commun. 5, 4767 (2014).
37. Doench, J. G. et al. Rational design of highly active sgRNAs for CRISPR-Cas9-mediated gene inactivation. Nat. Biotechnol. 32, 1262-1267 (2014).
38. Kuscu, C. et al. CRISPR-STOP: gene silencing through base-editing-induced nonsense mutations. Nat. Methods 14, 710-712 (2017).
39. Aguirre, A. J. et al. Genomic Copy Number Dictates a Gene-Independent Cell Response to CRISPR/Cas9 Targeting. Cancer Discov. 6, 914-929 (2016).
40. Ihry, R. J. et al. p53 inhibits CRISPR-Cas9 engineering in human pluripotent stem cells. Nat. Med. 24, 939-946 (2018).
41. Haapaniemi, E., Botla, S., Persson, J., Schmierer, B. & Taipale, J. CRISPR-Cas9 genome editing induces a p53-mediated DNA damage response. Nat. Med. 24, 927-930 (2018).
42. Bae, S., Park, J. & Kim, J.-S. Cas-OFFinder: a fast and versatile algorithm that searches for potential off-target sites of Cas9 RNA-guided endonucleases. Bioinformatics 30, 1473-1475 (2014).
43. Tsai, S. Q. et al. GUIDE-seq enables genome-wide profiling of off-target cleavage by CRISPR-Cas nucleases. Nat. Biotechnol. 33, 187-197 (2015).
44. Hsu, P. D. et al. DNA targeting specificity of RNA-guided Cas9 nucleases. Nat. Biotechnol. 31, 827-832 (2013).
45. Kim, D. et al. Digenome-seq: genome-wide profiling of CRISPR-Cas9 off-target effects in human cells. Nat. Methods 12, 237-243 (2015).
46. KulcsAr, P. I. et al. Crossing enhanced and high fidelity SpCas9 nucleases to optimize specificity and cleavage. Genome Biol. 18, 190 (2017).
47. KulcsAr, P. I. et al. Blackjack mutations improve the on-target activities of increased fidelity variants of SpCas9 with 5′G-extended sgRNAs. Nat. Commun. 11, 1223 (2020).
48. Slaymaker, I. M. et al. Rationally engineered Cas9 nucleases with improved specificity. Science 351, 84-88 (2016).
49. Kleinstiver, B. P. et al. High-fidelity CRISPR-Cas9 nucleases with no detectable genome-wide off-target effects. Nature 529, 490-495 (2016).
50. Chen, J. S. et al. Enhanced proofreading governs CRISPR-Cas9 targeting accuracy. Nature 550, 407-410 (2017).
51. Casini, A. et al. A highly specific SpCas9 variant is identified by in vivo screening in yeast. Nat. Biotechnol. 36, 265-271 (2018).
52. Bitinaite, J. et al. USER friendly DNA engineering and cloning method by uracil excision. Nucleic Acids Res. 35, 1992-2002 (2007).
53. Clement, K. et al. CRISPResso2 provides accurate and rapid genome editing sequence analysis. Nat. Biotechnol. 37, 224-226 (2019).
54. Wickham, H. Ggplot2: elegant graphics for data analysis. (Springer, 2009).

### Example 2

### “Double Tap” Gene Drive Uses Iterative Genome Targeting to Help Overcome Resistance Alleles

### INTRODUCTION

The rapid spread of homing CRISPR-based gene drives through populations can help curb the impact of vector-borne diseases worldwide 1-3. For example, mosquitos can be modified with beneficial genes to prevent carried pathogens 4-6 or with detrimental gene alterations to suppress the vector population 7-9. Gene drives also offer promising solutions in crop pest population control 10 and invasive species suppression 11,12, such as for the rodents 13,14 currently impacting island conservation efforts 15. Briefly, CRISPR gene drives operate by biasing their own inheritance from Mendelian (˜50%) toward super-Mendelian (>50%) by converting heterozygous germline cells to homozygosity. Gene-drive constructs encode both a Cas9 endonuclease and a guide RNA (gRNA) that targets the precise location where the gene-drive transgene is integrated in the genome. In a heterozygous individual, resulting from a gene-drive individual mating with a wildtype, the Cas9/gRNA complex cleaves the wildtype allele opposing the gene drive. The endogenous cell machinery repairs this double-stranded DNA break, which copies the drive element from the drive chromosome to the cleaved wildtype one 16,17. When this process occurs in the germline of an individual, the inheritance is strongly biased towards the gene-drive transgene.

To repair the double-stranded DNA break, the germline has a bias towards the efficient and highly accurate homology-directed repair (HDR) repair pathway, which uses the intact strand—in this case, the strand containing the gene-drive—as a template for repair. In some cases, however, alternative, error-prone DNA-repair pathways, such as non-homologous end-joining (NHEJ) and microhomology-mediated end-joining (MMEJ), can instead generate small insertions or deletions (indels) near the gRNA cleavage site, disrupting the gRNA recognition sequence and rendering these indels resistant to further cleavage 4, 18, 19. Since such mutations can no longer be targeted by the drive and are passed on to the progeny, they can effectively counteract the spread of a gene drive through a population and obstruct field applications of these tools 20. Additionally, when a gene drive is inherited from the mother, it has been shown that in both the fruit fly 19 and in Anopheles mosquitoes 4,5, the Cas9/gRNA complexes deposited in the egg can prematurely target the incoming wildtype male allele, before it can reach the proximity of the female chromosome which would be used as a template for HDR. This dynamic leads to the early generation of indels that prevent further gene-drive conversion during later germline development. In extreme instances, the offspring of these animals will carry ˜50% gene drive and ˜50% resistance alleles 19, making this maternal effect a substantial and problematic source of resistance alleles as a gene drive progresses within a population.

Previously, a trans-complementing gene drive (tGD) was built in Drosophila melanogaster (D.mel) that inserts a Cas9 transgene within the coding sequence of the yellow gene and a tandem-gRNA cassette at the white locus 19. The gRNA transgene encodes two gRNAs, one targeting yellow (y1-gRNA) at the location where Cas9 is inserted, and the other targeting white (w2-gRNA) at the gRNA cassette insertion site. When the separate Cas9 and gRNA lines are crossed, the Cas9 protein can complex with the two gRNAs to cleave the wildtype yellow and white alleles, which leads to each of the transgenes being copied onto the opposing chromosome by HDR. While this action leads to super-Mendelian inheritance of both transgenes, it was observed resistance alleles generated by the end-joining alternative repair pathways. In this previous work, these resistance alleles were analyzed by sequencing ˜500 flies containing mutations at either the yellow or white locus, and it was observed that there were specific indels that appeared at a higher frequency than others, consistent with other findings in human cells 21-23.

To circumvent this phenomenon a CRISPR-based homing gene drive was supplemented with additional gRNAs targeting the most common resistance alleles generated by the drive process. This modification should provide a second opportunity for allelic conversion through HDR by allowing the drive element to also cut a subset of the resistance alleles, improving gene-drive inheritance. To do this, the “double-tap” trans-complementing gene drive (DT-tGD) was built, which contains two extra gRNAs, one for yellow and one for white, each targeting one of the most prevalent resistance alleles formed at each locus by our original tGD(y1,w2) 19. The DT-tGD system was tested and its ability to improve drive efficiency at both loci was shown. The data further show that the DT-tGD can specifically target the resistance alleles using the added gRNAs, and that this targeting results in efficient HDR conversion. Further, the data show that the DT-tGD spreads more efficiently in caged populations than the tGD control, supporting its potential use for counteracting resistance alleles in field applications of this technology.

### Methods & Materials

All the studies presented in EXAMPLE 2 followed procedures and protocols approved by the Institutional Biosafety Committee from University of California San Diego, complying with all relevant ethical regulations for animal testing and research. Gene-drive experiments were performed in a high-security Arthropod Containment Level 2 (ACL2) barrier facility.

### Plasmid Construction

All plasmids were cloned using standard molecular biology techniques. Plasmids were constructed by Gibson assembly using NEBuilder HiFi DNA Assembly Master Mix (New England BioLabs Cat. #E2621) and transformed into NEB 10-beta electrocompetent E. coli (New England BioLabs Cat. #3020). Plasmid DNA was prepared using a Qiagen Plasmid Midi Kit (Qiagen Cat. #12143) and sequences were confirmed by Sanger sequencing at Genewiz. Primers used for cloning can be found in Table 2 and the validated sequences of all constructs have been deposited in the GenBank database; accession numbers are provided in the Data availability Statement.

### Generation of Transgenic Lines

Constructs were sent to Rainbow Transgenic Flies, Inc. for injection. All constructs were injected into our lab's isogenized Oregon-R (Or-R) strain to ensure consistent genetic background throughout experiments. Constructs were co-injected with a Cas9-expressing plasmid29 expressing previously validated gRNA-w230. Injected G₀ animals were mailed back, then outcrossed to Or-R in small batches (3-5 males×3-5 females) and screened the G₁ flies for a fluorescent marker (GFP expressed in the eyes), which was indicative of transgene insertion. homozygous lines from single transformants were generated by crossing to Or-R and the white phenotype was identified in subsequent generations. Stocks were sequenced by PCR and Sanger sequencing to ensure correct transgene insertion.

### Fly Rearing and Crosses

All flies were kept on standard cornmeal food with a 12/12 hour day/night cycle. Fly stocks were kept at 18° C., and all experimental crosses were conducted at 25° C. To phenotype and cross flies, they were anesthetized using CO₂. For all crosses, virgin females were crossed the same day that they eclosed. F₀ crosses were made in small batches of 3-5 virgin females crossed to 3-5 males. F₁ crosses were made in single pairs, left for 5 days, then the adults were removed. F₂ flies were counted as male or female and scored for fluorescent marker (DsRed and/or GFP) using a Leica M165 F2 Stereomicroscope with fluorescence. DsRed or GFP expression was used as indicative of transgene inheritance. All gene drive experiments were performed in a high-security ACL2 (Arthropod Containment Level 2) facility built for gene drive purposes in the Division of Biological Sciences at the University of California, San Diego. Crosses were made in shatter-proof polypropylene vials (Genesee Scientific Cat. #32-120) and all flies and vials were frozen for 48 hours before being removed from the facility, autoclaved, and discarded as biohazardous waste.

### Sequencing of Individual Resistance Alleles

To sequence resistance alleles, genomic DNA was extracted from individual males following the protocol described by Gloor and colleagues31: flies were mashed in 50 μl squishing buffer (10 mM Tris-CI pH 8.2, 1 mM EDTA, 25 mM NaCl, and 200 μg/ml freshly diluted Proteinase K), then incubated at 37° C. for 30 min, then 95° C. for 2 min to inactivate the Proteinase K. Each sample was diluted with 200 μL of water, then 1-5 uL was used in a 25 uL PCR reaction spanning the gRNA cut site in either the yellow or white gene. The amplicon was then sequenced by Sanger sequencing to determine the resistance allele present. Primers used for resistance allele sequencing can be found in Table 2.

### Caged Population Protocol

For the population experiments, bottles were seeded with 100 flies each: 1) 50 y^{EX1}, w^{EX1} virgin females; 2) 40 y^{EX1} w^{EX1} males; and 3) 10 males from a homozygous stock containing the vasa-Cas9-DsRed construct and either the tGD(y1,w2) control or the DT-gRNA(y1,w2, y1b, w2b). Each condition was performed in triplicate. Adult flies were left in the bottles for 5 days before being removed. The remaining eggs and larvae were allowed to develop until day 18 at which point all flies were anesthetized with CO₂, removed, and approximately 200 were chosen at random to seed the next generation. The remaining flies were phenotypically scored as male or female and for GFP and/or DsRed expression using a Leica M165 F2 Stereomicroscope with fluorescence, with the fluorescent markers being indicative of transgene inheritance. The bottles were maintained on this schedule for 15 generations. All experiments were done at 25° C. and flies were kept on standard cornmeal food with a 12/12 hour day/night cycle. Experiments were conducted in shatter-proof polypropylene bottles (Genesee Scientific Cat #: 32-129F) within the high-security ACL2 facility, maintaining the same precautions as previous other gene drive experiments.

### Caged Population Deep-Sequencing

To perform deep sequencing of the caged populations, 50 GFP−, DsRed− males were isolated from each cage at the generations F4, F8, and F15. For two samples additional GFP−, DsRed+ flies were supplemented (F8, Cage 2: 30 GFP−, DsRed−males and 12 GFP−, DsRed+males; F8, Cage 3: 39 GFP−, DsRed− males and 11 GFP−, DsRed+males). 50 OregonR WT males were used as an indel baseline control. Genomic DNA was extracted from each fly pool following the standard protocol in the DNeasy® Blood and Tissue Kit (Cat. No. 69504). After extraction, each sample was eluted with 300 uL of water, and about ˜500 ng of the extracted DNA was then used in a 25 uL PCR reaction as a template to amplify either the yellow or white targeted region using specific primers for each locus:

yellow F:
(SEQ ID NO: 1)
ACACTCTTTCCCTACACGACGCTCTTCCGATCTCTCTGCTAATTCCGTA
TCCAGATTGGC,
yellow R:
(SEQ ID NO: 2)
TGGAGTTCAGACGTGTGCTCTTCCGATCTGCCTATATCCACGGCAATGT
TAGC,
white F:
(SEQ ID NO: 3)
ACACTCTTTCCCTACACGACGCTCTTCCGATCTCTCTCTATTCGCAGTC
GGCTGATCTG,
white R:
(SEQ ID NO: 4)
TGGAGTTCAGACGTGTGCTCTTCCGATCTGGTCATCCTGCTGGACATAG
GC).

1 μL of the resulting PCR reaction was used as a template for the subsequent PCR reaction to attach Illumina barcodes. 3 μL of the barcoding PCR product was then run on a gel, and the amplicon band was first gel extracted using QIAquick® Gel Extraction Kit (Cat. No. 28704) and then further purified using Monarch@ PCR & DNA Cleanup Kit (5 μg) (Cat. No. T1030L). The pooled and purified DNA amplicons were quantified with the Qubit dsDNA high sensitivity kit (Thermo Fisher). Equal amounts of amplicon from each sample were pooled together and prepared based on the Illumina sequencing protocol. 1.8 pM of the pooled libraries were mixed with 1.8 pM PhiX with nine to one ratio and loaded on an Illumnina MiniSeq instrument using a mid output kit of 300 cycles. Data was analyzed using CRISPResso232 to determine the frequency of resistance alleles across different generations.

### Caged Populations Data Analysis

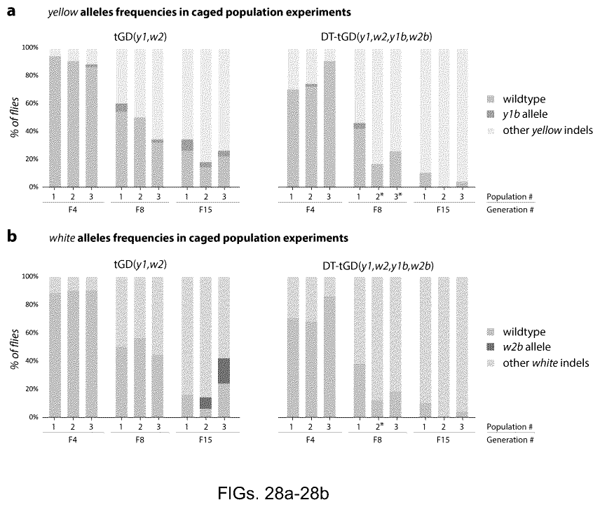

Using as a reference the data obtained from the OregonR wild type males to consider any indel occurrence with less than 100 occurrences as background and removed these sequences from downstream analysis. The frequency observed for the different alleles (wild-type, y1b or w2b, and other indels) was then used to estimate the number of flies present in the sampled pool. The easimate was done by first dividing the frequency of a specific allele by the sum of all the frequencies of the alleles above background (i.e. true alleles), then multiplying this number by the number of male flies that contributed an allele to the pool, and then rounding this number to the closest integer. The resulting estimates (i.e. number of flies contributing an allele to the pool) was used to generate the graphs in FIGS. 28a-28b.

### Graphical Representation of the Data and Statistical Analysis

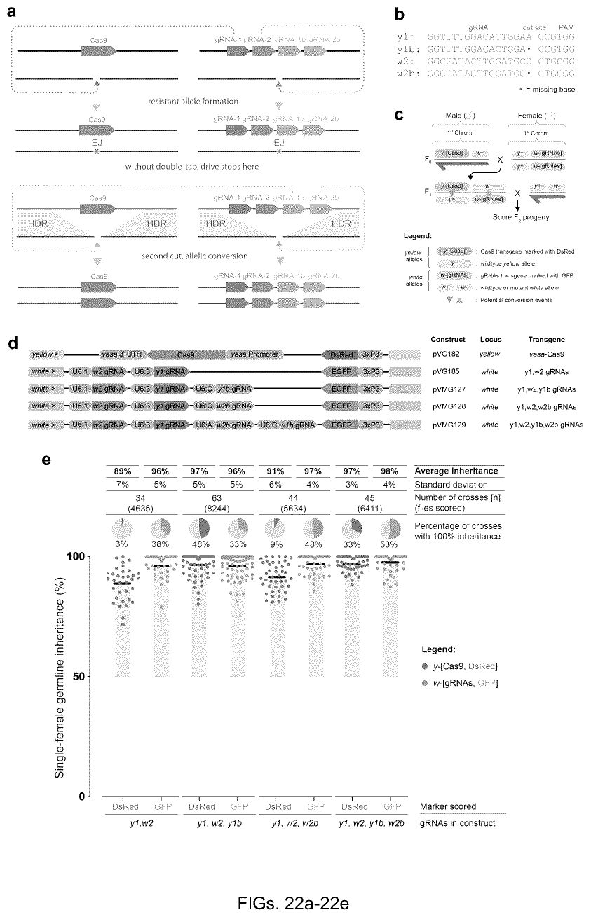

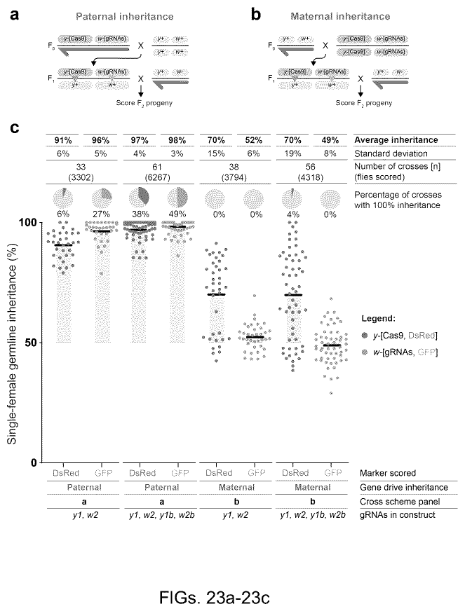

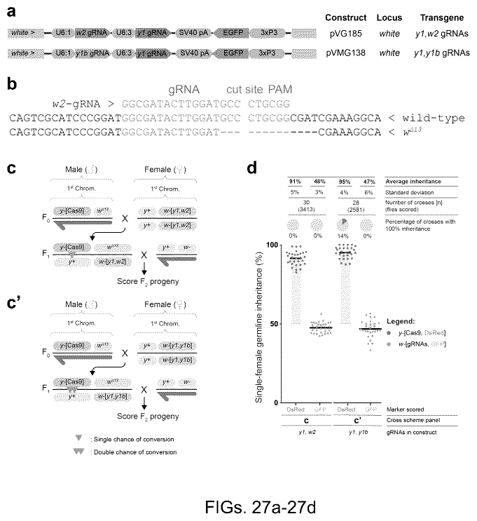

GraphPad Prism 9 and Adobe Illustrator were used to generate all the graphs. Statistical analyses were done using GraphPad Prism 9 and the StatKey analysis tool, version 2.1.1. For FIGS. 22a-22e and FIGS. 23a-23c a Kolmorgorov-Smirnov test was used to test for normal distribution then Mann-Whitney tests were used to test for differences in means of inheritance rates. Randomization tests were also performed for a difference in proportions to evaluate differences in the percentages of vials at 100% inheritance. In these analyses 10,000 randomizations were performed of these data. In Table 1 randomizations tests wetr again used for a difference in proportions with 10,000 randomizations to evaluate percentages of y1b and w2b alleles. For FIGS. 27a-27d a Kolmorgorov-Smirnov test was performed to test for normal distribution and t-tests were used to evaluate the differences in means of inheritance rates.

### Data Availability Statement

The plasmid sequences of the constructs generated are either deposited into the GenBank database. GenBank accession numbers for the deposited plasmids are the following: pVG182 vasa-Cas9 (MN551085)33, pVG185 tGD(y1,w2) (MN551090)19, pVMG127 DT-tGD(y1,w2, y1b) (OL630771), pVMG128 DT-tGD(y1,w2,w2b) (OL630772), pVMG129 DT-tGD(y1,w2, y1 b, w2b) (OL630773), pVMG130 C-tGD(w2, y1 b) (OL630774), pVMG131 C-tGD(y1,w2b) (OL630775), pVMG138 C-tGD(y1, y1 b) (OL630776). All raw and source data and information are available upon request.

### Results

### A. Double Tap Trans-Complementing Gene Drive Improves Inheritance Rates

To evaluate whether an additional gRNA would improve inheritance by recycling indels generated by the primary gRNA, double-tap versions (DT-tGD) of the previously-tested tGD targeting the genes yellow and white 19 was designed. Compared to tGD, this new arrangement includes two additional gRNAs within the construct inserted in the white gene (FIG. 22a). These additional gRNAs target the most prevalent resistance alleles generated at either the yellow or white loci (y1b or w2b) by the primary gRNAs (y1 or w2, respectively) (FIG. 22b)19. In the double-tap system, the primary gRNA (y1 or w2) cuts first and then, if a specific high-frequency indel is generated due to error-prone NHEJ or MMEJ repair, the secondary gRNA (y1b or w2b) can cleave the indel allele for another opportunity to copy the drive by HDR (FIG. 22a′). Given that the most common indels identified in the previous analysis 19 only lack 1 base pair, the two secondary gRNAs designed here to target the indel at the same location do so with a length of 19 nt instead of the canonical 20 nt (FIG. 22b).

To test the DT-tGD system, three gRNA-constructs were made to compare to the tGD(y1,w2) control (FIG. 22d). The control construct has two gRNAs, y1 and w2, driven by D.mel U6-3 and U6-1 promoters, respectively, along with a GFP marker expressed in the eye to track the presence of the transgene phenotypically. The first construct, DT-tGD(y1,w2, y1b), carries a secondary gRNA for yellow (y1b) driven by the Drosophila grimshawi(D.gri) U6-C promoter (FIG. 22d). The second construct, DT-tGD(y1,w2,w2b), carries a secondary gRNA for white (w2b), also driven by the D.gri-U6-C promoter (FIG. 22d). The third construct, DT-tGD(y1,w2, y1b, w2b), carries both the secondary gRNAs (y1b, w2b) driven by D.gri-U6-A and D.gri-U6-C, respectively (FIG. 22d). These different U6 promoters were chosen due to previous success in a gene-drive setting and to avoid the problematic recombination that has been shown to occur within the gene drive element if identical sequences are used 24. All of these gRNA constructs were then inserted at the same location of our tGD(y1,w2) control in the white locus and similarly marked with GFP so they could be combined with the same Cas9 line as the original tGD 19. This line carries a Cas9 gene driven by the germline-specific vasa promoter, inserted in yellow at the y1-gRNA cut site and marked with DsRed expressed in the eye (FIG. 22d).

To test these three double-tap constructs, genetic crosses were performed to combine the two tGD components by mating Cas9-expressing males to gRNA-expressing females. From their progeny, trans-heterozygous F₁ virgin females were then collected that should display gene-drive conversion in their germline and single-pair crossed them to wildtype (Oregon-R) males (FIG. 22c). The F₂ progeny from each cross was scored for presence of the DsRed (Cas9) and GFP (gRNA) markers. Given that the yellow (Cas9-DsRed) and white (gRNA-GFP) are in close proximity (˜1 cM) on the X chromosome, Mendelian inheritance would lead to ˜50% DsRed and ˜50% GFP F2 individuals; any individual carrying both elements signifies an allelic conversion event and a successful gene drive, with a small margin of error (˜0.5%) due to meiotic recombination between the two loci.

For the tGD(y1,w2) control, it was observed 89% inheritance of the Cas9-DsRed transgene and 96% inheritance of the gRNA-GFP transgene, in line with the previous characterization of this arrangement19. For the DT-tGD(y1,w2, y1b), Cas9-DsRed transgene inheritance improved significantly to 97% (compared to 89% for the control, p <0.0001, Mann Whitney test), suggesting that the additional y1b-gRNA increases inheritance of the transgene. No increase was observed for the inheritance of the gRNA-GFP transgene, which contained no secondary gRNA for this position and therefore displayed an average inheritance comparable with the control of 96%. For the DT-tGD(y1,w2,w2b), which instead carries an additional gRNA for white, an average inheritance of 97% was observed for the gRNA-GFP transgene (compared to 96% in the control). For this condition, the Cas9-DsRed transgene acted instead as an internal control displaying an average inheritance rate of 91% which is comparable with the control (89%). The four-gRNA DT-tGD(y1,w2, y1b, w2b) were also tested and improved inheritance rates of both transgenes were expected. Indeed, a significantly higher inheritance was observed for the Cas9-DsRed in yellow (97% compared to 89% for the control, p<0.0001, Mann Whitney test) and the gRNA-GFP transgene in white (98% compared to 96% for the control, p=0.1179, Mann Whitney test). From this analysis, it was concluded that the double-tap arrangement can improve drive efficiency at the yellow locus. Given that the w2-gRNA has, on its own, very high conversion rates (˜96%), the small range available for improvement did not allow to observe statistically significant differences in these experiments.

The double-tap should also increase the overall number of crosses generating 100% inheritance due to its two-step action. The fraction of vials (i.e., germlines) producing 100% inheritance for each transgene was compared. For DT-tGD(y1,w2, y1b), the fraction of vials producing 100% inheritance of the DsRed transgene climbed significantly from the tGD(y1, w2) control value of 3% to 48% (p<0.0001, randomization test for a difference in proportions). For DT-tGD(y1,w2,w2b), the fraction of vials displaying 100% GFP inheritance grew from 38% (control) to 48% with the double-tap (p=0.277, randomization test for a difference in proportions). Similarly for the four-gRNA DT-tGD(y1,w2, y1b, w2b), it was observed a consistent increase in both transgenes, with the fraction of crosses at 100% DsRed inheritance significantly increased from 3% for the control to 33% (p=0.0006, randomization test for a difference in proportions), and at 100% GFP inheritance increased from 38% for the control to 53% (p=0.133, randomization test for a difference in proportions). This additional analysis confirms that the double-tap can significantly improve inheritance at the yellow locus and, while all the observations are consistent with an improvement of inheritance at white, statistical significances for these comparisons were not observed.

### B. Double Tap Gene Drive Displays Maternal Effects Caused by Cas9/gRNA Deposition in the Egg

To tested whether the double-tap drive would similarly improve inheritance when both the Cas9 and the g RNAs are co-inherited from the same parent, in a condition similar to a full gene drive19, a homozygous fruit fly strain containing both the vasa-Cas9 and DT-tGD(y1,w2, y1b, w2b)-gRNAs on the same chromosome was generated (FIGS. 23a-23c). To first test the inheritance from a single parent without the additional confounding influence of maternal effects, males from this stock were taken and mated to wildtype virgin females. From the resulting progeny, F₁ virgins and single-pair crossed them to wildtype males were collected to evaluate the inheritance of the two transgenes (FIG. 23a). After scoring the F₂ offspring for the presence of the fluorophores, it as observed 97% average inheritance of the Cas9-DsRed transgene and 98% average inheritance of the gRNA-GFP transgene, both significantly increasing in comparison to the tGD(y1,w2) control (91% [p<0.0001, Mann Whitney test] for Cas9-DsRed and 96% [p=0.0093, Mann Whitney test] for gRNA-GFP) (FIG. 23c) and in line with these findings when the two elements were inherited separately from F₀ flies (FIG. 23e). When the fraction of vials wsd analyzed at 100% inheritance, it was observed significant increases compared to the control for both the Cas9-DsRed transgene, from 6% to 38% (p=0.0003, randomization test for a difference in proportions), and the gRNA-GFP transgene, from 27% to 49% (p=0.033, randomization test for a difference in proportions). These results suggest that a four-gRNA double-tap strategy significantly improves inheritance rates at both loci, further supporting the observation for the transgenes inherited separately from the F₀ and confirming the effect of the DT-tGD at the white locus described earlier (FIG. 22e). It is possible that this difference is due to the co-inheritance of the transgenes boosting the double-tap performance, or a statistical effect due to a higher number of crosses analyzed in the experiment in FIG. 23c.

The propagation of engineered gene-drive systems can suffer from a maternal effect caused by Cas9 protein and gRNA deposition in the egg by transgenic females, leading to the high frequency generation of indels4,5, 19. To evaluate if the double-tap system could alleviate this effect by recycling some of the generated indels, F₀ females from the Cas9+gRNA homozygous stock were crossed with wildtype males to obtain heterozygous F₁ females (FIG. 23b). These F₁ females were then single-pair crossed to wildtype males to evaluate the transmission of the two transgenes to the F₂ offspring (FIG. 23b). It was observed that the Cas9-DsRed transgene was inherited at only 70% on average, similar to the tGD(y1,w2) control (FIG. 23c). The gRNA-GFP transgene displayed an even stronger maternal effect, with an inheritance rate of 49%, which was similar to the control (52%) and our previous findings19 (FIG. 23c). These results suggest that the additional gRNAs in the double-tap arrangement are also deposited as Cas9/gRNA complexes in the egg and do not positively affect gene drive performance through maternal inheritance. The previous work showed that the primary gRNAs in this system (y1 and w2) are extremely efficient and, when inherited by the mother, target the paternal allele in the first hours of development19. The secondary gRNAs added to the double-tap arrangement are equally as efficient, given their similar sequences, and could therefore act in very rapid succession in the early stages of embryo development, effectively not overcoming the maternal effect.

C. Double Tap Secondary gRNAs Specifically Target Indels for Conversion

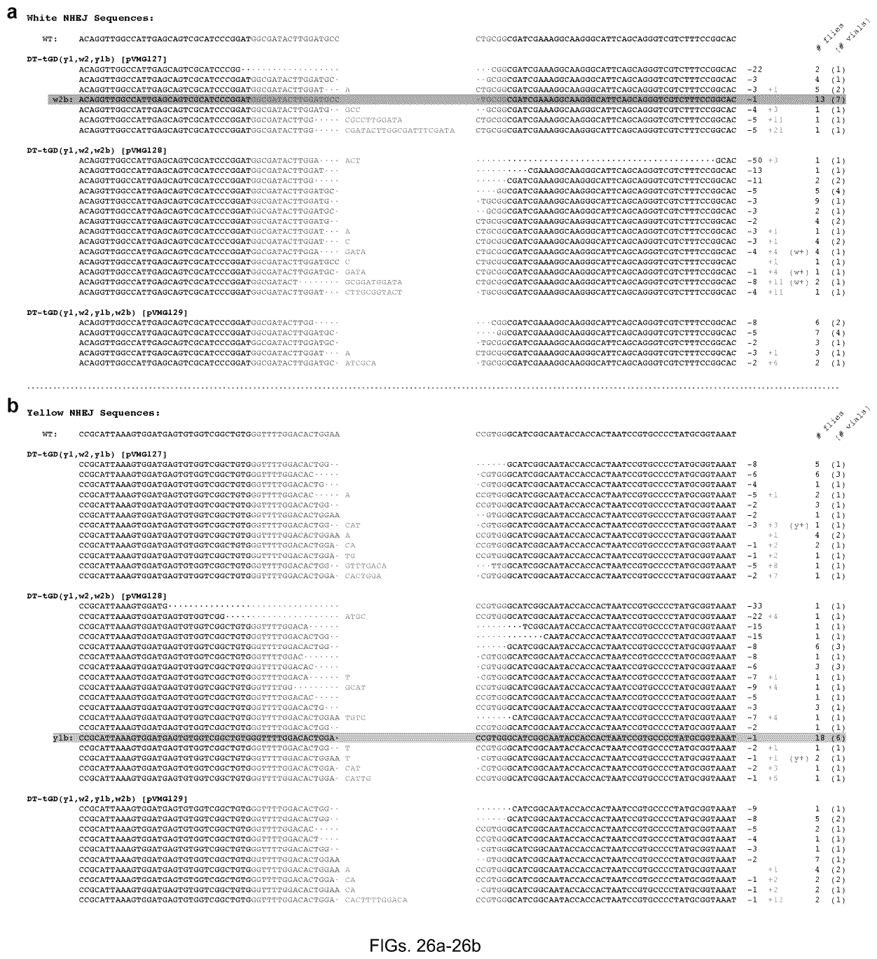

To rule out an unexpected mechanism contributing to the increased rate of transgene inheritance in the double-tap system, the makeup of the indels and the prevalence of the y1b and w2b sequences in the F₂ were evaluated. To do this, several DsRed− or GFP− F₂ males from the experiments performed in FIGS. 22a-22e were sequenced. Males were used because they have only one X chromosome containing the yellow and white locus and therefore allow for sequencing of one copy of each of these loci. From each condition, several male flies were isolated and the indel generated at either the yellow or white locus was genotyped. Indeed, the y1b sequence disappeared from conditions carrying the y1b-gRNA, while the w2b sequence disappeared from conditions carrying the w2b-gRNA (Table 1, FIGS. 26a-26b). These results suggest that the secondary gRNAs successfully target the intended indels, likely allowing for an additional round of gene-drive conversion.

TABLE 1
Summary of the indel sequence analysis

| | Alleles observed/Total (%) | |
|---|---|---|
| tGD gRNAs | y1b | w2b |

| | | | | |
|---|---|---|---|---|
| y1, w2 | 18/37 | — | 12/19 | — |
| | (49%) | | (63%) | |
| y1, w2, y1b | 0/28 | **** (p < | 13/27 | n.s. (p = |
| | (0%) | 0.0001) | (48%) | 0.241) |
| y1, w2, w2b | 18/45 | n.s. (p = | 0/38 | **** (p < |
| | (40%) | 0.291) | (0%) | 0.0001) |
| y1, w2, y1b, | 0/27 | **** (p < | 0/21 | **** (p < |
| w2b | (0%) | 0.0001) | (0%) | 0.0001) |

p values were calculated for the three experimental conditions in comparison with the control using a 1-tail randomization test for a difference in proportions.
**** p < 0.0001;
n.s. = not significant.

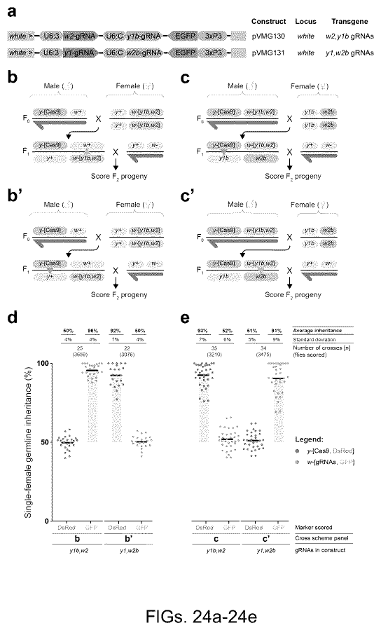

To further show that the secondary gRNAs in the double-tap system specifically target the intended indels, the wildtype alleles were challenged with constructs lacking one of the primary gRNAs (FIG. 24a). First, two control tGDs (C-tGD), one containing w2 and y1b gRNAs (without a yl) and one containing y1 and w2b gRNAs (without a w2) were generated, these constructs were otherwise the same as the tGDs described above and were inserted in white and marked with GFP (FIG. 24a). F₀ C-tGD(y1b, w2) virgins was then crossed to F₀ vasa-Cas9 males. To evaluate transmission of the two transgenes, trans-heterozygous F₁ virgins were collected and outcrossed to wildtype males in single pairs (FIG. 24b). Scoring the F₂ for DsRed and GFP expression, the gRNA-GFP transgene in white was inherited at super-Mendelian frequencies (96%) given the presence of the w2-gRNA, but the Cas9-DsRed transgene in yellow instead showed Mendelian inheritance (˜50%), suggesting that the y1b-gRNA is unable to target the wildtype yellow sequence (FIG. 24d). To evaluate the w2b-gRNA in the same way, the same cross were then performed using the C-tGD(y1,w2b) (FIG. 24b′). Similarly, the Cas9-DsRed transgene was inherited at ˜92% with the primary y1-gRNA, at about the same rate as the basic tGD(y1,w2) (FIG. 24d). The gRNA-GFP transgene instead showed Mendelian inheritance (˜50%), suggesting that the w2b-gRNA is unable to cut the wildtype white allele (FIG. 24d). These experiments show that the two secondary gRNAs (y1b and w2b) are unable to target the respective wildtype sequences, at least not at a level detectable in this system.

To demonstrate that the y1b- and w2b-gRNAs can specifically target the intended alleles to generate a gene drive via the conversion of these indels, a fruit fly line termed “y1b, w2b” was generated, which carries the two indel alleles (y1b, w2b) generated at the respective loci by previous rounds of gene drive using the primary gRNAs. These alleles in this fruit fly line should be efficiently cleaved by the secondary gRNAs of the same name. Homozygous lines combining each of the C-tGDs with vasa-Cas9 on the same chromosome were separately generated. Males from these vasa-Cas9,C-tGD stocks were then crossed to y1b, w2b females; from their offspring F₁ heterozygous virgins were collected and single-pair crossed to wildtype males to evaluate the transgene transmission to their F₂ progeny (FIGS. 24c-c′). For C-tGD(y1b, w2), it was observed that y1b-gRNA can cut the y1b allele, leading to a super-Mendelian average inheritance of 93% of the Cas9-DsRed transgene, while the w2-gRNA, however, is unable to cleave the w2b allele, resulting in Mendelian inheritance of the gRNA-GFP transgene (52%) (FIG. 24e). Similarly, when the F₂ generation of the C-tGD(y1,w2b) cross was analyzed, it as found that w2b-gRNA successfully triggers super-Mendelian inheritance of 91% of the gRNA-GFP transgene, while y1-gRNA does not seem to cut the y1b allele, leading to an observed Mendelian inheritance of the Cas9-DsRed transgene (51%) (FIG. 24e). Combined, these results show that each of the four gRNAs in the system specifically cleave the sequences they are meant to target, and all of them can generate a gene drive of the respective transgene.

D. Double Tap Improves Drive when the Number of gRNAs in the System is Held Constant

Given that the DT-tGD carries four gRNA-expressing genes while the control tGD(y1,w2) has only two, it was tested whether differences in the total number of gRNA-expressing genes could affect gene-drive efficiency and therefore the interpretation of our double-tap results. Since the effect of the double-tap strategy is stronger on the transgene inserted in yellow, this transgene was focused on for this analysis. To control the number of gRNA genes, an additional C-tGD carrying only two gRNAs, y1 and y1b, analogous to the tGD(y1,w2) was generated (FIG. 27a). To comparably test these constructs where two gRNAs are expressed but only one locus is cut, the action of the gRNA targeting white was disabled using a version of the Cas9 line in which the w2 cut site is destroyed by a 13 bp deletion that includes the PAM site (FIG. 27b). With this Cas9,w³ line, the w2-gRNA expressed by the tGD(y1,w2) construct can still bind to the available pool of Cas9, but it will not be able to cleave the genome at white. This makes the w2-gRNA gene a placeholder in this system, allowing to have the same number of gRNA-expressing genes across the two conditions without changing the number of cuts generated at one time.

To perform this experimental analysis, males from the Cas9, w^{Δ13} line were crossed to virgins from either the tGD(y1,w2) or tGD(y1, y1b) lines, F₁ virgins were collected and crossed to wildtype males to evaluate the inheritance of the respective constructs in the F₂ by scoring the fluorescent markers (FIGS. 27c-c′). The gRNA-GFP transgene inserted in white was inherited in a Mendelian fashion, given that the w2-gRNA is unable to cut the w^{Δ13} allele and that the tGD(y1, y1b) has gRNAs targeting only yellow (FIG. 27d). In contrast, both conditions showed super-Mendelian inheritance at yellow, with the DsRed-Cas9 transgene in the tGD(y1,w2) present in an average of 91% of the F₂ flies and the C-tGD(y1, y1b) at a significantly higher average inheritance of 95% (p=0.0018, unpaired t test) (FIG. 27d). In addition, the percentage of crosses with 100% DsRed flies also increased from 0% with tGD(y1,w2) to 14% using DT-tGD(y1, y1b) (FIG. 27d). These inheritance values are comparable to the previous experiments using tGD(y1,w2) and DT-tGD(y1,w2, y1b, w2b) (FIG. 22e), suggesting that the difference in gRNA-expressing constructs in our initial analysis is not responsible for the increase in inheritance observed for the DT-gRNA(y1,w2, y1b, w2b) construct. Together, these results confirm that the addition of secondary gRNAs to the double-tap system increases drive efficiency, which is not due to differences in the amount of gRNA-expressing genes in the double-tap transgene.

E. DT-tGD Outperforms Regular tGD when Spread in a Population

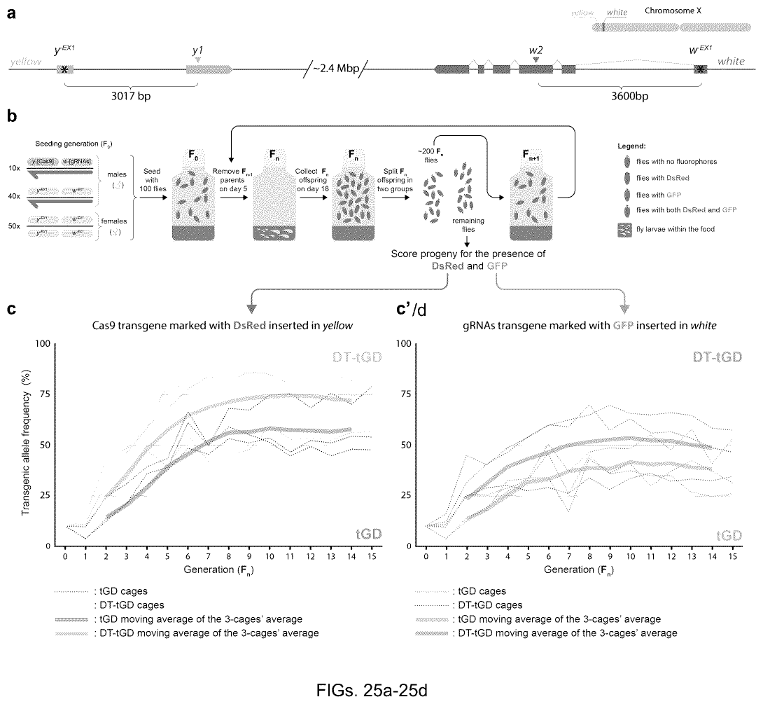

Because the double-tap strategy improved gene-drive performance, it was then tested whether the addition of secondary gRNAs would improve spread of the DT-tGD in a population. Given that the DT transgenes are inserted in either the yellowor white genes, to eliminate a fitness difference between the gene drive and the wildtype alleles a homozygous yellow-, white-fly line were used as the target population. For this purpose, a mutant line was generated by injecting gRNA- and Cas9-expressing plasmids targeting the first exon of yellow and white. These null alleles, y^{EX1} and w^{EX1} were generated at a considerable distance from the gene-drive insertion site so as to not influence the sequence-homology-dependent gene-drive process (FIG. 25a).

To test the performance of the double-tap strategy in a caged population setting, three bottles were seeded with: 1) 50 y^{EX1} w^{EX1} virgin females; 2) 40 y^{EX1} w^{EX1} males; and 3) 10 males from a homozygous stock containing the vasa-Cas9-DsRed construct and either the tGD(y1,w2) control or the DT-gRNA(y1,w2, y1b, w2b) (FIG. 25b). These bottles, each containing 100 flies, were incubated at 25° C. and the parental generation was removed after five days. The next generation in the form of eggs and larvae was left to develop until day 18, when the hatched flies were collected for phenotypic scoring and for seeding the next generation (FIG. 25b). To track the spread of the transgene in each population, a portion of the offspring was scored for the presence of the GFP and DsRed transgene markers at each generation. Indeed, the frequency of the transgenic alleles in each bottle increased over time until stabilizing between generation F₁₀ and F₁₅ (FIG. 25c). On average, the DT-gRNA(y1,w2, y1b, w2b) had a higher prevalence of both the Cas9-DsRed (FIG. 25c) and the gRNA-GFP (FIG. 25c′/d) transgene than the tGD(y1,w2) control, suggesting a positive effect of the secondary gRNAs.

In the caged population experiments, the percentage of transgenic alleles in each condition seemed to level off at different values much lower than 100%. Indeed, this behavior was observed given the strong maternal effect previously characterized at both loci19. This effect was more pronounced for w2 than for y1, consistent with the observations described in FIG. 23c and the lower values observed in the cage experiments for the gRNA-GFP transgene inserted in white (FIG. 25c′/d). Furthermore, while in the experimental setup gene drive conversion only happens in females as both transgenes are located in the X chromosome, gene drive arrangements targeting autosomal genes where conversion occurs in both sexes, could further benefit from a double-tap approach.

To confirm this was due to maternal effects and simultaneously evaluate the generation of indels as the tGDs spread, the targeted loci from pools of male individuals were deep sequenced, again for their simpler makeup of one allele per individual. Three time points: during the initial exponential spread (F4), when the gene-drive spread began to slow (F8), and at the end of the experiment to evaluate the final population makeup (F15) were sampled. It was found that the frequency of wildtype alleles diminished over time in all cages, reaching levels in the 0-26% range in the F15 generation, and indel alleles accumulated (FIGS. 28a-28b). The frequency of either the y1b or the w2b sequence was then analyzed in these pools; the y1b allele appears as early as the F4 generation in the tGD(y1,w2) cages and seems to accumulate over time, with all tGD(y1,w2) cages containing it (FIGS. 28a-28b). Differently, in the DT-tGD(y1,w2, y1b, w2b), the y1b allele was observed only at low frequencies and only in two instances (F4, population 2; F8, population 1). These indels disappeared by the F15 generation, suggesting that when y1b alleles are generated and escape action of the y1b-gRNA, they can be targeted in subsequent generations (FIGS. 28a-28b). The w2b allele followed a similar trend, consistent with the elimination of the w2b alleles under the action of the w2b-gRNA present in the double-tap construct. Although here, fairly high frequencies of the allele were observed, but only in two out of three tGD(y1,w2) populations in the F15 (FIGS. 28a-28b).

Surprisingly, the y1b and w2b alleles accumulate in the tGD(y1,w2) populations to a much lower frequency than expected, given that in above Table 1, these alleles were observed appearing with 49% (y1b) and 63% (w2b) in single-pair crosses. This may be explained by a qualitative difference between indel alleles generated through NHEJ/MMEJ in the late germline (see Table 1) and indel alleles generated in population experiments. In the latter case, the major source of indel generation is the maternal effect which acts in early embryos, as seen in the previous study19. Altogether these results suggest that the double-tap strategy can improve gene-drive performance as it spreads in a population by specifically recycling indel alleles for a second round of gene-drive conversion.

TABLE 2
Primer List for Primers Used in Studies in EXAMPLE 2

| | |
|---|---|
| Name | Primer |

Primers used for building constructs

| | |
|---|---|
| 373 | TTTGCTCACCTGTGATTGCTCC (SEQ ID NO: 5) |
| 451 | GTACGCGTATCGATAAGCTTTAAGATACATTGATGAGTTTGG (SEQ ID NO: 6) |
| 590 | AAGCTGGAGCTCCTGCAGGTTGTTGGTTGGCACACC (SEQ ID NO: 7) |
| V0181 | CCTGCAGGAGCTCCAGCTTTTGTTAAAAAGAACAATATCGATATACGATA |
| | TTTTAAGTTC (SEQ ID NO: 8) |
| V0421 | AACCGGTCCAGTGTCCAAAACCAAATGTAGCTCAAAAAATTGCGTATATA |
| | AATG (SEQ ID NO: 9) |
| V0422 | ACATTTGGTTTTGGACACTGGACCGGTTTTAGAGCTAGAAATAGCAAGTT |
| | AAAATAAGGC (SEQ ID NO: 10) |
| V0423 | GTATCTTAAAGCTTATCGATACGCGTACGCTAGCAATAAATGGCGACAGC |
| | AACAAAAAAC (SEQ ID NO: 11) |
| V0424 | CAGGCATCCAAGTATCGCCAAATGTAGCTCAAAAAATTGCGTATATAAAT |
| | G (SEQ ID NO: 12) |
| V0425 | GGCGATACTTGGATGCCTGGTTTTAGAGCTAGAAATAGCAAGTTAAAATA |
| | AGGC (SEQ ID NO: 13) |
| V0426 | ACAACCTGCAGGAGCTCCAGCTTTTGTCGTACAACTAGGGTTGCAACCA |
| | GGTAGTTGGAC (SEQ ID NO: 14) |
| V0433 | CTAAAACCAGGCATCCAAGTATCGCCGAAGATTAGATCCTTAAATCTGCT |
| | ATTTAAATGG (SEQ ID NO: 15) |
| V0434 | AACTTAAAATATCGTATATCGATATTGTTCTTTTTACAACAATTATAAAAAA |
| | AAGCGCAC (SEQ ID NO: 16) |
| V0435 | GTAAAAAGAACAATATCGATATACGATATTTTAAGTTCCTTTCGCGCTTAG |
| | CCATTACAC (SEQ ID NO: 17) |
| V0442 | GGAGCAATCACAGGTGAGCAAAAAAGACGTCTGCGGCGATCGAAAGG |
| | (SEQ ID NO: 18) |
| V0489 | GGAGCAATCACAGGTGAGCAAAAAAGCACCGACTCGG (SEQ ID NO: 19) |
| V0490 | CAGTATATATAGGAAAGATATCCGGGTGAACTTCGGTTTTGGACACTGGA |
| | CCG (SEQ ID NO: 20) |
| V0495 | GAAGTTCACCCGGATATCTTTCCTATATATACTG (SEQ ID NO: 21) |

Primers used for amplifying and sequencing individual white indels

| | |
|---|---|
| V0478 | GCTGGTCAACCGGACACGCGG (SEQ ID NO: 22) |
| V0659 | AGGGAGCCGATAAAGAGGTCATCC (SEQ ID NO: 23) |

Primers used for amplifying and sequencing individual yellow indels

| | |
|---|---|
| V0477 | TATTATCCAGAAAACAGACAGC (SEQ ID NO: 24) |
| V0657 | GGACATACCAAATATACCCTCC (SEQ ID NO: 25) |

Primers used for deep sequencing at white (adapter in green)

| | |
|---|---|
| V1442 | ACACTCTTTCCCTACACGACGCTCTTCCGATCTCTCTCTATTCGCAGTCG |
| | GCTGATCTG (SEQ ID NO: 3) |

### Discussion & Summary

This EXAMPLE 2 provides the double-tap homing gene-drive strategy to combat the most prevalent resistance alleles that prevent drive spread. This strategy uses an additional, secondary gRNA targeting these resistance alleles to recycle them as new templates for an additional round of gene conversion, ultimately improving gene-drive efficiency. A double-tap version of a previously tested trans-complementing gene drive targeting the yellow and white loci of fruit flies19 showed that the secondary gRNAs are specific in their targeting and improve the drive efficiency at both loci tested. The double-tap also improves the ability of the drive to spread in a population, with the double-tap reaching higher frequencies than the control.

Studies presented in EXAMPLE 2 confirms that the efficiency of the drive depends on the locus and gRNAs used. Of the two loci tested in EXAMPLE 2, the double-tap strategy performed better at yellow, likely due to the lower baseline conversion efficiency of yl-gRNA (89%) than w2-gRNA (96%). This generates more resistance alleles that can be further converted, which results in a more readily observable phenomenon for yellow. Additionally, this study employed only one additional secondary gRNA, yet a modest improvement in efficiency is observed. In the drive process, several resistance alleles are generated consistently, which could be targeted by the addition of multiple secondary or tertiary gRNAs to further improve conversion rates and approximate 100% efficiency.

The double-tap strategy also improves upon other proposed strategies that relied on the multiplexing of gRNAs to overcome resistance alleles. For example, two or more adjacent gRNA target sites have been employed to increase drive efficiency when either one of them would fail25,26. While this strategy allows for recycling resistance alleles, it also has the potential to generate non-homologous overhangs that can affect HDR rates, as shown in previous work19. The double-tap acts instead as a multiplexing system “in time” instead of “in space” and creates no homology mismatches while still allowing the drive element multiple chances to convert the wildtype allele. This feature of the double-tap system allows it to be seamlessly implemented in existing gene-drive systems to further boost their effectiveness.

Though this work addresses the drawback of indel formation slowing drive spread, another drawback of gene-drive systems in insects stems from the maternal effect caused by Cas9 and gRNA deposition in the egg which severely impairs drive efficiency. The double-tap does not seem to reduce this maternal effect, at least using the gRNAs tested in this study. It was believed that the strong maternal effect observed in this study is due to the highly efficient gRNAs employed. Use of less efficient gRNAs may lead to lessened maternal effect and should also greatly benefit from a double-tap approach.

While the main scope of the studies presented in EXAMPLE 2 was to demonstrate the feasibility of the strategy, the constructs were also evaluated for their potential to spread in caged population experiments to test their potential for field use. While the strong maternal effect in both the double-tap and control populations rapidly generated resistance alleles that stifled the spread of either drive, a higher level of spread for the double-tap than the control was nonetheless observed, further supporting the beneficial effect of the secondary gRNAs. This suggests that the double-tap strategy could be universally applied to increase the efficiency of CRISPR-based gene-drive systems suffering from resistance allele generation. For example, several mosquito systems4, 5, 7, 8 can partially circumvent the generation of resistance alleles by different strategies; implementing a double-tap approach should further increase their spread in a population. Additionally, secondary gRNAs could be used to specifically target problematic resistance alleles, such as those retaining target gene function and thus not suffering an imposed fitness disadvantage from the gene drive5.

Further, a double-tap strategy could be implemented in systems where HDR conversion is less efficient, such as primary human cells or mouse embryos. The delivery of secondary gRNAs in human cells could increase HDR-based transgenesis and perhaps benefit therapeutic uses requiring the HDR-based delivery of beneficial cargos27, while its use in mice could further boost transgenesis efficiency beyond the latest improvements28. Overall, the double-tap strategy can be widely applicable to diverse situations that could benefit from the use of secondary gRNAs to boost HDR efficiency or eliminate unwanted indels.

### REFERENCES FOR EXAMPLE 2

1. Scudellari, M. Self-destructing mosquitoes and sterilized rodents: the promise of gene drives. Naturevol. 571 160-162 (2019).
2. National Academies of Sciences, Engineering, and Medicine, Division on Earth and Life Studies, Board on Life Sciences & Committee on Gene Drive Research in Non-Human Organisms: Recommendations for Responsible Conduct. Gene Drives on the Horizon: Advancing Science, Navigating Uncertainty, and Aligning Research with Public Values. (National Academies Press, 2016).
3. James, A. A. Gene drive systems in mosquitoes: rules of the road. Trends Parasitol. 21, 64-67 (2005).
4. Gantz, V. M. et al. Highly efficient Cas9-mediated gene drive for population modification of the malaria vector mosquito Anopheles stephensi. Proc. Natl. Acad. Sci. U.S.A112, E6736-43 (2015).
5. Adolfi, A. et al. Efficient population modification gene-drive rescue system in the malaria mosquito Anopheles stephensi. Nat. Commun. 11, 5553 (2020).
6. Carballar-Lejarazú, R. et al. Next-generation gene drive for population modification of the malaria vector mosquito, Anopheles gambiae. Proc. Natl. Acad. Sci. U.S.A117, 22805-22814 (2020).
7. Hammond, A. et al. A CRISPR-Cas9 gene drive system targeting female reproduction in the malaria mosquito vector Anopheles gambiae. Nat. Biotechnol. 34, 78-83 (2016).
8. Kyrou, K. et al. A CRISPR-Cas9 gene drive targeting doublesex causes complete population suppression in caged Anopheles gambiae mosquitoes. Nat. Biotechnol. 36, 1062-1066 (2018).
9. Hammond, A. et al. Gene-drive suppression of mosquito populations in large cages as a bridge between lab and field. Nat. Commun. 12, 4589 (2021).
10. Courtier-Orgogozo, V., Morizot, B. & Boëte, C. Agricultural pest control with CRISPR-based gene drive: time for public debate. EMBO Rep. 18, 878-880 (2017).
11. Teem, J. L. et al. Genetic Biocontrol for Invasive Species. Front Bioeng Biotechnol8, 452 (2020).
12. Harvey-Samuel, T. et al. Culex quinquefasciatus: status as a threat to island avifauna and options for genetic control. CABI Agriculture and Bioscience2, 1-21 (2021).
13. Grunwald, H. A. et al. Super-Mendelian inheritance mediated by CRISPR-Cas9 in the female mouse germline. Nature566, 105-109 (2019).
14. Weitzel, A. J., Grunwald, H. A., Levina, R. & Gantz, V. M. Meiotic Cas9 expression mediates genotype conversion in the male and female mouse germline. bioRxiv (2021).
15. Godwin, J. et al. Rodent gene drives for conservation: opportunities and data needs. Proc. Biol. Sci. 286, 20191606 (2019).
16. Gantz, V. M. & Bier, E. The mutagenic chain reaction: A method for converting heterozygous to homozygous mutations. Sciencevol. 348 442-444 (2015).
17. Gantz, V. M. & Bier, E. The dawn of active genetics. Bioessays38, 50-63 (2016).
18. Champer, J. et al. Novel CRISPR/Cas9 gene drive constructs reveal insights into mechanisms of resistance allele formation and drive efficiency in genetically diverse populations. PLoS Genet. 13, e1006796 (2017).
19. López Del Amo, V. et al. A transcomplementing gene drive provides a flexible platform for laboratory investigation and potential field deployment. Nat. Commun. 11, 352 (2020).
20. Pham, T. B. et al. Experimental population modification of the malaria vector mosquito, Anopheles stephensi. PLoS Genet. 15, e1008440 (2019).
21. Chen, W. et al. Massively parallel profiling and predictive modeling of the outcomes of CRISPR/Cas9-mediated double-strand break repair. Nucleic Acids Res. 47, 7989-8003 (2019).
22. Tatiossian, K. J. et al. Rational Selection of CRISPR-Cas9 Guide RNAs for Homology-Directed Genome Editing. Mol. Ther. 29, 1057-1069 (2021).
23. Chakrabarti, A. M. et al. Target-Specific Precision of CRISPR-Mediated Genome Editing. Mol. Cell73, 699-713.e6 (2019).
24. Xu, X.-R. S. et al. Active Genetic Neutralizing Elements for Halting or Deleting Gene Drives. Mol. Cell (2020) doi:10.1016/j. molcel. 2020.09.003.
25. Champer, J. et al. Reducing resistance allele formation in CRISPR gene drive. Proc. Natl. Acad. Sci. U.S.A115, 5522-5527 (2018).
26. Oberhofer, G., Ivy, T. & Hay, B. A. Behavior of homing endonuclease gene drives targeting genes required for viability or female fertility with multiplexed guide RNAs. Proc. Natl. Acad. Sci. U.S.A. 115, E9343-E9352 (2018).
27. Hale, M. et al. Homology-Directed Recombination for Enhanced Engineering of Chimeric Antigen Receptor T Cells. Mol Ther Methods Clin Dev4, 192-203 (2017).
28. Quadros, R. M. et al. Easi-CRISPR: a robust method for one-step generation of mice carrying conditional and insertion alleles using long ssDNA donors and CRISPR ribonucleoproteins. Genome Biologyvol. 18 (2017).
29. Port, F., Chen, H.-M., Lee, T. & Bullock, S. L. An optimized CRISPR/Cas toolbox for efficient germline and somatic genome engineering in Drosophila. doi:10.1101/003541.
30. Bassett, A. R., Tibbit, C., Ponting, C. P. & Liu, J.-L. Highly Efficient Targeted Mutagenesis of Drosophila with the CRISPR/Cas9 System. Cell Rep. 6, 1178-1179 (2014).
31. Gloor, G. B. et al. Type I repressors of P element mobility. Genetics135, 81-95 (1993).
32. Clement, K. et al. CRISPResso2 provides accurate and rapid genome editing sequence analysis. Nat. Biotechnol. 37, 224-226 (2019).
33. López Del Amo, V. et al. Small-Molecule Control of Super-Mendelian Inheritance in Gene Drives. Cell Rep. 31, 107841 (2020).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A method for improving genome editing comprising designing and using multiple gRNAs, wherein a primary gRNA targeting a wild-type genomic sequence, and one or more secondary or tertiary gRNAs targeting indel sequence(s).

2. The method of claim 1, wherein said method improves homology-directed repair (HDR)-mediated precision genome editing efficiencies.

3. The method of claim 1, wherein the indel sequence(s) with high frequencies are targeted by end joining pathways.

4. The method of claim 1, wherein the indel sequence(s) are re-targeted for double-stand break (DSB) at a desired genomic locus to be processed by HDR using a same donor template.

5. The method of claim 1, wherein the secondary or tertiary gRNAs decreases unwanted indel products and increases desired precision genome editing outcome.

6. The method of claim 1, wherein the secondary or tertiary gRNAs does not introduce off-target DSBs at a level that impacts cell viability.

7. The method of claim 1, wherein said method improves HDR-mediated precision genome editing efficiency for installation of point mutations, small, insertions, deletions, or gene knock-in.

8. The method of claim 7, wherein deletions are deletions with ssODNs.

9. The method of claim 7, wherein gene knock-in is using dsDNA donor templates.

10. The method of claim 1, wherein said method is used in combination with an existing genome editing method to improve genome editing efficiency without perturbing gene expression levels or cell cycles.

11. The method of claim 10, wherein the existing genome editing method is blocking mutations.

12. The method of claim 10, wherein the existing genome editing method is an additional HDR-enhancing method to further improve precision genome editing rates and decreases unwanted indel rates.

13. The method of claim 1, wherein said method is used with RNP delivery to enhance HDR-mediated precision genome editing efficiencies and decrease unwanted indel frequencies.

14. The method of claim 1, wherein said method is used to improve the frequency of homozygous and heterozygous isogenic clones when using HDR to generate a disease-relevant model system or for disease modeling.

15. (canceled)

16. The method of claim 1, wherein said method is applied in a subject or a system that has HDR as a DNA repair mechanism and suffers from low HDR frequencies, and wherein the HDR frequencies in the subject are increased after applying said method.

17. (canceled)

18. The method of claim 16, wherein the subject is an animal, an insect, a plant, or fungi.

19.-20. (canceled)

21. The method of claim 16, wherein the system comprises a variety of human cells, mammalian cells, and/or a cell line comprising human cells or mammalian cells.

22. (canceled)

23. The method of claim 16, wherein the system comprises a mouse germline or mouse embryo.

24. The method of claim, wherein said method is used to improve CRISPR-based gene drive efficiency by recycling resistance alleles, wherein additional gRNAs are encoded into the gene drive that targets commonly generated resistant alleles.

25. (canceled)

26. The method of claim 1, wherein said method is used to boost efficient gene editing in an animal model for a human disease.


SEQUENCE LISTING (997 sequences)

SEQ ID NO: 1 - DNA, 60 bp, synthetic construct
  source 1..60: mol_type = other DNA
acactctttccctacacgacgctcttccgatctctctgctaattccgtatccagattggc

SEQ ID NO: 2 - DNA, 53 bp, synthetic construct
  source 1..53: mol_type = other DNA
tggagttcagacgtgtgctcttccgatctgcctatatccacggcaatgttagc

SEQ ID NO: 3 - DNA, 59 bp, synthetic construct
  source 1..59: mol_type = other DNA
acactctttccctacacgacgctcttccgatctctctctattcgcagtcggctgatctg

SEQ ID NO: 4 - DNA, 51 bp, synthetic construct
  source 1..51: mol_type = other DNA
tggagttcagacgtgtgctcttccgatctggtcatcctgctggacataggc

SEQ ID NO: 5 - DNA, 22 bp, synthetic construct
  source 1..22: mol_type = other DNA
tttgctcacctgtgattgctcc

SEQ ID NO: 6 - DNA, 42 bp, synthetic construct
  source 1..42: mol_type = other DNA
gtacgcgtatcgataagctttaagatacattgatgagtttgg

SEQ ID NO: 7 - DNA, 36 bp, synthetic construct
  source 1..36: mol_type = other DNA
aagctggagctcctgcaggttgttggttggcacacc

SEQ ID NO: 8 - DNA, 60 bp, synthetic construct
  source 1..60: mol_type = other DNA
cctgcaggagctccagcttttgttaaaaagaacaatatcgatatacgatattttaagttc

SEQ ID NO: 9 - DNA, 54 bp, synthetic construct
  source 1..54: mol_type = other DNA
aaccggtccagtgtccaaaaccaaatgtagctcaaaaaattgcgtatataaatg

SEQ ID NO: 10 - DNA, 60 bp, synthetic construct
  source 1..60: mol_type = other DNA
acatttggttttggacactggaccggttttagagctagaaatagcaagttaaaataaggc

SEQ ID NO: 11 - DNA, 60 bp, synthetic construct
  source 1..60: mol_type = other DNA
gtatcttaaagcttatcgatacgcgtacgctagcaataaatggcgacagcaacaaaaaac

SEQ ID NO: 12 - DNA, 51 bp, synthetic construct
  source 1..51: mol_type = other DNA
caggcatccaagtatcgccaaatgtagctcaaaaaattgcgtatataaatg

SEQ ID NO: 13 - DNA, 54 bp, synthetic construct
  source 1..54: mol_type = other DNA
ggcgatacttggatgcctggttttagagctagaaatagcaagttaaaataaggc

SEQ ID NO: 14 - DNA, 60 bp, synthetic construct
  source 1..60: mol_type = other DNA
acaacctgcaggagctccagcttttgtcgtacaactagggttgcaaccaggtagttggac

SEQ ID NO: 15 - DNA, 60 bp, synthetic construct
  source 1..60: mol_type = other DNA
ctaaaaccaggcatccaagtatcgccgaagattagatccttaaatctgctatttaaatgg

SEQ ID NO: 16 - DNA, 60 bp, synthetic construct
  source 1..60: mol_type = other DNA
aacttaaaatatcgtatatcgatattgttctttttacaacaattataaaaaaaagcgcac

SEQ ID NO: 17 - DNA, 60 bp, synthetic construct
  source 1..60: mol_type = other DNA
gtaaaaagaacaatatcgatatacgatattttaagttcctttcgcgcttagccattacac

SEQ ID NO: 18 - DNA, 47 bp, synthetic construct
  source 1..47: mol_type = other DNA
ggagcaatcacaggtgagcaaaaaagacgtctgcggcgatcgaaagg

SEQ ID NO: 19 - DNA, 37 bp, synthetic construct
  source 1..37: mol_type = other DNA
ggagcaatcacaggtgagcaaaaaagcaccgactcgg

SEQ ID NO: 20 - DNA, 53 bp, synthetic construct
  source 1..53: mol_type = other DNA
cagtatatataggaaagatatccgggtgaacttcggttttggacactggaccg

SEQ ID NO: 21 - DNA, 34 bp, synthetic construct
  source 1..34: mol_type = other DNA
gaagttcacccggatatctttcctatatatactg

SEQ ID NO: 22 - DNA, 21 bp, synthetic construct
  source 1..21: mol_type = other DNA
gctggtcaaccggacacgcgg

SEQ ID NO: 23 - DNA, 24 bp, synthetic construct
  source 1..24: mol_type = other DNA
agggagccgataaagaggtcatcc

SEQ ID NO: 24 - DNA, 22 bp, synthetic construct
  source 1..22: mol_type = other DNA
tattatccagaaaacagacagc

SEQ ID NO: 25 - DNA, 22 bp, synthetic construct
  source 1..22: mol_type = other DNA
ggacataccaaatataccctcc

SEQ ID NO: 26 - DNA, 35 bp, synthetic construct
  source 1..35: mol_type = other DNA
gttccagagcctcccagagctgcagatagaaatca

SEQ ID NO: 27 - DNA, 35 bp, synthetic construct
  source 1..35: mol_type = other DNA
gatccagagcctcccagagctgcagatagaaatca

SEQ ID NO: 28 - DNA, 35 bp, synthetic construct
  source 1..35: mol_type = other DNA
gttccagagcctcccagagctgcagatagaaaaca

SEQ ID NO: 29 - DNA, 35 bp, synthetic construct
  source 1..35: mol_type = other DNA
gttccagagcctcccagagctgcagataggaatca

SEQ ID NO: 30 - DNA, 35 bp, synthetic construct
  source 1..35: mol_type = other DNA
gttacagagcctcccagagctgcagatagaaatca

SEQ ID NO: 31 - DNA, 35 bp, synthetic construct
  source 1..35: mol_type = other DNA
gttccagagcctcccaagagctgcagatagaaatc

SEQ ID NO: 32 - DNA, 27 bp, synthetic construct
  source 1..27: mol_type = other DNA
gttccagagcctcccagatagaaatca

SEQ ID NO: 33 - DNA, 35 bp, synthetic construct
  source 1..35: mol_type = other DNA
gttccagagcctcccaaagagctgcagatagaaat

SEQ ID NO: 34 - DNA, 35 bp, synthetic construct
  source 1..35: mol_type = other DNA
gttccagagcctcccacagagctgcagatagaaat

SEQ ID NO: 35 - DNA, 35 bp, synthetic construct
  source 1..35: mol_type = other DNA
gttccagagcctcccaaaagagctgcagatagaaa

SEQ ID NO: 36 - DNA, 20 bp, synthetic construct
  source 1..20: mol_type = other DNA
gctattctcgcagctcacca

SEQ ID NO: 37 - DNA, 52 bp, synthetic construct
  source 1..52: mol_type = other DNA
gcgcgcccggctattctcgcagctcaccatggatgatgatatcgccgcgctc

SEQ ID NO: 38 - DNA, 49 bp, synthetic construct
  source 1..49: mol_type = other DNA
gcgcgcccggctattctcgcagctcatggatgatgatatcgccgcgctc

SEQ ID NO: 39 - DNA, 53 bp, synthetic construct
  source 1..53: mol_type = other DNA
gcgcgcccggctattctcgcagctcaaccatggatgatgatatcgccgcgctc

SEQ ID NO: 40 - DNA, 44 bp, synthetic construct
  source 1..44: mol_type = other DNA
gcgcgcccggctattctcgcatggatgatgatatcgccgcgctc

SEQ ID NO: 41 - DNA, 50 bp, synthetic construct
  source 1..50: mol_type = other DNA
gcgcgcccggctattctcgcagctccatggatgatgatatcgccgcgctc

SEQ ID NO: 42 - DNA, 50 bp, synthetic construct
  source 1..50: mol_type = other DNA
gcgcgcccggctattctcgcagctcaatggatgatgatatcgccgcgctc

SEQ ID NO: 43 - DNA, 49 bp, synthetic construct
  source 1..49: mol_type = other DNA
gcgcgcccggctattctcgcagcccatggatgatgatatcgccgcgctc

SEQ ID NO: 44 - DNA, 51 bp, synthetic construct
  source 1..51: mol_type = other DNA
gcgcgcccggctattctcgcagctcccatggatgatgatatcgccgcgctc

SEQ ID NO: 45 - DNA, 53 bp, synthetic construct
  source 1..53: mol_type = other DNA
gcgcgcccggctattctcgcagctcacatggatgatgatatcgccgcgcgctc

SEQ ID NO: 46 - DNA, 48 bp, synthetic construct
  source 1..48: mol_type = other DNA
gcgcgcccggctattctcgcagccatggatgatgatatcgccgcgctc

SEQ ID NO: 47 - DNA, 47 bp, synthetic construct
  source 1..47: mol_type = other DNA
gcgcgcccggctattctcgcagcatggatgatgatatcgccgcgctc

SEQ ID NO: 48 - DNA, 40 bp, synthetic construct
  source 1..40: mol_type = other DNA
ccggctattctcgcagctcaccatggatgatgatatcgcc

SEQ ID NO: 49 - DNA, 40 bp, synthetic construct
  source 1..40: mol_type = other DNA
ccggctattctcgcagctcaaccatggatgatgatatcgc

SEQ ID NO: 50 - DNA, 40 bp, synthetic construct
  source 1..40: mol_type = other DNA
ccggctattctcgcagctcaaaccatggatgatgatatcg

SEQ ID NO: 51 - DNA, 37 bp, synthetic construct
  source 1..37: mol_type = other DNA
ccggctattctcgcagctcatggatgatgatatcgcc

SEQ ID NO: 52 - DNA, 38 bp, synthetic construct
  source 1..38: mol_type = other DNA
ccggctattctcgcagctcaatggatgatgatatcgcc

SEQ ID NO: 53 - DNA, 39 bp, synthetic construct
  source 1..39: mol_type = other DNA
ccggctattctcgcagctcacatggatgatgatatcgcc

SEQ ID NO: 54 - DNA, 38 bp, synthetic construct
  source 1..38: mol_type = other DNA
ccggctattctcgcagctccatggatgatgatatcgcc

SEQ ID NO: 55 - DNA, 19 bp, synthetic construct
  source 1..19: mol_type = other DNA
ccggctattctcgcagctc

SEQ ID NO: 56 - DNA, 39 bp, synthetic construct
  source 1..39: mol_type = other DNA
ccggctattctcgcagctcaaatggatgatgatatcgcc

SEQ ID NO: 57 - DNA, 40 bp, synthetic construct
  source 1..40: mol_type = other DNA
ccggctattctcgcagctcaaaaccatggatgatgatatc

SEQ ID NO: 58 - DNA, 32 bp, synthetic construct
  source 1..32: mol_type = other DNA
ccggctattctcgcagctcagatgatatcgcc

SEQ ID NO: 59 - DNA, 36 bp, synthetic construct
  source 1..36: mol_type = other DNA
ccggctattctcgcagctcaggatgatgatatcgcc

SEQ ID NO: 60 - DNA, 33 bp, synthetic construct
  source 1..33: mol_type = other DNA
ccggctattctcgcagctcatgatgatatcgcc

SEQ ID NO: 61 - DNA, 34 bp, synthetic construct
  source 1..34: mol_type = other DNA
ccggctattctcgcagctcaatgatgatatcgcc

SEQ ID NO: 62 - DNA, 38 bp, synthetic construct
  source 1..38: mol_type = other DNA
ccggctattctcgcagcaccatggatgatgatatcgcc

SEQ ID NO: 63 - DNA, 39 bp, synthetic construct
  source 1..39: mol_type = other DNA
ccggctattctcgcagctcccatggatgatgatatcgcc

SEQ ID NO: 64 - DNA, 24 bp, synthetic construct
  source 1..24: mol_type = other DNA
ccggctatggatgatgatatcgcc

SEQ ID NO: 65 - DNA, 25 bp, synthetic construct
  source 1..25: mol_type = other DNA
ccggctattctcgcagctcatcgcc

SEQ ID NO: 66 - DNA, 33 bp, synthetic construct
  source 1..33: mol_type = other DNA
ccggctattctcgccatggatgatgatatcgcc

SEQ ID NO: 67 - DNA, 40 bp, synthetic construct
  source 1..40: mol_type = other DNA
ccggctattctcgcagctcatccatggatgatgatatcgc

SEQ ID NO: 68 - DNA, 31 bp, synthetic construct
  source 1..31: mol_type = other DNA
ccggctattctcgcaggatgatgatatcgcc

SEQ ID NO: 69 - DNA, 37 bp, synthetic construct
  source 1..37: mol_type = other DNA
ccggctattctcgcagcccatggatgatgatatcgcc

SEQ ID NO: 70 - DNA, 32 bp, synthetic construct
  source 1..32: mol_type = other DNA
ccggctattctcgcatggatgatgatatcgcc

SEQ ID NO: 71 - DNA, 17 bp, synthetic construct
  source 1..17: mol_type = other DNA
ccggctatgatatcgcc

SEQ ID NO: 72 - DNA, 27 bp, synthetic construct
  source 1..27: mol_type = other DNA
ccggctattctcgcagctcatatcgcc

SEQ ID NO: 73 - DNA, 29 bp, synthetic construct
  source 1..29: mol_type = other DNA
ccggctattctcgcagctcagatatcgcc

SEQ ID NO: 74 - DNA, 22 bp, synthetic construct
  source 1..22: mol_type = other DNA
ccggctattctcgcagctcgcc

SEQ ID NO: 75 - DNA, 28 bp, synthetic construct
  source 1..28: mol_type = other DNA
ccggctattctcgcagctcaatatcgcc

SEQ ID NO: 76 - DNA, 26 bp, synthetic construct
  source 1..26: mol_type = other DNA
ccggctattctcgcagctcaatcgcc

SEQ ID NO: 77 - DNA, 14 bp, synthetic construct
  source 1..14: mol_type = other DNA
ccggctattctcgc

SEQ ID NO: 78 - DNA, 30 bp, synthetic construct
  source 1..30: mol_type = other DNA
ccggctattctcgcagctcatgatatcgcc

SEQ ID NO: 79 - DNA, 36 bp, synthetic construct
  source 1..36: mol_type = other DNA
ccggctattctcgcagccatggatgatgatatcgcc

SEQ ID NO: 80 - DNA, 35 bp, synthetic construct
  source 1..35: mol_type = other DNA
ccggctattctcgcagctcagatgatgatatcgcc

SEQ ID NO: 81 - DNA, 17 bp, synthetic construct
  source 1..17: mol_type = other DNA
ccggctattctcgcagc

SEQ ID NO: 82 - DNA, 14 bp, synthetic construct
  source 1..14: mol_type = other DNA
ccggctatatcgcc

SEQ ID NO: 83 - DNA, 30 bp, synthetic construct
  source 1..30: mol_type = other DNA
ccggctattctcatggatgatgatatcgcc

SEQ ID NO: 84 - DNA, 20 bp, synthetic construct
  source 1..20: mol_type = other DNA
gccgacatctctatggtgaa

SEQ ID NO: 85 - DNA, 52 bp, synthetic construct
  source 1..52: mol_type = other DNA
tagccgaatgccgacatctctatggtgaatggagacacttcaacattgacaa

SEQ ID NO: 86 - DNA, 53 bp, synthetic construct
  source 1..53: mol_type = other DNA
tagccgaatgccgacatctctatggttgaatggagacacttcaacattgacaa

SEQ ID NO: 87 - DNA, 45 bp, synthetic construct
  source 1..45: mol_type = other DNA
tagccgaatgccgacatctctatggagacacttcaacattgacaa

SEQ ID NO: 88 - DNA, 50 bp, synthetic construct
  source 1..50: mol_type = other DNA
tagccgaatgccgacatctctatggaatggagacacttcaacattgacaa

SEQ ID NO: 89 - DNA, 50 bp, synthetic construct
  source 1..50: mol_type = other DNA
tagccgaatgccgacatctctatggtatggagacacttcaacattgacaa

SEQ ID NO: 90 - DNA, 49 bp, synthetic construct
  source 1..49: mol_type = other DNA
tagccgaatgccgacatctctatgaatggagacacttcaacattgacaa

SEQ ID NO: 91 - DNA, 51 bp, synthetic construct
  source 1..51: mol_type = other DNA
tagccgaatgccgacatctctatgggaatggagacacttcaacattgacaa

SEQ ID NO: 92 - DNA, 51 bp, synthetic construct
  source 1..51: mol_type = other DNA
tagccgaatgccgacatctctatggtaatggagacacttcaacattgacaa

SEQ ID NO: 93 - DNA, 48 bp, synthetic construct
  source 1..48: mol_type = other DNA
tagccgaatgccgacatctctatggtggagacacttcaacattgacaa

SEQ ID NO: 94 - DNA, 31 bp, synthetic construct
  source 1..31: mol_type = other DNA
tagccgaatggagacacttcaacattgacaa

SEQ ID NO: 95 - DNA, 53 bp, synthetic construct
  source 1..53: mol_type = other DNA
tagccgaatgccgacatctctatggtagaatggagacacttcaacattgacaa

SEQ ID NO: 96 - DNA, 46 bp, synthetic construct
  source 1..46: mol_type = other DNA
tagccgaatgccgacatctctatgggagacacttcaacattgacaa

SEQ ID NO: 97 - DNA, 40 bp, synthetic construct
  source 1..40: mol_type = other DNA
atgttgaagtgtctccattcaccatagagatgtcggcatt

SEQ ID NO: 98 - DNA, 40 bp, synthetic construct
  source 1..40: mol_type = other DNA
atgttgaagtgtctccattcaaccatagagatgtcggcat

SEQ ID NO: 99 - DNA, 33 bp, synthetic construct
  source 1..33: mol_type = other DNA
atgttgaagtgtctccatagagatgtcggcatt

SEQ ID NO: 100 - DNA, 37 bp, synthetic construct
  source 1..37: mol_type = other DNA
atgttgaagtgtctccattcatagagatgtcggcatt

SEQ ID NO: 101 - DNA, 36 bp, synthetic construct
  source 1..36: mol_type = other DNA
atgttgaagtgtctccattcaagagatgtcggcatt

SEQ ID NO: 102 - DNA, 38 bp, synthetic construct
  source 1..38: mol_type = other DNA
atgttgaagtgtctccattcaatagagatgtcggcatt

SEQ ID NO: 103 - DNA, 37 bp, synthetic construct
  source 1..37: mol_type = other DNA
atgttgaagtgtcacgttccatagagatgtcggcatt

SEQ ID NO: 104 - DNA, 39 bp, synthetic construct
  source 1..39: mol_type = other DNA
atgttgaagtgtctccattcacatagagatgtcggcatt

SEQ ID NO: 105 - DNA, 35 bp, synthetic construct
  source 1..35: mol_type = other DNA
atgttgaagtgtctccattcagagatgtcggcatt

SEQ ID NO: 106 - DNA, 34 bp, synthetic construct
  source 1..34: mol_type = other DNA
atgttgaagtgtctccattcaagatgtcggcatt

SEQ ID NO: 107 - DNA, 38 bp, synthetic construct
  source 1..38: mol_type = other DNA
atgttgaagtgtctccattccatagagatgtcggcatt

SEQ ID NO: 108 - DNA, 33 bp, synthetic construct
  source 1..33: mol_type = other DNA
atgttgaagtgtctccattcagatgtcggcatt

SEQ ID NO: 109 - DNA, 32 bp, synthetic construct
  source 1..32: mol_type = other DNA
atgttgaagtgtctccattcaatgtcggcatt

SEQ ID NO: 110 - DNA, 39 bp, synthetic construct
  source 1..39: mol_type = other DNA
atgttgaagtgtctccattcccatagagatgtcggcatt

SEQ ID NO: 111 - DNA, 39 bp, synthetic construct
  source 1..39: mol_type = other DNA
atgttgaagtgtctccattaccatagagatgtcggcatt

SEQ ID NO: 112 - DNA, 19 bp, synthetic construct
  source 1..19: mol_type = other DNA
atgttgaagtgtcggcatt

SEQ ID NO: 113 - DNA, 33 bp, synthetic construct
  source 1..33: mol_type = other DNA
atgttgaagtgtcaccatagagatgtcggcatt

SEQ ID NO: 114 - DNA, 30 bp, synthetic construct
  source 1..30: mol_type = other DNA
atgttgaagtgtctccattcagtcggcatt

SEQ ID NO: 115 - DNA, 20 bp, synthetic construct
  source 1..20: mol_type = other DNA
atgttgaagtgtctccattc

SEQ ID NO: 116 - DNA, 20 bp, synthetic construct
  source 1..20: mol_type = other DNA
ggaactctcgagatggcaga

SEQ ID NO: 117 - DNA, 52 bp, synthetic construct
  source 1..52: mol_type = other DNA
tagggacttggaactctcgagatggcagatggaatcccacagacttgaagtg

SEQ ID NO: 118 - DNA, 45 bp, synthetic construct
  source 1..45: mol_type = other DNA
tagggacttggaactctcgagatggaatcccacagacttgaagtg

SEQ ID NO: 119 - DNA, 53 bp, synthetic construct
  source 1..53: mol_type = other DNA
tagggacttggaactctcgagatggccagatggaatcccacagacttgaagtg

SEQ ID NO: 120 - DNA, 53 bp, synthetic construct
  source 1..53: mol_type = other DNA
tagggacttggaactctcgagatggctagatggaatcccacagacttgaagtg

SEQ ID NO: 121 - DNA, 53 bp, synthetic construct
  source 1..53: mol_type = other DNA
tagggacttggaactctcgagatggcaagatggaatcccacagacttgaagtg

SEQ ID NO: 122 - DNA, 43 bp, synthetic construct
  source 1..43: mol_type = other DNA
tagggacttggaactctcgatggaatcccacagacttgaagtg

SEQ ID NO: 123 - DNA, 49 bp, synthetic construct
  source 1..49: mol_type = other DNA
tagggacttggaactctcgagatggatggaatcccacagacttgaagtg

SEQ ID NO: 124 - DNA, 53 bp, synthetic construct
  source 1..53: mol_type = other DNA
tagggacttggaactctcgagatggcgagatggaatcccacagacttgaagtg

SEQ ID NO: 125 - DNA, 51 bp, synthetic construct
  source 1..51: mol_type = other DNA
tagggacttggaactctcgagatggagatggaatcccacagacttgaagtg

SEQ ID NO: 126 - DNA, 51 bp, synthetic construct
  source 1..51: mol_type = other DNA
tagggacttggaactctcgagatggcgatggaatcccacagacttgaagtg

SEQ ID NO: 127 - DNA, 48 bp, synthetic construct
  source 1..48: mol_type = other DNA
tagggacttggaactctcgagatgatggaatcccacagacttgaagtg

SEQ ID NO: 128 - DNA, 40 bp, synthetic construct
  source 1..40: mol_type = other DNA
aagtctgtgggattccatctgccatctcgagagttccaag

SEQ ID NO: 129 - DNA, 40 bp, synthetic construct
  source 1..40: mol_type = other DNA
aagtctgtgggattccatctggccatctcgagagttccaa

SEQ ID NO: 130 - DNA, 37 bp, synthetic construct
  source 1..37: mol_type = other DNA
aagtctgtgggattccatctgtctcgagagttccaag

SEQ ID NO: 131 - DNA, 39 bp, synthetic construct
  source 1..39: mol_type = other DNA
aagtctgtgggattccatctccatctcgagagttccaag

SEQ ID NO: 132 - DNA, 38 bp, synthetic construct
  source 1..38: mol_type = other DNA
aagtctgtgggattccatctgatctcgagagttccaag

SEQ ID NO: 133 - DNA, 35 bp, synthetic construct
  source 1..35: mol_type = other DNA
aagtctgtgggattccatctctcgagagttccaag

SEQ ID NO: 134 - DNA, 37 bp, synthetic construct
  source 1..37: mol_type = other DNA
aagtctgtgggattccatctatctcgagagttccaag

SEQ ID NO: 135 - DNA, 36 bp, synthetic construct
  source 1..36: mol_type = other DNA
aagtctgtgggattccatctgctcgagagttccaag

SEQ ID NO: 136 - DNA, 39 bp, synthetic construct
  source 1..39: mol_type = other DNA
aagtctgtgggattccatcgccatctcgagagttccaag

SEQ ID NO: 137 - DNA, 39 bp, synthetic construct
  source 1..39: mol_type = other DNA
aagtctgtgggattccatctgcatctcgagagttccaag

SEQ ID NO: 138 - DNA, 38 bp, synthetic construct
  source 1..38: mol_type = other DNA
aagtctgtgggattccatctcatctcgagagttccaag

SEQ ID NO: 139 - DNA, 34 bp, synthetic construct
  source 1..34: mol_type = other DNA
aagtctgtgggattgccatctcgagagttccaag

SEQ ID NO: 140 - DNA, 34 bp, synthetic construct
  source 1..34: mol_type = other DNA
aagtctgtgggattccatctgcgagagttccaag

SEQ ID NO: 141 - DNA, 38 bp, synthetic construct
  source 1..38: mol_type = other DNA
aagtctgtgggattccatgccatctcgagagttccaag

SEQ ID NO: 142 - DNA, 35 bp, synthetic construct
  source 1..35: mol_type = other DNA
aagtctgtgggattccatctgtcgagagttccaag

SEQ ID NO: 143 - DNA, 40 bp, synthetic construct
  source 1..40: mol_type = other DNA
aagtctgtgggattccatcttgccatctcgagagttccaa

SEQ ID NO: 144 - DNA, 20 bp, synthetic construct
  source 1..20: mol_type = other DNA
ggaatcccttctgcagcacc

SEQ ID NO: 145 - DNA, 52 bp, synthetic construct
  source 1..52: mol_type = other DNA
gcacctcatggaatcccttctgcagcacctggatcgcttttccgagcttctg

SEQ ID NO: 146 - DNA, 53 bp, synthetic construct
  source 1..53: mol_type = other DNA
gcacctcatggaatcccttctgcagccacctggatcgcttttccgagcttctg

SEQ ID NO: 147 - DNA, 43 bp, synthetic construct
  source 1..43: mol_type = other DNA
gcacctcatggaatcccttctggatcgcttttccgagcttctg

SEQ ID NO: 148 - DNA, 40 bp, synthetic construct
  source 1..40: mol_type = other DNA
gcacctcatggaatccctggatcgcttttccgagcttctg

SEQ ID NO: 149 - DNA, 53 bp, synthetic construct
  source 1..53: mol_type = other DNA
gcacctcatggaatcccttctgcagctacctggatcgcttttccgagcttctg

SEQ ID NO: 150 - DNA, 53 bp, synthetic construct
  source 1..53: mol_type = other DNA
gcacctcatggaatcccttctgcagcaacctggatcgcttttccgagcttctg

SEQ ID NO: 151 - DNA, 50 bp, synthetic construct
  source 1..50: mol_type = other DNA
gcacctcatggaatcccttctgcagcctggatcgcttttccgagcttctg

SEQ ID NO: 152 - DNA, 50 bp, synthetic construct
  source 1..50: mol_type = other DNA
gcacctcatggaatcccttctgcaacctggatcgcttttccgagcttctg

SEQ ID NO: 153 - DNA, 41 bp, synthetic construct
  source 1..41: mol_type = other DNA
gcacctcatggaatcccttctgcagcttttccgagcttctg

SEQ ID NO: 154 - DNA, 49 bp, synthetic construct
  source 1..49: mol_type = other DNA
gcacctcatggaatcccttctgcagctggatcgcttttccgagcttctg

SEQ ID NO: 155 - DNA, 49 bp, synthetic construct
  source 1..49: mol_type = other DNA
gcacctcatggaatcccttctgcacctggatcgcttttccgagcttctg

SEQ ID NO: 156 - DNA, 40 bp, synthetic construct
  source 1..40: mol_type = other DNA
catggaatcccttctgcagcacctggatcgcttttccgag

SEQ ID NO: 157 - DNA, 37 bp, synthetic construct
  source 1..37: mol_type = other DNA
catggaatcccttctgcacctggatcgcttttccgag

SEQ ID NO: 158 - DNA, 40 bp, synthetic construct
  source 1..40: mol_type = other DNA
catggaatcccttctgcagccacctggatcgcttttccga

SEQ ID NO: 159 - DNA, 38 bp, synthetic construct
  source 1..38: mol_type = other DNA
catggaatcccttctgcagcctggatcgcttttccgag

SEQ ID NO: 160 - DNA, 39 bp, synthetic construct
  source 1..39: mol_type = other DNA
catggaatcccttctgcagacctggatcgcttttccgag

SEQ ID NO: 161 - DNA, 38 bp, synthetic construct
  source 1..38: mol_type = other DNA
catggaatcccttctgcaacctggatcgcttttccgag

SEQ ID NO: 162 - DNA, 34 bp, synthetic construct
  source 1..34: mol_type = other DNA
catggaatcccttcacctggatcgcttttccgag

SEQ ID NO: 163 - DNA, 36 bp, synthetic construct
  source 1..36: mol_type = other DNA
catggaatcccttctgacctggatcgcttttccgag

SEQ ID NO: 164 - DNA, 29 bp, synthetic construct
  source 1..29: mol_type = other DNA
catggaatcccttctgcagcttttccgag

SEQ ID NO: 165 - DNA, 39 bp, synthetic construct
  source 1..39: mol_type = other DNA
catggaatcccttctgcagccctggatcgcttttccgag

SEQ ID NO: 166 - DNA, 37 bp, synthetic construct
  source 1..37: mol_type = other DNA
catggaatcccttctgcagctggatcgcttttccgag

SEQ ID NO: 167 - DNA, 40 bp, synthetic construct
  source 1..40: mol_type = other DNA
catggaatcccttctgcagctacctggatcgcttttccga

SEQ ID NO: 168 - DNA, 34 bp, synthetic construct
  source 1..34: mol_type = other DNA
catggaatcccttctgcagcatcgcttttccgag

SEQ ID NO: 169 - DNA, 31 bp, synthetic construct
  source 1..31: mol_type = other DNA
catggaatcccttctggatcgcttttccgag

SEQ ID NO: 170 - DNA, 35 bp, synthetic construct
  source 1..35: mol_type = other DNA
catggaatcccttctacctggatcgcttttccgag

SEQ ID NO: 171 - DNA, 40 bp, synthetic construct
  source 1..40: mol_type = other DNA
catggaatcccttctgcagcgacctggatcgcttttccga

SEQ ID NO: 172 - DNA, 40 bp, synthetic construct
  source 1..40: mol_type = other DNA
catggaatcccttctgcagcaacctggatcgcttttccga

SEQ ID NO: 173 - DNA, 31 bp, synthetic construct
  source 1..31: mol_type = other DNA
catggaatcccttctgcagcgcttttccgag

SEQ ID NO: 174 - DNA, 16 bp, synthetic construct
  source 1..16: mol_type = other DNA
ggatcgcttttccgag

SEQ ID NO: 175 - DNA, 32 bp, synthetic construct
  source 1..32: mol_type = other DNA
catggaatcccttctgcagccgcttttccgag

SEQ ID NO: 176 - DNA, 35 bp, synthetic construct
  source 1..35: mol_type = other DNA
catggaatcccttctgcagcgatcgcttttccgag

SEQ ID NO: 177 - DNA, 28 bp, synthetic construct
  source 1..28: mol_type = other DNA
catggaatccctggatcgcttttccgag

SEQ ID NO: 178 - DNA, 33 bp, synthetic construct
  source 1..33: mol_type = other DNA
catggaatcccttctgcagctcgcttttccgag

SEQ ID NO: 179 - DNA, 33 bp, synthetic construct
  source 1..33: mol_type = other DNA
catggaatcccttacctggatcgcttttccgag

SEQ ID NO: 180 - DNA, 40 bp, synthetic construct
  source 1..40: mol_type = other DNA
caagcactacctacgtcagcacctgggaccccgccaccgt

SEQ ID NO: 181 - DNA, 38 bp, synthetic construct
  source 1..38: mol_type = other DNA
catggaatcccttctgcagcttctggcggtctcaagca

SEQ ID NO: 182 - DNA, 36 bp, synthetic construct
  source 1..36: mol_type = other DNA
catggaatcccttctgcagcggatcgcttttccgag

SEQ ID NO: 183 - DNA, 35 bp, synthetic construct
  source 1..35: mol_type = other DNA
catggaatcccttccacctggatcgcttttccgag

SEQ ID NO: 184 - DNA, 26 bp, synthetic construct
  source 1..26: mol_type = other DNA
catggaacctggatcgcttttccgag

SEQ ID NO: 185 - DNA, 20 bp, synthetic construct
  source 1..20: mol_type = other DNA
ggaacgttcgcttttccgag

SEQ ID NO: 186 - DNA, 38 bp, synthetic construct
  source 1..38: mol_type = other DNA
catggaatcccttctgccacctggatcgcttttccgag

SEQ ID NO: 187 - DNA, 40 bp, synthetic construct
  source 1..40: mol_type = other DNA
gatggaatcccttctgcagcacctggatcgcttttccgag

SEQ ID NO: 188 - DNA, 23 bp, synthetic construct
  source 1..23: mol_type = other DNA
catggaatcccttctgcagcgag

SEQ ID NO: 189 - DNA, 31 bp, synthetic construct
  source 1..31: mol_type = other DNA
catggaatcccttctgcatcgcttttccgag

SEQ ID NO: 190 - DNA, 40 bp, synthetic construct
  source 1..40: mol_type = other DNA
catggaatcccttctgcagcacctggatggcttttccgag

SEQ ID NO: 191 - DNA, 31 bp, synthetic construct
  source 1..31: mol_type = other DNA
catggaatcccacctggatcgcttttccgag

SEQ ID NO: 192 - DNA, 24 bp, synthetic construct
  source 1..24: mol_type = other DNA
catggaatcccttctgcagccgag

SEQ ID NO: 193 - DNA, 35 bp, synthetic construct
  source 1..35: mol_type = other DNA
catggaatcccttctggaacgttcgcttttccgag

SEQ ID NO: 194 - DNA, 20 bp, synthetic construct
  source 1..20: mol_type = other DNA
catggtgcacctgactcctg

SEQ ID NO: 195 - DNA, 52 bp, synthetic construct
  source 1..52: mol_type = other DNA
aacagacaccatggtgcacctgactcctgaggagaagtctgccgttactgcc

SEQ ID NO: 196 - DNA, 45 bp, synthetic construct
  source 1..45: mol_type = other DNA
aacagacaccatggtgcacctgaggagaagtctgccgttactgcc

SEQ ID NO: 197 - DNA, 49 bp, synthetic construct
  source 1..49: mol_type = other DNA
aacagacaccatggtgcacctgactgaggagaagtctgccgttactgcc

SEQ ID NO: 198 - DNA, 51 bp, synthetic construct
  source 1..51: mol_type = other DNA
aacagacaccatggtgcacctgactctgaggagaagtctgccgttactgcc

SEQ ID NO: 199 - DNA, 53 bp, synthetic construct
  source 1..53: mol_type = other DNA
aacagacaccatggtgcacctgactccctgaggagaagtctgccgttactgcc

SEQ ID NO: 200 - DNA, 42 bp, synthetic construct
  source 1..42: mol_type = other DNA
aacagacaccatggtgcacctgagaagtctgccgttactgcc

SEQ ID NO: 201 - DNA, 49 bp, synthetic construct
  source 1..49: mol_type = other DNA
aacagacaccatggtgcacctgactcaggagaagtctgccgttactgcc

SEQ ID NO: 202 - DNA, 40 bp, synthetic construct
  source 1..40: mol_type = other DNA
aacagacaccatggtgcacctgaagtctgccgttactgcc

SEQ ID NO: 203 - DNA, 39 bp, synthetic construct
  source 1..39: mol_type = other DNA
aacagacaccatggtgaggagaagtctgccgttactgcc

SEQ ID NO: 204 - DNA, 39 bp, synthetic construct
  source 1..39: mol_type = other DNA
aacagacaccatggtgcacctgactctgccgttactgcc

SEQ ID NO: 205 - DNA, 50 bp, synthetic construct
  source 1..50: mol_type = other DNA
aacagacaccatggtgcacctgacttgaggagaagtctgccgttactgcc

SEQ ID NO: 206 - DNA, 40 bp, synthetic construct
  source 1..40: mol_type = other DNA
caccatggtgcacctgactcctgaggagaagtctgccgtt

SEQ ID NO: 207 - DNA, 39 bp, synthetic construct
  source 1..39: mol_type = other DNA
caccatggtgcacctgactctgaggagaagtctgccgtt

SEQ ID NO: 208 - DNA, 33 bp, synthetic construct
  source 1..33: mol_type = other DNA
caccatggtgcacctgaggagaagtctgccgtt

SEQ ID NO: 209 - DNA, 40 bp, synthetic construct
  source 1..40: mol_type = other DNA
caccatggtgcatctgactcctgaggagaagactgctgtc

SEQ ID NO: 210 - DNA, 38 bp, synthetic construct
  source 1..38: mol_type = other DNA
caccatggtgcacctgactcgaggagaagtctgccgtt

SEQ ID NO: 211 - DNA, 40 bp, synthetic construct
  source 1..40: mol_type = other DNA
caccatggtgcacctgactctcctgaggagaagtctgccg

SEQ ID NO: 212 - DNA, 37 bp, synthetic construct
  source 1..37: mol_type = other DNA
caccatggtgcacctgactgaggagaagtctgccgtt

SEQ ID NO: 213 - DNA, 36 bp, synthetic construct
  source 1..36: mol_type = other DNA
caccatggtgcacctgctgaggagaagtctgccgtt

SEQ ID NO: 214 - DNA, 38 bp, synthetic construct
  source 1..38: mol_type = other DNA
caccatggtgcacctgacctgaggagaagtctgccgtt

SEQ ID NO: 215 - DNA, 40 bp, synthetic construct
  source 1..40: mol_type = other DNA
caccatggtgcacctgactccctgaggagaagtctgccgt

SEQ ID NO: 216 - DNA, 35 bp, synthetic construct
  source 1..35: mol_type = other DNA
caccatggtgcacctgactcgagaagtctgccgtt

SEQ ID NO: 217 - DNA, 38 bp, synthetic construct
  source 1..38: mol_type = other DNA
caccatggtgcacctgtcctgaggagaagtctgccgtt

SEQ ID NO: 218 - DNA, 39 bp, synthetic construct
  source 1..39: mol_type = other DNA
caccatggtgcacctgactctgaggagaagactgctgtc

SEQ ID NO: 219 - DNA, 37 bp, synthetic construct
  source 1..37: mol_type = other DNA
caccatggtgcacctgcctgaggagaagtctgccgtt

SEQ ID NO: 220 - DNA, 40 bp, synthetic construct
  source 1..40: mol_type = other DNA
caccatggtgcatctgactcctgaggagaagtctgccgtt

SEQ ID NO: 221 - DNA, 36 bp, synthetic construct
  source 1..36: mol_type = other DNA
caccatggtgcacctgactcggagaagtctgccgtt

SEQ ID NO: 222 - DNA, 33 bp, synthetic construct
  source 1..33: mol_type = other DNA
caccatggtgcacctgaggagaagactgctgtc

SEQ ID NO: 223 - DNA, 40 bp, synthetic construct
  source 1..40: mol_type = other DNA
caccatggtgcacctgacttctgaggagaagtctgccgtt

SEQ ID NO: 224 - DNA, 33 bp, synthetic construct
  source 1..33: mol_type = other DNA
caccatggtgcacctgactcgaagtctgccgtt

SEQ ID NO: 225 - DNA, 40 bp, synthetic construct
  source 1..40: mol_type = other DNA
caccatggtgcacctgactcctcctgaggagaagtctgcc

SEQ ID NO: 226 - DNA, 40 bp, synthetic construct
  source 1..40: mol_type = other DNA
caccagggtgcacctgactcctgaggagaagtctgccgtt

SEQ ID NO: 227 - DNA, 20 bp, synthetic construct
  source 1..20: mol_type = other DNA
acttctcctcaggagtcagg

SEQ ID NO: 228 - DNA, 52 bp, synthetic construct
  source 1..52: mol_type = other DNA
taacggcagacttctcctcaggagtcaggtgcaccatggtgtctgtttgagg

SEQ ID NO: 229 - DNA, 45 bp, synthetic construct
  source 1..45: mol_type = other DNA
taacggcagacttctcctcaggtgcaccatggtgtctgtttgagg

SEQ ID NO: 230 - DNA, 53 bp, synthetic construct
  source 1..53: mol_type = other DNA
taacggcagacttctcctcaggagtccaggtgcaccatggtgtctgtttgagg

SEQ ID NO: 231 - DNA, 48 bp, synthetic construct
  source 1..48: mol_type = other DNA
taacggcagacttctcctcaggaggtgcaccatggtgtctgtttgagg

SEQ ID NO: 232 - DNA, 47 bp, synthetic construct
  source 1..47: mol_type = other DNA
taacggcagacttctcctcaggagtgcaccatggtgtctgtttgagg

SEQ ID NO: 233 - DNA, 53 bp, synthetic construct
  source 1..53: mol_type = other DNA
taacggcagacttctcctcaggagtctaggtgcaccatggtgtctgtttgagg

SEQ ID NO: 234 - DNA, 53 bp, synthetic construct
  source 1..53: mol_type = other DNA
taacggcagacttctcctcaggagtcaaggtgcaccatggtgtctgtttgagg

SEQ ID NO: 235 - DNA, 51 bp, synthetic construct
  source 1..51: mol_type = other DNA
taacggcagacttctcctcaggagtaggtgcaccatggtgtctgtttgagg

SEQ ID NO: 236 - DNA, 51 bp, synthetic construct
  source 1..51: mol_type = other DNA
taacggcagacttctcctcaggagtcggtgcaccatggtgtctgtttgagg

SEQ ID NO: 237 - DNA, 53 bp, synthetic construct
  source 1..53: mol_type = other DNA
taacggcagacttctcctcaggagtcgaggtgcaccatggtgtctgtttgagg

SEQ ID NO: 238 - DNA, 35 bp, synthetic construct
  source 1..35: mol_type = other DNA
taacggcagacttctcctcaggagtctgtttgagg

SEQ ID NO: 239 - DNA, 40 bp, synthetic construct
  source 1..40: mol_type = other DNA
acagacaccatggtgcacctgactcctgaggagaagtctg

SEQ ID NO: 240 - DNA, 33 bp, synthetic construct
  source 1..33: mol_type = other DNA
acagacaccatggtgcacctgaggagaagtctg

SEQ ID NO: 241 - DNA, 36 bp, synthetic construct
  source 1..36: mol_type = other DNA
acagacaccatggtgcacctcctgaggagaagtctg

SEQ ID NO: 242 - DNA, 40 bp, synthetic construct
  source 1..40: mol_type = other DNA
acagacaccatggtgcatctgactcctgaggagaagactg

SEQ ID NO: 243 - DNA, 40 bp, synthetic construct
  source 1..40: mol_type = other DNA
acagacaccatggtgcacctggactcctgaggagaagtct

SEQ ID NO: 244 - DNA, 34 bp, synthetic construct
  source 1..34: mol_type = other DNA
acagacaccatggtgcaccttgaggagaagtctg

SEQ ID NO: 245 - DNA, 39 bp, synthetic construct
  source 1..39: mol_type = other DNA
acagacaccatggtgcacctactcctgaggagaagtctg

SEQ ID NO: 246 - DNA, 39 bp, synthetic construct
  source 1..39: mol_type = other DNA
acagacaccatggtgcaccgactcctgaggagaagtctg

SEQ ID NO: 247 - DNA, 30 bp, synthetic construct
  source 1..30: mol_type = other DNA
acagacaccatggtgcacctgagaagtctg

SEQ ID NO: 248 - DNA, 32 bp, synthetic construct
  source 1..32: mol_type = other DNA
acagacaccatggactcctgaggagaagtctg

SEQ ID NO: 249 - DNA, 35 bp, synthetic construct
  source 1..35: mol_type = other DNA
acagacaccatggtgcacctctgaggagaagtctg

SEQ ID NO: 250 - DNA, 37 bp, synthetic construct
  source 1..37: mol_type = other DNA
acagacaccatggtgcaccttcctgaggagaagtctg

SEQ ID NO: 251 - DNA, 35 bp, synthetic construct
  source 1..35: mol_type = other DNA
acagacaccatggtgcactcctgaggagaagtctg

SEQ ID NO: 252 - DNA, 31 bp, synthetic construct
  source 1..31: mol_type = other DNA
acagacaccatggtgcacctggagaagtctg

SEQ ID NO: 253 - DNA, 38 bp, synthetic construct
  source 1..38: mol_type = other DNA
acagacaccatggtgcacgactcctgaggagaagtctg

SEQ ID NO: 254 - DNA, 37 bp, synthetic construct
  source 1..37: mol_type = other DNA
acagacaccatggtgcacctgcctgaggagaagtctg

SEQ ID NO: 255 - DNA, 20 bp, synthetic construct
  source 1..20: mol_type = other DNA
acagacaccatggtgcacct

SEQ ID NO: 256 - DNA, 40 bp, synthetic construct
  source 1..40: mol_type = other DNA
acagacaccatggtgcacctagactcctgaggagaagtct

SEQ ID NO: 257 - DNA, 40 bp, synthetic construct
  source 1..40: mol_type = other DNA
acagacaccatggtgcatctgactcctgaggagaagtctg

SEQ ID NO: 258 - DNA, 38 bp, synthetic construct
  source 1..38: mol_type = other DNA
acagacaccatggtgcacctctcctgaggagaagtctg

SEQ ID NO: 259 - DNA, 33 bp, synthetic construct
  source 1..33: mol_type = other DNA
acagacaccatgggactcctgaggagaagtctg

SEQ ID NO: 260 - DNA, 31 bp, synthetic construct
  source 1..31: mol_type = other DNA
acagacaccatgactcctgaggagaagtctg

SEQ ID NO: 261 - DNA, 34 bp, synthetic construct
  source 1..34: mol_type = other DNA
acagacaccatggtgactcctgaggagaagtctg

SEQ ID NO: 262 - DNA, 36 bp, synthetic construct
  source 1..36: mol_type = other DNA
acagacaccatggtgcgactcctgaggagaagtctg

SEQ ID NO: 263 - DNA, 40 bp, synthetic construct
  source 1..40: mol_type = other DNA
acagacaccatggtgcacctggactcctgaggagaagact

SEQ ID NO: 264 - DNA, 23 bp, synthetic construct
  source 1..23: mol_type = other DNA
acagactcctgaggagaagtctg

SEQ ID NO: 265 - DNA, 35 bp, synthetic construct
  source 1..35: mol_type = other DNA
acagacaccatggtgcacctgtgaggagaagtctg

SEQ ID NO: 266 - DNA, 40 bp, synthetic construct
  source 1..40: mol_type = other DNA
acagacaccatggtgcaccttgactcctgaggagaagtct

SEQ ID NO: 267 - DNA, 28 bp, synthetic construct
  source 1..28: mol_type = other DNA
acagacaccatggtgcacctgaagtctg

SEQ ID NO: 268 - DNA, 29 bp, synthetic construct
  source 1..29: mol_type = other DNA
acagacaccatggtgcacctagaagtctg

SEQ ID NO: 269 - DNA, 16 bp, synthetic construct
  source 1..16: mol_type = other DNA
acagacaccatggtgc

SEQ ID NO: 270 - DNA, 40 bp, synthetic construct
  source 1..40: mol_type = other DNA
acagacaccagggtgcacctgactcctgaggagaagtctg

SEQ ID NO: 271 - DNA, 35 bp, synthetic construct
  source 1..35: mol_type = other DNA
acagacaccatggtggactcctgaggagaagtctg

SEQ ID NO: 272 - DNA, 40 bp, synthetic construct
  source 1..40: mol_type = other DNA
acagacaccatggtgcacctgagactcctgaggagaagtc

SEQ ID NO: 273 - DNA, 24 bp, synthetic construct
  source 1..24: mol_type = other DNA
acagacaccatgaggagaagtctg

SEQ ID NO: 274 - DNA, 26 bp, synthetic construct
  source 1..26: mol_type = other DNA
acagacgactcctgaggagaagtctg

SEQ ID NO: 275 - DNA, 38 bp, synthetic construct
  source 1..38: mol_type = other DNA
acagacaccatggtgcacttctcctgaggagaagtctg

SEQ ID NO: 276 - DNA, 37 bp, synthetic construct
  source 1..37: mol_type = other DNA
acagacaccatggtgcagactcctgaggagaagtctg

SEQ ID NO: 277 - DNA, 34 bp, synthetic construct
  source 1..34: mol_type = other DNA
acagacaccatggtgcacctggaggagaagtctg

SEQ ID NO: 278 - DNA, 20 bp, synthetic construct
  source 1..20: mol_type = other DNA
gactcctgaggagaagtctg

SEQ ID NO: 279 - DNA, 27 bp, synthetic construct
  source 1..27: mol_type = other DNA
acagacaccatggtgcacctaagtctg

SEQ ID NO: 280 - DNA, 40 bp, synthetic construct
  source 1..40: mol_type = other DNA
acagacaccatggtgcacctgactcctgaggagaagactg

SEQ ID NO: 281 - DNA, 33 bp, synthetic construct
  source 1..33: mol_type = other DNA
acagacaccatggtgcacctgaggagaagactg

SEQ ID NO: 282 - DNA, 20 bp, synthetic construct
  source 1..20: mol_type = other DNA
gtaacggcagacttctcctc

SEQ ID NO: 283 - DNA, 52 bp, synthetic construct
  source 1..52: mol_type = other DNA
cacagggcagtaacggcagacttctcctcaggagtcaggtgcaccatggtgt

SEQ ID NO: 284 - DNA, 49 bp, synthetic construct
  source 1..49: mol_type = other DNA
cacagggcagtaacggcagacttctcaggagtcaggtgcaccatggtgt

SEQ ID NO: 285 - DNA, 47 bp, synthetic construct
  source 1..47: mol_type = other DNA
cacagggcagtaacggcagacttcaggagtcaggtgcaccatggtgt

SEQ ID NO: 286 - DNA, 51 bp, synthetic construct
  source 1..51: mol_type = other DNA
cacagggcagtaacggcagacttctctcaggagtcaggtgcaccatggtgt

SEQ ID NO: 287 - DNA, 46 bp, synthetic construct
  source 1..46: mol_type = other DNA
cacagggcagtaacggcagactcaggagtcaggtgcaccatggtgt

SEQ ID NO: 288 - DNA, 40 bp, synthetic construct
  source 1..40: mol_type = other DNA
cacagggcagtaacggcaggagtcaggtgcaccatggtgt

SEQ ID NO: 289 - DNA, 53 bp, synthetic construct
  source 1..53: mol_type = other DNA
cacagggcagtaacggcagacttctccctcaggagtcaggtgcaccatggtgt

SEQ ID NO: 290 - DNA, 42 bp, synthetic construct
  source 1..42: mol_type = other DNA
cacagggcagtaacggcagacttctcaggtgcaccatggtgt

SEQ ID NO: 291 - DNA, 40 bp, synthetic construct
  source 1..40: mol_type = other DNA
cacagggcagtaacggcagacttcaggtgcaccatggtgt

SEQ ID NO: 292 - DNA, 53 bp, synthetic construct
  source 1..53: mol_type = other DNA
cacagggcagtaacggcagacttctctctcaggagtcaggtgcaccatggtgt

SEQ ID NO: 293 - DNA, 39 bp, synthetic construct
  source 1..39: mol_type = other DNA
cacagggcagtaacggcagagtcaggtgcaccatggtgt

SEQ ID NO: 294 - DNA, 40 bp, synthetic construct
  source 1..40: mol_type = other DNA
tggtgcatctgactcctgaggagaagtctgccgttactgc

SEQ ID NO: 295 - DNA, 40 bp, synthetic construct
  source 1..40: mol_type = other DNA
tggtgcacctgactcctgaggagaagtctgccgttactgc

SEQ ID NO: 296 - DNA, 39 bp, synthetic construct
  source 1..39: mol_type = other DNA
tggtgcacctgactcctgagagaagtctgccgttactgc

SEQ ID NO: 297 - DNA, 37 bp, synthetic construct
  source 1..37: mol_type = other DNA
tggtgcacctgactcctgagaagtctgccgttactgc

SEQ ID NO: 298 - DNA, 40 bp, synthetic construct
  source 1..40: mol_type = other DNA
tggtgcatctgactcctgaggagaagactgctgtcaatgc

SEQ ID NO: 299 - DNA, 38 bp, synthetic construct
  source 1..38: mol_type = other DNA
tggtgcacctgactcctggagaagtctgccgttactgc

SEQ ID NO: 300 - DNA, 38 bp, synthetic construct
  source 1..38: mol_type = other DNA
tggtgcacctgactcctgaggaagtctgccgttactgc

SEQ ID NO: 301 - DNA, 34 bp, synthetic construct
  source 1..34: mol_type = other DNA
tggtgcacctgactcctgagtctgccgttactgc

SEQ ID NO: 302 - DNA, 28 bp, synthetic construct
  source 1..28: mol_type = other DNA
tggtgcacctgactcctgccgttactgc

SEQ ID NO: 303 - DNA, 40 bp, synthetic construct
  source 1..40: mol_type = other DNA
tggtgcacctgactcctgagggagaagtctgccgttactg

SEQ ID NO: 304 - DNA, 35 bp, synthetic construct
  source 1..35: mol_type = other DNA
tggtgcacctgactcctgaagtctgccgttactgc

SEQ ID NO: 305 - DNA, 35 bp, synthetic construct
  source 1..35: mol_type = other DNA
tggtgcacctgactcctgaggtctgccgttactgc

SEQ ID NO: 306 - DNA, 40 bp, synthetic construct
  source 1..40: mol_type = other DNA
tggtgcacctgactcctgaggagaagactgctgtcaatgc

SEQ ID NO: 307 - DNA, 40 bp, synthetic construct
  source 1..40: mol_type = other DNA
tggtgcacctgactcctgaggagagaagtctgccgttact

SEQ ID NO: 308 - DNA, 36 bp, synthetic construct
  source 1..36: mol_type = other DNA
tggtgcacctgactcctgagagtctgccgttactgc

SEQ ID NO: 309 - DNA, 35 bp, synthetic construct
  source 1..35: mol_type = other DNA
tggtgcacctgactcgagaagtctgccgttactgc

SEQ ID NO: 310 - DNA, 31 bp, synthetic construct
  source 1..31: mol_type = other DNA
tggtgcacctggagaagtctgccgttactgc

SEQ ID NO: 311 - DNA, 21 bp, synthetic construct
  source 1..21: mol_type = other DNA
tggtgcacctgccgttactgc

SEQ ID NO: 312 - DNA, 30 bp, synthetic construct
  source 1..30: mol_type = other DNA
tggtgcacctgagaagtctgccgttactgc

SEQ ID NO: 313 - DNA, 36 bp, synthetic construct
  source 1..36: mol_type = other DNA
tggtgcacctgactccgagaagtctgccgttactgc

SEQ ID NO: 314 - DNA, 32 bp, synthetic construct
  source 1..32: mol_type = other DNA
tggtgcacctgagagaagtctgccgttactgc

SEQ ID NO: 315 - DNA, 20 bp, synthetic construct
  source 1..20: mol_type = other DNA
ctgactcctgaggagaagtc

SEQ ID NO: 316 - DNA, 52 bp, synthetic construct
  source 1..52: mol_type = other DNA
atggtgcacctgactcctgaggagaagtctgccgttactgccctgtggggca

SEQ ID NO: 317 - DNA, 53 bp, synthetic construct
  source 1..53: mol_type = other DNA
atggtgcacctgactcctgaggagaaagtctgccgttactgccctgtggggca

SEQ ID NO: 318 - DNA, 40 bp, synthetic construct
  source 1..40: mol_type = other DNA
atggtgcacctgactcctgccgttactgccctgtggggca

SEQ ID NO: 319 - DNA, 49 bp, synthetic construct
  source 1..49: mol_type = other DNA
atggtgcacctgactcctgaggagtctgccgttactgccctgtggggca

SEQ ID NO: 320 - DNA, 51 bp, synthetic construct
  source 1..51: mol_type = other DNA
atggtgcacctgactcctgaggagaatctgccgttactgccctgtggggca

SEQ ID NO: 321 - DNA, 51 bp, synthetic construct
  source 1..51: mol_type = other DNA
atggtgcacctgactcctgaggagagtctgccgttactgccctgtggggca

SEQ ID NO: 322 - DNA, 53 bp, synthetic construct
  source 1..53: mol_type = other DNA
atggtgcacctgactcctgaggagaatgtctgccgttactgccctgtggggca

SEQ ID NO: 323 - DNA, 47 bp, synthetic construct
  source 1..47: mol_type = other DNA
atggtgcacctgactcctgaggtctgccgttactgccctgtggggca

SEQ ID NO: 324 - DNA, 46 bp, synthetic construct
  source 1..46: mol_type = other DNA
atggtgcacctgactcctgagtctgccgttactgccctgtggggca

SEQ ID NO: 325 - DNA, 49 bp, synthetic construct
  source 1..49: mol_type = other DNA
atggtgcacctgactcctgaggagaatgccgttactgccctgtggggca

SEQ ID NO: 326 - DNA, 49 bp, synthetic construct
  source 1..49: mol_type = other DNA
atggtgcacctgactcctgaggagactgccgttactgccctgtggggca

SEQ ID NO: 327 - DNA, 40 bp, synthetic construct
  source 1..40: mol_type = other DNA
catctgactcctgaggagaagtctgccgttactgccctgt

SEQ ID NO: 328 - DNA, 40 bp, synthetic construct
  source 1..40: mol_type = other DNA
cacctgactcctgaggagaagtctgccgttactgccctgt

SEQ ID NO: 329 - DNA, 40 bp, synthetic construct
  source 1..40: mol_type = other DNA
catctgactcctgaggagaagactgctgtcaatgccctgt

SEQ ID NO: 330 - DNA, 40 bp, synthetic construct
  source 1..40: mol_type = other DNA
cacctgactcctgaggagaaagtctgccgttactgccctg

SEQ ID NO: 331 - DNA, 39 bp, synthetic construct
  source 1..39: mol_type = other DNA
cacctgactcctgaggagagtctgccgttactgccctgt

SEQ ID NO: 332 - DNA, 40 bp, synthetic construct
  source 1..40: mol_type = other DNA
cacctgactcctgaggagaagactgctgtcaatgccctgt

SEQ ID NO: 333 - DNA, 37 bp, synthetic construct
  source 1..37: mol_type = other DNA
cacctgactcctgaggagtctgccgttactgccctgt

SEQ ID NO: 334 - DNA, 20 bp, synthetic construct
  source 1..20: mol_type = other DNA
taacggcagacttctcctca

SEQ ID NO: 335 - DNA, 52 bp, synthetic construct
  source 1..52: mol_type = other DNA
acagggcagtaacggcagacttctcctcaggagtcaggtgcaccatggtgtc

SEQ ID NO: 336 - DNA, 49 bp, synthetic construct
  source 1..49: mol_type = other DNA
acagggcagtaacggcagacttctcaggagtcaggtgcaccatggtgtc

SEQ ID NO: 337 - DNA, 47 bp, synthetic construct
  source 1..47: mol_type = other DNA
acagggcagtaacggcagacttcaggagtcaggtgcaccatggtgtc

SEQ ID NO: 338 - DNA, 40 bp, synthetic construct
  source 1..40: mol_type = other DNA
acagggcagtaacggcaggagtcaggtgcaccatggtgtc

SEQ ID NO: 339 - DNA, 53 bp, synthetic construct
  source 1..53: mol_type = other DNA
acagggcagtaacggcagacttctccctcaggagtcaggtgcaccatggtgtc

SEQ ID NO: 340 - DNA, 50 bp, synthetic construct
  source 1..50: mol_type = other DNA
acagggcagtaacggcagacttctccaggagtcaggtgcaccatggtgtc

SEQ ID NO: 341 - DNA, 50 bp, synthetic construct
  source 1..50: mol_type = other DNA
acagggcagtaacggcagacttcttcaggagtcaggtgcaccatggtgtc

SEQ ID NO: 342 - DNA, 42 bp, synthetic construct
  source 1..42: mol_type = other DNA
acagggcagtaacggcagacttctcaggtgcaccatggtgtc

SEQ ID NO: 343 - DNA, 49 bp, synthetic construct
  source 1..49: mol_type = other DNA
acagggcagtaacggcagacttctccggagtcaggtgcaccatggtgtc

SEQ ID NO: 344 - DNA, 51 bp, synthetic construct
  source 1..51: mol_type = other DNA
acagggcagtaacggcagacttctctcaggagtcaggtgcaccatggtgtc

SEQ ID NO: 345 - DNA, 51 bp, synthetic construct
  source 1..51: mol_type = other DNA
acagggcagtaacggcagacttctcccaggagtcaggtgcaccatggtgtc

SEQ ID NO: 346 - DNA, 40 bp, synthetic construct
  source 1..40: mol_type = other DNA
atggtgcatctgactcctgaggagaagtctgccgttactg

SEQ ID NO: 347 - DNA, 40 bp, synthetic construct
  source 1..40: mol_type = other DNA
atggtgcacctgactcctgaggagaagtctgccgttactg

SEQ ID NO: 348 - DNA, 40 bp, synthetic construct
  source 1..40: mol_type = other DNA
atggtgcatctgactcctgaggagaagactgctgtcaatg

SEQ ID NO: 349 - DNA, 37 bp, synthetic construct
  source 1..37: mol_type = other DNA
atggtgcacctgactcctgagaagtctgccgttactg

SEQ ID NO: 350 - DNA, 39 bp, synthetic construct
  source 1..39: mol_type = other DNA
atggtgcacctgactcctgagagaagtctgccgttactg

SEQ ID NO: 351 - DNA, 40 bp, synthetic construct
  source 1..40: mol_type = other DNA
atggtgcacctgactcctgagggagaagtctgccgttact

SEQ ID NO: 352 - DNA, 39 bp, synthetic construct
  source 1..39: mol_type = other DNA
atggtgcacctgactcctgggagaagtctgccgttactg

SEQ ID NO: 353 - DNA, 40 bp, synthetic construct
  source 1..40: mol_type = other DNA
atggtgcacctgactcctgaggggagaagtctgccgttac

SEQ ID NO: 354 - DNA, 40 bp, synthetic construct
  source 1..40: mol_type = other DNA
atggtgcacctgactcctgaggagaagactgctgtcaatg

SEQ ID NO: 355 - DNA, 38 bp, synthetic construct
  source 1..38: mol_type = other DNA
atggtgcacctgactcctggagaagtctgccgttactg

SEQ ID NO: 356 - DNA, 37 bp, synthetic construct
  source 1..37: mol_type = other DNA
atggtgcacctgactccggagaagtctgccgttactg

SEQ ID NO: 357 - DNA, 36 bp, synthetic construct
  source 1..36: mol_type = other DNA
atggtgcacctgactcggagaagtctgccgttactg

SEQ ID NO: 358 - DNA, 35 bp, synthetic construct
  source 1..35: mol_type = other DNA
atggtgcacctgactcctgaggtctgccgttactg

SEQ ID NO: 359 - DNA, 30 bp, synthetic construct
  source 1..30: mol_type = other DNA
atggtgcacctgagaagtctgccgttactg

SEQ ID NO: 360 - DNA, 36 bp, synthetic construct
  source 1..36: mol_type = other DNA
atggtgcacctgactcctgagagtctgccgttactg

SEQ ID NO: 361 - DNA, 33 bp, synthetic construct
  source 1..33: mol_type = other DNA
atggtgcacctgactcctgagctgccgttactg

SEQ ID NO: 362 - DNA, 38 bp, synthetic construct
  source 1..38: mol_type = other DNA
atggtgcacctgactcctgaggaagtctgccgttactg

SEQ ID NO: 363 - DNA, 32 bp, synthetic construct
  source 1..32: mol_type = other DNA
atggtgcacctgactcctgagtgccgttactg

SEQ ID NO: 364 - DNA, 34 bp, synthetic construct
  source 1..34: mol_type = other DNA
atggtgcacctgacggagaagtctgccgttactg

SEQ ID NO: 365 - DNA, 31 bp, synthetic construct
  source 1..31: mol_type = other DNA
atggtgcacctggagaagtctgccgttactg

SEQ ID NO: 366 - DNA, 20 bp, synthetic construct
  source 1..20: mol_type = other DNA
gaacacaaagcatagactgc

SEQ ID NO: 367 - DNA, 52 bp, synthetic construct
  source 1..52: mol_type = other DNA
aggaaactggaacacaaagcatagactgcggggcgggccagcctgaatagct

SEQ ID NO: 368 - DNA, 43 bp, synthetic construct
  source 1..43: mol_type = other DNA
aggaaactggaacacaaagcggggcgggccagcctgaatagct

SEQ ID NO: 369 - DNA, 53 bp, synthetic construct
  source 1..53: mol_type = other DNA
aggaaactggaacacaaagcatagacctgcggggcgggccagcctgaatagct

SEQ ID NO: 370 - DNA, 49 bp, synthetic construct
  source 1..49: mol_type = other DNA
aggaaactggaacacaaagcatagacggggcgggccagcctgaatagct

SEQ ID NO: 371 - DNA, 51 bp, synthetic construct
  source 1..51: mol_type = other DNA
aggaaactggaacacaaagcatagatgcggggcgggccagcctgaatagct

SEQ ID NO: 372 - DNA, 51 bp, synthetic construct
  source 1..51: mol_type = other DNA
aggaaactggaacacaaagcatagacgcggggcgggccagcctgaatagct

SEQ ID NO: 373 - DNA, 48 bp, synthetic construct
  source 1..48: mol_type = other DNA
aggaaactggaacacaaagcatagcggggcgggccagcctgaatagct

SEQ ID NO: 374 - DNA, 47 bp, synthetic construct
  source 1..47: mol_type = other DNA
aggaaactggaacacaaagcatgcggggcgggccagcctgaatagct

SEQ ID NO: 375 - DNA, 38 bp, synthetic construct
  source 1..38: mol_type = other DNA
aggaaactggaacacaaagcgggccagcctgaatagct

SEQ ID NO: 376 - DNA, 46 bp, synthetic construct
  source 1..46: mol_type = other DNA
aggaaactggaacacaaagcataggggcgggccagcctgaatagct

SEQ ID NO: 377 - DNA, 49 bp, synthetic construct
  source 1..49: mol_type = other DNA
aggaaactggaacacaaagcatatgcggggcgggccagcctgaatagct

SEQ ID NO: 378 - DNA, 40 bp, synthetic construct
  source 1..40: mol_type = other DNA
tcaggctggcccgccccgcagtctatgctttgtgttccag

SEQ ID NO: 379 - DNA, 39 bp, synthetic construct
  source 1..39: mol_type = other DNA
tcaggctggcccgccccgcatctatgctttgtgttccag

SEQ ID NO: 380 - DNA, 40 bp, synthetic construct
  source 1..40: mol_type = other DNA
tcaggctggcccgccccgcaagtctatgctttgtgttcca

SEQ ID NO: 381 - DNA, 39 bp, synthetic construct
  source 1..39: mol_type = other DNA
tcaggctggcccgccccgcgtctatgctttgtgttccag

SEQ ID NO: 382 - DNA, 40 bp, synthetic construct
  source 1..40: mol_type = other DNA
tcaggctggcccgccccgcaggtctatgctttgtgttcca

SEQ ID NO: 383 - DNA, 38 bp, synthetic construct
  source 1..38: mol_type = other DNA
tcaggctggcccgccccgcactatgctttgtgttccag

SEQ ID NO: 384 - DNA, 31 bp, synthetic construct
  source 1..31: mol_type = other DNA
tcaggctggcccgccccgctttgtgttccag

SEQ ID NO: 385 - DNA, 37 bp, synthetic construct
  source 1..37: mol_type = other DNA
tcaggctggcccgccccgcatatgctttgtgttccag

SEQ ID NO: 386 - DNA, 40 bp, synthetic construct
  source 1..40: mol_type = other DNA
tcaggctggcccgccccgcatgtctatgctttgtgttcca

SEQ ID NO: 387 - DNA, 20 bp, synthetic construct
  source 1..20: mol_type = other DNA
gtctatgctttgtgttccag

SEQ ID NO: 388 - DNA, 32 bp, synthetic construct
  source 1..32: mol_type = other DNA
tcaggctggcccgtctatgctttgtgttccag

SEQ ID NO: 389 - DNA, 37 bp, synthetic construct
  source 1..37: mol_type = other DNA
tcaggctggcccgccccgtctatgctttgtgttccag

SEQ ID NO: 390 - DNA, 40 bp, synthetic construct
  source 1..40: mol_type = other DNA
tcaggctggcccgccccgcacgtctatgctttgtgttcca

SEQ ID NO: 391 - DNA, 35 bp, synthetic construct
  source 1..35: mol_type = other DNA
tcaggctggcccgccccgcatgctttgtgttccag

SEQ ID NO: 392 - DNA, 28 bp, synthetic construct
  source 1..28: mol_type = other DNA
tcaggctggtctatgctttgtgttccag

SEQ ID NO: 393 - DNA, 18 bp, synthetic construct
  source 1..18: mol_type = other DNA
tcaggctttgtgttccag

SEQ ID NO: 394 - DNA, 30 bp, synthetic construct
  source 1..30: mol_type = other DNA
tcaggctggcgtctatgctttgtgttccag

SEQ ID NO: 395 - DNA, 27 bp, synthetic construct
  source 1..27: mol_type = other DNA
tcaggctgtctatgctttgtgttccag

SEQ ID NO: 396 - DNA, 23 bp, synthetic construct
  source 1..23: mol_type = other DNA
tcagtctatgctttgtgttccag

SEQ ID NO: 397 - DNA, 14 bp, synthetic construct
  source 1..14: mol_type = other DNA
gctttgtgttccag

SEQ ID NO: 398 - DNA, 23 bp, synthetic construct
  source 1..23: mol_type = other DNA
tcaggctatgctttgtgttccag

SEQ ID NO: 399 - DNA, 34 bp, synthetic construct
  source 1..34: mol_type = other DNA
tcaggctggcccgcgtctatgctttgtgttccag

SEQ ID NO: 400 - DNA, 30 bp, synthetic construct
  source 1..30: mol_type = other DNA
tcaggctggcccgccccgcatgtgttccag

SEQ ID NO: 401 - DNA, 31 bp, synthetic construct
  source 1..31: mol_type = other DNA
tcaggctggccgtctatgctttgtgttccag

SEQ ID NO: 402 - DNA, 36 bp, synthetic construct
  source 1..36: mol_type = other DNA
tcaggctggcccgccccgcaatgctttgtgttccag

SEQ ID NO: 403 - DNA, 36 bp, synthetic construct
  source 1..36: mol_type = other DNA
tcaggctggcccgcccgtctatgctttgtgttccag

SEQ ID NO: 404 - DNA, 20 bp, synthetic construct
  source 1..20: mol_type = other DNA
tcaggctggcccgccccgca

SEQ ID NO: 405 - DNA, 26 bp, synthetic construct
  source 1..26: mol_type = other DNA
tcaggcgtctatgctttgtgttccag

SEQ ID NO: 406 - DNA, 38 bp, synthetic construct
  source 1..38: mol_type = other DNA
tcaggctggcccgccccggtctatgctttgtgttccag

SEQ ID NO: 407 - DNA, 34 bp, synthetic construct
  source 1..34: mol_type = other DNA
tcaggctggcccgccccgcagctttgtgttccag

SEQ ID NO: 408 - DNA, 32 bp, synthetic construct
  source 1..32: mol_type = other DNA
tcaggctggcccgccccgcatttgtgttccag

SEQ ID NO: 409 - DNA, 19 bp, synthetic construct
  source 1..19: mol_type = other DNA
tctatgctttgtgttccag

SEQ ID NO: 410 - DNA, 24 bp, synthetic construct
  source 1..24: mol_type = other DNA
tcaggtctatgctttgtgttccag

SEQ ID NO: 411 - DNA, 22 bp, synthetic construct
  source 1..22: mol_type = other DNA
tcaggctggctttgtgttccag

SEQ ID NO: 412 - DNA, 31 bp, synthetic construct
  source 1..31: mol_type = other DNA
tcaggctggcccgccccgcattgtgttccag

SEQ ID NO: 413 - DNA, 40 bp, synthetic construct
  source 1..40: mol_type = other DNA
tcaggctggcccgccccgcagtctgtgctttgtgttccag

SEQ ID NO: 414 - DNA, 18 bp, synthetic construct
  source 1..18: mol_type = other DNA
ctatgctttgtgttccag

SEQ ID NO: 415 - DNA, 26 bp, synthetic construct
  source 1..26: mol_type = other DNA
tcaggctggcccgctttgtgttccag

SEQ ID NO: 416 - DNA, 33 bp, synthetic construct
  source 1..33: mol_type = other DNA
tcaggctggcccggtctatgctttgtgttccag

SEQ ID NO: 417 - DNA, 36 bp, synthetic construct
  source 1..36: mol_type = other DNA
tcaggctggcccgccccgctatgctttgtgttccag

SEQ ID NO: 418 - DNA, 29 bp, synthetic construct
  source 1..29: mol_type = other DNA
tcaggctgggtctatgctttgtgttccag

SEQ ID NO: 419 - DNA, 20 bp, synthetic construct
  source 1..20: mol_type = other DNA
ggcccagactgagcacgtga

SEQ ID NO: 420 - DNA, 52 bp, synthetic construct
  source 1..52: mol_type = other DNA
aatccttggggcccagactgagcacgtgatggcagaggaaaggaagccctgc

SEQ ID NO: 421 - DNA, 44 bp, synthetic construct
  source 1..44: mol_type = other DNA
aatccttggggcccagactgatggcagaggaaaggaagccctgc

SEQ ID NO: 422 - DNA, 42 bp, synthetic construct
  source 1..42: mol_type = other DNA
aatccttggggcccagactgagcagaggaaaggaagccctgc

SEQ ID NO: 423 - DNA, 50 bp, synthetic construct
  source 1..50: mol_type = other DNA
aatccttggggcccagactgagcacgatggcagaggaaaggaagccctgc

SEQ ID NO: 424 - DNA, 50 bp, synthetic construct
  source 1..50: mol_type = other DNA
aatccttggggcccagactgagcatgatggcagaggaaaggaagccctgc

SEQ ID NO: 425 - DNA, 37 bp, synthetic construct
  source 1..37: mol_type = other DNA
aatccttggggcccagactgaggaaaggaagccctgc

SEQ ID NO: 426 - DNA, 51 bp, synthetic construct
  source 1..51: mol_type = other DNA
aatccttggggcccagactgagcacggatggcagaggaaaggaagccctgc

SEQ ID NO: 427 - DNA, 51 bp, synthetic construct
  source 1..51: mol_type = other DNA
aatccttggggcccagactgagcactgatggcagaggaaaggaagccctgc

SEQ ID NO: 428 - DNA, 53 bp, synthetic construct
  source 1..53: mol_type = other DNA
aatccttggggcccagactgagcacggtgatggcagaggaaaggaagccctgc

SEQ ID NO: 429 - DNA, 41 bp, synthetic construct
  source 1..41: mol_type = other DNA
aatccttggggcccagactggcagaggaaaggaagccctgc

SEQ ID NO: 430 - DNA, 33 bp, synthetic construct
  source 1..33: mol_type = other DNA
aatccttggggcccagaggaaaggaagccctgc

SEQ ID NO: 431 - DNA, 40 bp, synthetic construct
  source 1..40: mol_type = other DNA
tggggcccagactgagcacgtgatggcagaggaaaggaag

SEQ ID NO: 432 - DNA, 40 bp, synthetic construct
  source 1..40: mol_type = other DNA
tggggcccagactgagcacgtgatggcaaaggaaaggaag

SEQ ID NO: 433 - DNA, 38 bp, synthetic construct
  source 1..38: mol_type = other DNA
tggggcccagactgagcatgatggcagaggaaaggaag

SEQ ID NO: 434 - DNA, 39 bp, synthetic construct
  source 1..39: mol_type = other DNA
tggggcccagactgagcactgatggcagaggaaaggaag

SEQ ID NO: 435 - DNA, 38 bp, synthetic construct
  source 1..38: mol_type = other DNA
tggggcccagactgagcatgatggcaaaggaaaggaag

SEQ ID NO: 436 - DNA, 38 bp, synthetic construct
  source 1..38: mol_type = other DNA
tggggcccagactgagcacgatggcagaggaaaggaag

SEQ ID NO: 437 - DNA, 39 bp, synthetic construct
  source 1..39: mol_type = other DNA
tggggcccagactgagcactgatggcaaaggaaaggaag

SEQ ID NO: 438 - DNA, 40 bp, synthetic construct
  source 1..40: mol_type = other DNA
tggggcccagactgagcacggtgatggcagaggaaaggaa

SEQ ID NO: 439 - DNA, 32 bp, synthetic construct
  source 1..32: mol_type = other DNA
tggggcccagactgatggcagaggaaaggaag

SEQ ID NO: 440 - DNA, 38 bp, synthetic construct
  source 1..38: mol_type = other DNA
tggggcccagactgagcacgatggcaaaggaaaggaag

SEQ ID NO: 441 - DNA, 30 bp, synthetic construct
  source 1..30: mol_type = other DNA
tggggcccagactgagcagaggaaaggaag

SEQ ID NO: 442 - DNA, 40 bp, synthetic construct
  source 1..40: mol_type = other DNA
tggggcccagactgagcacggtgatggcaaaggaaaggaa

SEQ ID NO: 443 - DNA, 40 bp, synthetic construct
  source 1..40: mol_type = other DNA
tggggcccagactgagcacgttgatggcagaggaaaggaa

SEQ ID NO: 444 - DNA, 32 bp, synthetic construct
  source 1..32: mol_type = other DNA
tggggcccagactgatggcaaaggaaaggaag

SEQ ID NO: 445 - DNA, 30 bp, synthetic construct
  source 1..30: mol_type = other DNA
tggggcccagactgagcaaaggaaaggaag

SEQ ID NO: 446 - DNA, 40 bp, synthetic construct
  source 1..40: mol_type = other DNA
tggggcccagactgagcacgatgatggcagaggaaaggaa

SEQ ID NO: 447 - DNA, 40 bp, synthetic construct
  source 1..40: mol_type = other DNA
tggggcccagactgagcacgttgatggcaaaggaaaggaa

SEQ ID NO: 448 - DNA, 34 bp, synthetic construct
  source 1..34: mol_type = other DNA
tggggcccagactgagcacgcagaggaaaggaag

SEQ ID NO: 449 - DNA, 36 bp, synthetic construct
  source 1..36: mol_type = other DNA
tggggcccagactgagtgatggcagaggaaaggaag

SEQ ID NO: 450 - DNA, 37 bp, synthetic construct
  source 1..37: mol_type = other DNA
tggggcccagactgagctgatggcagaggaaaggaag

SEQ ID NO: 451 - DNA, 34 bp, synthetic construct
  source 1..34: mol_type = other DNA
tggggcccagactgagcacgcaaaggaaaggaag

SEQ ID NO: 452 - DNA, 39 bp, synthetic construct
  source 1..39: mol_type = other DNA
tggggcccagactgagcacggatggcaaaggaaaggaag

SEQ ID NO: 453 - DNA, 39 bp, synthetic construct
  source 1..39: mol_type = other DNA
tggggcccagactgagcaatgatggcagaggaaaggaag

SEQ ID NO: 454 - DNA, 26 bp, synthetic construct
  source 1..26: mol_type = other DNA
tggggctgatggcagaggaaaggaag

SEQ ID NO: 455 - DNA, 39 bp, synthetic construct
  source 1..39: mol_type = other DNA
tggggcccagactgagcacggatggcagaggaaaggaag

SEQ ID NO: 456 - DNA, 25 bp, synthetic construct
  source 1..25: mol_type = other DNA
tggggcccagactgaggaaaggaag

SEQ ID NO: 457 - DNA, 29 bp, synthetic construct
  source 1..29: mol_type = other DNA
tggggcccagactggcagaggaaaggaag

SEQ ID NO: 458 - DNA, 36 bp, synthetic construct
  source 1..36: mol_type = other DNA
tggggcccagactgagtgatggcaaaggaaaggaag

SEQ ID NO: 459 - DNA, 40 bp, synthetic construct
  source 1..40: mol_type = other DNA
tggggcccagactgagcacgatgatggcaaaggaaaggaa

SEQ ID NO: 460 - DNA, 27 bp, synthetic construct
  source 1..27: mol_type = other DNA
tggggcctgatggcagaggaaaggaag

SEQ ID NO: 461 - DNA, 37 bp, synthetic construct
  source 1..37: mol_type = other DNA
tggggcccagactgaggtgatggcagaggaaaggaag

SEQ ID NO: 462 - DNA, 37 bp, synthetic construct
  source 1..37: mol_type = other DNA
tggggcccagactgagctgatggcaaaggaaaggaag

SEQ ID NO: 463 - DNA, 20 bp, synthetic construct
  source 1..20: mol_type = other DNA
tacctgaaccgtatatccta

SEQ ID NO: 464 - DNA, 52 bp, synthetic construct
  source 1..52: mol_type = other DNA
gccccctggtacctgaaccgtatatcctatggccctgactggaaggatttct

SEQ ID NO: 465 - DNA, 47 bp, synthetic construct
  source 1..47: mol_type = other DNA
gccccctggtacctgaaccgtatatggccctgactggaaggatttct

SEQ ID NO: 466 - DNA, 45 bp, synthetic construct
  source 1..45: mol_type = other DNA
gccccctggtacctgaaccgtatggccctgactggaaggatttct

SEQ ID NO: 467 - DNA, 51 bp, synthetic construct
  source 1..51: mol_type = other DNA
gccccctggtacctgaaccgtatatctatggccctgactggaaggatttct

SEQ ID NO: 468 - DNA, 49 bp, synthetic construct
  source 1..49: mol_type = other DNA
gccccctggtacctgaaccgtatatatggccctgactggaaggatttct

SEQ ID NO: 469 - DNA, 53 bp, synthetic construct
  source 1..53: mol_type = other DNA
gccccctggtacctgaaccgtatatccctatggccctgactggaaggatttct

SEQ ID NO: 470 - DNA, 30 bp, synthetic construct
  source 1..30: mol_type = other DNA
gccccctggtacctgactggaaggatttct

SEQ ID NO: 471 - DNA, 49 bp, synthetic construct
  source 1..49: mol_type = other DNA
gccccctggtacctgaaccgtatctatggccctgactggaaggatttct

SEQ ID NO: 472 - DNA, 49 bp, synthetic construct
  source 1..49: mol_type = other DNA
gccccctggtacctgaaccgtatatctggccctgactggaaggatttct

SEQ ID NO: 473 - DNA, 50 bp, synthetic construct
  source 1..50: mol_type = other DNA
gccccctggtacctgaaccgtatactatggccctgactggaaggatttct

SEQ ID NO: 474 - DNA, 50 bp, synthetic construct
  source 1..50: mol_type = other DNA
gccccctggtacctgaaccgtatattatggccctgactggaaggatttct

SEQ ID NO: 475 - DNA, 40 bp, synthetic construct
  source 1..40: mol_type = other DNA
tggtacctgaaccgtatatcctatggccctgactggaagg

SEQ ID NO: 476 - DNA, 39 bp, synthetic construct
  source 1..39: mol_type = other DNA
tggtacctgaaccgtatatctatggccctgactggaagg

SEQ ID NO: 477 - DNA, 35 bp, synthetic construct
  source 1..35: mol_type = other DNA
tggtacctgaaccgtatatggccctgactggaagg

SEQ ID NO: 478 - DNA, 33 bp, synthetic construct
  source 1..33: mol_type = other DNA
tggtacctgaaccgtatggccctgactggaagg

SEQ ID NO: 479 - DNA, 37 bp, synthetic construct
  source 1..37: mol_type = other DNA
tggtacctgaaccgtatctatggccctgactggaagg

SEQ ID NO: 480 - DNA, 38 bp, synthetic construct
  source 1..38: mol_type = other DNA
tggtacctgaaccgtatactatggccctgactggaagg

SEQ ID NO: 481 - DNA, 39 bp, synthetic construct
  source 1..39: mol_type = other DNA
tggtacctgaaccgtatacctatggccctgactggaagg

SEQ ID NO: 482 - DNA, 40 bp, synthetic construct
  source 1..40: mol_type = other DNA
tggtacctgaaccgtatatccctatggccctgactggaag

SEQ ID NO: 483 - DNA, 36 bp, synthetic construct
  source 1..36: mol_type = other DNA
tggtacctgaaccgtactatggccctgactggaagg

SEQ ID NO: 484 - DNA, 18 bp, synthetic construct
  source 1..18: mol_type = other DNA
tggtacctgactggaagg

SEQ ID NO: 485 - DNA, 32 bp, synthetic construct
  source 1..32: mol_type = other DNA
tggtacctgaacctatggccctgactggaagg

SEQ ID NO: 486 - DNA, 40 bp, synthetic construct
  source 1..40: mol_type = other DNA
tggtacctgaaccgtatatctcctatggccctgactggaa

SEQ ID NO: 487 - DNA, 37 bp, synthetic construct
  source 1..37: mol_type = other DNA
tggtacctgaaccgtatatatggccctgactggaagg

SEQ ID NO: 488 - DNA, 38 bp, synthetic construct
  source 1..38: mol_type = other DNA
tggtacctgaaccgtatatcatggccctgactggaagg

SEQ ID NO: 489 - DNA, 38 bp, synthetic construct
  source 1..38: mol_type = other DNA
tggtacctgaaccgtatcctatggccctgactggaagg

SEQ ID NO: 490 - DNA, 31 bp, synthetic construct
  source 1..31: mol_type = other DNA
tggtacctgaaccgtatatctgactggaagg

SEQ ID NO: 491 - DNA, 19 bp, synthetic construct
  source 1..19: mol_type = other DNA
tggtacctgaactggaagg

SEQ ID NO: 492 - DNA, 40 bp, synthetic construct
  source 1..40: mol_type = other DNA
tggtacctgaaccgtatatctctatggccctgactggaag

SEQ ID NO: 493 - DNA, 35 bp, synthetic construct
  source 1..35: mol_type = other DNA
tggtacctgaaccgtctatggccctgactggaagg

SEQ ID NO: 494 - DNA, 17 bp, synthetic construct
  source 1..17: mol_type = other DNA
tggccctgactggaagg

SEQ ID NO: 495 - DNA, 24 bp, synthetic construct
  source 1..24: mol_type = other DNA
tggtacctggccctgactggaagg

SEQ ID NO: 496 - DNA, 40 bp, synthetic construct
  source 1..40: mol_type = other DNA
tggtacctgaaccgtatattctatggccctgactggaagg

SEQ ID NO: 497 - DNA, 35 bp, synthetic construct
  source 1..35: mol_type = other DNA
tggtacctgaaccgtatatcgccctgactggaagg

SEQ ID NO: 498 - DNA, 37 bp, synthetic construct
  source 1..37: mol_type = other DNA
tggtacctgaaccgtacctatggccctgactggaagg

SEQ ID NO: 499 - DNA, 25 bp, synthetic construct
  source 1..25: mol_type = other DNA
tggtacctgaaccctgactggaagg

SEQ ID NO: 500 - DNA, 20 bp, synthetic construct
  source 1..20: mol_type = other DNA
tggtacctgaaccgtatatc

SEQ ID NO: 501 - DNA, 30 bp, synthetic construct
  source 1..30: mol_type = other DNA
tggtacctgaaccgtatatcgactggaagg

SEQ ID NO: 502 - DNA, 33 bp, synthetic construct
  source 1..33: mol_type = other DNA
tggtacctgaaccgtatatccctgactggaagg

SEQ ID NO: 503 - DNA, 32 bp, synthetic construct
  source 1..32: mol_type = other DNA
tggtacctgaaccgtatatcctgactggaagg

SEQ ID NO: 504 - DNA, 26 bp, synthetic construct
  source 1..26: mol_type = other DNA
tggtacctatggccctgactggaagg

SEQ ID NO: 505 - DNA, 12 bp, synthetic construct
  source 1..12: mol_type = other DNA
ctgactggaagg

SEQ ID NO: 506 - DNA, 20 bp, synthetic construct
  source 1..20: mol_type = other DNA
gtatatcctatggccctgac

SEQ ID NO: 507 - DNA, 52 bp, synthetic construct
  source 1..52: mol_type = other DNA
acctgaaccgtatatcctatggccctgactggaaggatttctacatagtgga

SEQ ID NO: 508 - DNA, 53 bp, synthetic construct
  source 1..53: mol_type = other DNA
acctgaaccgtatatcctatggcccttgactggaaggatttctacatagtgga

SEQ ID NO: 509 - DNA, 48 bp, synthetic construct
  source 1..48: mol_type = other DNA
acctgaaccgtatatcctatggccctggaaggatttctacatagtgga

SEQ ID NO: 510 - DNA, 42 bp, synthetic construct
  source 1..42: mol_type = other DNA
acctgaaccgtatatcctatggaaggatttctacatagtgga

SEQ ID NO: 511 - DNA, 51 bp, synthetic construct
  source 1..51: mol_type = other DNA
acctgaaccgtatatcctatggcccgactggaaggatttctacatagtgga

SEQ ID NO: 512 - DNA, 52 bp, synthetic construct
  source 1..52: mol_type = other DNA
acctgaaccgtatatcctatggccctactggaaggatttctacatagtggga

SEQ ID NO: 513 - DNA, 47 bp, synthetic construct
  source 1..47: mol_type = other DNA
acctgaaccgtatatcctatggactggaaggatttctacatagtgga

SEQ ID NO: 514 - DNA, 47 bp, synthetic construct
  source 1..47: mol_type = other DNA
acctgaaccgtatatcctatggcctggaaggatttctacatagtgga

SEQ ID NO: 515 - DNA, 40 bp, synthetic construct
  source 1..40: mol_type = other DNA
acctgaaccgtatatcctggaaggatttctacatagtgga

SEQ ID NO: 516 - DNA, 53 bp, synthetic construct
  source 1..53: mol_type = other DNA
acctgaaccgtatatcctatggccctagactggaaggatttctacatagtgga

SEQ ID NO: 517 - DNA, 38 bp, synthetic construct
  source 1..38: mol_type = other DNA
acctgaaccgtatatcctatggatttctacatagtgga

SEQ ID NO: 518 - DNA, 40 bp, synthetic construct
  source 1..40: mol_type = other DNA
accgtatatcctatggccctgactggaaggatttctacgt

SEQ ID NO: 519 - DNA, 40 bp, synthetic construct; identical to SEQ ID NO: 518
  source 1..40: mol_type = other DNA

SEQ ID NO: 520 - DNA, 40 bp, synthetic construct
  source 1..40: mol_type = other DNA
accgtatatcctatggcccttgactggaaggatttctacg

SEQ ID NO: 521 - DNA, 34 bp, synthetic construct
  source 1..34: mol_type = other DNA
accgtatatcctatgactggaaggatttctacgt

SEQ ID NO: 522 - DNA, 30 bp, synthetic construct
  source 1..30: mol_type = other DNA
accgtatatcctatggaaggatttctacgt

SEQ ID NO: 523 - DNA, 39 bp, synthetic construct
  source 1..39: mol_type = other DNA
accgtatatcctatggcctgactggaaggatttctacgt

SEQ ID NO: 524 - DNA, 38 bp, synthetic construct
  source 1..38: mol_type = other DNA
accgtatatcctatggctgactggaaggatttctacgt

SEQ ID NO: 525 - DNA, 39 bp, synthetic construct
  source 1..39: mol_type = other DNA
accgtatatcctatggcccgactggaaggatttctacgt

SEQ ID NO: 526 - DNA, 34 bp, synthetic construct
  source 1..34: mol_type = other DNA
accgtatatcctatggctggaaggatttctacgt

SEQ ID NO: 527 - DNA, 35 bp, synthetic construct
  source 1..35: mol_type = other DNA
accgtatatcctattgactggaaggatttctacgt

SEQ ID NO: 528 - DNA, 40 bp, synthetic construct
  source 1..40: mol_type = other DNA
accgtatatcctatggccctttgactggaaggatttctac

SEQ ID NO: 529 - DNA, 36 bp, synthetic construct
  source 1..36: mol_type = other DNA
accgtatatcctatgtgactggaaggatttctacgt

SEQ ID NO: 530 - DNA, 37 bp, synthetic construct
  source 1..37: mol_type = other DNA
accgtatatcctatggtgactggaaggatttctacgt

SEQ ID NO: 531 - DNA, 36 bp, synthetic construct
  source 1..36: mol_type = other DNA
accgtatatcctatggccctggaaggatttctacgt

SEQ ID NO: 532 - DNA, 38 bp, synthetic construct
  source 1..38: mol_type = other DNA
accgtatatcctatggccctgactggaaggatttctac

SEQ ID NO: 533 - DNA, 35 bp, synthetic construct
  source 1..35: mol_type = other DNA
accgtatatcctatggactggaaggatttctacgt

SEQ ID NO: 534 - DNA, 28 bp, synthetic construct
  source 1..28: mol_type = other DNA
accgtatatcctggaaggatttctacgt

SEQ ID NO: 535 - DNA, 38 bp, synthetic construct
  source 1..38: mol_type = other DNA
accgtatatcctatggccgactggaaggatttctacgt

SEQ ID NO: 536 - DNA, 39 bp, synthetic construct
  source 1..39: mol_type = other DNA
accgtatatcctatggccctactggaaggatttctacgt

SEQ ID NO: 537 - DNA, 15 bp, synthetic construct
  source 1..15: mol_type = other DNA
gaaggatttctacgt

SEQ ID NO: 538 - DNA, 20 bp, synthetic construct
  source 1..20: mol_type = other DNA
tatacggttcaggtaccagg

SEQ ID NO: 539 - DNA, 52 bp, synthetic construct
  source 1..52: mol_type = other DNA
gccataggatatacggttcaggtaccagggggcagagagaagggcccggaag

SEQ ID NO: 540 - DNA, 45 bp, synthetic construct
  source 1..45: mol_type = other DNA
gccataggatatacggttcagggggcagagagaagggcccggaag

SEQ ID NO: 541 - DNA, 53 bp, synthetic construct
  source 1..53: mol_type = other DNA
gccataggatatacggttcaggtacccagggggcagagagaagggcccggaag

SEQ ID NO: 542 - DNA, 44 bp, synthetic construct
  source 1..44: mol_type = other DNA
gccataggatatacggttcaggggcagagagaagggcccggaag

SEQ ID NO: 543 - DNA, 43 bp, synthetic construct
  source 1..43: mol_type = other DNA
gccataggatatacggttcagggcagagagaagggcccggaag

SEQ ID NO: 544 - DNA, 42 bp, synthetic construct
  source 1..42: mol_type = other DNA
gccataggatatacggttcaggcagagagaagggcccggaag

SEQ ID NO: 545 - DNA, 53 bp, synthetic construct
  source 1..53: mol_type = other DNA
gccataggatatacggttcaggtacctagggggcagagagaagggcccggaag

SEQ ID NO: 546 - DNA, 38 bp, synthetic construct
  source 1..38: mol_type = other DNA
gccataggatatacggttcagagagaagggcccggaag

SEQ ID NO: 547 - DNA, 53 bp, synthetic construct
  source 1..53: mol_type = other DNA
gccataggatatacggttcaggtaccaagggggcagagagaagggcccggaag

SEQ ID NO: 548 - DNA, 49 bp, synthetic construct
  source 1..49: mol_type = other DNA
gccataggatatacggttcaggtagggggcagagagaagggcccggaag

SEQ ID NO: 549 - DNA, 51 bp, synthetic construct
  source 1..51: mol_type = other DNA
gccataggatatacggttcaggtacagggggcagagagaagggcccggaag

SEQ ID NO: 550 - DNA, 40 bp, synthetic construct
  source 1..40: mol_type = other DNA
ggcccttctctctgccccctggtacctgaaccgtatatcc

SEQ ID NO: 551 - DNA, 39 bp, synthetic construct
  source 1..39: mol_type = other DNA
ggcccttctctctgccccctgtacctgaaccgtatatcc

SEQ ID NO: 552 - DNA, 39 bp, synthetic construct
  source 1..39: mol_type = other DNA
ggcccttctctctgccccctggtacctgaaccgtatatc

SEQ ID NO: 553 - DNA, 33 bp, synthetic construct
  source 1..33: mol_type = other DNA
ggcccttctctctgccccctgaaccgtatatcc

SEQ ID NO: 554 - DNA, 39 bp, synthetic construct
  source 1..39: mol_type = other DNA
ggcccttctctctgcccccggtacctgaaccgtatatcc

SEQ ID NO: 555 - DNA, 37 bp, synthetic construct
  source 1..37: mol_type = other DNA
ggcccttctctctgccccctgcctgaaccgtatatcc

SEQ ID NO: 556 - DNA, 20 bp, synthetic construct
  source 1..20: mol_type = other DNA
ggcccttctctctgccccct

SEQ ID NO: 557 - DNA, 38 bp, synthetic construct
  source 1..38: mol_type = other DNA
ggcccttctctctgccccctgacctgaaccgtatatcc

SEQ ID NO: 558 - DNA, 34 bp, synthetic construct
  source 1..34: mol_type = other DNA
ggcccttctctctgccccctggaaccgtatatcc

SEQ ID NO: 559 - DNA, 40 bp, synthetic construct
  source 1..40: mol_type = other DNA
ggcccttctctctgccccctggggtacctgaaccgtatat

SEQ ID NO: 560 - DNA, 37 bp, synthetic construct
  source 1..37: mol_type = other DNA
ggcccttctctctgccccctacctgaaccgtatatcc

SEQ ID NO: 561 - DNA, 32 bp, synthetic construct
  source 1..32: mol_type = other DNA
ggcccttctctctgcccctgaaccgtatatcc

SEQ ID NO: 562 - DNA, 35 bp, synthetic construct
  source 1..35: mol_type = other DNA
ggcccttctctctgccccctgtgaaccgtatatcc

SEQ ID NO: 563 - DNA, 30 bp, synthetic construct
  source 1..30: mol_type = other DNA
ggcccttctctctgcctgaaccgtatatcc

SEQ ID NO: 564 - DNA, 26 bp, synthetic construct
  source 1..26: mol_type = other DNA
ggcccttctctctgaaccgtatatcc

SEQ ID NO: 565 - DNA, 36 bp, synthetic construct
  source 1..36: mol_type = other DNA
ggcccttctctctgccccctgctgaaccgtatatcc

SEQ ID NO: 566 - DNA, 32 bp, synthetic construct
  source 1..32: mol_type = other DNA
ggcccttctctctgtacctgaaccgtatatcc

SEQ ID NO: 567 - DNA, 26 bp, synthetic construct
  source 1..26: mol_type = other DNA
ggcccttctctctgccccgtatatcc

SEQ ID NO: 568 - DNA, 35 bp, synthetic construct
  source 1..35: mol_type = other DNA
ggcccttctctctgccccctctgaaccgtatatcc

SEQ ID NO: 569 - DNA, 14 bp, synthetic construct
  source 1..14: mol_type = other DNA
tgaaccgtatatcc

SEQ ID NO: 570 - DNA, 27 bp, synthetic construct
  source 1..27: mol_type = other DNA
ggcccttctctctgcccccgtatatcc

SEQ ID NO: 571 - DNA, 20 bp, synthetic construct
  source 1..20: mol_type = other DNA
tccttccagtcagggccata

SEQ ID NO: 572 - DNA, 52 bp, synthetic construct
  source 1..52: mol_type = other DNA
atgtagaaatccttccagtcagggccataggatatacggttcaggtaccagg

SEQ ID NO: 573 - DNA, 44 bp, synthetic construct
  source 1..44: mol_type = other DNA
atgtagaaatccttccagtcaggatatacggttcaggtaccagg

SEQ ID NO: 574 - DNA, 53 bp, synthetic construct
  source 1..53: mol_type = other DNA
atgtagaaatccttccagtcagggcccataggatatacggttcaggtaccagg

SEQ ID NO: 575 - DNA, 45 bp, synthetic construct
  source 1..45: mol_type = other DNA
atgtagaaatccttccagtcagggatatacggttcaggtaccagg

SEQ ID NO: 576 - DNA, 53 bp, synthetic construct
  source 1..53: mol_type = other DNA
atgtagaaatccttccagtcagggcctataggatatacggttcaggtaccagg

SEQ ID NO: 577 - DNA, 51 bp, synthetic construct
  source 1..51: mol_type = other DNA
atgtagaaatccttccagtcagggcctaggatatacggttcaggtaccagg

SEQ ID NO: 578 - DNA, 51 bp, synthetic construct
  source 1..51: mol_type = other DNA
atgtagaaatccttccagtcagggcataggatatacggttcaggtaccagg

SEQ ID NO: 579 - DNA, 30 bp, synthetic construct
  source 1..30: mol_type = other DNA
atgtagaaatccttccagtcaggtaccagg

SEQ ID NO: 580 - DNA, 53 bp, synthetic construct
  source 1..53: mol_type = other DNA
atgtagaaatccttccagtcagggccaataggatatacggttcaggtaccagg

SEQ ID NO: 581 - DNA, 40 bp, synthetic construct
  source 1..40: mol_type = other DNA
atgtagaaatccttccaggatatacggttcaggtaccagg

SEQ ID NO: 582 - DNA, 46 bp, synthetic construct
  source 1..46: mol_type = other DNA
atgtagaaatccttccagtcaggggatatacggttcaggtaccagg

SEQ ID NO: 583 - DNA, 40 bp, synthetic construct
  source 1..40: mol_type = other DNA
acctgaaccgtatatcctatggccctgactggaaggattt

SEQ ID NO: 584 - DNA, 40 bp, synthetic construct
  source 1..40: mol_type = other DNA
acctgaaccgtatatcctatgggccctgactggaaggatt

SEQ ID NO: 585 - DNA, 32 bp, synthetic construct
  source 1..32: mol_type = other DNA
acctgaaccgtatatcctgactggaaggattt

SEQ ID NO: 586 - DNA, 39 bp, synthetic construct
  source 1..39: mol_type = other DNA
acctgaaccgtatatcctatgccctgactggaaggattt

SEQ ID NO: 587 - DNA, 33 bp, synthetic construct
  source 1..33: mol_type = other DNA
acctgaaccgtatatccctgactggaaggattt

SEQ ID NO: 588 - DNA, 31 bp, synthetic construct
  source 1..31: mol_type = other DNA
acctgaaccgtatatcctatgggaaggattt

SEQ ID NO: 589 - DNA, 34 bp, synthetic construct
  source 1..34: mol_type = other DNA
acctgaaccgtatatcctatgactggaaggattt

SEQ ID NO: 590 - DNA, 18 bp, synthetic construct
  source 1..18: mol_type = other DNA
acctgactggaaggattt

SEQ ID NO: 591 - DNA, 30 bp, synthetic construct
  source 1..30: mol_type = other DNA
acctgaaccgtatatcctatggaaggattt

SEQ ID NO: 592 - DNA, 38 bp, synthetic construct
  source 1..38: mol_type = other DNA
acctgaaccgtatatcctggccctgactggaaggattt

SEQ ID NO: 593 - DNA, 32 bp, synthetic construct
  source 1..32: mol_type = other DNA
acctgaaccgtataccctgactggaaggattt

SEQ ID NO: 594 - DNA, 20 bp, synthetic construct
  source 1..20: mol_type = other DNA
agtcagggccataggatata

SEQ ID NO: 595 - DNA, 52 bp, synthetic construct
  source 1..52: mol_type = other DNA
aatccttccagtcagggccataggatatacggttcaggtaccagggggcaga

SEQ ID NO: 596 - DNA, 53 bp, synthetic construct
  source 1..53: mol_type = other DNA
aatccttccagtcagggccataggattatacggttcaggtaccagggggcaga

SEQ ID NO: 597 - DNA, 50 bp, synthetic construct
  source 1..50: mol_type = other DNA
aatccttccagtcagggccataggatacggttcaggtaccagggggcaga

SEQ ID NO: 598 - DNA, 45 bp, synthetic construct
  source 1..45: mol_type = other DNA
aatccttccagtcagggccatacggttcaggtaccagggggcaga

SEQ ID NO: 599 - DNA, 44 bp, synthetic construct
  source 1..44: mol_type = other DNA
aatccttccagtcagggccataggttcaggtaccagggggcaga

SEQ ID NO: 600 - DNA, 38 bp, synthetic construct
  source 1..38: mol_type = other DNA
aatccttccagtcagggccataggtaccagggggcaga

SEQ ID NO: 601 - DNA, 51 bp, synthetic construct
  source 1..51: mol_type = other DNA
aatccttccagtcagggccataggaatacggttcaggtaccagggggcaga

SEQ ID NO: 602 - DNA, 51 bp, synthetic construct
  source 1..51: mol_type = other DNA
aatccttccagtcagggccataggattacggttcaggtaccagggggcaga

SEQ ID NO: 603 - DNA, 30 bp, synthetic construct
  source 1..30: mol_type = other DNA
aatccttccagtcaggtaccagggggcaga

SEQ ID NO: 604 - DNA, 48 bp, synthetic construct
  source 1..48: mol_type = other DNA
aatccttccagtcagggccataggacggttcaggtaccagggggcaga

SEQ ID NO: 605 - DNA, 47 bp, synthetic construct
  source 1..47: mol_type = other DNA
aatccttccagtcagggccatatacggttcaggtaccagggggcaga

SEQ ID NO: 606 - DNA, 40 bp, synthetic construct
  source 1..40: mol_type = other DNA
ccctggtacctgaaccgtatatcctatggccctgactgga

SEQ ID NO: 607 - DNA, 40 bp, synthetic construct
  source 1..40: mol_type = other DNA
ccctggtacctgaaccgtataatcctatggccctgactgg

SEQ ID NO: 608 - DNA, 33 bp, synthetic construct
  source 1..33: mol_type = other DNA
ccctggtacctgaaccgtatggccctgactgga

SEQ ID NO: 609 - DNA, 32 bp, synthetic construct
  source 1..32: mol_type = other DNA
ccctggtacctgaacctatggccctgactgga

SEQ ID NO: 610 - DNA, 38 bp, synthetic construct
  source 1..38: mol_type = other DNA
ccctggtacctgaaccgtatcctatggccctgactgga

SEQ ID NO: 611 - DNA, 37 bp, synthetic construct
  source 1..37: mol_type = other DNA
ccctggtacctgaaccgtatatatggccctgactgga

SEQ ID NO: 612 - DNA, 39 bp, synthetic construct
  source 1..39: mol_type = other DNA
ccctggtacctgaaccgtatacctatggccctgactgga

SEQ ID NO: 613 - DNA, 38 bp, synthetic construct
  source 1..38: mol_type = other DNA
ccctggtacctgaaccgtatactatggccctgactgga

SEQ ID NO: 614 - DNA, 39 bp, synthetic construct
  source 1..39: mol_type = other DNA
ccctggtacctgaaccgtattcctatggccctgactgga

SEQ ID NO: 615 - DNA, 18 bp, synthetic construct
  source 1..18: mol_type = other DNA
ccctggtacctgactgga

SEQ ID NO: 616 - DNA, 40 bp, synthetic construct
  source 1..40: mol_type = other DNA
ccctggtacctgaaccgtatatatcctatggccctgactg

SEQ ID NO: 617 - DNA, 35 bp, synthetic construct
  source 1..35: mol_type = other DNA
ccctggtacctgaaccgtatatggccctgactgga

SEQ ID NO: 618 - DNA, 26 bp, synthetic construct
  source 1..26: mol_type = other DNA
ccctggtacctatggccctgactgga

SEQ ID NO: 619 - DNA, 19 bp, synthetic construct
  source 1..19: mol_type = other DNA
ccctggtacctgaactgga

SEQ ID NO: 620 - DNA, 33 bp, synthetic construct
  source 1..33: mol_type = other DNA
ccctggtacctgaatcctatggccctgactgga

SEQ ID NO: 621 - DNA, 40 bp, synthetic construct
  source 1..40: mol_type = other DNA
ccctggtacctgaaccgtataaatcctatggccctgactg

SEQ ID NO: 622 - DNA, 32 bp, synthetic construct
  source 1..32: mol_type = other DNA
ccctggtacctgatcctatggccctgactgga

SEQ ID NO: 623 - DNA, 37 bp, synthetic construct
  source 1..37: mol_type = other DNA
ccctggtacctgaaccgtatctatggccctgactgga

SEQ ID NO: 624 - DNA, 20 bp, synthetic construct
  source 1..20: mol_type = other DNA
gaagaccaaggatagactgc

SEQ ID NO: 625 - DNA, 52 bp, synthetic construct
  source 1..52: mol_type = other DNA
gggcaaagtgaagaccaaggatagactgctgggcttgacagcatggagggtg

SEQ ID NO: 626 - DNA, 39 bp, synthetic construct
  source 1..39: mol_type = other DNA
gggcaaagtgaagaccaaggatagacagcatggagggtg

SEQ ID NO: 627 - DNA, 53 bp, synthetic construct
  source 1..53: mol_type = other DNA
gggcaaagtgaagaccaaggatagacctgctgggcttgacagcatggagggtg

SEQ ID NO: 628 - DNA, 49 bp, synthetic construct
  source 1..49: mol_type = other DNA
gggcaaagtgaagaccaaggatagactgggcttgacagcatggagggtg

SEQ ID NO: 629 - DNA, 51 bp, synthetic construct
  source 1..51: mol_type = other DNA
gggcaaagtgaagaccaaggatagacgctgggcttgacagcatggagggtg

SEQ ID NO: 630 - DNA, 51 bp, synthetic construct
  source 1..51: mol_type = other DNA
gggcaaagtgaagaccaaggatagatgctgggcttgacagcatggagggtg

SEQ ID NO: 631 - DNA, 40 bp, synthetic construct
  source 1..40: mol_type = other DNA
gggcaaagtgaagaccaagggcttgacagcatggagggtg

SEQ ID NO: 632 - DNA, 48 bp, synthetic construct
  source 1..48: mol_type = other DNA
gggcaaagtgaagaccaaggatagctgggcttgacagcatggagggtg

SEQ ID NO: 633 - DNA, 39 bp, synthetic construct
  source 1..39: mol_type = other DNA
gggcaaagtgaagaccaaggcttgacagcatggagggtg

SEQ ID NO: 634 - DNA, 47 bp, synthetic construct
  source 1..47: mol_type = other DNA
gggcaaagtgaagaccaaggatgctgggcttgacagcatggagggtg

SEQ ID NO: 635 - DNA, 49 bp, synthetic construct
  source 1..49: mol_type = other DNA
gggcaaagtgaagaccaaggataggctgggcttgacagcatggagggtg

SEQ ID NO: 636 - DNA, 40 bp, synthetic construct
  source 1..40: mol_type = other DNA
agtgaagaccaaggatagactgctgggcttgacagcatgg

SEQ ID NO: 637 - DNA, 39 bp, synthetic construct
  source 1..39: mol_type = other DNA
agtgaagaccaaggatagatgctgggcttgacagcatgg

SEQ ID NO: 638 - DNA, 36 bp, synthetic construct
  source 1..36: mol_type = other DNA
agtgaagaccaaggattgctgggcttgacagcatgg

SEQ ID NO: 639 - DNA, 40 bp, synthetic construct
  source 1..40: mol_type = other DNA
agtgaagaccaaggatagacctgctgggcttgacagcatg

SEQ ID NO: 640 - DNA, 35 bp, synthetic construct
  source 1..35: mol_type = other DNA
agtgaagaccaaggatgctgggcttgacagcatgg

SEQ ID NO: 641 - DNA, 37 bp, synthetic construct
  source 1..37: mol_type = other DNA
agtgaagaccaaggatctgctgggcttgacagcatgg

SEQ ID NO: 642 - DNA, 37 bp, synthetic construct
  source 1..37: mol_type = other DNA
agtgaagaccaaggatatgctgggcttgacagcatgg

SEQ ID NO: 643 - DNA, 34 bp, synthetic construct
  source 1..34: mol_type = other DNA
agtgaagaccaaggtgctgggcttgacagcatgg

SEQ ID NO: 644 - DNA, 39 bp, synthetic construct
  source 1..39: mol_type = other DNA
agtgaagaccaaggatagacgctgggcttgacagcatgg

SEQ ID NO: 645 - DNA, 27 bp, synthetic construct
  source 1..27: mol_type = other DNA
agtgaagaccaaggatagacagcatgg

SEQ ID NO: 646 - DNA, 38 bp, synthetic construct
  source 1..38: mol_type = other DNA
agtgaagaccaaggatagtgctgggcttgacagcatgg

SEQ ID NO: 647 - DNA, 36 bp, synthetic construct
  source 1..36: mol_type = other DNA
agtgaagaccaaggactgctgggcttgacagcatgg

SEQ ID NO: 648 - DNA, 39 bp, synthetic construct
  source 1..39: mol_type = other DNA
agtgaagaccaaggatagactgctgggcttgacagcatg

SEQ ID NO: 649 - DNA, 37 bp, synthetic construct
  source 1..37: mol_type = other DNA
agtgaagaccaaggatagactgggcttgacagcatgg

SEQ ID NO: 650 - DNA, 35 bp, synthetic construct
  source 1..35: mol_type = other DNA
agtgaagaccaaggctgctgggcttgacagcatgg

SEQ ID NO: 651 - DNA, 38 bp, synthetic construct
  source 1..38: mol_type = other DNA
agtgaagaccaaggatactgctgggcttgacagcatgg

SEQ ID NO: 652 - DNA, 31 bp, synthetic construct
  source 1..31: mol_type = other DNA
agtgaagaccatgctgggcttgacagcatgg

SEQ ID NO: 653 - DNA, 33 bp, synthetic construct
  source 1..33: mol_type = other DNA
agtgaagaccaagtgctgggcttgacagcatgg

SEQ ID NO: 654 - DNA, 28 bp, synthetic construct
  source 1..28: mol_type = other DNA
agtgaagaccaagggcttgacagcatgg

SEQ ID NO: 655 - DNA, 27 bp, synthetic construct
  source 1..27: mol_type = other DNA
agtgaagaccaaggcttgacagcatgg

SEQ ID NO: 656 - DNA, 30 bp, synthetic construct
  source 1..30: mol_type = other DNA
agtgaagaccaaggatagacgacagcatgg

SEQ ID NO: 657 - DNA, 22 bp, synthetic construct
  source 1..22: mol_type = other DNA
agtgctgggcttgacagcatgg

SEQ ID NO: 658 - DNA, 20 bp, synthetic construct
  source 1..20: mol_type = other DNA
tgctgggcttgacagcatgg

SEQ ID NO: 659 - DNA, 23 bp, synthetic construct
  source 1..23: mol_type = other DNA
agtgaagaccaaggacagcatgg

SEQ ID NO: 660 - DNA, 34 bp, synthetic construct
  source 1..34: mol_type = other DNA
agtgaagaccaagctgctgggcttgacagcatgg

SEQ ID NO: 661 - DNA, 14 bp, synthetic construct
  source 1..14: mol_type = other DNA
agtgaagaccaagg

SEQ ID NO: 662 - DNA, 38 bp, synthetic construct
  source 1..38: mol_type = other DNA
agtgaagaccaaggatagacctgggcttgacagcatgg

SEQ ID NO: 663 - DNA, 23 bp, synthetic construct
  source 1..23: mol_type = other DNA
agtgaagaccaaggatagcatgg

SEQ ID NO: 664 - DNA, 39 bp, synthetic construct
  source 1..39: mol_type = other DNA
agtgaagaccaaggatagctgctgggcttgacagcatgg

SEQ ID NO: 665 - DNA, 31 bp, synthetic construct
  source 1..31: mol_type = other DNA
agtgaagaccaagctgggcttgacagcatgg

SEQ ID NO: 666 - DNA, 31 bp, synthetic construct
  source 1..31: mol_type = other DNA
agtgaagaccaaggatagactgacagcatgg

SEQ ID NO: 667 - DNA, 40 bp, synthetic construct
  source 1..40: mol_type = other DNA
agtgaagaccaaggatagacgtgctgggcttgacagcatg

SEQ ID NO: 668 - DNA, 29 bp, synthetic construct
  source 1..29: mol_type = other DNA
agtgaagactgctgggcttgacagcatgg

SEQ ID NO: 669 - DNA, 36 bp, synthetic construct
  source 1..36: mol_type = other DNA
agtgaagaccaaggatagctgggcttgacagcatgg

SEQ ID NO: 670 - DNA, 32 bp, synthetic construct
  source 1..32: mol_type = other DNA
agtgaagaccaaggctgggcttgacagcatgg

SEQ ID NO: 671 - DNA, 20 bp, synthetic construct
  source 1..20: mol_type = other DNA
ggagctcaatgatcgcttgg

SEQ ID NO: 672 - DNA, 52 bp, synthetic construct
  source 1..52: mol_type = other DNA
ggacctgcaggagctcaatgatcgcttggcggtctacatcgaccgtgtgcgc

SEQ ID NO: 673 - DNA, 53 bp, synthetic construct
  source 1..53: mol_type = other DNA
ggacctgcaggagctcaatgatcgctttggcggtctacatcgaccgtgtgcgc

SEQ ID NO: 674 - DNA, 47 bp, synthetic construct
  source 1..47: mol_type = other DNA
ggacctgcaggagctcaatgatcgcggtctacatcgaccgtgtgcgc

SEQ ID NO: 675 - DNA, 51 bp, synthetic construct
  source 1..51: mol_type = other DNA
ggacctgcaggagctcaatgatcgctggcggtctacatcgaccgtgtgcgc

SEQ ID NO: 676 - DNA, 45 bp, synthetic construct
  source 1..45: mol_type = other DNA
ggacctgcaggagctcaatgatcggtctacatcgaccgtgtgcgc

SEQ ID NO: 677 - DNA, 44 bp, synthetic construct
  source 1..44: mol_type = other DNA
ggacctgcaggagctcaatggcggtctacatcgaccgtgtgcgc

SEQ ID NO: 678 - DNA, 43 bp, synthetic construct
  source 1..43: mol_type = other DNA
ggacctgcaggagctcaatgatcgctacatcgaccgtgtgcgc

SEQ ID NO: 679 - DNA, 35 bp, synthetic construct
  source 1..35: mol_type = other DNA
ggacctgcaggagctcaatgatcgaccgtgtgcgc

SEQ ID NO: 680 - DNA, 48 bp, synthetic construct
  source 1..48: mol_type = other DNA
ggacctgcaggagctcaatgatcggcggtctacatcgaccgtgtgcgc

SEQ ID NO: 681 - DNA, 41 bp, synthetic construct
  source 1..41: mol_type = other DNA
ggacctgcaggagctcaatgatctacatcgaccgtgtgcgc

SEQ ID NO: 682 - DNA, 47 bp, synthetic construct
  source 1..47: mol_type = other DNA
ggacctgcaggagctcaatgatggcggtctacatcgaccgtgtgcgc

SEQ ID NO: 683 - DNA, 40 bp, synthetic construct
  source 1..40: mol_type = other DNA
gcaggagctcaatgatcgcttggcggtctacatcgaccgt

SEQ ID NO: 684 - DNA, 40 bp, synthetic construct
  source 1..40: mol_type = other DNA
gcaggagctcaatgatcgctttggcggtctacatcgaccg

SEQ ID NO: 685 - DNA, 39 bp, synthetic construct
  source 1..39: mol_type = other DNA
gcaggagctcaatgatcgctggcggtctacatcgaccgt

SEQ ID NO: 686 - DNA, 35 bp, synthetic construct
  source 1..35: mol_type = other DNA
gcaggagctcaatgatcgcggtctacatcgaccgt

SEQ ID NO: 687 - DNA, 23 bp, synthetic construct
  source 1..23: mol_type = other DNA
gcaggagctcaatgatcgaccgt

SEQ ID NO: 688 - DNA, 32 bp, synthetic construct
  source 1..32: mol_type = other DNA
gcaggagctcaatggcggtctacatcgaccgt

SEQ ID NO: 689 - DNA, 40 bp, synthetic construct
  source 1..40: mol_type = other DNA
gcaggagctcaatgatcgctcttggcggtctacatcgacc

SEQ ID NO: 690 - DNA, 31 bp, synthetic construct
  source 1..31: mol_type = other DNA
gcaggagctcaatgatcgctacatcgaccgt

SEQ ID NO: 691 - DNA, 35 bp, synthetic construct
  source 1..35: mol_type = other DNA
gcaggagctcaatgttggcggtctacatcgaccgt

SEQ ID NO: 692 - DNA, 35 bp, synthetic construct
  source 1..35: mol_type = other DNA
gcaggagctcaatgatggcggtctacatcgaccgt

SEQ ID NO: 693 - DNA, 35 bp, synthetic construct
  source 1..35: mol_type = other DNA
gcaggagctcaatgatcgctgtctacatcgaccgt

SEQ ID NO: 694 - DNA, 39 bp, synthetic construct
  source 1..39: mol_type = other DNA
gcaggagctcaatgatcgttggcggtctacatcgaccgt

SEQ ID NO: 695 - DNA, 38 bp, synthetic construct
  source 1..38: mol_type = other DNA
gcaggagctcaatgatcgtggcggtctacatcgaccgt

SEQ ID NO: 696 - DNA, 20 bp, synthetic construct
  source 1..20: mol_type = other DNA
gcaggagctcaatgatcgct

SEQ ID NO: 697 - DNA, 38 bp, synthetic construct
  source 1..38: mol_type = other DNA
gcaggagctcaatgatcgctgcggtctacatcgaccgt

SEQ ID NO: 698 - DNA, 36 bp, synthetic construct
  source 1..36: mol_type = other DNA
gcaggagctcaatgattggcggtctacatcgaccgt

SEQ ID NO: 699 - DNA, 33 bp, synthetic construct
  source 1..33: mol_type = other DNA
gcaggagctcaatgatcggtctacatcgaccgt

SEQ ID NO: 700 - DNA, 31 bp, synthetic construct
  source 1..31: mol_type = other DNA
gcaggagctcatggcggtctacatcgaccgt

SEQ ID NO: 701 - DNA, 33 bp, synthetic construct
  source 1..33: mol_type = other DNA
gcaggagctcaattggcggtctacatcgaccgt

SEQ ID NO: 702 - DNA, 19 bp, synthetic construct
  source 1..19: mol_type = other DNA
gcaggagctcaatgaccgt

SEQ ID NO: 703 - DNA, 19 bp, synthetic construct
  source 1..19: mol_type = other DNA
gcaggagctcaatgatcgt

SEQ ID NO: 704 - DNA, 36 bp, synthetic construct
  source 1..36: mol_type = other DNA
gcaggagctcaatgatcgctggtctacatcgaccgt

SEQ ID NO: 705 - DNA, 33 bp, synthetic construct
  source 1..33: mol_type = other DNA
gcaggagctcaatgatcgctctacatcgaccgt

SEQ ID NO: 706 - DNA, 40 bp, synthetic construct
  source 1..40: mol_type = other DNA
gcaggagctcaatgatcgcttttggcggtctacatcgacc

SEQ ID NO: 707 - DNA, 30 bp, synthetic construct
  source 1..30: mol_type = other DNA
gcaggagctcaatgatcgctcatcgaccgt

SEQ ID NO: 708 - DNA, 29 bp, synthetic construct
  source 1..29: mol_type = other DNA
gcaggagctcaatgatctacatcgaccgt

SEQ ID NO: 709 - DNA, 27 bp, synthetic construct
  source 1..27: mol_type = other DNA
gcaggagctcaatgatcgctcgaccgt

SEQ ID NO: 710 - DNA, 37 bp, synthetic construct
  source 1..37: mol_type = other DNA
gcaggagctcaatgatttggcggtctacatcgaccgt

SEQ ID NO: 711 - DNA, 30 bp, synthetic construct
  source 1..30: mol_type = other DNA
gcaggagctctggcggtctacatcgaccgt

SEQ ID NO: 712 - DNA, 26 bp, synthetic construct
  source 1..26: mol_type = other DNA
gcaggagctcaatgatcgctgaccgt

SEQ ID NO: 713 - DNA, 32 bp, synthetic construct
  source 1..32: mol_type = other DNA
gcaggagctcattggcggtctacatcgaccgt

SEQ ID NO: 714 - DNA, 38 bp, synthetic construct
  source 1..38: mol_type = other DNA
gcaggagctcaatgatcttggcggtctacatcgaccgt

SEQ ID NO: 715 - DNA, 28 bp, synthetic construct
  source 1..28: mol_type = other DNA
gcaggagctggcggtctacatcgaccgt

SEQ ID NO: 716 - DNA, 22 bp, synthetic construct
  source 1..22: mol_type = other DNA
gcaggagctctacatcgaccgt

SEQ ID NO: 717 - DNA, 29 bp, synthetic construct
  source 1..29: mol_type = other DNA
gcaggagcttggcggtctacatcgaccgt

SEQ ID NO: 718 - DNA, 37 bp, synthetic construct
  source 1..37: mol_type = other DNA
gcaggagctcaatgatctggcggtctacatcgaccgt

SEQ ID NO: 719 - DNA, 14 bp, synthetic construct
  source 1..14: mol_type = other DNA
tctacatcgaccgt

SEQ ID NO: 720 - DNA, 32 bp, synthetic construct
  source 1..32: mol_type = other DNA
gcaggagctcaatgatcgcttacatcgaccgt

SEQ ID NO: 721 - DNA, 28 bp, synthetic construct
  source 1..28: mol_type = other DNA
gcaggagctcaatgatcgcatcgaccgt

SEQ ID NO: 722 - DNA, 40 bp, synthetic construct
  source 1..40: mol_type = other DNA
gcaggagctcaatgatcgctgtggcggtctacatcgaccg

SEQ ID NO: 723 - DNA, 27 bp, synthetic construct
  source 1..27: mol_type = other DNA
gcaggagtggcggtctacatcgaccgt

SEQ ID NO: 724 - DNA, 20 bp, synthetic construct
  source 1..20: mol_type = other DNA
tttctatctgcagctctggg

SEQ ID NO: 725 - DNA, 52 bp, synthetic construct
  source 1..52: mol_type = other DNA
cagcaatgatttctatctgcagctctgggaggctctggaacagtggacaaaa

SEQ ID NO: 726 - DNA, 53 bp, synthetic construct
  source 1..53: mol_type = other DNA
cagcaatgatttctatctgcagctcttgggaggctctggaacagtggacaaaa

SEQ ID NO: 727 - DNA, 42 bp, synthetic construct
  source 1..42: mol_type = other DNA
cagcaatgatttctatctgcagctctggaacagtggacaaaa

SEQ ID NO: 728 - DNA, 44 bp, synthetic construct
  source 1..44: mol_type = other DNA
cagcaatgatttctatctgcagctctctggaacagtggacaaaa

SEQ ID NO: 729 - DNA, 43 bp, synthetic construct
  source 1..43: mol_type = other DNA
cagcaatgatttctatctgcaggctctggaacagtggacaaaa

SEQ ID NO: 730 - DNA, 34 bp, synthetic construct
  source 1..34: mol_type = other DNA
cagcaatgatttctatctggaacagtggacaaaa

SEQ ID NO: 731 - DNA, 51 bp, synthetic construct
  source 1..51: mol_type = other DNA
cagcaatgatttctatctgcagctcgggaggctctggaacagtggacaaaa

SEQ ID NO: 732 - DNA, 51 bp, synthetic construct
  source 1..51: mol_type = other DNA
cagcaatgatttctatctgcagctctggaggctctggaacagtggacaaaa

SEQ ID NO: 733 - DNA, 53 bp, synthetic construct
  source 1..53: mol_type = other DNA
cagcaatgatttctatctgcagctctagggaggctctggaacagtggacaaaa

SEQ ID NO: 734 - DNA, 39 bp, synthetic construct
  source 1..39: mol_type = other DNA
cagcaatgatttctatctgctctggaacagtggacaaaa

SEQ ID NO: 735 - DNA, 47 bp, synthetic construct
  source 1..47: mol_type = other DNA
cagcaatgatttctatctgcagggaggctctggaacagtggacaaaa

SEQ ID NO: 736 - DNA, 40 bp, synthetic construct
  source 1..40: mol_type = other DNA
cagcaatgatttctatctgcagctggaacagtggacaaaa

SEQ ID NO: 737 - DNA, 40 bp, synthetic construct
  source 1..40: mol_type = other DNA
ccactgttccagagcctcccagagctgcagatagaaatca

SEQ ID NO: 738 - DNA, 40 bp, synthetic construct
  source 1..40: mol_type = other DNA
ccactgttccagagcctcccaagagctgcagatagaaatc

SEQ ID NO: 739 - DNA, 32 bp, synthetic construct
  source 1..32: mol_type = other DNA
ccactgttccagagcctcccagatagaaatca

SEQ ID NO: 740 - DNA, 40 bp, synthetic construct
  source 1..40: mol_type = other DNA
ccactgttccagagcctcccaaagagctgcagatagaaat

SEQ ID NO: 741 - DNA, 39 bp, synthetic construct
  source 1..39: mol_type = other DNA
ccactgttccagagcctcccaagctgcagatagaaatca

SEQ ID NO: 742 - DNA, 40 bp, synthetic construct
  source 1..40: mol_type = other DNA
ccactgttccagagcctcccatagagctgcagatagaaat

SEQ ID NO: 743 - DNA, 40 bp, synthetic construct
  source 1..40: mol_type = other DNA
ccactgttccagagcctcccacagagctgcagatagaaat

SEQ ID NO: 744 - DNA, 38 bp, synthetic construct
  source 1..38: mol_type = other DNA
ccactgttccagagcctcccagctgcagatagaaatca

SEQ ID NO: 745 - DNA, 35 bp, synthetic construct
  source 1..35: mol_type = other DNA
ccactgttccagagcctccctgcagatagaaatca

SEQ ID NO: 746 - DNA, 40 bp, synthetic construct
  source 1..40: mol_type = other DNA
ccactgttccagagcctcccaagagctgcagatagaaaac

SEQ ID NO: 747 - DNA, 39 bp, synthetic construct
  source 1..39: mol_type = other DNA
ccactgttccagagcctcccgagctgcagatagaaatca

SEQ ID NO: 748 - DNA, 37 bp, synthetic construct
  source 1..37: mol_type = other DNA
ccactgttccagagcctcccactgcagatagaaatca

SEQ ID NO: 749 - DNA, 20 bp, synthetic construct
  source 1..20: mol_type = other DNA
aggctgcagctcccactggg

SEQ ID NO: 750 - DNA, 52 bp, synthetic construct
  source 1..52: mol_type = other DNA
ctctgccccaggctgcagctcccactgggaggtggaggaccttggcacccac

SEQ ID NO: 751 - DNA, 53 bp, synthetic construct
  source 1..53: mol_type = other DNA
ctctgccccaggctgcagctcccacttgggaggtggaggaccttggcacccac

SEQ ID NO: 752 - DNA, 51 bp, synthetic construct
  source 1..51: mol_type = other DNA
ctctgccccaggctgcagctcccacgggaggtggaggaccttggcacccac

SEQ ID NO: 753 - DNA, 51 bp, synthetic construct
  source 1..51: mol_type = other DNA
ctctgccccaggctgcagctcccactggaggtggaggaccttggcacccac

SEQ ID NO: 754 - DNA, 39 bp, synthetic construct
  source 1..39: mol_type = other DNA
ctctgccccaggctgcaggtggaggaccttggcacccac

SEQ ID NO: 755 - DNA, 46 bp, synthetic construct
  source 1..46: mol_type = other DNA
ctctgccccaggctgcagctcccaggtggaggaccttggcacccac

SEQ ID NO: 756 - DNA, 50 bp, synthetic construct
  source 1..50: mol_type = other DNA
ctctgccccaggctgcagctcccagggaggtggaggaccttggcacccac

SEQ ID NO: 757 - DNA, 50 bp, synthetic construct
  source 1..50: mol_type = other DNA
ctctgccccaggctgcagctcccactgaggtggaggaccttggcacccac

SEQ ID NO: 758 - DNA, 50 bp, synthetic construct
  source 1..50: mol_type = other DNA
ctctgccccaggctgcagctcccacggaggtggaggaccttggcacccac

SEQ ID NO: 759 - DNA, 25 bp, synthetic construct
  source 1..25: mol_type = other DNA
ctctgccccaggctgcagctcccac

SEQ ID NO: 760 - DNA, 45 bp, synthetic construct
  source 1..45: mol_type = other DNA
ctctgccccaggctgcagctcccactggaggaccttggcacccac

SEQ ID NO: 761 - DNA, 40 bp, synthetic construct
  source 1..40: mol_type = other DNA
cccaggctgcagctcccactgggaggtggaggaccttggc

SEQ ID NO: 762 - DNA, 40 bp, synthetic construct
  source 1..40: mol_type = other DNA
cccaggctgcagctcccacttgggaggtggaggaccttgg

SEQ ID NO: 763 - DNA, 39 bp, synthetic construct
  source 1..39: mol_type = other DNA
cccaggctgcagctcccatgggaggtggaggaccttggc

SEQ ID NO: 764 - DNA, 38 bp, synthetic construct
  source 1..38: mol_type = other DNA
cccaggctgcagctccctgggaggtggaggaccttggc

SEQ ID NO: 765 - DNA, 37 bp, synthetic construct
  source 1..37: mol_type = other DNA
cccaggctgcagctcctgggaggtggaggaccttggc

SEQ ID NO: 766 - DNA, 39 bp, synthetic construct
  source 1..39: mol_type = other DNA
cccaggctgcagctcccacgggaggtggaggaccttggc

SEQ ID NO: 767 - DNA, 40 bp, synthetic construct
  source 1..40: mol_type = other DNA
cccaggctgcagctcccactctgggaggtggaggaccttg

SEQ ID NO: 768 - DNA, 36 bp, synthetic construct
  source 1..36: mol_type = other DNA
cccaggctgcagctctgggaggtggaggaccttggc

SEQ ID NO: 769 - DNA, 40 bp, synthetic construct
  source 1..40: mol_type = other DNA
cccaggctgcagctcccactttgggaggtggaggaccttg

SEQ ID NO: 770 - DNA, 30 bp, synthetic construct
  source 1..30: mol_type = other DNA
cccaggctgtgggaggtggaggaccttggc

SEQ ID NO: 771 - DNA, 40 bp, synthetic construct
  source 1..40: mol_type = other DNA
cccaggctgcagctcccacttggaggtggaggaccttggc

SEQ ID NO: 772 - DNA, 20 bp, synthetic construct
  source 1..20: mol_type = other DNA
gtaaacaaagcatagactga

SEQ ID NO: 773 - DNA, 52 bp, synthetic construct
  source 1..52: mol_type = other DNA
acactcagagtaaacaaagcatagactgaggggtacaatcctactctagtcc

SEQ ID NO: 774 - DNA, 48 bp, synthetic construct
  source 1..48: mol_type = other DNA
acactcagagtaaacaaagcatagaggggtacaatcctactctagtcc

SEQ ID NO: 775 - DNA, 46 bp, synthetic construct
  source 1..46: mol_type = other DNA
acactcagagtaaacaaagcataggggtacaatcctactctagtcc

SEQ ID NO: 776 - DNA, 53 bp, synthetic construct
  source 1..53: mol_type = other DNA
acactcagagtaaacaaagcatagacctgaggggtacaatcctactctagtcc

SEQ ID NO: 777 - DNA, 42 bp, synthetic construct
  source 1..42: mol_type = other DNA
acactcagagtaaacaaagcatagacaatcctactctagtcc

SEQ ID NO: 778 - DNA, 51 bp, synthetic construct
  source 1..51: mol_type = other DNA
acactcagagtaaacaaagcatagacgaggggtacaatcctactctagtcc

SEQ ID NO: 779 - DNA, 51 bp, synthetic construct
  source 1..51: mol_type = other DNA
acactcagagtaaacaaagcatagatgaggggtacaatcctactctagtcc

SEQ ID NO: 780 - DNA, 41 bp, synthetic construct
  source 1..41: mol_type = other DNA
acactcagagtaaacaaaggggtacaatcctactctagtcc

SEQ ID NO: 781 - DNA, 47 bp, synthetic construct
  source 1..47: mol_type = other DNA
acactcagagtaaacaaagcatgaggggtacaatcctactctagtcc

SEQ ID NO: 782 - DNA, 53 bp, synthetic construct
  source 1..53: mol_type = other DNA
acactcagagtaaacaaagcatagacttgaggggtacaatcctactctagtcc

SEQ ID NO: 783 - DNA, 40 bp, synthetic construct
  source 1..40: mol_type = other DNA
acactcagagtaaacaaagcatacaatcctactctagtcc

SEQ ID NO: 784 - DNA, 40 bp, synthetic construct
  source 1..40: mol_type = other DNA
gagtaggattgtacccctcagtctatgctttgtttactct

SEQ ID NO: 785 - DNA, 40 bp, synthetic construct
  source 1..40: mol_type = other DNA
gagtaggattgtacccctcaggtctatgctttgtttactc

SEQ ID NO: 786 - DNA, 36 bp, synthetic construct
  source 1..36: mol_type = other DNA
gagtaggattgtacccctctatgctttgtttactct

SEQ ID NO: 787 - DNA, 34 bp, synthetic construct
  source 1..34: mol_type = other DNA
gagtaggattgtacccctcagctttgtttactct

SEQ ID NO: 788 - DNA, 37 bp, synthetic construct
  source 1..37: mol_type = other DNA
gagtaggattgtacccctcagatgctttgtttactct

SEQ ID NO: 789 - DNA, 39 bp, synthetic construct
  source 1..39: mol_type = other DNA
gagtaggattgtacccctcatctatgctttgtttactct

SEQ ID NO: 790 - DNA, 38 bp, synthetic construct
  source 1..38: mol_type = other DNA
gagtaggattgtacccctcagtatgctttgtttactct

SEQ ID NO: 791 - DNA, 39 bp, synthetic construct
  source 1..39: mol_type = other DNA
gagtaggattgtacccctcgtctatgctttgtttactct

SEQ ID NO: 792 - DNA, 30 bp, synthetic construct
  source 1..30: mol_type = other DNA
gagtaggattgtctatgctttgtttactct

SEQ ID NO: 793 - DNA, 34 bp, synthetic construct
  source 1..34: mol_type = other DNA
gagtaggattgtacctctatgctttgtttactct

SEQ ID NO: 794 - DNA, 34 bp, synthetic construct
  source 1..34: mol_type = other DNA
gagtaggattgtacgtctatgctttgtttactct

SEQ ID NO: 795 - DNA, 39 bp, synthetic construct
  source 1..39: mol_type = other DNA
gagtaggattgtacccctcagctatgctttgtttactct

SEQ ID NO: 796 - DNA, 34 bp, synthetic construct
  source 1..34: mol_type = other DNA
gagtaggattgtacccctatgctttgtttactct

SEQ ID NO: 797 - DNA, 35 bp, synthetic construct
  source 1..35: mol_type = other DNA
gagtaggattgtacccctcaggctttgtttactct

SEQ ID NO: 798 - DNA, 36 bp, synthetic construct
  source 1..36: mol_type = other DNA
gagtaggattgtacccctcaatgctttgtttactct

SEQ ID NO: 799 - DNA, 40 bp, synthetic construct
  source 1..40: mol_type = other DNA
gagtaggattgtacccctcaagtctatgctttgtttactc

SEQ ID NO: 800 - DNA, 35 bp, synthetic construct
  source 1..35: mol_type = other DNA
gagtaggattgtacccctcatgctttgtttactct

SEQ ID NO: 801 - DNA, 38 bp, synthetic construct
  source 1..38: mol_type = other DNA
gagtaggattgtacccctcactatgctttgtttactct

SEQ ID NO: 802 - DNA, 37 bp, synthetic construct
  source 1..37: mol_type = other DNA
gagtaggattgtacccctcatatgctttgtttactct

SEQ ID NO: 803 - DNA, 35 bp, synthetic construct
  source 1..35: mol_type = other DNA
gagtaggattgtaccctctatgctttgtttactct

SEQ ID NO: 804 - DNA, 36 bp, synthetic construct
  source 1..36: mol_type = other DNA
gagtaggattgtacccctcagtgctttgtttactct

SEQ ID NO: 805 - DNA, 28 bp, synthetic construct
  source 1..28: mol_type = other DNA
gagtaggattgtatgctttgtttactct

SEQ ID NO: 806 - DNA, 38 bp, synthetic construct
  source 1..38: mol_type = other DNA
gagtaggattgtacccctgtctatgctttgtttactct

SEQ ID NO: 807 - DNA, 33 bp, synthetic construct
  source 1..33: mol_type = other DNA
gagtaggattgtacccctcagtttgtttactct

SEQ ID NO: 808 - DNA, 29 bp, synthetic construct
  source 1..29: mol_type = other DNA
gagtaggatgtctatgctttgtttactct

SEQ ID NO: 809 - DNA, 27 bp, synthetic construct
  source 1..27: mol_type = other DNA
gagtaggatctatgctttgtttactct

SEQ ID NO: 810 - DNA, 40 bp, synthetic construct
  source 1..40: mol_type = other DNA
gagtaggattgtacccctcatgtctatgctttgtttactc

SEQ ID NO: 811 - DNA, 20 bp, synthetic construct
  source 1..20: mol_type = other DNA
gagtaggattgtacccctct

SEQ ID NO: 812 - DNA, 35 bp, synthetic construct
  source 1..35: mol_type = other DNA
gagtaggattgtaccgtctatgctttgtttactct

SEQ ID NO: 813 - DNA, 31 bp, synthetic construct
  source 1..31: mol_type = other DNA
gagtaggattgtactatgctttgtttactct

SEQ ID NO: 814 - DNA, 22 bp, synthetic construct
  source 1..22: mol_type = other DNA
gagtctatgctttgtttactct

SEQ ID NO: 815 - DNA, 20 bp, synthetic construct
  source 1..20: mol_type = other DNA
gagtaggattgtacccctca

SEQ ID NO: 816 - DNA, 26 bp, synthetic construct
  source 1..26: mol_type = other DNA
gagtaggattgtactttgtttactct

SEQ ID NO: 817 - DNA, 23 bp, synthetic construct
  source 1..23: mol_type = other DNA
gagtaggatgctttgtttactct

SEQ ID NO: 818 - DNA, 20 bp, synthetic construct
  source 1..20: mol_type = other DNA
gagtaggatttgtttactct

SEQ ID NO: 819 - DNA, 29 bp, synthetic construct
  source 1..29: mol_type = other DNA
gagtaggattgtacccctttgtttactct

SEQ ID NO: 820 - DNA, 20 bp, synthetic construct
  source 1..20: mol_type = other DNA
gtcatcttagtcattacctg

SEQ ID NO: 821 - DNA, 52 bp, synthetic construct
  source 1..52: mol_type = other DNA
gggaaccgtgtcatcttagtcattacctgaggtgttccaacattgagtatat

SEQ ID NO: 822 - DNA, 51 bp, synthetic construct
  source 1..51: mol_type = other DNA
gggaaccgtgtcatcttagtcattactgaggtgttccaacattgagtatat

SEQ ID NO: 823 - DNA, 48 bp, synthetic construct
  source 1..48: mol_type = other DNA
gggaaccgtgtcatcttagtcattgaggtgttccaacattgagtatat

SEQ ID NO: 824 - DNA, 53 bp, synthetic construct
  source 1..53: mol_type = other DNA
gggaaccgtgtcatcttagtcattaccctgaggtgttccaacattgagtatat

SEQ ID NO: 825 - DNA, 40 bp, synthetic construct
  source 1..40: mol_type = other DNA
gggaaccgtgtcatctgaggtgttccaacattgagtatat

SEQ ID NO: 826 - DNA, 40 bp, synthetic construct
  source 1..40: mol_type = other DNA
gggaaccgtgtcatcttaggtgttccaacattgagtatat

SEQ ID NO: 827 - DNA, 47 bp, synthetic construct
  source 1..47: mol_type = other DNA
gggaaccgtgtcatcttagtcatgaggtgttccaacattgagtatat

SEQ ID NO: 828 - DNA, 47 bp, synthetic construct
  source 1..47: mol_type = other DNA
gggaaccgtgtcatcttagtcattaggtgttccaacattgagtatat

SEQ ID NO: 829 - DNA, 46 bp, synthetic construct
  source 1..46: mol_type = other DNA
gggaaccgtgtcatcttagtctgaggtgttccaacattgagtatat

SEQ ID NO: 830 - DNA, 39 bp, synthetic construct
  source 1..39: mol_type = other DNA
gggaaccgtgtcatcttagtgttccaacattgagtatat

SEQ ID NO: 831 - DNA, 40 bp, synthetic construct
  source 1..40: mol_type = other DNA
gggaaccgtgtcatcttagtcattccaacattgagtatat

SEQ ID NO: 832 - DNA, 40 bp, synthetic construct
  source 1..40: mol_type = other DNA
agttacaacgaacacctcaggtaatgactaagatgactgc

SEQ ID NO: 833 - DNA, 39 bp, synthetic construct
  source 1..39: mol_type = other DNA
agttacaacgaacacctcagtaatgactaagatgactgc

SEQ ID NO: 834 - DNA, 38 bp, synthetic construct
  source 1..38: mol_type = other DNA
agttacaacgaacacctcgtaatgactaagatgactgc

SEQ ID NO: 835 - DNA, 40 bp, synthetic construct
  source 1..40: mol_type = other DNA
agttacaacgaacacctcagggtaatgactaagatgactg

SEQ ID NO: 836 - DNA, 35 bp, synthetic construct
  source 1..35: mol_type = other DNA
agttacaacgaacacctcaggactaagatgactgc

SEQ ID NO: 837 - DNA, 38 bp, synthetic construct
  source 1..38: mol_type = other DNA
agttacaacgaacacctcagaatgactaagatgactgc

SEQ ID NO: 838 - DNA, 37 bp, synthetic construct
  source 1..37: mol_type = other DNA
agttacaacgaacacctcagatgactaagatgactgc

SEQ ID NO: 839 - DNA, 33 bp, synthetic construct
  source 1..33: mol_type = other DNA
agttacaacgaacgtaatgactaagatgactgc

SEQ ID NO: 840 - DNA, 36 bp, synthetic construct
  source 1..36: mol_type = other DNA
agttacaacgaacacctcagtgactaagatgactgc

SEQ ID NO: 841 - DNA, 34 bp, synthetic construct
  source 1..34: mol_type = other DNA
agttacaacgaacacctcagactaagatgactgc

SEQ ID NO: 842 - DNA, 28 bp, synthetic construct
  source 1..28: mol_type = other DNA
agttacaacgaacacctcagatgactgc

SEQ ID NO: 843 - DNA, 39 bp, synthetic construct
  source 1..39: mol_type = other DNA
agttacaacgaacacctcaggaatgactaagatgactgc

SEQ ID NO: 844 - DNA, 33 bp, synthetic construct
  source 1..33: mol_type = other DNA
agttacaacgaacacctcagctaagatgactgc

SEQ ID NO: 845 - DNA, 40 bp, synthetic construct
  source 1..40: mol_type = other DNA
agttacaacgaacacctcagagtaatgactaagatgactg

SEQ ID NO: 846 - DNA, 37 bp, synthetic construct
  source 1..37: mol_type = other DNA
agttacaacgaacacctgtaatgactaagatgactgc

SEQ ID NO: 847 - DNA, 36 bp, synthetic construct
  source 1..36: mol_type = other DNA
agttacaacgaacacctcagggactaagatgactgc

SEQ ID NO: 848 - DNA, 28 bp, synthetic construct
  source 1..28: mol_type = other DNA
agttacaacgaacacctaagatgactgc

SEQ ID NO: 849 - DNA, 35 bp, synthetic construct
  source 1..35: mol_type = other DNA
agttacaacgaacacgtaatgactaagatgactgc

SEQ ID NO: 850 - DNA, 35 bp, synthetic construct
  source 1..35: mol_type = other DNA
agttacaacgaacacctcatgactaagatgactgc

SEQ ID NO: 851 - DNA, 30 bp, synthetic construct
  source 1..30: mol_type = other DNA
agttacaacggtaatgactaagatgactgc

SEQ ID NO: 852 - DNA, 38 bp, synthetic construct
  source 1..38: mol_type = other DNA
agttacaacgaacacctcaggatgactaagatgactgc

SEQ ID NO: 853 - DNA, 35 bp, synthetic construct
  source 1..35: mol_type = other DNA
agttacaacgaacacctaatgactaagatgactgc

SEQ ID NO: 854 - DNA, 37 bp, synthetic construct
  source 1..37: mol_type = other DNA
agttacaacgaacacctcaggtgactaagatgactgc

SEQ ID NO: 855 - DNA, 25 bp, synthetic construct
  source 1..25: mol_type = other DNA
agttacaacgaactaagatgactgc

SEQ ID NO: 856 - DNA, 32 bp, synthetic construct
  source 1..32: mol_type = other DNA
agttacaacgaagtaatgactaagatgactgc

SEQ ID NO: 857 - DNA, 40 bp, synthetic construct
  source 1..40: mol_type = other DNA
agttacaacgaacacctcagttaatgactaagatgactgc

SEQ ID NO: 858 - DNA, 36 bp, synthetic construct
  source 1..36: mol_type = other DNA
agttacaacgaacaccgtaatgactaagatgactgc

SEQ ID NO: 859 - DNA, 40 bp, synthetic construct
  source 1..40: mol_type = other DNA
agttacaacgaacacctcagtgtaatgactaagatgactg

SEQ ID NO: 860 - DNA, 37 bp, synthetic construct
  source 1..37: mol_type = other DNA
agttacaacgaacacctctaatgactaagatgactgc

SEQ ID NO: 861 - DNA, 34 bp, synthetic construct
  source 1..34: mol_type = other DNA
agttacaacgaacacctcaggctaagatgactgc

SEQ ID NO: 862 - DNA, 27 bp, synthetic construct
  source 1..27: mol_type = other DNA
agttacaacgaacactaagatgactgc

SEQ ID NO: 863 - DNA, 32 bp, synthetic construct
  source 1..32: mol_type = other DNA
agttacaacgaacacctcagtaagatgactgc

SEQ ID NO: 864 - DNA, 26 bp, synthetic construct
  source 1..26: mol_type = other DNA
agttacaacgaacacctcaggactgc

SEQ ID NO: 865 - DNA, 34 bp, synthetic construct
  source 1..34: mol_type = other DNA
agttacaacgaacagtaatgactaagatgactgc

SEQ ID NO: 866 - DNA, 29 bp, synthetic construct
  source 1..29: mol_type = other DNA
agttacaacgtaatgactaagatgactgc

SEQ ID NO: 867 - DNA, 30 bp, synthetic construct
  source 1..30: mol_type = other DNA
agttacaacgaacacctctaagatgactgc

SEQ ID NO: 868 - DNA, 40 bp, synthetic construct
  source 1..40: mol_type = other DNA
agttacaacgaacacctcaggtgtaatgactaagatgact

SEQ ID NO: 869 - DNA, 31 bp, synthetic construct
  source 1..31: mol_type = other DNA
agttacaacgagtaatgactaagatgactgc

SEQ ID NO: 870 - DNA, 40 bp, synthetic construct
  source 1..40: mol_type = other DNA
agttacaacgaacacctcagcgtaatgactaagatgactg

SEQ ID NO: 871 - DNA, 36 bp, synthetic construct
  source 1..36: mol_type = other DNA
agttacaacgaacacctcaatgactaagatgactgc

SEQ ID NO: 872 - DNA, 20 bp, synthetic construct
  source 1..20: mol_type = other DNA
cataccatattggagatgag

SEQ ID NO: 873 - DNA, 52 bp, synthetic construct
  source 1..52: mol_type = other DNA
aagggccgccataccatattggagatgagggtggcaaggcgctgacgcggac

SEQ ID NO: 874 - DNA, 53 bp, synthetic construct
  source 1..53: mol_type = other DNA
aagggccgccataccatattggagattgagggtggcaaggcgctgacgcggac

SEQ ID NO: 875 - DNA, 47 bp, synthetic construct
  source 1..47: mol_type = other DNA
aagggccgccataccatattggagggtggcaaggcgctgacgcggac

SEQ ID NO: 876 - DNA, 49 bp, synthetic construct
  source 1..49: mol_type = other DNA
aagggccgccataccatattggagagggtggcaaggcgctgacgcggac

SEQ ID NO: 877 - DNA, 44 bp, synthetic construct
  source 1..44: mol_type = other DNA
aagggccgccataccatattgggtggcaaggcgctgacgcggac

SEQ ID NO: 878 - DNA, 40 bp, synthetic construct
  source 1..40: mol_type = other DNA
aagggccgccataccatattggcaaggcgctgacgcggac

SEQ ID NO: 879 - DNA, 43 bp, synthetic construct
  source 1..43: mol_type = other DNA
aagggccgccataccatattggtggcaaggcgctgacgcggac

SEQ ID NO: 880 - DNA, 49 bp, synthetic construct
  source 1..49: mol_type = other DNA
aagggccgccataccatattggagatggtggcaaggcgctgacgcggac

SEQ ID NO: 881 - DNA, 51 bp, synthetic construct
  source 1..51: mol_type = other DNA
aagggccgccataccatattggagagagggtggcaaggcgctgacgcggac

SEQ ID NO: 882 - DNA, 51 bp, synthetic construct
  source 1..51: mol_type = other DNA
aagggccgccataccatattggagatagggtggcaaggcgctgacgcggac

SEQ ID NO: 883 - DNA, 53 bp, synthetic construct
  source 1..53: mol_type = other DNA
aagggccgccataccatattggagatagagggtggcaaggcgctgacgcggac

SEQ ID NO: 884 - DNA, 40 bp, synthetic construct
  source 1..40: mol_type = other DNA
gtcagcgccttgccaccctcatctccaatatggtatggcg

SEQ ID NO: 885 - DNA, 40 bp, synthetic construct
  source 1..40: mol_type = other DNA
gtcagcgccttgccaccctcaatctccaatatggtatggc

SEQ ID NO: 886 - DNA, 40 bp, synthetic construct
  source 1..40: mol_type = other DNA
gtcagcgccttgccaccctcaaatctccaatatggtatgg

SEQ ID NO: 887 - DNA, 28 bp, synthetic construct
  source 1..28: mol_type = other DNA
gtcagcgccttgccaccctcatatggcg

SEQ ID NO: 888 - DNA, 37 bp, synthetic construct
  source 1..37: mol_type = other DNA
gtcagcgccttgccaccctcaccaatatggtatggcg

SEQ ID NO: 889 - DNA, 33 bp, synthetic construct
  source 1..33: mol_type = other DNA
gtcagcgccttgccaccctcatatggtatggcg

SEQ ID NO: 890 - DNA, 29 bp, synthetic construct
  source 1..29: mol_type = other DNA
gtcagcgccttgccaccctcagtatggcg

SEQ ID NO: 891 - DNA, 30 bp, synthetic construct
  source 1..30: mol_type = other DNA
gtcagcgccttgccaccctcaggtatggcg

SEQ ID NO: 892 - DNA, 32 bp, synthetic construct
  source 1..32: mol_type = other DNA
gtcagcgccttgccaccctcaatggtatggcg

SEQ ID NO: 893 - DNA, 31 bp, synthetic construct
  source 1..31: mol_type = other DNA
gtcagcgccttgccaccctcatggtatggcg

SEQ ID NO: 894 - DNA, 40 bp, synthetic construct
  source 1..40: mol_type = other DNA
gtcagcgccttgccaccctcaaaatctccaatatggtatg

SEQ ID NO: 895 - DNA, 26 bp, synthetic construct
  source 1..26: mol_type = other DNA
gtcagcgccttgccaccctcatggcg

SEQ ID NO: 896 - DNA, 39 bp, synthetic construct
  source 1..39: mol_type = other DNA
gtcagcgccttgccaccctcactccaatatggtatggcg

SEQ ID NO: 897 - DNA, 39 bp, synthetic construct
  source 1..39: mol_type = other DNA
gtcagcgccttgccaccctctctccaatatggtatggcg

SEQ ID NO: 898 - DNA, 40 bp, synthetic construct
  source 1..40: mol_type = other DNA
gtcagcgccttgccaccctcatctacaatatggtatggcg

SEQ ID NO: 899 - DNA, 33 bp, synthetic construct
  source 1..33: mol_type = other DNA
aatgccgacatctctatggtgaatggagacact

SEQ ID NO: 900 - DNA, 34 bp, synthetic construct
  source 1..34: mol_type = other DNA
aatgccgacatctctatggttgaatggagacact

SEQ ID NO: 901 - DNA, 50 bp, synthetic construct
  source 1..50: mol_type = other DNA
gacaccatggtgcacctgactcctgaggagaagtctgccgttactgccct

SEQ ID NO: 902 - DNA, 50 bp, synthetic construct
  source 1..50: mol_type = other DNA
agggcagtaacggcagacttctcctcaggagtcaggtgcaccatggtgtc

SEQ ID NO: 903 - DNA, 50 bp, synthetic construct
  source 1..50: mol_type = other DNA
gccccctggtacctgaaccgtatatcctatggccctgactggaaggattt

SEQ ID NO: 904 - DNA, 50 bp, synthetic construct
  source 1..50: mol_type = other DNA
aaatccttccagtcagggccataggatatacggttcaggtaccagggggc

SEQ ID NO: 905 - DNA, 33 bp, synthetic construct
  source 1..33: mol_type = other DNA
caccatggtgcacctgactcctgaggagaagtc

SEQ ID NO: 906 - DNA, 32 bp, synthetic construct
  source 1..32: mol_type = other DNA
caccatggtgcacctgactctgaggagaagtc

SEQ ID NO: 907 - DNA, 33 bp, synthetic construct
  source 1..33: mol_type = other DNA
cagtaacggcagacttctcctcaggagtcaggt

SEQ ID NO: 908 - DNA, 30 bp, synthetic construct
  source 1..30: mol_type = other DNA
cagtaacggcagacttctcaggagtcaggt

SEQ ID NO: 909 - DNA, 32 bp, synthetic construct
  source 1..32: mol_type = other DNA
cagtaacggcagacttctctcaggagtcaggt

SEQ ID NO: 910 - DNA, 34 bp, synthetic construct
  source 1..34: mol_type = other DNA
cagtaacggcagacttctccctcaggagtcaggt

SEQ ID NO: 911 - DNA, 33 bp, synthetic construct
  source 1..33: mol_type = other DNA
accgtatatcctatggccctgactggaaggatt

SEQ ID NO: 912 - DNA, 34 bp, synthetic construct
  source 1..34: mol_type = other DNA
accgtatatcctatggcccttgactggaaggatt

SEQ ID NO: 913 - DNA, 33 bp, synthetic construct
  source 1..33: mol_type = other DNA
tccagtcagggccataggatatacggttcaggt

SEQ ID NO: 914 - DNA, 34 bp, synthetic construct
  source 1..34: mol_type = other DNA
tccagtcagggccataggattatacggttcaggt

SEQ ID NO: 915 - DNA, 40 bp, synthetic construct
  source 1..40: mol_type = other DNA
gcagtcatcttagtcattacctgaggtgttcgttgtaact

SEQ ID NO: 916 - DNA, 39 bp, synthetic construct
  source 1..39: mol_type = other DNA
gcagtcatcttagtcattactgaggtgttcgttgtaact

SEQ ID NO: 917 - DNA, 23 bp, synthetic construct
  source 1..23: mol_type = other DNA
gccgacatctctatggtgaatgg

SEQ ID NO: 918 - DNA, 23 bp, synthetic construct
  source 1..23: mol_type = other DNA
gctgtcatctctatggtgcatgg

SEQ ID NO: 919 - DNA, 23 bp, synthetic construct
  source 1..23: mol_type = other DNA
gcagacatctgaatggtgaatgg

SEQ ID NO: 920 - DNA, 23 bp, synthetic construct
  source 1..23: mol_type = other DNA
gcagacaactttatggtgaatgg

SEQ ID NO: 921 - DNA, 23 bp, synthetic construct
  source 1..23: mol_type = other DNA
ccgacatctctatggttgaatgg

SEQ ID NO: 922 - DNA, 23 bp, synthetic construct
  source 1..23: mol_type = other DNA
ccgacatctggatggttgaatgg

SEQ ID NO: 923 - DNA, 23 bp, synthetic construct
  source 1..23: mol_type = other DNA
ccgacatctctatggatgaatgg

SEQ ID NO: 924 - DNA, 23 bp, synthetic construct
  source 1..23: mol_type = other DNA
cccacatctccatgattgaatgg

SEQ ID NO: 925 - DNA, 22 bp, synthetic construct
  source 1..22: mol_type = other DNA
cagtaacggcagacttctcagg

SEQ ID NO: 926 - DNA, 22 bp, synthetic construct
  source 1..22: mol_type = other DNA
cagtaactgcagacttctcagg

SEQ ID NO: 927 - DNA, 23 bp, synthetic construct
  source 1..23: mol_type = other DNA
tttctatctgcagctctgggagg

SEQ ID NO: 928 - DNA, 23 bp, synthetic construct
  source 1..23: mol_type = other DNA
tttccatctgcagctctgggagg

SEQ ID NO: 929 - DNA, 23 bp, synthetic construct
  source 1..23: mol_type = other DNA
tttctatctgtggctctgggagg

SEQ ID NO: 930 - DNA, 23 bp, synthetic construct
  source 1..23: mol_type = other DNA
tttctgtctgtagctctgggagg

SEQ ID NO: 931 - DNA, 23 bp, synthetic construct
  source 1..23: mol_type = other DNA
ttctatctgcagctcttgggagg

SEQ ID NO: 932 - DNA, 23 bp, synthetic construct
  source 1..23: mol_type = other DNA
ttgtatttgcagctattgggagg

SEQ ID NO: 933 - DNA, 23 bp, synthetic construct
  source 1..23: mol_type = other DNA
attcatctgcagctcttgggagg

SEQ ID NO: 934 - DNA, 23 bp, synthetic construct
  source 1..23: mol_type = other DNA
ttctttcaggagctcttgggagg

SEQ ID NO: 935 - DNA, 23 bp, synthetic construct
  source 1..23: mol_type = other DNA
ggttttggacactggaaccgtgg

SEQ ID NO: 936 - DNA, 22 bp, synthetic construct
  source 1..22: mol_type = other DNA
ggttttggacactggaccgtgg

SEQ ID NO: 937 - DNA, 23 bp, synthetic construct
  source 1..23: mol_type = other DNA
ggcgatacttggatgccctgcgg

SEQ ID NO: 938 - DNA, 22 bp, synthetic construct
  source 1..22: mol_type = other DNA
ggcgatacttggatgcctgcgg

SEQ ID NO: 939 - DNA, 100 bp, unidentified
  source 1..100: mol_type = unassigned DNA
acaggttggccattgagcagtcgcatcccggatggcgatacttggatgccctgcggcgatcgaaaggcaagggcattcagcagggtcgtctttccggcac

SEQ ID NO: 940 - DNA, 78 bp, synthetic construct
  source 1..78: mol_type = other DNA
acaggttggccattgagcagtcgcatcccggcggcgatcgaaaggcaagggcattcagcagggtcgtctttccggcac

SEQ ID NO: 941 - DNA, 97 bp, synthetic construct
  source 1..97: mol_type = other DNA
acaggttggccattgagcagtcgcatcccggatggcgatacttggatgcgcggcgatcgaaaggcaagggcattcagcagggtcgtctttccggcac

SEQ ID NO: 942 - DNA, 98 bp, synthetic construct
  source 1..98: mol_type = other DNA
acaggttggccattgagcagtcgcatcccggatggcgatacttggatactgcggcgatcgaaaggcaagggcattcagcagggtcgtctttccggcac

SEQ ID NO: 943 - DNA, 99 bp, synthetic construct
  source 1..99: mol_type = other DNA
acaggttggccattgagcagtcgcatcccggatggcgatacttggatgcctgcggcgatcgaaaggcaagggcattcagcagggtcgtctttccggcac

SEQ ID NO: 944 - DNA, 99 bp, synthetic construct
  source 1..99: mol_type = other DNA
acaggttggccattgagcagtcgcatcccggatggcgatacttggatggccgcggcgatcgaaaggcaagggcattcagcagggtcgtctttccggcac

SEQ ID NO: 945 - DNA, 106 bp, synthetic construct
  source 1..106: mol_type = other DNA
acaggttggccattgagcagtcgcatcccggatggcgatacttggcgccttggatactgcggcgatcgaaaggcaagggcattcagcagggtcgtctttccggcac

SEQ ID NO: 946 - DNA, 116 bp, synthetic construct
  source 1..116: mol_type = other DNA
acaggttggccattgagcagtcgcatcccggatggcgatacttggcgatacttggcgatttcgatactgcggcgatcgaaaggcaagggcattcagcagggtcgtctttccggcac

SEQ ID NO: 947 - DNA, 53 bp, synthetic construct
  source 1..53: mol_type = other DNA
acaggttggccattgagcagtcgcatcccggatggcgatacttggaactgcac

SEQ ID NO: 948 - DNA, 87 bp, synthetic construct
  source 1..87: mol_type = other DNA
acaggttggccattgagcagtcgcatcccggatggcgatacttggatcgaaaggcaagggcattcagcagggtcgtctttccggcac

SEQ ID NO: 949 - DNA, 89 bp, synthetic construct
  source 1..89: mol_type = other DNA
acaggttggccattgagcagtcgcatcccggatggcgatacttggcgatcgaaaggcaagggcattcagcagggtcgtctttccggcac

SEQ ID NO: 950 - DNA, 95 bp, synthetic construct
  source 1..95: mol_type = other DNA
acaggttggccattgagcagtcgcatcccggatggcgatacttggatgcggcgatcgaaaggcaagggcattcagcagggtcgtctttccggcac

SEQ ID NO: 951 - DNA, 97 bp, synthetic construct
  source 1..97: mol_type = other DNA
acaggttggccattgagcagtcgcatcccggatggcgatacttggatgtgcggcgatcgaaaggcaagggcattcagcagggtcgtctttccggcac

SEQ ID NO: 952 - DNA, 98 bp, synthetic construct
  source 1..98: mol_type = other DNA
acaggttggccattgagcagtcgcatcccggatggcgatacttggatgctgcggcgatcgaaaggcaagggcattcagcagggtcgtctttccggcac

SEQ ID NO: 953 - DNA, 98 bp, synthetic construct
  source 1..98: mol_type = other DNA
acaggttggccattgagcagtcgcatcccggatggcgatacttggatcctgcggcgatcgaaaggcaagggcattcagcagggtcgtctttccggcac

SEQ ID NO: 954 - DNA, 100 bp, synthetic construct
  source 1..100: mol_type = other DNA
acaggttggccattgagcagtcgcatcccggatggcgatacttggagatactgcggcgatcgaaaggcaagggcattcagcagggtcgtctttccggcac

SEQ ID NO: 955 - DNA, 101 bp, synthetic construct
  source 1..101: mol_type = other DNA
acaggttggccattgagcagtcgcatcccggatggcgatacttggatgcccctgcggcgatcgaaaggcaagggcattcagcagggtcgtctttccggcac

SEQ ID NO: 956 - DNA, 103 bp, synthetic construct
  source 1..103: mol_type = other DNA
acaggttggccattgagcagtcgcatcccggatggcgatacttggatgcgatactgcggcgatcgaaaggcaagggcattcagcagggtcgtctttccggcac

SEQ ID NO: 957 - DNA, 103 bp, synthetic construct
  source 1..103: mol_type = other DNA
acaggttggccattgagcagtcgcatcccggatggcgatactgcggatggatactgcggcgatcgaaaggcaagggcattcagcagggtcgtctttccggcac

SEQ ID NO: 958 - DNA, 107 bp, synthetic construct
  source 1..107: mol_type = other DNA
acaggttggccattgagcagtcgcatcccggatggcgatacttggatcttgcggtacttgcggcgatcgaaaggcaagggcattcagcagggtcgtctttccggcac

SEQ ID NO: 959 - DNA, 92 bp, synthetic construct
  source 1..92: mol_type = other DNA
acaggttggccattgagcagtcgcatcccggatggcgatacttggcggcgatcgaaaggcaagggcattcagcagggtcgtctttccggcac

SEQ ID NO: 960 - DNA, 104 bp, synthetic construct
  source 1..104: mol_type = other DNA
acaggttggccattgagcagtcgcatcccggatggcgatacttggatgcatcgcatgcggcgatcgaaaggcaagggcattcagcagggtcgtctttccggcac

SEQ ID NO: 961 - DNA, 100 bp, unidentified
  source 1..100: mol_type = unassigned DNA
ccgcattaaagtggatgagtgtggtcggctgtgggttttggacactggaaccgtgggcatcggcaataccaccactaatccgtgcccctatgcggtaaat

SEQ ID NO: 962 - DNA, 92 bp, synthetic construct
  source 1..92: mol_type = other DNA
ccgcattaaagtggatgagtgtggtcggctgtgggttttggacactgggcatcggcaataccaccactaatccgtgcccctatgcggtaaat

SEQ ID NO: 963 - DNA, 94 bp, synthetic construct
  source 1..94: mol_type = other DNA
ccgcattaaagtggatgagtgtggtcggctgtgggttttggacaccgtgggcatcggcaataccaccactaatccgtgcccctatgcggtaaat

SEQ ID NO: 964 - DNA, 96 bp, synthetic construct
  source 1..96: mol_type = other DNA
ccgcattaaagtggatgagtgtggtcggctgtgggttttggacactgcgtgggcatcggcaataccaccactaatccgtgcccctatgcggtaaat

SEQ ID NO: 965 - DNA, 96 bp, synthetic construct
  source 1..96: mol_type = other DNA
ccgcattaaagtggatgagtgtggtcggctgtgggttttggacacaccgtgggcatcggcaataccaccactaatccgtgcccctatgcggtaaat

SEQ ID NO: 966 - DNA, 98 bp, synthetic construct
  source 1..98: mol_type = other DNA
ccgcattaaagtggatgagtgtggtcggctgtgggttttggacactggccgtgggcatcggcaataccaccactaatccgtgcccctatgcggtaaat

SEQ ID NO: 967 - DNA, 98 bp, synthetic construct
  source 1..98: mol_type = other DNA
ccgcattaaagtggatgagtgtggtcggctgtgggttttggacactggaagtgggcatcggcaataccaccactaatccgtgcccctatgcggtaaat

SEQ ID NO: 968 - DNA, 100 bp, synthetic construct
  source 1..100: mol_type = other DNA
ccgcattaaagtggatgagtgtggtcggctgtgggttttggacactggcatcgtgggcatcggcaataccaccactaatccgtgcccctatgcggtaaat

SEQ ID NO: 969 - DNA, 101 bp, synthetic construct
  source 1..101: mol_type = other DNA
ccgcattaaagtggatgagtgtggtcggctgtgggttttggacactggaaaccgtgggcatcggcaataccaccactaatccgtgcccctatgcggtaaat

SEQ ID NO: 970 - DNA, 101 bp, synthetic construct
  source 1..101: mol_type = other DNA
ccgcattaaagtggatgagtgtggtcggctgtgggttttggacactggacaccgtgggcatcggcaataccaccactaatccgtgcccctatgcggtaaat

SEQ ID NO: 971 - DNA, 101 bp, synthetic construct
  source 1..101: mol_type = other DNA
ccgcattaaagtggatgagtgtggtcggctgtgggttttggacactggatgccgtgggcatcggcaataccaccactaatccgtgcccctatgcggtaaat

SEQ ID NO: 972 - DNA, 103 bp, synthetic construct
  source 1..103: mol_type = other DNA
ccgcattaaagtggatgagtgtggtcggctgtgggttttggacactgggtttgacattggcatcggcaataccaccactaatccgtgcccctatgcggtaaat

SEQ ID NO: 973 - DNA, 105 bp, synthetic construct
  source 1..105: mol_type = other DNA
ccgcattaaagtggatgagtgtggtcggctgtgggttttggacactggacactggacgtgggcatcggcaataccaccactaatccgtgcccctatgcggtaaat

SEQ ID NO: 974 - DNA, 67 bp, synthetic construct
  source 1..67: mol_type = other DNA
ccgcattaaagtggatgccgtgggcatcggcaataccaccactaatccgtgcccctatgcggtaaat

SEQ ID NO: 975 - DNA, 82 bp, synthetic construct
  source 1..82: mol_type = other DNA
ccgcattaaagtggatgagtgtggtcggatgcccgtgggcatcggcaataccaccactaatccgtgcccctatgcggtaaat

SEQ ID NO: 976 - DNA, 85 bp, synthetic construct
  source 1..85: mol_type = other DNA
ccgcattaaagtggatgagtgtggtcggctgtgggttttggacatcggcaataccaccactaatccgtgcccctatgcggtaaat

SEQ ID NO: 977 - DNA, 85 bp, synthetic construct
  source 1..85: mol_type = other DNA
ccgcattaaagtggatgagtgtggtcggctgtgggttttggacactggcaataccaccactaatccgtgcccctatgcggtaaat

SEQ ID NO: 978 - DNA, 92 bp, synthetic construct
  source 1..92: mol_type = other DNA
ccgcattaaagtggatgagtgtggtcggctgtgggttttggaccgtgggcatcggcaataccaccactaatccgtgcccctatgcggtaaat

SEQ ID NO: 979 - DNA, 94 bp, synthetic construct
  source 1..94: mol_type = other DNA
ccgcattaaagtggatgagtgtggtcggctgtgggttttggacatcgtgggcatcggcaataccaccactaatccgtgcccctatgcggtaaat

SEQ ID NO: 980 - DNA, 95 bp, synthetic construct
  source 1..95: mol_type = other DNA
ccgcattaaagtggatgagtgtggtcggctgtgggttttgggcatccgtgggcatcggcaataccaccactaatccgtgcccctatgcggtaaat

SEQ ID NO: 981 - DNA, 95 bp, synthetic construct
  source 1..95: mol_type = other DNA
ccgcattaaagtggatgagtgtggtcggctgtgggttttggacacccgtgggcatcggcaataccaccactaatccgtgcccctatgcggtaaat

SEQ ID NO: 982 - DNA, 97 bp, synthetic construct
  source 1..97: mol_type = other DNA
ccgcattaaagtggatgagtgtggtcggctgtgggttttggacactgccgtgggcatcggcaataccaccactaatccgtgcccctatgcggtaaat

SEQ ID NO: 983 - DNA, 97 bp, synthetic construct
  source 1..97: mol_type = other DNA
ccgcattaaagtggatgagtgtggtcggctgtgggttttggacactggaatgtccatcggcaataccaccactaatccgtgcccctatgcggtaaat

SEQ ID NO: 984 - DNA, 99 bp, synthetic construct
  source 1..99: mol_type = other DNA
ccgcattaaagtggatgagtgtggtcggctgtgggttttggacactggaccgtgggcatcggcaataccaccactaatccgtgcccctatgcggtaaat

SEQ ID NO: 985 - DNA, 99 bp, synthetic construct
  source 1..99: mol_type = other DNA
ccgcattaaagtggatgagtgtggtcggctgtgggttttggacactggtccgtgggcatcggcaataccaccactaatccgtgcccctatgcggtaaat

SEQ ID NO: 986 - DNA, 100 bp, synthetic construct
  source 1..100: mol_type = other DNA
ccgcattaaagtggatgagtgtggtcggctgtgggttttggacactggaatcgtgggcatcggcaataccaccactaatccgtgcccctatgcggtaaat

SEQ ID NO: 987 - DNA, 101 bp, synthetic construct
  source 1..101: mol_type = other DNA
ccgcattaaagtggatgagtgtggtcggctgtgggttttggacactggacatcgtgggcatcggcaataccaccactaatccgtgcccctatgcggtaaat

SEQ ID NO: 988 - DNA, 104 bp, synthetic construct
  source 1..104: mol_type = other DNA
ccgcattaaagtggatgagtgtggtcggctgtgggttttggacactggacattgccgtgggcatcggcaataccaccactaatccgtgcccctatgcggtaaat

SEQ ID NO: 989 - DNA, 91 bp, synthetic construct
  source 1..91: mol_type = other DNA
ccgcattaaagtggatgagtgtggtcggctgtgggttttggacactggcatcggcaataccaccactaatccgtgcccctatgcggtaaat

SEQ ID NO: 990 - DNA, 96 bp, synthetic construct
  source 1..96: mol_type = other DNA
ccgcattaaagtggatgagtgtggtcggctgtgggttttggacactccgtgggcatcggcaataccaccactaatccgtgcccctatgcggtaaat

SEQ ID NO: 991 - DNA, 97 bp, synthetic construct
  source 1..97: mol_type = other DNA
ccgcattaaagtggatgagtgtggtcggctgtgggttttggacactggcgtgggcatcggcaataccaccactaatccgtgcccctatgcggtaaat

SEQ ID NO: 992 - DNA, 101 bp, synthetic construct
  source 1..101: mol_type = other DNA
ccgcattaaagtggatgagtgtggtcggctgtgggttttggacactggaacacgtgggcatcggcaataccaccactaatccgtgcccctatgcggtaaat

SEQ ID NO: 993 - DNA, 52 bp, synthetic construct
  source 1..52: mol_type = other DNA
cagtcgcatcccggatggcgatacttggatgccctgcggcgatcgaaaggca

SEQ ID NO: 994 - DNA, 39 bp, synthetic construct
  source 1..39: mol_type = other DNA
cagtcgcatcccggatggcgatacttggatcgaaaggca

SEQ ID NO: 995 - DNA, 48 bp, synthetic construct
  source 1..48: mol_type = other DNA
gaaatccttccagtcagggccataggatatacggttcaggtaccaggg

SEQ ID NO: 996 - DNA, 41 bp, synthetic construct
  source 1..41: mol_type = other DNA
gaaatccttccagtcagggccatacggttcaggtaccaggg

SEQ ID NO: 997 - DNA, 111 bp, synthetic construct
  source 1..111: mol_type = other DNA
ccgcattaaagtggatgagtgtggtcggctgtgggttttggacactggacacttttggacaccgtgggcatcggcaataccaccactaatccgtgcccctatgcggtaaat